(19) United States
(12) Patent Application Publication
(10) Pub. No.: US 2023/0066946 A1
(43) Pub. Date: Mar. 2, 2023

(54) 1,5-DIPHENYLPYRAZOLYL-3-OXYALKYL ACIDS AND 1-PHENYL-5-THIENYLPYRAZOLYL-3-OXYALKYL ACIDS AND THE USE THEREOF FOR CONTROL OF UNWANTED PLANT GROWTH

(71) Applicant: Bayer Aktiengesellschaft, Leverkusen (DE)

(72) Inventors: Harald JAKOBI, Frankfurt (DE); Michael Gerhard HOFFMANN, Konstanz (DE); Anna Maria REINGRUBER, Heppenheim (DE); Estella BUSCATO ARSEQUELL, SANT CUGAT DEL VALLES (ES); Thomas MULLER, Frankfurt (DE); Gilbert Ebai BESONG, Bad Duerkheim (DE); Erin Nicole SMITH, Duncraig (AU); Elisabeth ASMUS, Hoesbach (DE); Hansjorg DIETRICH, Liederbach am Taunus (DE); Elmar GATZWEILER, Bad Nauheim (DE); Anu Bheemaiah MACHETTIRA, Frankfurt am Main (DE); Christopher Hugh ROSINGER, Hofheim (DE); Dirk SCHMUTZLER, Hattersheim (DE)

(21) Appl. No.: 17/787,255

(22) PCT Filed: Dec. 16, 2020
(86) PCT No.: PCT/EP2020/086397

§ 371 Date: Jun. 18, 2022

(30) Foreign Application Priority Data
Dec. 19, 2019 (EP) 19218173.3

(51) Int. Cl. C07D 231/22; C07D 409/04; A01N 43/56

(52) U.S. Cl. CPC C07D 231/22; C07D 409/04; A01N 43/56

(57) ABSTRACT

The present invention relates to novel herbicidally active, substituted 1,5-diphenylpyrazolyl-3-oxyalkyl acids and 1-phenyl-5-thienylpyrazolyl-3-oxyalkyl acids and derivatives thereof according to the general formula (I) or agrochemically acceptable salts thereof, to processes for preparation thereof and to the use thereof for control of broadleaved weeds and weed grasses in crops of useful plants and for general control of broadleaved weeds and weed grasses in areas of the environment where plant growth is troublesome. The derivatives of the 1,5-diphenylpyrazolyl-3-oxyalkyl acids and 1-phenyl-5-thienylpyrazolyl-3-oxyalkyl acids especially include the esters, salts and amides thereof.

The present invention relates to novel herbicidally active, substituted 1,5-diphenylpyrazolyl-3-oxyalkyl acids and 1-phenyl-5-thienylpyrazolyl-3-oxyalkyl acids and derivatives thereof according to the general formula (I) or agrochemically acceptable salts thereof, to processes for preparation thereof and to the use thereof for control of broadleaved weeds and weed grasses in crops of useful plants and for general control of broadleaved weeds and weed grasses in areas of the environment where plant growth is troublesome.

The derivatives of the 1,5-diphenylpyrazolyl-3-oxyalkyl acids and 1-phenyl-5-thienylpyrazolyl-3-oxyalkyl acids especially include the esters, salts and amides thereof.

The prior art discloses biological effects of substituted 1,5-diphenylpyrazolyl-3-oxyacetic acids and substituted 1-phenyl-5-thienylpyrazolyl-3-oxyalkyl acids and processes for preparing these compounds. DE 2828529 A1 describes the preparation and the lipid-lowering effect of 1,5-diphenylpyrazolyl-3-oxyacetic acids. CN 101284815 discloses 1,5-diphenylpyrazolyl-3-oxyacetic acid derivatives as bactericidally active agrochemicals. Journal of Heterocyclic Chemistry (2012), 49(6), 1370-1375 describes further syntheses and the fungicidal action of 1,5-diphenylpyrazolyl-3-oxyacetic acids. The synthesis of substituted 1-phenyl-5-thienylpyrazolyl-3-oxyalkyl acids and the pharmaceutical action thereof as FXR and LXR modulators are described in WO 2008/073825 A1.

Substituted 1,5-diphenylpyrazolyl-3-oxyalkyl acids and substituted 1-phenyl-5-thienylpyrazolyl-3-oxyalkyl acids are unknown as herbicides to date.

It is a feature of all 1,5-diphenylpyrazolyl-3-oxyalkyl acid derivatives and all 1-phenyl-5-thienylpyrazolyl-3-oxyalkyl acid derivatives from the abovementioned sources that they are unsubstituted in the 4 position of the pyrazole.

By contrast, a common feature of the 1,5-diphenylpyrazolyl-3-oxyalkyl acids and 1-phenyl-5-thienylpyrazolyl-3-oxyalkyl acids according to the invention is a further substituent in the 4 position of the pyrazole ring. The present invention thus provides 1,5-diphenylpyrazolyl-3-oxyalkyl acids and 1-phenyl-5-thienylpyrazolyl-3-oxyalkyl acids substituted exclusively in the 4 position of the pyrazole (R³ hydrogen), and derivatives thereof.

WO 2008/083233 A2 describes such 1,5-diphenylpyrazolyl-3-oxyalkyl acids substituted in 4 position of the pyrazole and derivatives thereof as substances suitable for breaking up cell aggregates. Ethyl [(4-chloro-1,5-diphenyl-1H-pyrazol-3-yl)oxy]acetate is specifically disclosed.

In addition, the synthesis of some 4-chloro-1,5-diphenylpyrazolyl-3-oxyacetic acids and ethyl esters thereof is described in European Journal of Organic Chemistry (2011), 2011 (27), 5323-5330, specifically:
[(4-chloro-1,5-diphenyl-1H-pyrazol-3-yl)oxy]acetic acid
{[4-chloro-5-(4-methylphenyl)-1-phenyl-1H-pyrazol-3-yl]oxy}acetic acid
{[4-chloro-5-(3-chlorophenyl)-1-phenyl-1H-pyrazol-3-yl]oxy}acetic acid
ethyl [(4-chloro-1,5-diphenyl-1H-pyrazol-3-yl)oxy]acetate
ethyl {[4-chloro-5-(4-methylphenyl)-1-phenyl-1H-pyrazol-3-yl]oxy}acetate
ethyl {[4-chloro-5-(3-chlorophenyl)-1-phenyl-1H-pyrazol-3-yl]oxy}acetate.

WO 2008/141154 discloses: 2-(4-chloro-1,5-diphenylpyrazol-3-yl)oxypropanoic acid and 2-(4-bromo-1,5-diphenylpyrazol-3-yl)oxypropanoic acid.

There is no description of the herbicidal action of these compounds.

The 1-phenyl-5-thienylpyrazolyl-3-oxyacetic acids or 1-phenyl-5-thienylpyrazolyl-3-oxyalkyl acids and derivatives thereof that are encompassed by this application are unknown to date.

It is an object of the present invention to provide novel pyrazole derivatives, namely of 1,5-diphenylpyrazolyl-3-oxyalkyl acids and 1-phenyl-5-thienylpyrazolyl-3-oxyalkyl acids and derivatives thereof, which can be used as herbicides or plant growth regulators, having satisfactory herbicidal action and a broad spectrum of activity against harmful plants and/or having high selectivity in crops of useful plants.

The object is achieved by substituted pyrazolyl-3-oxoalkyl acids featuring a variable substituent in the 4 position of the pyrazole ring, i.e. by 4-substituted 1,5-diphenylpyrazolyl-3-oxyalkyl acid derivatives and 1-phenyl-5-thienylpyrazolyl-3-oxyalkyl acid derivatives, having very good herbicidal action and also very good selectivity.

Surprisingly, these compounds are highly effective against a broad range of economically important weed grasses and broadleaved weeds. At the same time, the compounds exhibit good crop plant compatibility. Therefore, given good efficacy against harmful plants, they can be used selectively in crop plants.

The present invention therefore provides substituted 1,5-diphenylpyrazolyl-3-oxyalkyl acids and 1-phenyl-5-thienylpyrazolyl-3-oxyalkyl acids of the general formula (I)

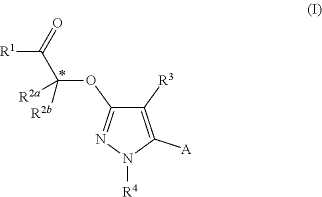

and the agrochemically acceptable salts thereof, where

A is selected from the group consisting of A1-A3,

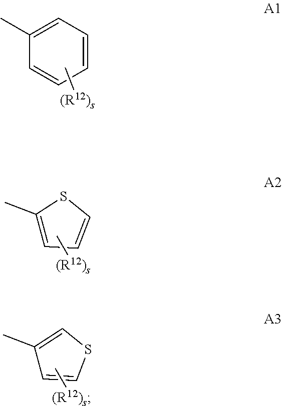

R¹ is selected from the group consisting of
OR^{1a} and
NR⁹R¹⁰; where
   R^{1a} is selected from the group consisting of
      hydrogen;
      methyl, ethyl which is unsubstituted or substituted by one or more substituents selected from the group consisting of halogen, (C₃-C₆)-cycloalkyl, (C₁-C₄)-trialkylsilyl, (C₁-C₆)-alkoxy, cyano and nitro;
      (C₂-C₆)-alkenyl, (C₂-C₆)-haloalkenyl;
      (C₂-C₆)-alkynyl,
      (C₃-C₆)-cycloalkyl which is unsubstituted or substituted by one or more substituents selected from the group consisting of halogen, (C₁-C₆)-alkyl, (C₃-C₆)-cycloalkyl;
      (C₁-C₄)-alkyl-SO—(C₁-C₄)-alkyl, (C₁-C₄)-alkyl-SO₂—(C₁-C₄)-alkyl;
      heterocyclyl, heteroaryl and aryl which is unsubstituted or substituted by one or more substituents selected from the group consisting of halogen, (C₁-C₆)-alkyl, (C₁-C₆)-haloalkyl;
      heterocyclyl-(C₁-C₄)-alkyl, heteroaryl-(C₁-C₄)-alkyl and aryl-(C₁-C₄)-alkyl, where the heterocyclyl, heteroaryl and aryl is unsubstituted or substituted by one or more substituents selected from the group consisting of halogen, (C₁-C₆)-alkyl, (C₁-C₆)-haloalkyl;
   R⁹ is selected from the group consisting of hydrogen, (C₁-C₁₂)-alkyl;
   R¹⁰ is selected from the group consisting of
      hydrogen;
      aryl, heteroaryl, heterocyclyl, which is unsubstituted or substituted by one or more substituents selected from the group consisting of halogen, (C₁-C₆)-alkyl, (C₁-C₆)-haloalkyl;
      (C₃-C₇)-cycloalkyl-(C₁-C₄)-alkyl, heterocyclyl-(C₁-C₄)-alkyl, heteroaryl-(C₁-C₄)-alkyl, aryl-(C₁-C₄)-alkyl, aryl-(C₁-C₄)-alkoxy;
      where the cycloalkyl, heterocyclyl, heteroaryl and aryl is unsubstituted or substituted by one or more substituents selected from the group consisting of halogen, (C₁-C₆)-alkyl, (C₁-C₆)-haloalkyl;
      (C₁-C₁₂)-alkyl; (C₃-C₈)-cycloalkyl, (C₂-C₁₂)-alkenyl, (C₅-C₈)-cycloalkenyl, (C₂-C₁₂)-alkynyl;
      where the abovementioned alkyl, cycloalkenyl, alkenyl, cycloalkenyl and alkynyl radicals are unsubstituted or each independently substituted by m radicals selected from the group consisting of cyano, nitro, OR⁵, S(O)ₙR⁵, SO₂NR⁶R⁷, C(O)OR⁸, CONR⁶R⁸, COR⁶, NR⁶R⁸, NR⁶COR⁸, NR⁶CONR⁸R⁸, NR⁶CO₂R⁸, NR⁶SO₂R⁸, NR⁶SO₂NR⁶R⁸, C(R⁶)═NOR⁸;
      (C₁-C₁₂)-haloalkyl;
      S(O)ₙR⁵, cyano, nitro, OR⁵, SO₂NR⁶R⁷, CO₂R⁸, COR⁸, NR⁶R⁸, NR⁶COR⁸, NR⁶CO₂R⁸, NR⁶SO₂R⁸;
         or
   R⁹ and R¹⁰ together with the nitrogen atom to which they are attached form a saturated, partially or fully unsaturated five-, six- or seven-membered ring which is optionally mono- to hexasubstituted by radicals from the group consisting of halogen, (C₁-C₆)-alkyl, halo-(C₁-C₆)-alkyl, OR⁵, S(O)ₙR⁵, CO₂R⁸, CONR⁶R⁸, COR⁶ and C(R⁶)═NOR⁸ and which, in addition to this nitrogen atom, contains r carbon atoms, o oxygen atoms, p sulfur atoms and q elements from the group consisting of NR⁷, CO and NCOR⁷ as ring atoms;
   R⁵ is (C₁-C₆)-alkyl, (C₃-C₆)-cycloalkyl, (C₁-C₆)-haloalkyl or aryl;
   R⁶ is hydrogen or R⁵;
   R⁷ is hydrogen, (C₁-C₆)-alkyl, (C₃-C₆)-cycloalkyl, (C₃-C₄)-alkenyl or (C₃-C₄)-alkynyl;
   R⁸ is hydrogen, (C₁-C₆)-alkyl, (C₃-C₆)-cycloalkyl, (C₃-C₄)-alkenyl or (C₃-C₄)-alkynyl;

R^{2a} is selected from the group consisting of
hydrogen;
methyl;

R^{2b} is hydrogen;

R³ is selected from the group consisting of
halogen, cyano, isocyano, NO₂;
(C₁-C₆)-alkyl, (C₃-C₆)-cycloalkyl, (C₁-C₆)-haloalkyl, (C₁-C₆)-alkylcarbonyl, (C₁-C₆)-haloalkylcarbonyl, (C₁-C₄)-alkyloxycarbonyl;
(C₂-C₃)-alkenyl, (C₂-C₃)-haloalkenyl;
(C₂-C₃)-alkynyl, (C₂-C₃)-haloalkynyl;
(C₁-C₂)-alkyl-S(O)ₙ and (C₁-C₂)-haloalkyl-S(O)ₙ;
CHO;
NH₂;

R⁴ is a phenyl, where the phenyl radical is unsubstituted or mono- or polysubstituted by a radical selected from the group consisting of
halogen, cyano, isocyano, nitro;
(C₁-C₆)-alkyl, (C₁-C₆)-haloalkyl, (C₁-C₃)-haloalkoxy;
(C₂-C₃)-alkenyl, (C₂-C₃)-haloalkenyl, (C₁-C₆)-alkoxy;
(C₂-C₃)-alkynyl, (C₂-C₃)-haloalkynyl, (C₁-C₄)-alkyl-S(O)ₙ;
CHO, (C₁-C₄)-alkyloxycarbonyl and NH₂;

R¹² is selected from the group consisting of
halogen, cyano, isocyano, NO₂;
(C₁-C₆)-alkyl, (C₁-C₆)-haloalkyl, (C₁-C₆)-alkylcarbonyl, (C₁-C₆)-haloalkylcarbonyl, (C₁-C₄)-alkyloxycarbonyl, (C₁-C₆)-alkoxy, (C₁-C₃)-haloalkoxy, (C₁-C₄)-alkyl-S(O)ₙ;
(C₂-C₃)-alkenyl, (C₂-C₃)-haloalkenyl;
(C₂-C₃)-alkynyl, (C₂-C₃)-haloalkynyl;
NH₂;

and where the indices are as follows:

m is 0, 1 or 2;

n is 0, 1 or 2;

o is 0, 1 or 2;

p is 0 or 1;

q is 0 or 1;

r is 3, 4, 5 or 6; and

s is 0, 1, 2, 3, 4 or 5, excluding the following compounds:

[(4-chloro-1,5-diphenyl-1H-pyrazol-3-yl)oxy]acetic acid, {[4-chloro-5-(4-methylphenyl)-1-phenyl-1H-pyrazol-3-yl]oxy}acetic acid, {[4-chloro-5-(3-chlorophenyl)-1-phenyl-1H-pyrazol-3-yl]oxy}acetic acid, ethyl [(4-chloro-1,5-diphenyl-1H-pyrazol-3-yl)oxy]acetate, ethyl {[4-chloro-5-(4-methylphenyl)-1-phenyl-1H-pyrazol-3-yl]oxy}acetate, ethyl {[4-chloro-5-(3-chlorophenyl)-1-phenyl-1H-pyrazol-3-yl]oxy}acetate, 2-(4-chloro-1,5-diphenylpyrazol-3-yl)oxypropanoic acid, 2-(4-bromo-1,5-diphenylpyrazol-3-yl)oxypropanoic acid.

### Definitions

In the definitions of the symbols used in the formulae above, collective terms were used which generally represent the following substituents:

Halogen: fluorine, chlorine, bromine or iodine, preferably fluorine, chlorine or bromine, and more preferably fluorine or chlorine.

Alkyl: saturated straight-chain or branched hydrocarbon radical having 1 to 8, preferably 1 to 6 and more preferably 1 to 4 carbon atoms, for example (but not limited to) C₁-C₆-alkyl such as methyl, ethyl, propyl (n-propyl), 1-methylethyl (isopropyl), butyl (n-butyl), 1-methylpropyl (sec-butyl), 2-methylpropyl (isobutyl), 1,1-dimethylethyl (tert-butyl), pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. This group is in particular a C₁-C₄-alkyl group, e.g. a methyl, ethyl, propyl, 1-methylethyl (isopropyl), butyl, 1-methylpropyl (sec-butyl), 2-methylpropyl (isobutyl) or 1,1-dimethylethyl (tert-butyl) group. Unless defined otherwise, for example for alkylsulfanyl, alkylsulfinyl, alkylsulfonyl, haloalkyl or haloalkylsulfanyl, this definition also applies to alkyl as part of a composite substituent, for example cycloalkylalkyl or hydroxyalkyl.

Alkenyl: unsaturated straight-chain or branched hydrocarbon groups having 2 to 8, preferably 2 to 6 and more preferably 2 to 4 carbon atoms and a double bond in any position, for example (but not limited to) C₂-C₆-alkenyl, such as vinyl, allyl, (E)-2-methylvinyl, (Z)-2-methylvinyl, isopropenyl, homoallyl, (E)-but-2-enyl, (Z)-but-2-enyl, (E)-but-1-enyl, (Z)-but-1-enyl, 2-methylprop-2-enyl, 1-methylprop-2-enyl, 2-methylprop-1-enyl, (E)-1-methylprop-1-enyl, (Z)-1-methylprop-1-enyl, pent-4-enyl, (E)-pent-3-enyl, (Z)-pent-3-enyl, (E)-pent-2-enyl, (Z)-pent-2-enyl, (E)-pent-1-enyl, (Z)-pent-1-enyl, 3-methylbut-3-enyl, 2-methylbut-3-enyl, 1-methylbut-3-enyl, 3-methylbut-2-enyl, (E)-2-methylbut-2-enyl, (Z)-2-methylbut-2-enyl, (E)-1-methylbut-2-enyl, (Z)-1-methylbut-2-enyl, (E)-3-methylbut-1-enyl, (Z)-3-methylbut-1-enyl, (E)-2-methylbut-1-enyl, (Z)-2-methylbut-1-enyl, (E)-1-methylbut-1-enyl, (Z)-1-methylbut-1-enyl, 1,1-dimethylprop-2-enyl, 1-ethylprop-1-enyl, 1-propylvinyl, 1-isopropylvinyl, (E)-3,3-dimethylprop-1-enyl, (Z)-3,3-dimethylprop-1-enyl, hex-5-enyl, (E)-hex-4-enyl, (Z)-hex-4-enyl, (E)-hex-3-enyl, (Z)-hex-3-enyl, (E)-hex-2-enyl, (Z)-hex-2-enyl, (E)-hex-1-enyl, (Z)-hex-1-enyl, 4-methylpent-4-enyl, 3-methylpent-4-enyl, 2-methylpent-4-enyl, 1-methylpent-4-enyl, 4-methylpent-3-enyl, (E)-3-methylpent-3-enyl, (Z)-3-methylpent-3-enyl, (E)-2-methylpent-3-enyl, (Z)-2-methylpent-3-enyl, (E)-1-methylpent-3-enyl, (Z)-1-methylpent-3-enyl, (E)-4-methylpent-2-enyl, (Z)-4-methylpent-2-enyl, (E)-3-methylpent-2-enyl, (Z)-3-methylpent-2-enyl, (E)-2-methylpent-2-enyl, (Z)-2-methylpent-2-enyl, (E)-1-methylpent-2-enyl, (Z)-1-methylpent-2-enyl, (E)-4-methylpent-1-enyl, (Z)-4-methylpent-1-enyl, (E)-3-methylpent-1-enyl, (Z)-3-methylpent-1-enyl, (E)-2-methylpent-1-enyl, (Z)-2-methylpent-1-enyl, (E)-1-methylpent-1-enyl, (Z)-1-methylpent-1-enyl, 3-ethylbut-3-enyl, 2-ethylbut-3-enyl, 1-ethylbut-3-enyl, (E)-3-ethylbut-2-enyl, (Z)-3-ethylbut-2-enyl, (E)-2-ethylbut-2-enyl, (Z)-2-ethylbut-2-enyl, (E)-1-ethylbut-2-enyl, (Z)-1-ethylbut-2-enyl, (E)-3-ethylbut-1-enyl, (Z)-3-ethylbut-1-enyl, 2-ethylbut-1-enyl, (E)-1-ethylbut-1-enyl, (Z)-1-ethylbut-1-enyl, 2-propylprop-2-enyl, 1-propylprop-2-enyl, 2-isopropylprop-2-enyl, 1-isopropylprop-2-enyl, (E)-2-propylprop-1-enyl, (Z)-2-propylprop-1-enyl, (E)-1-propylprop-1-enyl, (Z)-1-propylprop-1-enyl, (E)-2-isopropylprop-1-enyl, (Z)-2-isopropylprop-1-enyl, (E)-1-isopropylprop-1-enyl, (Z)-1-isopropylprop-1-enyl, 1-(1,1-dimethylethyl)ethenyl, buta-1,3-dienyl, penta-1,4-dienyl, hexa-1,5-dienyl or methylhexadienyl. This group is in particular vinyl or allyl. Unless defined otherwise, this definition also applies to alkenyl as part of a composite substituent, for example haloalkenyl.

Alkynyl: straight-chain or branched hydrocarbon groups having 2 to 8, preferably 2 to 6 and more preferably 2 to 4 carbon atoms and a triple bond in any position, for example (but not limited to) C₂-C₆-alkynyl, such as ethynyl, prop-1-ynyl, prop-2-ynyl, but-1-ynyl, but-2-ynyl, but-3-ynyl, 1-methylprop-2-ynyl, pent-1-ynyl, pent-2-ynyl, pent-3-ynyl, pent-4-ynyl, 2-methylbut-3-ynyl, 1-methylbut-3-ynyl, 1-methylbut-2-ynyl, 3-methylbut-1-ynyl, 1-ethylprop-2-ynyl, hex-1-ynyl, hex-2-ynyl, hex-3-ynyl, hex-4-ynyl, hex-5-ynyl, 3-methylpent-4-ynyl, 2-methylpent-4-ynyl, 1-methylpent-4-ynyl, 2-methylpent-3-ynyl, 1-methylpent-3-ynyl, 4-methylpent-2-ynyl, 1-methylpent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl or 3,3-dimethylbut-1-ynyl. The alkynyl group is in particular ethynyl, prop-1-ynyl or prop-2-ynyl. Unless defined otherwise, this definition also applies to alkynyl as part of a composite substituent, for example haloalkynyl.

Alkoxy: saturated straight-chain or branched alkoxy radicals having 1 to 8, preferably 1 to 6 and more preferably 1 to 4 carbon atoms, for example (but not limited to) C₁-C₆-alkoxy such as methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 2,2-dimethylpropoxy, 1-ethylpropoxy, 1,1-dimethylpropoxy, 1,2-dimethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy. Unless defined otherwise, this definition also applies to alkoxy as part of a composite substituent, for example haloalkoxy, alkynylalkoxy.

Alkoxycarbonyl: an alkoxy group which has 1 to 8, preferably 1 to 6 and more preferably 1 to 4 carbon atoms (as specified above) and is bonded to the skeleton via a carbonyl group (—C(═O)—). Unless defined otherwise, this definition also applies to alkoxycarbonyl as part of a composite substituent, for example cycloalkylalkoxycarbonyl.

Cycloalkyl: monocyclic, saturated hydrocarbyl groups having 3 to 10, preferably 3 to 8 and more preferably 3 to 6 carbon ring members, for example (but not limited to) cyclopropyl, cyclopentyl and cyclohexyl. Unless defined otherwise, this definition also applies to cycloalkyl as part of a composite substituent, for example cycloalkylalkyl.

Cycloalkenyl: monocyclic, partly unsaturated hydrocarbyl groups having 3 to 10, preferably 3 to 8 and more preferably 3 to 6 carbon ring members, for example (but not limited to) cyclopropenyl, cyclopentenyl and cyclohexenyl. Unless defined otherwise, this definition also applies to cycloalkenyl as part of a composite substituent, for example cycloalkenylalkyl.

Cycloalkoxy: monocyclic, saturated cycloalkyloxy radicals having 3 to 10, preferably 3 to 8 and more preferably 3 to 6 carbon ring members, for example (but not limited to) cyclopropyloxy, cyclopentyloxy and cyclohexyloxy. Unless defined otherwise, this definition also applies to cycloalkoxy as part of a composite substituent, for example cycloalkoxyalkyl.

Haloalkyl: straight-chain or branched alkyl groups having 1 to 8, preferably 1 to 6 and more preferably 1 to 4 carbon atoms (as described above), where some or all of the hydrogen atoms in these groups are replaced by halogen atoms as described above, for example (but not limited to) C₁-C₃-haloalkyl such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and 1,1,1-trifluoroprop-2-yl. Unless defined otherwise, this definition also applies to haloalkyl as part of a composite substituent, for example haloalkylaminoalkyl.

Haloalkenyl and haloalkynyl are defined analogously to haloalkyl, except that, instead of alkyl groups, alkenyl and alkynyl groups, respectively, are present as part of the substituent.

Haloalkoxy: straight-chain or branched alkoxy groups having 1 to 8, preferably 1 to 6 and more preferably 1 to 4 carbon atoms (as described above), where some or all of the hydrogen atoms in these groups are replaced by halogen atoms as described above, for example (but not limited to) C₁-C₃-haloalkoxy such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy and 1,1,1-trifluoroprop-2-oxy.

Unless defined otherwise, this definition also applies to haloalkoxy as part of a composite substituent, for example haloalkoxyalkyl.

Aryl: mono-, bi- or tricyclic aromatic or partially aromatic group having 6 to 14 carbon atoms, for example (but not limited to) phenyl, naphthyl, tetrahydronaphthyl, indenyl and indanyl. The bond to the parent general structure may be via any desired suitable ring member of the aryl radical. Aryl is preferably selected from phenyl, 1-naphthyl and 2-naphthyl. Particular preference is given to phenyl.

Heteroaryl: 5- or 6-membered cyclic aromatic group having at least 1 heteroatom, or else optionally 2, 3, 4 or 5 heteroatoms, where the heteroatoms are each independently selected from the group of S, N and O, where the group may also be part of a bi- or tricyclic system having up to 14 ring members, where the ring system may be formed with one or two further cycloalkyl, cycloalkenyl, heterocyclyl, aryl and/or heteroaryl radicals, and where benzofused 5- or 6-membered heteroaryl groups are preferred. The bonding to the parent general structure may be via any desired suitable ring member of the heteroaryl radical. Examples of 5-membered heteroaryl groups bonded to the skeleton via one of the carbon ring members are fur-2-yl, fur-3-yl, thien-2-yl, thien-3-yl, pyrrol-2-yl, pyrrol-3-yl, isoxazol-3-yl, isoxazol-4-yl, isoxazol-5-yl, isothiazol-3-yl, isothiazol-4-yl, isothiazol-5-yl, pyrazol-3-yl, pyrazol-4-yl, pyrazol-5-yl, oxazol-2-yl, oxazol-4-yl, oxazol-5-yl, thiazol-2-yl, thiazol-4-yl, thiazol-5-yl, imidazol-2-yl, imidazol-4-yl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl. Examples of 5-membered heteroaryl groups bonded to the skeleton via a nitrogen ring member are pyrrol-1-yl, pyrazol-1-yl, 1,2,4-triazol-1-yl, imidazol-1-yl, 1,2,3-triazol-1-yl and 1,3,4-triazol-1-yl.

Examples of 6-membered heteroaryl groups are pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl, 1,3,5-triazin-2-yl, 1,2,4-triazin-3-yl and 1,2,4,5-tetrazin-3-yl. Examples of benzofused 5-membered heteroaryl groups are indol-1-yl, indol-2-yl, indol-3-yl, indol-4-yl, indol-5-yl, indol-6-yl, indol-7-yl, benzimidazol-1-yl, benzimidazol-2-yl, benzimidazol-4-yl, benzimidazol-5-yl, indazol-1-yl, indazol-3-yl, indazol-4-yl, indazol-5-yl, indazol-6-yl, indazol-7-yl, indazol-2-yl, 1-benzofuran-2-yl, 1-benzofuran-3-yl, 1-benzofuran-4-yl, 1-benzofuran-5-yl, 1-benzofuran-6-yl, 1-benzofuran-7-yl, 1-benzothiophen-2-yl, 1-benzothiophen-3-yl, 1-benzothiophen-4-yl, 1-benzothiophen-5-yl, 1-benzothiophen-6-yl, 1-benzothiophen-7-yl, 1,3-benzothiazol-2-yl, 1,3-benzothiazol-4-yl, 1,3-benzothiazol-5-yl, 1,3-benzothiazol-6-yl, 1,3-benzothiazol-7-yl, 1,3-benzoxazol-2-yl, 1,3-benzoxazol-4-yl, 1,3-benzoxazol-5-yl, 1,3-benzoxazol-6-yl and 1,3-benzoxazol-7-yl, Examples of benzofused 6-membered heteroaryl groups are quinolin-2-yl, quinolin-3-yl, quinolin-4-yl, quinolin-5-yl, quinolin-6-yl, quinolin-7-yl, quinolin-8-yl, isoquinolin-1-yl, isoquinolin-3-yl, isoquinolin-4-yl, isoquinolin-5-yl, isoquinolin-6-yl, isoquinolin-7-yl and isoquinolin-8-yl. Further examples of 5- or 6-membered heteroaryl radicals that are part of a bicyclic ring system are 1,2,3,4-tetrahydroquinolin-1-yl, 1,2,3,4-tetrahydroquinolin-2-yl, 1,2,3,4-tetrahydroquinolin-7-yl, 1,2,3,4-tetrahydroquinolin-8-yl, 1,2,3,4-tetrahydroisoquinolin-1-yl, 1,2,3,4-tetrahydroisoquinolin-2-yl, 1,2,3,4-tetrahydroisoquinolin-5-yl, 1,2,3,4-tetrahydroisoquinolin-6-yl and 1,2,3,4-tetrahydroisoquinolin-7-yl. Unless defined otherwise, this definition also applies to heteroaryl as part of a composite substituent, for example heteroarylalkyl.

Heterocyclyl: three- to seven-membered, saturated or partly unsaturated heterocyclic group having at least one, optionally up to four, heteroatom(s) and/or hetero group(s) independently selected from the group consisting of N, O, S, S(═O), S(═O)₂ and di-(C₁-C₄)alkylsilyl, where the group may be benzofused. The bond to the parent general structure may be via a ring carbon atom or, if possible, via a ring nitrogen atom of the heterocyclic group. Saturated heterocyclic groups in this context are, for example (but not limited to), oxiranyl, aziridinyl, tetrahydrofuran-2-yl, tetrahydrofuran-3-yl, tetrahydrothien-2-yl, tetrahydrothien-3-yl, pyrrolidin-2-yl, pyrrolidin-3-yl, isoxazolidin-3-yl, isoxazolidin-4-yl, isoxazolidin-5-yl, isothiazolidin-3-yl, isothiazolidin-4-yl, isothiazolidin-5-yl, pyrazolidin-3-yl, pyrazolidin-4-yl, pyrazolidin-5-yl, oxazolidin-2-yl, oxazolidin-4-yl, oxazolidin-5-yl, thiazolidin-2-yl, thiazolidin-4-yl, thiazolidin-5-yl, imidazolidin-2-yl, imidazolidin-4-yl, 1,2,4-oxadiazolidin-3-yl, 1,2,4-oxadiazolidin-5-yl, 1,3,4-oxadiazolidin-2-yl, 1,2,4-thiadiazolidin-3-yl, 1,2,4-thiadiazolidin-5-yl, 1,3,4-thiadiazolidin-2-yl, 1,2,4-triazolidin-3-yl, 1,3,4-triazolidin-2-yl, piperidin-2-yl, piperidin-3-yl, piperidin-4-yl, 1,3-dioxan-5-yl, tetrahydropyran-2-yl, tetrahydropyran-4-yl, tetrahydrothien-2-yl, hexahydropyridazin-3-yl, hexahydropyridazin-4-yl, hexahydropyrimidin-2-yl, hexahydropyrimidin-4-yl, hexahydropyrimidin-5-yl, piperazin-2-yl, 1,3,5-hexahydrotriazin-2-yl and 1,2,4-hexahydrotriazin-3-yl. Partly unsaturated heterocyclic groups in this context are, for example (but not limited to), 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl. Examples of benzofused heterocyclic groups are indolin-1-yl, indolin-2-yl, indolin-3-yl, isoindolin-1-yl, isoindolin-2-yl, 2,3-dihydrobenzofuran-2-yl and 2,3-dihydrobenzofuran-3-yl. Unless defined otherwise, this definition also applies to heterocyclyl as part of a composite substituent, for example heterocyclylalkyl.

Not included are combinations which contravene the laws of nature and which the person skilled in the art would therefore rule out on the basis of their expert knowledge. For example, ring structures having three or more adjacent oxygen atoms are excluded.

Preference is given to compounds of the general formula (I) and agrochemically acceptable salts thereof where

A is selected from the group consisting of A1-A3,

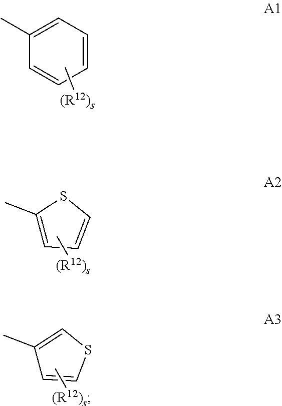

R¹ is selected from the group consisting of
OR^{1a} and
NR⁹R¹⁰; where
   R^{1a} is selected from the group consisting of
      hydrogen;
      methyl, ethyl which is unsubstituted or substituted by one or more substituents selected from the group consisting of halogen, (C₃-C₆)-cycloalkyl, (C₁-C₄)-trialkylsilyl, (C₁-C₄)-alkoxy, cyano and nitro;
      (C₂-C₆)-alkenyl, (C₂-C₆)-haloalkenyl;
      aryl-(C₁-C₄)-alkyl, where the aryl is unsubstituted or substituted by one or more substituents selected from the group consisting of halogen, (C₁-C₄)-alkyl, (C₁-C₄)-haloalkyl;
   R⁹ is selected from the group consisting of hydrogen, (C₁-C₆)-alkyl;
   R¹⁰ is selected from the group consisting of
      hydrogen;
      (C₃-C₇)-cycloalkyl-(C₁-C₄)-alkyl, aryl-(C₁-C₄)-alkyl, aryl-(C₁-C₄)-alkoxy,
         where the cycloalkyl and aryl is unsubstituted or substituted by one or more substituents selected from the group consisting of halogen, (C₁-C₄)-alkyl, (C₁-C₄)-haloalkyl;
      (C₁-C₆)-alkyl, (C₂-C₆)-alkenyl, (C₂-C₆)-alkynyl; (C₃-C₈)-cycloalkyl;
         where the abovementioned alkyl, alkenyl, alkynyl and cycloalkenyl radicals are unsubstituted or are each independently substituted by m radicals selected from the group consisting of cyano, C(O)OR⁸;
      (C₁-C₆)-haloalkyl
   R⁸ is hydrogen, (C₁-C₆)-alkyl, (C₃-C₆)-cycloalkyl;

R^{2a} is selected from the group consisting of
hydrogen;
methyl;

R^{2b} is hydrogen;

R³ is selected from the group consisting of
fluorine, chlorine, bromine, iodine, cyano, isocyano, NO₂;
(C₁-C₆)-alkyl, (C₃-C₆)-cycloalkyl, (C₁-C₆)-haloalkyl, (C₁-C₄)-alkyloxycarbonyl;
(C₂-C₃)-alkynyl, (C₂-C₃)-haloalkynyl;

R⁴ is phenyl, where the phenyl radical is unsubstituted or mono- or polysubstituted by a radical selected from the group consisting of
fluorine, chlorine, bromine;
methyl, ethyl;
methoxy, ethoxy;

R¹² is selected from the group consisting of
halogen, cyano, nitro;
(C₁-C₆)-alkyl, (C₁-C₆)-haloalkyl, (C₁-C₃)-haloalkoxy;
(C₁-C₆)-alkoxy;

and where the indices are as follows:

m is 0, 1 or 2;

s is 0, 1, 2, 3,

excluding the compounds:

[(4-chloro-1,5-diphenyl-1H-pyrazol-3-yl)oxy]acetic acid, {[4-chloro-5-(4-methylphenyl)-1-phenyl-1H-pyrazol-3-yl]oxy}acetic acid, {[4-chloro-5-(3-chlorophenyl)-1-phenyl-1H-pyrazol-3-yl]oxy}acetic acid, ethyl [(4-chloro-1,5-diphenyl-1H-pyrazol-3-yl)oxy]acetate, ethyl {[4-chloro-5-(4-methylphenyl)-1-phenyl-1H-pyrazol-3-yl]oxy}acetate, ethyl {[4-chloro-5-(3-chlorophenyl)-1-phenyl-1H-pyrazol-3-yl]oxy}acetate, 2-(4-chloro-1,5-diphenyl-pyrazol-3-yl)oxypropanoic acid, 2-(4-bromo-1,5-diphenylpyrazol-3-yl)oxypropanoic acid.

Particular preference is given to compounds of the general formula (I) and agrochemically acceptable salts thereof where

A is A1;

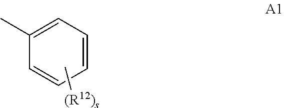

R¹ is selected from the group consisting of
OR^{1a} and
NR⁹R¹⁰; where
   R^{1a} is selected from the group consisting of
      hydrogen;
      methyl, ethyl, trimethylsilylmethyl;
      1-propenyl, 2-propenyl;
      benzyl, 1-phenylethyl, 2-phenylethyl, where the phenyl radical in each of the three groups mentioned is unsubstituted or substituted by halogen;
   R⁹ is hydrogen;
   R¹⁰ is selected from the group consisting of
      hydrogen;
      cyclopropylmethyl;
      benzyl, 1-phenylethyl, 2-phenylethyl, benzyloxy, where the phenyl radical in each of the four groups mentioned is unsubstituted or substituted by halogen;
      methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, where the abovementioned radicals are unsubstituted or are each independently monosubstituted by a C(O)OR⁸ radical;
      cyclopropyl, cyclobutyl, cyclopentyl, where the three radicals mentioned are unsubstituted or are each independently monosubstituted by a C(O)OR⁸ radical;
      1-propenyl, 2-propenyl, 2-methyl-2-propenyl, prop-2-yn-1-yl, but-2-yn-1-yl;
   R⁸ is hydrogen, methyl, ethyl;

R^{2a} is selected from the group consisting of
hydrogen;
methyl;

R^{2b} is hydrogen;

R³ is selected from the group consisting of
fluorine, chlorine, bromine, iodine, cyano, NO₂;
trifluoromethyl;
ethynyl;
C(O)Omethyl;

R⁴ is phenyl, where the phenyl radical is unsubstituted or mono- or polysubstituted by a radical selected from the group consisting of
fluorine, chlorine, bromine;
methyl, ethyl;
methoxy, ethoxy;

R¹² is selected from the group consisting of
fluorine, chlorine, NO₂;
trifluoromethyl, methoxy, ethoxy;

and where the index is as follows:

s is 1, 2, 3,

excluding the compounds:

[(4-chloro-1,5-diphenyl-1H-pyrazol-3-yl)oxy]acetic acid, {[4-chloro-5-(4-methylphenyl)-1-phenyl-1H-pyrazol-3-yl]oxy}acetic acid, {[4-chloro-5-(3-chlorophenyl)-1-phenyl-1H-pyrazol-3-yl]oxy}acetic acid, ethyl [(4-chloro-1,5-diphenyl-1H-pyrazol-3-yl)oxy]acetate, ethyl {[4-chloro-5-(4-methylphenyl)-1-phenyl-1H-pyrazol-3-yl]oxy}acetate, ethyl {[4-chloro-5-(3-chlorophenyl)-1-phenyl-1H-pyrazol-3-yl]oxy}acetate, 2-(4-chloro-1,5-diphenyl-pyrazol-3-yl)oxypropanoic acid, 2-(4-bromo-1,5-diphenylpyrazol-3-yl)oxypropanoic acid.

Very particular preference is given to compounds of the general formula (I) and agrochemically acceptable salts thereof where

A is A1

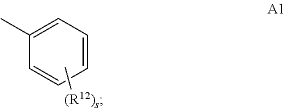

R¹ is selected from the group consisting of
OR^{1a} and
NR⁹R¹⁰; where
   R^{1a} is selected from the group consisting of
      hydrogen;
      methyl, ethyl;
      2-propenyl;
   R⁹ is hydrogen;
   R¹⁰ is selected from the group consisting of
      cyclopentyl monosubstituted by C(O)OR^{B};
      cyclopropylmethyl;
      CH₂C(O)OR^{B}, CH₂CH₂C(O)OR^{B};
      2-propenyl, prop-2-yn-1-yl;
   R⁸ is hydrogen, methyl, ethyl;

R^{2a} is selected from the group consisting of
hydrogen;
methyl;

R^{2b} is hydrogen;

R³ is selected from the group consisting of
chlorine, bromine, iodine, cyano, NO₂;

R⁴ is phenyl, where the phenyl radical is unsubstituted or mono- or polysubstituted by a radical selected from the group consisting of
fluorine, chlorine;

R¹² is selected from the group consisting of
fluorine, chlorine;

and where the index is as follows:

s is 1, 2,

excluding the compounds:

[(4-chloro-1,5-diphenyl-1H-pyrazol-3-yl)oxy]acetic acid, {[4-chloro-5-(4-methylphenyl)-1-phenyl-1H-pyrazol-3-yl]oxy}acetic acid, {[4-chloro-5-(3-chlorophenyl)-1-phenyl-1H-pyrazol-3-yl]oxy}acetic acid, ethyl [(4-chloro-1,5-diphenyl-1H-pyrazol-3-yl)oxy]acetate, ethyl {[4-chloro-5-(4-methylphenyl)-1-phenyl-1H-pyrazol-3-yl]oxy}acetate, ethyl {[4-chloro-5-(3-chlorophenyl)-1-phenyl-1H-pyrazol-3-yl]oxy}acetate, 2-(4-chloro-1,5-diphenyl-pyrazol-3-yl)oxypropanoic acid, 2-(4-bromo-1,5-diphenylpyrazol-3-yl)oxypropanoic acid.

The present compounds of the general formula (I) have, at the second carbon of the alkyl acid structure, a chiral carbon atom which, in the structure shown below, is indicated by the marker (*):

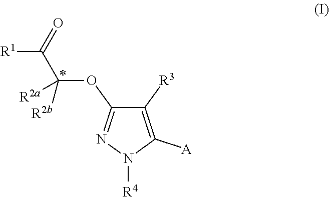

According to the rules of Cahn, Ingold and Prelog (CIP rules), this carbon atom can have either an (R) configuration or an (S) configuration.

The present invention encompasses compounds of the general formula (I) both with (S) and with (R) configuration, meaning that the present invention encompasses the compounds of the general formula (I) in which the carbon atom in question has

(1) an (R) configuration; or

(2) an (S) configuration.

In addition, the scope of the present invention also encompasses

(3) any mixtures of compounds of the general formula (I) having an (R) configuration (compounds of the general formula (I-(R)) with compounds of the general formula (I) having an (S) configuration (compounds of the general formula (I-S)), the present invention also encompassing a racemic mixture of the compounds of the general formula (I) having (R) and (S) configuration.

However, within the context of the present invention, preference is given particularly to compounds of the general formula (I) having (R) configuration with a selectivity of 60 to 100%, preferably 80 to 100%, especially 90 to 100%, very particularly 95 to 100%, where the particular (R) compound is present with an enantioselectivity of in each case more than 50% ee, preferably 60 to 100% ee, especially 80 to 100% ee, very particularly 90 to 100% ee, most preferably 95 to 100% ee, based on the total content of (R) compound in question.

Accordingly, the present invention relates especially to compounds of the general formula (I*) in which the stereochemical configuration on the carbon atom marked by (*) is present with a stereochemical purity of 60 to 100% (R), preferably 80 to 100% (R), especially 90 to 100% (R), very particularly 95 to 100% (R).

In addition, depending on the respective radicals chosen, further stereoelements may be present in the compounds of the general formula (I) according to the invention.

Preference is given to the compounds listed in the tables below. The compounds of the general formula (I) having (R) configuration are marked accordingly in the column which lists the radical R^{2a}. For example, if R^{2a}=alkyl, the preferred stereochemical configuration at the carbon atom marked (*) of the general formula (I) is the (R) configuration.

Taking account of the Cahn, Ingold and Prelog rule, at the carbon atom marked by (*),

there may also be a situation in which, owing to the priority of the substituents in question, the (S) configuration is preferred at the carbon atom marked by (*).

This is the case, for example, when the R^{2a} radical corresponds to a (C₁-C₆)-alkoxy radical.

Therefore, in the context of the present invention, preference is given especially to compounds of the general formula (I) that correspond in terms of their spatial arrangement to those compounds of the general formula (I) with R^{2a}=methyl having R configuration with a selectivity of 60% to 100%, preferably 80% to 100%, especially 90% to 100%, very particularly 95% to 100%, where

the respective (R) analogue compound is present with an enantioselectivity of more than 50% ee in each case,

preferably 60% to 100% ee, especially 80% to 100% ee, very particularly 90% to 100% ee, most

preferably 95% to 100% ee, based on the total content of (R) analogue compound in question.

Therefore, the present invention especially relates to compounds of the general formula (I) in which the stereochemical configuration on the carbon atom marked by (*) is present with a stereochemical purity of 60% to 100% (R or R analogue), preferably 80% to 100% (R or R analogue), especially 90% to 100% (R or R analogue), very particularly 95% to 100% (R or R analogue).

TABLE I
Esters
(I)

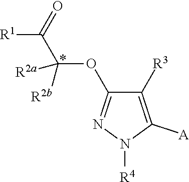

| Ex. No. | R¹ | R^{2a} | R^{2b} | R³ | R⁴ | A |
|---|---|---|---|---|---|---|
| I-001 | MeO | H | H | Cl | 4-Cl—Ph | Ph |
| I-002 | MeO | H | H | Cl | Ph | 3-F—Ph |
| I-003 | MeO | H | H | Cl | 3-F—Ph | 3-F—Ph |
| I-004 | MeO | H | H | Cl | Ph | 4-Cl—Ph |
| I-005 | MeO | H | H | Cl | 3-F—Ph | 4-Cl—Ph |
| I-006 | MeO | H | H | Cl | 4-F—Ph | 4-F—Ph |
| I-007 | MeO | H | H | Cl | 4-Cl—Ph | 4-F—Ph |
| I-008 | MeO | H | H | Cl | Ph | 4-F—Ph |
| I-009 | MeO | H | H | CN | 4-F—Ph | 4-F—Ph |
| I-010 | MeO | H | H | CN | 4-F—Ph | 4-Cl—Ph |
| I-011 | MeO | H | H | CN | 4-F—Ph | Ph |
| I-012 | MeO | H | H | CN | 3-F—Ph | 4-F—Ph |
| I-013 | MeO | H | H | CN | 4-Cl—Ph | 4-F—Ph |
| I-014 | MeO | H | H | CN | Ph | 4-F—Ph |
| I-015 | MeO | H | H | Cl | 2-F—Ph | Ph |
| I-016 | MeO | H | H | Cl | 3-F—Ph | Ph |
| I-017 | MeO | H | H | CN | 3-F—Ph | 4-Cl—Ph |
| I-018 | MeO | H | H | CN | Ph | 4-Cl—Ph |
| I-019 | MeO | H | H | CN | 4-MeO—Ph | 3-F—Ph |
| I-020 | MeO | H | H | CN | 4-Me—Ph | 3-F—Ph |
| I-021 | MeO | H | H | CN | Ph | 3-F—Ph |
| I-022 | MeO | H | H | CN | 2-F—Ph | Ph |
| I-023 | MeO | H | H | CN | 3-F—Ph | Ph |
| I-024 | MeO | H | H | CN | 4-Cl—Ph | Ph |
| I-025 | MeO | H | H | Cl | 4-F—Ph | 3,4-diF—Ph |
| I-026 | MeO | H | H | Cl | 3-ClPh | 3,4-diF—Ph |
| I-027 | MeO | H | H | Cl | 3,4-diCl—Ph | 3,4-diF—Ph |
| I-028 | MeO | H | H | Cl | Ph | 4-EtO-3-F—Ph |
| I-029 | MeO | H | H | Cl | 4-Cl—Ph | 3-F—Ph |
| I-030 | MeO | H | H | Cl | 3-F—Ph | 4-MeO—Ph |
| I-031 | MeO | H | H | Cl | 3-F—Ph | 2,4-diCl—Ph |
| I-032 | MeO | H | H | CN | 3-F—Ph | 4-MeO—Ph |
| I-033 | MeO | H | H | CN | 4-Cl—Ph | 4-MeO—Ph |
| I-034 | MeO | H | H | CN | Ph | 4-MeO—Ph |
| I-035 | MeO | H | H | Cl | 2,4-diCl—Ph | 4-F—Ph |
| I-036 | MeO | H | H | CN | 4-F—Ph | 3,4-diF—Ph |
| I-037 | MeO | H | H | CN | 3-ClPh | 4-MeO—Ph |
| I-038 | MeO | H | H | CN | 3-F—Ph | 3-ClPh |
| I-039 | MeO | H | H | CN | 4-MeO—Ph | 3-ClPh |
| I-040 | MeO | H | H | CN | 4-Me—Ph | 3-ClPh |
| I-041 | MeO | H | H | CN | 3-ClPh | 4-Cl—Ph |
| I-042 | MeO | H | H | CN | 3-Cl-4-F—Ph | 4-EtO-3-F—Ph |
| I-043 | MeO | H | H | CN | 3-F—Ph | 4-EtO-3-F—Ph |
| I-044 | MeO | H | H | CN | 2,4-diCl—Ph | 4-EtO-3-F—Ph |
| I-045 | MeO | H | H | CN | Ph | 4-EtO-3-F—Ph |
| I-046 | MeO | H | H | CN | 3-ClPh | 3-F—Ph |
| I-047 | MeO | H | H | CN | 3-F—Ph | 3-F—Ph |
| I-048 | MeO | H | H | CN | 4-Cl—Ph | 3-F—Ph |
| I-049 | MeO | H | H | CN | 3-Cl-4-EtO—Ph | 3-ClPh |
| I-050 | MeO | H | H | CN | 3-Cl-4-EtO—Ph | 4-Cl—Ph |
| I-051 | MeO | H | H | CN | 3-ClPh | 3,4-diF—Ph |
| I-052 | MeO | H | H | CF₃ | Ph | 4-F—Ph |
| I-053 | MeO | (R)—Me | H | CF₃ | Ph | 4-F—Ph |
| I-054 | MeO | H | H | Br | 2-F—Ph | Ph |
| I-055 | MeO | H | H | Br | 2-F—Ph | 3,4-diF—Ph |
| I-056 | EtO | H | H | Br | Ph | 4-F—Ph |
| I-057 | OH | H | H | Br | Ph | 4-F—Ph |
| I-058 | EtO | H | H | I | 2-F—Ph | Ph |
| I-059 | EtO | H | H | F | 2-F—Ph | Ph |
| I-060 | EtO | H | H | Cl | 2-F—Ph | Ph |
| I-061 | MeO | Me | H | CN | 2-F—Ph | Ph |
| I-062 | MeO | H | H | CO₂Me | 2-F—Ph | Ph |
| I-063 | MeO | H | H | CCH | 2-F—Ph | Ph |
| I-064 | MeO | Me | H | CN | Ph | 4-F—Ph |
| I-065 | MeO | H | H | CO₂Me | Ph | 4-F—Ph |
| I-066 | MeO | H | H | CCH | Ph | 4-F—Ph |
| I-067 | MeO | Me | H | CN | 3-F—Ph | 3-F—Ph |
| I-068 | MeO | H | H | CO₂Me | 3-F—Ph | 3-F—Ph |
| I-069 | MeO | H | H | CCH | 3-F—Ph | 3-F—Ph |
| I-070 | EtO | H | H | Br | Ph | Ph |
| I-071 | EtO | H | H | Br | Ph | 4-Cl—Ph |
| I-072 | EtO | H | H | Br | 4-F—Ph | Ph |
| I-073 | EtO | H | H | Br | 4-Cl—Ph | Ph |
| I-074 | EtO | H | H | Br | 4-Cl—Ph | 4-Cl—Ph |
| I-075 | EtO | H | H | NO₂ | 2-F—Ph | Ph |
| I-076 | EtO | H | H | Br | 4-F—Ph | 4-F—Ph |
| I-077 | EtO | H | H | Br | 4-F—Ph | 4-Cl—Ph |
| I-078 | EtO | H | H | Br | 4-Cl—Ph | 4-F—Ph |
| I-079 | EtO | H | H | Br | 2,4-diF—Ph | Ph |
| I-080 | EtO | H | H | Br | 2,4-diF—Ph | 4-Cl—Ph |
| I-081 | EtO | H | H | Br | 2,4-diFPh | 4-F—Ph |
| I-082 | OH | H | H | Br | 2-F—Ph | Ph |
| I-082 | OH | H | H | Br | 2-F—Ph | Ph |
| I-083 | MeO | H | H | Br | 4-F—Ph | 3-CF₃—Ph |
| I-084 | MeO | H | H | Br | 2-F—Ph | 2,3,4-triF—Ph |
| I-085 | EtO | H | H | Br | Ph | 2,4-diF—Ph |
| I-086 | EtO | H | H | Br | 4-F—Ph | 2,4-diF—Ph |
| I-087 | EtO | H | H | Br | 4-Cl—Ph | 2,4-diF—Ph |
| I-088 | EtO | H | H | Br | 2,4-diF—Ph | 2,4-diF—Ph |
| I-089 | EtO | H | H | Br | 2-MeO—Ph | 4-F—Ph |
| I-090 | EtO | H | H | Br | 2-MeO—Ph | 4-Cl—Ph |
| I-091 | EtO | H | H | Br | 2-MeO—Ph | 2,4-diF—Ph |
| I-092 | EtO | H | H | Br | 2-MeO—Ph | Ph |
| I-093 | EtO | H | H | Cl | 2,4,5-triF—Ph | Ph |
| I-094 | MeO | H | H | Cl | 2-F—Ph | 5-F-2-Thienyl |
| I-095 | MeO | H | H | Br | 2-F—Ph | 5-F-2-Thienyl |
| I-096 | OH | H | H | Cl | 2,4,5-triF—Ph | Ph |
| I-097 | EtO | H | H | Cl | 2,4,5-triF—Ph | 3-F—Ph |
| I-098 | EtO | H | H | Cl | 2,4-diF—Ph | 3-F—Ph |
| I-099 | MeO | H | H | NO₂ | 2-F—Ph | 4-NO₂—Ph |
| I-100 | EtO | H | H | Cl | 2,4-diF—Ph | Ph |
| I-101 | MeO | H | H | Br | Ph | 4-F—Ph |
| I-102 | Me₃SiCH₂O | H | H | Br | 2-F—Ph | 3,4-diF—Ph |
| I-103 | OH | Me | H | CN | Ph | 4-F—Ph |
| I-104 | MeO | Me | H | CN | 3-F—Ph | 3-F—Ph |
| I-105 | MeO | Me | H | CN | Ph | 4-F—Ph |
| I-106 | OH | Me | H | CN | 3-F—Ph | 3-F—Ph |
| I-107 | MeO | H | H | Br | 4-F—Ph | 3-F—Ph |
| I-108 | MeO | H | H | NO₂ | 4-F—Ph | 3-F—Ph |
| I-109 | MeO | H | H | I | 4-F—Ph | 3-F—Ph |
| I-110 | MeO | H | H | Cl | 4-F—Ph | 3-F—Ph |
| I-111 | 3-F—PhCH₂O | H | H | Br | Ph | 4-F—Ph |
| I-112 | PhCH₂O | H | H | Br | Ph | 4-F—Ph |
| I-113 | CH₂═CHCH₂O | H | H | Br | Ph | 4-F—Ph |
| I-114 | MeO | H | H | Br | 3-F—Ph | Ph |
| I-115 | MeO | H | H | I | 3-F—Ph | Ph |
| I-116 | OH | Me | H | CN | Ph | 4-F—Ph |
| I-117 | OH | Me | H | CN | 3-F—Ph | 3-F—Ph |
| I-118 | OH | H | H | I | 4-F—Ph | 3-F—Ph |
| I-119 | OH | H | H | Br | 4-F—Ph | 3-F—Ph |
| I-120 | OH | H | H | Cl | 4-F—Ph | 3-F—Ph |
| I-121 | OH | H | H | NO₂ | 4-F—Ph | 3-F—Ph |
| I-122 | MeO | H | H | NO₂ | 3-F—Ph | Ph |
| I-123 | 3-F—PhCH₂O | H | H | Br | 4-F—Ph | 3-F—Ph |
| I-124 | PhCH₂O | H | H | I | 4-F—Ph | 3-F—Ph |
| I-125 | MeO | (R)—Me | H | CN | 3-F—Ph | 3-F—Ph |
| I-126 | MeO | (S)—Me | H | CN | 3-F—Ph | 3-F—Ph |
| I-127 | OH | H | H | Br | 3-F—Ph | Ph |
| I-128 | OH | H | H | I | 3-F—Ph | Ph |
| I-129 | OH | H | H | NO₂ | 3-F—Ph | Ph |
| I-130 | MeO | (R)—Me | H | CN | Ph | 4-F—Ph |
| I-131 | EtO | H | H | Cl | 2-F—Ph | 3,4-diF—Ph |
| I-132 | MeO | Me | H | Br | Ph | 4-F—Ph |
| I-133 | EtO | H | H | Br | 2-F—Ph | 3,4-diF—Ph |
| I-134 | EtO | H | H | Br | 2,6-diF—Ph | 3,4-diF—Ph |
| I-135 | MeO | Me | Me | Br | Ph | 4-F—Ph |
| I-136 | OH | H | H | Br | 2-F—Ph | 3,4-diF—Ph |
| I-137 | OH | H | H | Br | 2,6-diF—Ph | 3,4-diF—Ph |
| I-138 | OH | Me | H | Br | Ph | 4-F—Ph |
| I-139 | OH | Me | H | Cl | Ph | 4-F—Ph |
| I-140 | OH | Me | Me | Br | Ph | 4-F—Ph |
| I-141 | MeO | (S)—Me | H | CN | Ph | 4-F—Ph |
| I-142 | EtO | H | H | Cl | 2,6-diF—Ph | 3,4-diF—Ph |
| I-143 | EtO | H | H | NO₂ | 2-F—Ph | 3,4-diF—Ph |
| I-144 | EtO | H | H | NO₂ | 2,6-diF—Ph | 3,4-diF—Ph |
| I-145 | MeO | Me | H | Cl | Ph | 4-F—Ph |
| I-146 | CH₂═CHCH₂O | H | H | Cl | 2,6-diF—Ph | 3,4-diF—Ph |
| I-147 | CH₂═CHCH₂O | H | H | Cl | 2-F—Ph | 3,4-diF—Ph |
| I-148 | CH₂═CHCH₂O | H | H | NO₂ | 2-F—Ph | 3,4-diF—Ph |
| I-149 | CH₂═CHCH₂O | H | H | NO₂ | 2,6-diF—Ph | 3,4-diF—Ph |
| I-150 | EtO | H | H | Br | 4-Cl-2-F—Ph | 2,4-diF—Ph |
| I-151 | EtO | H | H | Br | 4-Cl-2-F—Ph | 4-Cl-2-F—Ph |
| I-152 | EtO | H | H | Br | 2,4-diCl—Ph | 4-Cl-2-F—Ph |
| I-153 | EtO | H | H | Br | 2,4-diClPh | 2,4-diF—Ph |
| I-154 | EtO | H | H | Br | 4-Cl-2-F—Ph | 2,4-diCl—Ph |
| I-155 | EtO | H | H | Br | 4-Cl-2-F—Ph | 2-Cl-4-F—Ph |
| I-156 | EtO | H | H | Br | 4-Cl-2-F—Ph | 4-F—Ph |
| I-157 | EtO | H | H | Br | 4-Cl-2-F—Ph | 4-Cl—Ph |
| I-158 | EtO | H | H | Br | 2,4-diCl—Ph | 4-F—Ph |
| I-159 | EtO | H | H | Br | 2,4-diCl—Ph | 4-Cl—Ph |
| I-160 | EtO | H | H | Br | 4-F—Ph | 4-Cl-2-F—Ph |
| I-161 | EtO | H | H | Br | 4-Cl—Ph | 4-Cl-2-F—Ph |
| I-162 | EtO | H | H | Br | 2-Cl-4-F—Ph | 4-Cl-2-F—Ph |
| I-163 | EtO | H | H | Br | 2,4-diF—Ph | 4-Cl-2-F—Ph |
| I-164 | MeO | Me | H | Cl | Ph | 3-F—Ph |
| I-165 | MeO | Me | H | Cl | Ph | 3,5-diF—Ph |
| I-166 | MeO | Me | H | Cl | Ph | 2-F—Ph |
| I-167 | OH | Me | H | Br | 2,6-diF—Ph | 3,4-diF—Ph |
| I-168 | OH | Me | H | Br | 2-F—Ph | 3,4-diF—Ph |
| I-169 | MeO | Me | H | Br | 2,6-diF—Ph | 3,4-diF—Ph |
| I-170 | MeO | Me | H | Br | 2-F—Ph | 3,4-diF—Ph |
| I-171 | MeO | Me | H | CN | 2-F—Ph | 3,4-diF—Ph |
| I-172 | MeO | (S)—Me | H | CN | 2-F—Ph | 3,4-diF—Ph |
| I-173 | MeO | (R)—Me | H | CN | 2-F—Ph | 3,4-diF—Ph |
| I-174 | MeO | (R)—Me | H | Br | 3-F—Ph | Ph |
| I-175 | OH | (R)—Me | H | Br | 3-F—Ph | Ph |
| I-176 | MeO | (S)—Me | H | Br | 3-F—Ph | Ph |
| I-177 | OH | (S)—Me | H | Br | 3-F—Ph | Ph |
| I-178 | MeO | (R)—Me | H | CN | 2,6-diF—Ph | 3,4-diF—Ph |
| I-179 | MeO | (S)—Me | H | CN | 2,6-diF—Ph | 3,4-diF—Ph |
| I-180 | EtO | H | H | Br | 2-F—Ph | 2-F—Ph |
| I-181 | MeO | H | H | Br | 2,5-diF—Ph | 3,4-diF—Ph |
| I-182 | MeO | (R)—Me | H | Br | 2,5-diF—Ph | 3,4-diF—Ph |
| I-183 | MeO | H | H | CN | 2,5-diF—Ph | 3,4-diF—Ph |
| I-184 | MeO | (R)—Me | H | Br | 2-F—Ph | 3,4-diF—Ph |
| I-185 | MeO | (R)—Me | H | CN | 2,5-diF—Ph | 3,4-diF—Ph |

TABLE II
Salts
(I)

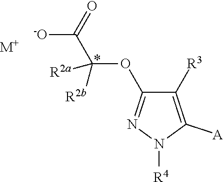

| Ex. No. | M⁺ | R^{2a} | R^{2b} | R³ | R⁴ | A |
|---|---|---|---|---|---|---|
| II-01 | K⁺ | H | H | I | 3-F—Ph | Ph |
| II-02 | Na⁺ | H | H | I | 3-F—Ph | Ph |
| II-03 | Li⁺ | H | H | I | 3-F—Ph | Ph |
| II-04 | morpholin-4-ium | H | H | I | 3-F—Ph | Ph |
| II-05 | 2-propenylammonium | H | H | I | 3-F—Ph | Ph |
| II-06 | propylammonium | H | H | I | 3-F—Ph | Ph |
| II-07 | tripropylammonium | H | H | I | 3-F—Ph | Ph |
| II-08 | ethyl(methyl)ammonium | H | H | I | 3-F—Ph | Ph |
| II-09 | tert-butylammonium | H | H | I | 3-F—Ph | Ph |
| II-10 | 2-phenylethylammonium | H | H | I | 3-F—Ph | Ph |
| II-11 | piperidin-1-ium | H | H | I | 3-F—Ph | Ph |
| II-12 | octylammonium | H | H | I | 3-F—Ph | Ph |
| II-13 | benzylammonium | H | H | I | 3-F—Ph | Ph |
| II-14 | triethylammonium | H | H | I | 3-F—Ph | Ph |

TABLE III
Amides
(I)

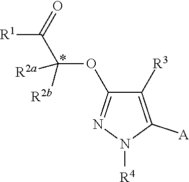

| Ex. No. | R¹ | R^{2a} | R^{2b} | R³ | R⁴ | A |
|---|---|---|---|---|---|---|
| III-001 | 2-methylprop-2-en-1-ylamino | H | H | Br | 2-F—Ph | Ph |
| III-002 | cyclopropylamino | H | H | Br | 2-F—Ph | Ph |
| III-003 | 2-propenylamino | H | H | Br | 2-F—Ph | Ph |
| III-004 | 2-propenylamino | H | H | Br | Ph | 4-F—Ph |
| III-005 | cyclopropylmethylamino | H | H | Br | Ph | 4-F—Ph |
| III-006 | prop-2-yn-1-ylamino | H | H | Br | Ph | 4-F—Ph |
| III-007 | 2-methylpropylamino | H | H | Br | Ph | 4-F—Ph |
| III-008 | 2-propenylamino | H | H | I | 4-F—Ph | 3-F—Ph |
| III-009 | butylamino | H | H | I | 4-F—Ph | 3-F—Ph |
| III-010 | benzylamino | H | H | I | 4-F—Ph | 3-F—Ph |
| III-011 | cyclopropylamino | H | H | I | 4-F—Ph | 3-F—Ph |
| III-012 | cyclopropylmethylamino | H | H | I | 4-F—Ph | 3-F—Ph |
| III-013 | 2-methylpropylamino | H | H | I | 4-F—Ph | 3-F—Ph |
| III-014 | 2-propenyl(methyl)amino | H | H | I | 4-F—Ph | 3-F—Ph |
| III-015 | 2-propenylamino | H | H | Br | 4-F—Ph | 3-F—Ph |
| III-016 | prop-2-yn-1-ylamino | H | H | Br | 4-F—Ph | 3-F—Ph |
| III-017 | but-2-yn-1-ylamino | H | H | Br | 4-F—Ph | 3-F—Ph |
| III-018 | cyclopropylamino | H | H | Br | 4-F—Ph | 3-F—Ph |
| III-019 | (3-ethoxy-3-oxopropyl)amino | H | H | Br | 4-F—Ph | 3-F—Ph |
| III-020 | 2-propenylamino | Me | H | CN | Ph | 4-F—Ph |
| III-021 | (3-methoxy-3-oxopropyl)amino | Me | H | CN | Ph | 4-F—Ph |
| III-022 | 2-propenylamino | H | H | Cl | 4-F—Ph | 3-F—Ph |
| III-023 | prop-2-yn-1-ylamino | H | H | Cl | 4-F—Ph | 3-F—Ph |
| III-024 | cyclopropylamino | H | H | Cl | 4-F—Ph | 3-F—Ph |
| III-025 | cyclopropylmethylamino | H | H | Cl | 4-F—Ph | 3-F—Ph |
| III-026 | (3-methoxy-3-oxopropyl)amino | H | H | Cl | 4-F—Ph | 3-F—Ph |
| III-027 | (2-methoxy-2-oxoethyl)amino | H | H | Cl | 4-F—Ph | 3-F—Ph |
| III-028 | cyclopropylamino | H | H | NO₂ | 4-F—Ph | Ph |
| III-029 | (2-methoxy-2-oxoethyl)amino | H | H | NO₂ | 3-F—Ph | Ph |
| III-030 | benzylamino | H | H | NO₂ | 3-F—Ph | Ph |
| III-031 | 2-propenylamino | H | H | NO₂ | 3-F—Ph | Ph |
| III-032 | 2-phenylethylamino | H | H | Br | 3-F—Ph | Ph |
| III-033 | (3-ethoxy-3-oxopropyl)amino | H | H | Br | 3-F—Ph | Ph |
| III-034 | hexylamino | H | H | Br | 3-F—Ph | Ph |
| III-035 | cyclopropylmethylamino | H | H | Br | 3-F—Ph | Ph |
| III-036 | cyclopropylamino | H | H | Br | 3-F—Ph | Ph |
| III-037 | 2-propenylamino | H | H | Br | 3-F—Ph | Ph |
| III-038 | benzyloxyamino | H | H | I | 3-F—Ph | Ph |
| III-039 | pentylamino | H | H | NO₂ | 4-F—Ph | 3-F—Ph |
| III-040 | cyclopropylmethylamino | H | H | NO₂ | 4-F—Ph | 3-F—Ph |
| III-041 | cyclopropylamino | H | H | NO₂ | 4-F—Ph | 3-F—Ph |
| III-042 | prop-2-yn-1-ylamino | H | H | NO₂ | 4-F—Ph | 3-F—Ph |
| III-043 | 2-propenylamino | H | H | NO₂ | 4-F—Ph | 3-F—Ph |
| III-044 | 3,3,3-trifluoropropylamino | H | H | Br | 3-F—Ph | 3-F—Ph |
| III-045 | 2-propenylamino | Me | H | Br | Ph | 4-F—Ph |
| III-046 | propylamino | Me | H | Br | Ph | 4-F—Ph |
| III-047 | cyclopropylmethylamino | Me | H | Br | Ph | 4-F—Ph |
| III-048 | 2-methylpropylamino | Me | H | Br | Ph | 4-F—Ph |
| III-049 | 2-propenylamino | Me | H | Cl | Ph | 4-F—Ph |
| III-050 | propylamino | Me | H | Cl | Ph | 4-F—Ph |
| III-051 | cyclopropylmethylamino | Me | H | Cl | Ph | 4-F—Ph |
| III-052 | 2-methylpropylamino | Me | H | Cl | Ph | 4-F—Ph |
| III-053 | 2-propenylamino | H | H | Br | 2-F—Ph | 3,4-diF—Ph |
| III-054 | 2-propenylamino | H | H | Br | 2,6-diF—Ph | 3,4-diF—Ph |
| III-055 | [3-(methoxycarbonyl)cyclopentyl]amino | Me | H | Br | Ph | 4-F—Ph |
| III-056 | (2-methoxy-2-oxoethyl)amino | Me | H | Br | Ph | 4-F—Ph |
| III-057 | (3-methoxy-3-oxopropyl)amino | Me | H | Br | Ph | 4-F—Ph |
| III-058 | (2-methoxy-2-oxoethyl)amino | Me | H | Cl | Ph | 4-F—Ph |
| III-059 | [3-(methoxycarbonyl)cyclopentyl]amino | Me | H | Cl | Ph | 4-F—Ph |
| III-060 | (3-methoxy-3-oxopropyl)amino | Me | H | Cl | Ph | 4-F—Ph |
| III-061 | (3-methoxy-3-oxopropyl)amino | Me | Me | Br | Ph | 4-F—Ph |
| III-062 | [3-(methoxycarbonyl)cyclopentyl]amino | Me | Me | Br | Ph | 4-F—Ph |
| III-063 | 2-propenylamino | Me | Me | Br | Ph | 4-F—Ph |
| III-064 | propylamino | Me | Me | Br | Ph | 4-F—Ph |
| III-065 | (2-methoxy-2-oxoethyl)amino | Me | Me | Br | Ph | 4-F—Ph |
| III-066 | 2-propenylamino | H | H | NO₂ | 2-F—Ph | 3,4-diF—Ph |
| III-067 | 2-propenylamino | H | H | Cl | 2-F—Ph | 3,4-diF—Ph |
| III-068 | 2-propenylamino | H | H | Cl | 2,6-diF—Ph | 3,4-diF—Ph |
| III-069 | 2-propenylamino | H | H | NO₂ | 2,6-diF—Ph | 3,4-diF—Ph |

A further aspect of the invention relates to the preparation of the compounds of the general formula (I) according to the invention. The compounds according to the invention can be prepared in various ways.

Compounds according to the invention can be prepared, for example, by the synthesis method shown in Scheme 1 below from substituted 1,5-diphenyl-1H-pyrazol-3-ols or 1-phenyl-5-thienyl-1H-pyrazol-3-ols (II).

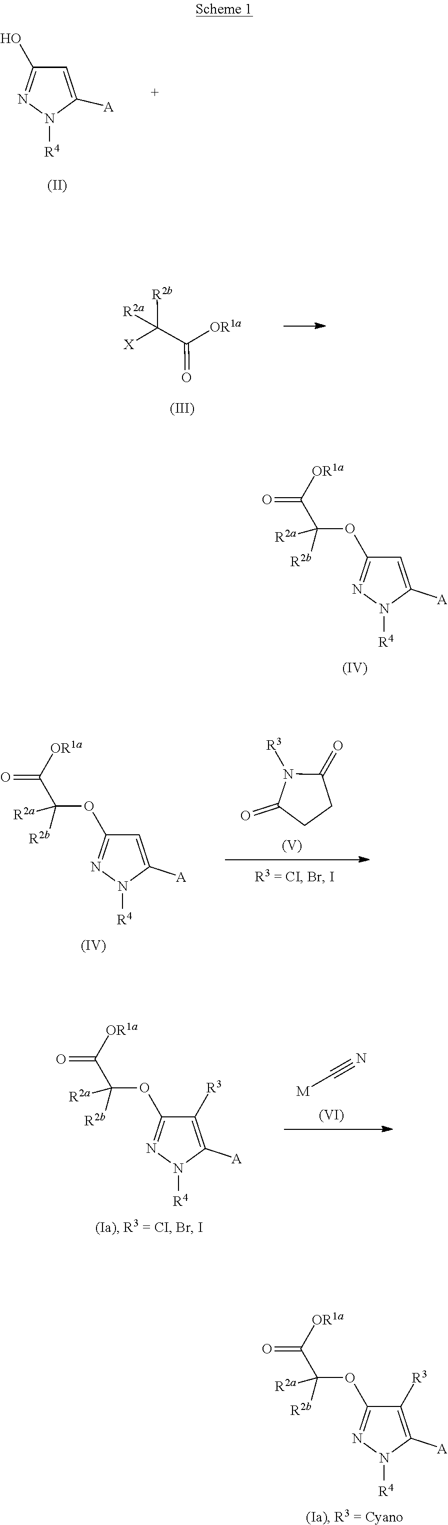

Scheme 1 describes the synthesis of the compound of the general formula (Ia, R³═Cl, Br, I) by reaction of a substituted pyrazole of the general formula (IV) with an electrophilic halogenating reagent of the general formula V, for example N-chlorosuccinimide (V, R³═Cl), N-bromosuccinimide (V, R³═Br) or N-iodosuccinimide (V, R³═I). In an analogous manner, it is also possible to use other electrophilic reagents, for example electrophilic nitrating reagents such as nitrating acid, nitronium tetrafluoroborate or ammonium nitrate/trifluoroacetic acid (when R³═NO₂) or electrophilic fluorinating reagents, such as DAST, Selectfluor or N-fluorobenzenesulfonimide (when R³═F). The reaction preferably takes place within the temperature range between 0° C. and 120° C. in an appropriate solvent, for example N,N-dimethylformamide, 1,2-dichloroethane or acetonitrile.

A compound of the general formula (Ib; R³═CN) can be prepared, for example, by reaction of a compound of the formula (Ia; R³═Cl, Br, I, preferably R³═Br, I) in a suitable solvent with a metal cyanide M-CN (VI with addition of a suitable amount of a transition metal catalyst, especially palladium catalysts such as palladium(0)tetrakis(triphenylphosphine) or palladium diacetate or bis(triphenylphosphine)palladium(II) dichloride or nickel catalysts such as nickel(II) acetylacetonate or bis(triphenylphosphine)nickel(II) chloride, preferably at elevated temperature in an organic solvent, for example 1,2-dimethoxyethane or N,N-dimethylformamide. The “M” radical represents, for example, magnesium, zinc, lithium or sodium. Cross-coupling methods that are suitable in general are those described in R. D. Larsen, Organometallics in Process Chemistry 2004 Springer Verlag, in I. Tsuji, Palladium Reagents and Catalysts 2004 Wiley, and in M. Belier, C. Bolm, Transition Metals for Organic Synthesis 2004 VCH-Wiley. Further suitable synthesis methods are described in Chem. Rev. 2006, 106, 2651; Platinum Metals Review, 2009, 53, 183; Platinum Metals Review 2008, 52, 172 and Acc. Chem. Res. 2008, 41, 1486.

The compound of the general formula (IV) can be prepared by alkylation of 3-hydroxypyrazoles of the general formula (II) with a halide of the general formula (III) in the presence of a base, by or analogously to methods known to the person skilled in the art. Preferably, the base is a carbonate salt of an alkali metal selected from the group consisting of lithium, sodium, potassium and caesium. The reaction preferably takes place within the temperature range between room temperature and 150° C. in an appropriate solvent, for example dichloromethane, acetonitrile, N,N-dimethylformamide or ethyl acetate. See J. Med. Chem. 2011, 54(16), 5820-5835 and WO2010/010154. The “X” radical represents, for example, chlorine, bromine or iodine.

The 3-hydroxypyrazoles (II) can be prepared, for example, analogously to methods known from the literature in two stages from substituted 3-phenyl- or thienylpropionic acid derivatives of the general formula (VII) (Scheme 2; see, for example: Adv. Synth. Catal. 2014, 356, 3135-3147).

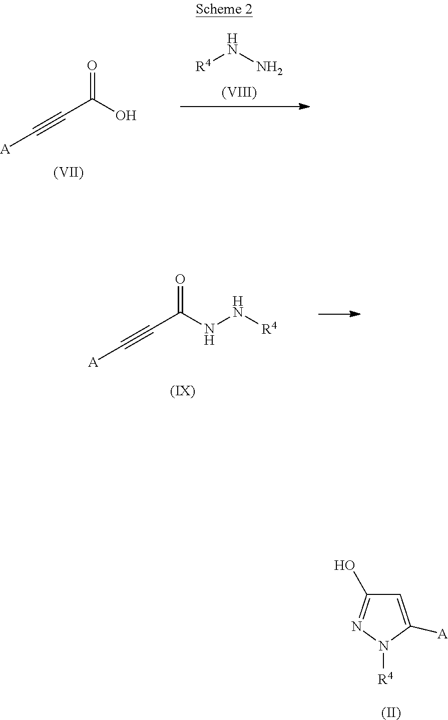

In the first step (Scheme 2), the compounds of the general formula (IX) are synthesized via an amide coupling of a substituted propionic acid of the general formula (VII) with an arylhydrazine of the general formula (VIII) in the presence of an amide coupling reagent, for example propanephosphonic anhydride (T3P), dicyclohexylcarbodiimide, N-(3-dimethylaminopropyl)-N′-ethylcarbodiimide, N,N′-carbonyldiimidazole, 2-chloro-1,3-dimethylimidazolium chloride or 2-chloro-1-methylpyridinium iodide (see Chemistry of Peptide Synthesis, Ed. N. Leo Benoiton, Taylor & Francis, 2006, ISBN-10: 1-57444-454-9). Polymer-bound reagents, for example polymer-bound dicyclohexylcarbodiimide, are also suitable for this coupling reaction. The reaction takes place preferably within the temperature range between 0° C. and 80° C., in a suitable solvent, for example dichloromethane, tetrahydrofuran, acetonitrile, N,N-dimethylformamide or ethyl acetate, and in the presence of a base, for example triethylamine, N,N-diisopropylethylamine or 1,8-diazabicyclo[5.4.0]undec-7-ene (see Scheme 2). For T3P peptide coupling conditions see Organic Process Research & Development 2009, 13, 900-906.

In the second step (Scheme 2), compounds of the general formula (IX) are cyclized in the presence of a couple halide, for example, copper(I) iodide, copper(I) bromide, or of a base such as sodium methoxide, or of an acid such as methanesulfonic acid, to give 3-hydroxypyrazoles of the general formula (II). The reaction preferably takes place in the temperature range between 0° C. and 120° C. in a suitable solvent such as 1,2-dichloroethane, acetonitrile, N,N-dimethylformamide, n-propanol, n-butanol or ethyl acetate.

The 3-hydroxypyrazoles (II) may also be prepared, for example, from protected 3-hydroxypyrazoles of the general formula (X). The protecting group R here is preferably a benzyl group or a trialkylsilyl group.

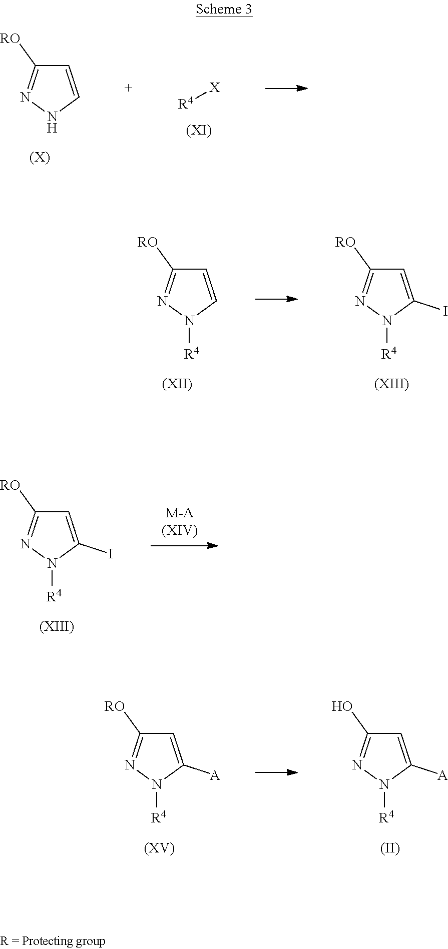

In the first step of Scheme 3, compounds of the general formula (XII) are prepared by an N-arylation of protected 3-hydroxypyrazoles of the general formula (X) with an aryl halide of the general formula (XI) in the presence of a copper halide, for example copper(I) iodide. The reaction takes place preferably within the temperature range between 0° C. and 120° C., in an appropriate solvent, for example acetonitrile or N,N-dimethylformamide, and in the presence of a base, for example triethylamine or caesium carbonate. The compounds of the general formula (XII) can be prepared according to methods analogously known to the person skilled in the art (e.g. Chem. Med. Chem. 2015, 10, 1184-1199). The “X” radical in compounds of the general formula (XI) is, for example, chlorine, bromine or iodine.

In the second step, 5-iodopyrazoles of the general formula (XIII) are prepared from compounds of the general formula (XII). The reaction is effected in the presence of a strong base, for example n-butyllithium or lithium diisopropylamide, and iodine. The reaction preferably takes place within a temperature range between −78° C. and −60° C., in an appropriate solvent, for example diethyl ether or tetrahydrofuran.

A compound of the formula (XV) can be prepared, for example, by reaction of a compound of the formula (XIII) in a suitable solvent with a compound M-A (XIV) with addition of an appropriate amount of a transition metal catalyst, especially a palladium catalyst such as palladium diacetate or bis(triphenylphosphine)palladium(II) dichloride, or a nickel catalyst such as nickel(II) acetylacetonate or bis(triphenylphosphine)nickel(II) chloride, preferably at elevated temperature in an organic solvent such as 1,2-dimethoxyethane. The “M” radical represents, for example, B(OR^{b})(OR^{c}), where the R^{b} and R^{c} radicals are independently, for example, hydrogen or (C₁-C₄)-alkyl, or, if the radicals R^{b} and R^{c} are bonded to one another, together are ethylene or propylene. Deprotecting a compound of the formula (XV) by standard methods that are well known to the person skilled in the art finally gives 3-hydroxypyrazoles of the general formula (II) which can be converted further, for example as described in Scheme 1, to the compounds according to the invention.

Compounds according to the invention can also be prepared, for example, by the synthesis method shown in Scheme 4 below from substituted 5-amino-1-phenyl-1H-pyrazol-3-ols of the general formula (XVI).

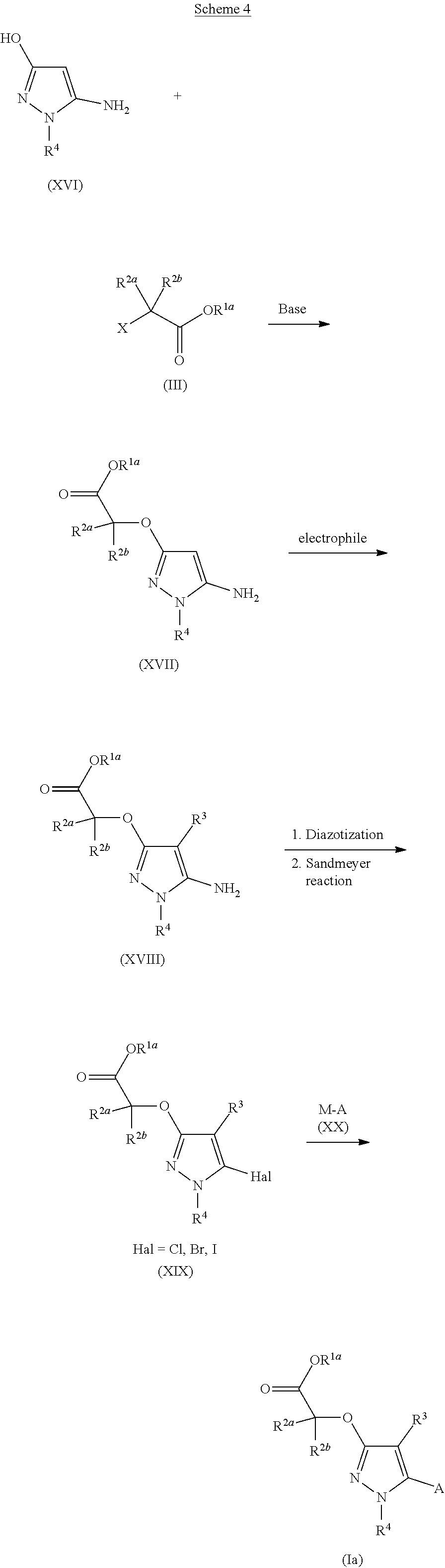

Scheme 4 describes the synthesis of compounds of the formula (Ia) by or analogously to methods known to the person skilled in the art, by reaction of a compound of the general formula (XIX) in which Hal is preferably bromine or iodine, more preferably iodine, with a compound M-A (XX) with addition of an appropriate amount of a transition metal catalyst, especially a palladium catalyst such as palladium diacetate or bis(triphenylphosphine)palladium(II) dichloride, or a nickel catalyst such as nickel(II) acetylacetonate or bis(triphenylphosphine)nickel(II) chloride, preferably at elevated temperature in an organic solvent such as 1,2-dimethoxyethane or dioxane. The “M” radical represents, for example, Mg-Hal, Zn-Hal, Sn((C₁-C₄)-alkyl)₃, lithium, copper or B(OR^{b})(OR^{c}), where the R^{b} and R^{c} radicals are independently, for example, hydrogen, (C₁-C₄)-alkyl, or, if the radicals R^{b} and R^{c} are bonded to one another, together are ethylene or propylene.

Compounds of the general formula (XIX) can be prepared by diazotization and subsequent Sandmeyer reaction of 5-aminopyrazoles of the general formula (XVIII) with the customary organic and inorganic nitrites, for example 1,1-dimethylethyl nitrite, tert-butyl nitrite or isoamyl nitrite, in the presence of usable reagents, for example mixtures of copper(I) and copper(II) bromide/chloride, iodine or diiodomethane (Scheme 4). The reaction preferably takes place within the temperature range between 0° C. and 120° C. in an appropriate solvent, for example dichloromethane, acetonitrile or N,N-dimethylformamide.

The compound of the general formula (XVIII) is synthesized by or analogously to methods known to the person skilled in the art, by reaction of a substituted pyrazole of the general formula (XVII) with an electrophilic reagent, for example an electrophilic halogenating reagent such as N-chlorosuccinimide (when R³═Cl), N-bromosuccinimide (when R³═Br), N-iodosuccinimide (when R³═I), or an electrophilic nitrating reagent such as nitrating acid, nitronium tetrafluoroborate, ammonium nitrate/trifluoroacetic acid (when R³═NO₂), or an electrophilic fluorinating reagent, such as DAST, Selectfluor, N-fluorobenzenesulfonimide (when R³═F). The reaction preferably takes place within the temperature range between 0° C. and 120° C. in an appropriate solvent, for example N,N-dimethylformamide, 1,2-dichloroethane or acetonitrile. A compound of the general formula (XVIII; with R³═CN) can be prepared, for example, by reaction of a compound of the formula (XVIII; with R³=halogen, preferably R³═Br, I) in a suitable solvent with a metal cyanide, for example zinc cyanide, with addition of a suitable amount of a transition metal catalyst, especially palladium catalysts such as palladium(0)tetrakis(triphenylphosphine) or palladium diacetate or bis(triphenylphosphine)palladium(II) dichloride or nickel catalysts such as nickel(II) acetylacetonate or bis(triphenylphosphine)nickel(II) chloride, preferably at elevated temperature in an organic solvent, for example 1,2-dimethoxyethane or N,N-dimethylformamide. Cross-coupling methods that are suitable in general are those described in R. D. Larsen, Organometallics in Process Chemistry 2004 Springer Verlag, in I. Tsuji, Palladium Reagents and Catalysts 2004 Wiley, and in M. Belier, C. Bolm, Transition Metals for Organic Synthesis 2004 VCH-Wiley. Further suitable synthesis methods are described in Chem. Rev. 2006, 106, 2651; Platinum Metals Review, 2009, 53, 183; Platinum Metals Review 2008, 52, 172 and Acc. Chem. Res. 2008, 41, 1486.

The compound of the general formula (XVII) can be synthesized by alkylation of substituted 5-amino-1-phenyl-1H-pyrazol-3-ols of the general formula (XVI) with a halide of the general formula (III) in the presence of a base, by or analogously to methods known to the person skilled in the art (see Scheme 4). The base may be a carbonate salt of an alkali metal (for example lithium, sodium, potassium or caesium). The reaction preferably takes place within the temperature range between room temperature and 150° C. in an appropriate solvent, for example dichloromethane, acetonitrile, N,N-dimethylformamide or ethyl acetate. The “X” radical in the compound of the general formula (III) is, for example, chlorine, bromine or iodine. The compounds of the general formula (XVI) are commercially available or known from the literature.

The above-described compounds of the general formula (Ia) can be used to prepare, by the standard methods that are well known to the person skilled in the art, inventive compounds of the general formula (Ic) with R^{1a}═H, and (Id) with R¹═NR⁹R¹⁰.

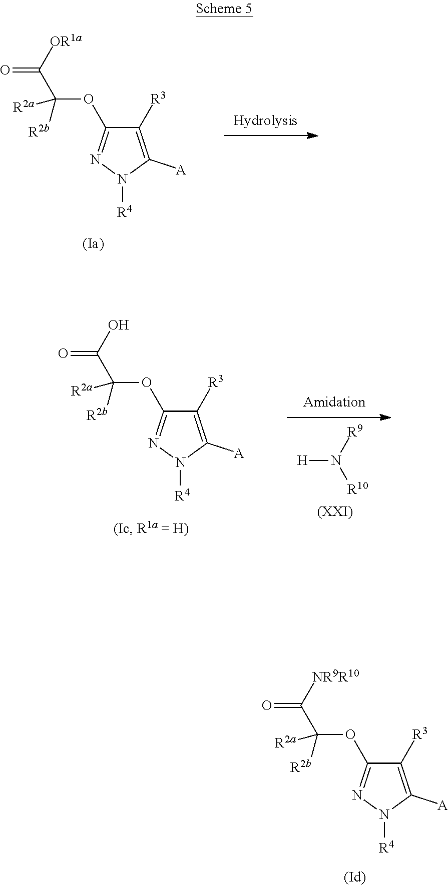

As shown in Scheme 5, an acid of the general formula (Ic) can be prepared by hydrolysis of an ester of the general formula (Ia), by or analogously to methods known to the person skilled in the art. The hydrolysis can be carried out in the presence of a base or a Lewis acid. The base may be a hydroxide salt of an alkali metal (for example lithium, sodium or potassium), and the hydrolysis reaction preferably takes place within the temperature range between room temperature and 120° C.

The inventive compounds of the general formula (Id) are synthesized, for example, via an amide coupling of an acid of the general formula (Ic) with an amine of the general formula (XXI) in the presence of an amide coupling reagent, for example propanephosphonic anhydride (T3P), dicyclohexylcarbodiimide, N-(3-dimethylaminopropyl)-N′-ethylcarbodiimide, N,N′-carbonyldiimidazole, 2-chloro-1,3-dimethylimidazolium chloride or 2-chloro-1-methylpyridinium iodide (see Chemistry of Peptide Synthesis, Ed. N. Leo Benoiton, Taylor & Francis, 2006, ISBN-10: 1-57444-454-9). Polymer-bound reagents, for example polymer-bound dicyclohexylcarbodiimide, are also suitable for this coupling reaction. The reaction takes place preferably within the temperature range between 0° C. and 80° C., in a suitable solvent, for example dichloromethane, acetonitrile, N,N-dimethylformamide or ethyl acetate, and in the presence of a base, for example triethylamine, N,N-diisopropylethylamine or 1,8-diazabicyclo[5.4.0]undec-7-ene. For T3P peptide coupling conditions see Organic Process Research & Development 2009, 13, 900-906.

The inventive compounds of the formula (I) (and/or salts thereof), referred to collectively as “compounds of the invention” hereinafter, have excellent herbicidal efficacy against a broad spectrum of economically important monocotyledonous and dicotyledonous annual harmful plants.

The present invention therefore also provides a method for controlling unwanted plants or for regulating the growth of plants, preferably in plant crops, in which one or more compound(s) of the invention is/are applied to the plants (for example harmful plants such as monocotyledonous or dicotyledonous weeds or unwanted crop plants), the seed (for example grains, seeds or vegetative propagules such as tubers or shoot parts with buds) or the area on which the plants grow (for example the area under cultivation). The compounds of the invention can be deployed, for example, prior to sowing (if appropriate also by incorporation into the soil), prior to emergence or after emergence. Specific examples of some representatives of the monocotyledonous and dicotyledonous weed flora which can be controlled by the compounds of the invention are as follows, though the enumeration is not intended to impose a restriction to particular species.

Monocotyledonous harmful plants of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Dicotyledonous weeds of the genera: Abutilon, Amaranthus, Ambrosia, Anoda, Anthemis, Aphanes, Artemisia, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

When the compounds of the invention are applied to the soil surface before germination, either the weed seedlings are prevented completely from emerging or the weeds grow until they have reached the cotyledon stage, but then stop growing.

If the active ingredients are applied post-emergence to the green parts of the plants, growth stops after the treatment, and the harmful plants remain at the growth stage at the time of application, or they die completely after a certain time, so that in this manner competition by the weeds, which is harmful to the crop plants, is eliminated very early and in a sustained manner.

The compounds of the invention can be selective in crops of useful plants and can also be employed as non-selective herbicides.

By virtue of their herbicidal and plant growth regulatory properties, the active ingredients can also be used to control harmful plants in crops of genetically modified plants which are known or are yet to be developed. In general, the transgenic plants are characterized by particular advantageous properties, for example by resistances to certain active ingredients used in the agrochemical industry, in particular certain herbicides, resistances to plant diseases or pathogens of plant diseases, such as certain insects or microorganisms such as fungi, bacteria or viruses. Other specific characteristics relate, for example, to the harvested material with regard to quantity, quality, storability, composition and specific constituents. For instance, there are known transgenic plants with an elevated starch content or altered starch quality, or those with a different fatty acid composition in the harvested material. Further particular properties lie in tolerance or resistance to abiotic stress factors, for example heat, cold, drought, salinity and ultraviolet radiation.

Preference is given to using the inventive compounds of the formula (I) or salts thereof in economically important transgenic crops of useful and ornamental plants.

The compounds of the formula (I) can be used as herbicides in crops of useful plants which are resistant, or have been made resistant by genetic engineering, to the phytotoxic effects of the herbicides.

Conventional ways of producing novel plants which have modified properties in comparison to existing plants consist, for example, in traditional cultivation methods and the generation of mutants. Alternatively, novel plants with altered properties can be generated with the aid of recombinant methods (see, for example, EP 0221044, EP 0131624). What has been described are, for example, several cases of genetic modifications of crop plants for the purpose of modifying the starch synthesized in the plants (e.g. WO 92/011376 A, WO 92/014827 A, WO 91/019806 A), transgenic crop plants which are resistant to certain herbicides of the glufosinate type (cf., for example, EP 0242236 A, EP 0242246 A) or of the glyphosate type (WO 92/000377 A) or of the sulfonylurea type (EP 0257993 A, U.S. Pat. No. 5,013,659) or to combinations or mixtures of these herbicides through “gene stacking”, such as transgenic crop plants, for example maize or soya with the trade name or the designation Optimum™ GAT™ (Glyphosate ALS Tolerant),
transgenic crop plants, for example cotton, capable of producing Bacillus thuringiensis toxins (Bt toxins), which make the plants resistant to particular pests (EP 0142924 A, EP 0193259 A),
transgenic crop plants having a modified fatty acid composition (WO 91/013972 A),
genetically modified crop plants having novel constituents or secondary metabolites, for example novel phytoalexins, which cause an increase in disease resistance (EP 0309862 A, EP 0464461 A)
genetically modified plants having reduced photorespiration, which have higher yields and higher stress tolerance (EP 0305398 A)
transgenic crop plants which produce pharmaceutically or diagnostically important proteins (“molecular pharming”)
transgenic crop plants which feature higher yields or better quality
transgenic crop plants which are distinguished by a combination, for example of the abovementioned novel properties (“gene stacking”).

Numerous molecular biology techniques which can be used to produce novel transgenic plants with modified properties are known in principle; see, for example, I. Potrykus and G. Spangenberg (eds), Gene Transfer to Plants, Springer Lab Manual (1995), Springer Verlag Berlin, Heidelberg or Christou, “Trends in Plant Science” 1 (1996) 423-431).

For such genetic manipulations, nucleic acid molecules which allow mutagenesis or sequence alteration by recombination of DNA sequences can be introduced into plasmids. With the aid of standard methods, it is possible, for example, to undertake base exchanges, remove part sequences or add natural or synthetic sequences. For the connection of the DNA fragments to one another, it is possible to add adapters or linkers to the fragments; see, for example, Sambrook et al., 1989, Molecular Cloning, A Laboratory Manual, 2nd ed., Cold Spring Harbor Laboratory Press, Cold Spring Harbor, N.Y.; or Winnacker “Gene und Klone” [Genes and Clones], VCH Weinheim, 2nd edition, 1996.

For example, the generation of plant cells with a reduced activity of a gene product can be achieved by expressing at least one corresponding antisense RNA, a sense RNA for achieving a cosuppression effect, or by expressing at least one suitably constructed ribozyme which specifically cleaves transcripts of the abovementioned gene product. To this end, it is firstly possible to use DNA molecules which encompass the entire coding sequence of a gene product inclusive of any flanking sequences which may be present, and also DNA molecules which only encompass portions of the coding sequence, in which case it is necessary for these portions to be long enough to have an antisense effect in the cells. It is also possible to use DNA sequences which have a high degree of homology to the coding sequences of a gene product, but are not completely identical to them.

When expressing nucleic acid molecules in plants, the protein synthesized may be localized in any desired compartment of the plant cell. However, to achieve localization in a particular compartment, it is possible, for example, to join the coding region to DNA sequences which ensure localization in a particular compartment. Such sequences are known to those skilled in the art (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106). The nucleic acid molecules can also be expressed in the organelles of the plant cells.

The transgenic plant cells can be regenerated by known techniques to give rise to entire plants. In principle, the transgenic plants may be plants of any desired plant species, i.e. not only monocotyledonous but also dicotyledonous plants. Obtainable in this way are transgenic plants having properties altered by overexpression, suppression or inhibition of homologous (=natural) genes or gene sequences or expression of heterologous (=foreign) genes or gene sequences.

The compounds (I) of the invention can be used with preference in transgenic crops which are resistant to growth regulators, for example 2,4-D, dicamba, or to herbicides which inhibit essential plant enzymes, for example acetolactate synthases (ALS), EPSP synthases, glutamine synthases (GS) or hydroxyphenylpyruvate dioxygenases (HPPD), or to herbicides from the group of the sulfonylureas, the glyphosates, glufosinates or benzoylisoxazoles and analogous active ingredients, or to any desired combinations of these active ingredients.

The compounds of the invention can be used with particular preference in transgenic crop plants which are resistant to a combination of glyphosates and glufosinates, glyphosates and sulfonylureas or imidazolinones. Most preferably, the compounds of the invention can be used in transgenic crop plants such as maize or soya with the trade name or the designation Optimum™ GAT™ (glyphosate ALS tolerant), for example.

When the active ingredients of the invention are employed in transgenic crops, not only do the effects towards harmful plants observed in other crops occur, but frequently also effects which are specific to the application in the particular transgenic crop, for example an altered or specifically widened spectrum of weeds which can be controlled, altered application rates which can be used for the application, preferably good combinability with the herbicides to which the transgenic crop is resistant, and influencing of growth and yield of the transgenic crop plants.

The invention therefore also relates to the use of the inventive compounds of the formula (I) as herbicides for controlling harmful plants in transgenic crop plants.

The compounds of the invention can be applied in the form of wettable powders, emulsifiable concentrates, sprayable solutions, dusting products or granules in the customary formulations. The invention therefore also provides herbicidal and plant-growth-regulating compositions which comprise the compounds of the invention.

The compounds of the invention can be formulated in various ways, according to the biological and/or physicochemical parameters required. Possible formulations include, for example: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), dispersions based on oil or water, oil-miscible solutions, capsule suspensions (CS), dusting products (DP), dressings, granules for scattering and soil application, granules (GR) in the form of microgranules, spray granules, absorption and adsorption granules, water-dispersible granules (WG), water-soluble granules (SG), ULV formulations, microcapsules and waxes. These individual formulation types are known in principle and are described, for example, in: Winnacker-Kuchler, “Chemische Technologie” [Chemical Technology], Volume 7, C. Hanser Verlag Munich, 4th Ed. 1986, Wade van Valkenburg, “Pesticide Formulations”, Marcel Dekker, N.Y., 1973, K. Martens, “Spray Drying” Handbook, 3rd Ed. 1979, G. Goodwin Ltd. London.

The necessary formulation auxiliaries such as inert materials, surfactants, solvents and further additives are likewise known and are described, for example, in: Watkins, “Handbook of Insecticide Dust Diluents and Carriers”, 2nd ed., Darland Books, Caldwell N.J., H. v. Olphen, “Introduction to Clay Colloid Chemistry”, 2nd ed., J. Wiley & Sons, N.Y., C. Marsden, “Solvents Guide”, 2nd ed., Interscience, N.Y. 1963, McCutcheon's “Detergents and Emulsifiers Annual”, MC Publ. Corp., Ridgewood N.J., Sisley and Wood, “Encyclopedia of Surface Active Agents”, Chem. Publ. Co. Inc., N.Y. 1964, Schönfeldt, “Grenzflächenaktive Äthylenoxidaddukte” [Interface-active Ethylene Oxide Adducts], Wiss. Verlagsgesell., Stuttgart 1976, Winnacker-Küchler, “Chemische Technologie”, volume 7, C. Hanser Verlag Munich, 4th ed. 1986.

On the basis of these formulations, it is also possible to produce combinations with other active ingredients, for example insecticides, acaricides, herbicides, fungicides, and also with safeners, fertilizers and/or growth regulators, for example in the form of a finished formulation or as a tank mix.

Combination partners usable for the compounds of the invention in mixed formulations or in a tankmix are, for example, known active ingredients based on inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoene desaturase, photosystem I, photosystem II or protoporphyrinogen oxidase, as known, for example, from Weed Research 26 (1986) 441-445 or “The Pesticide Manual”, 16th edition, The British Crop Protection Council and the Royal Soc. of Chemistry, 2006, and literature cited therein. Known herbicides or plant growth regulators which can be combined with the compounds of the invention are, for example, the following, where said active ingredients are referred to either by their “common name” in accordance with the International Organization for Standardization (ISO) or by the chemical name or by the code number. They always encompass all the use forms, for example acids, salts, esters and also all isomeric forms such as stereoisomers and optical isomers, even if they are not mentioned explicitly.

Examples of such herbicidal mixing partners are:

acetochlor, acifluorfen, acifluorfen-sodium, aclonifen, alachlor, allidochlor, alloxydim, alloxydim-sodium, ametryn, amicarbazone, amidochlor, amidosulfuron, 4-amino-3-chloro-5-fluoro-6-(7-fluoro-1H-indol-6-yl)pyridine-2-carboxylic acid, aminocyclopyrachlor, aminocyclopyrachlor-potassium, aminocyclopyrachlor-methyl, aminopyralid, amitrole, ammoniumsulfamate, anilofos, asulam, atrazine, azafenidin, azimsulfuron, beflubutamid, benazolin, benazolin-ethyl, benfluralin, benfuresate, bensulfuron, bensulfuron-methyl, bensulide, bentazone, benzobicyclon, benzofenap, bicyclopyron, bifenox, bilanafos, bilanafos-sodium, bispyribac, bispyribac-sodium, bixlozone, bromacil, bromobutide, bromofenoxim, bromoxynil, bromoxynil-butyrate, -potassium, -heptanoate and -octanoate, busoxinone, butachlor, butafenacil, butamifos, butenachlor, butralin, butroxydim, butylate, cafenstrole, carbetamide, carfentrazone, carfentrazone-ethyl, chloramben, chlorbromuron, 1-{2-chloro-3-[(3-cyclopropyl-5-hydroxy-1-methyl-1H-pyrazol-4-yl)carbonyl]-6-(trifluoromethyl)phenyl}piperidin-2-one, 4-{2-chloro-3-[(3,5-dimethyl-1H-pyrazol-1-yl)methyl]-4-(methylsulfonyl)benzoyl}-1,3-dimethyl-1H-pyrazol-5-yl-1,3-dimethyl-1H-pyrazole-4-carboxylate, chlorfenac, chlorfenac-sodium, chlorfenprop, chlorflurenol, chlorflurenol-methyl, chloridazon, chlorimuron, chlorimuron-ethyl, 2-[2-chloro-4-(methylsulfonyl)-3-(morpholin-4-ylmethyl)benzoyl]-3-hydroxycyclohex-2-en-1-one, 4-{2-chloro-4-(methylsulfonyl)-3-[(2,2,2-trifluoroethoxy)methyl]benzoyl}-1-ethyl-1H-pyrazol-5-yl 1,3-dimethyl-1H-pyrazole-4-carboxylate, chlorophthalim, chlorotoluron, chlorthal-dimethyl, chlorsulfuron, 3-[5-chloro-4-(trifluoromethyl)pyridin-2-yl]-4-hydroxy-1-methylimidazolidin-2-one, cinidon, cinidon-ethyl, cinmethylin, cinosulfuron, clacyfos, clethodim, clodinafop, clodinafop-propargyl, clomazone, clomeprop, clopyralid, cloransulam, cloransulam-methyl, cumyluron, cyanamide, cyanazine, cycloate, cyclopyranil, cyclopyrimorate, cyclosulfamuron, cycloxydim, cyhalofop, cyhalofop-butyl, cyprazine, 2,4-D, 2,4-D-butotyl, -butyl, -dimethylammonium, -diolamin, -ethyl, 2-ethylhexyl, -isobutyl, -isooctyl, -isopropylammonium, -potassium, -triisopropanolammonium and -trolamine, 2,4-DB, 2,4-DB-butyl, -dimethylammonium, isooctyl, -potassium and -sodium, daimuron (dymron), dalapon, dazomet, n-decanol, desmedipham, detosyl-pyrazolate (DTP), dicamba, dichlobenil, dichlorprop, dichlorprop-P, diclofop, diclofop-methyl, diclofop-P-methyl, diclosulam, difenzoquat, diflufenican, diflufenzopyr, diflufenzopyr-sodium, dimefuron, dimepiperate, dimethachlor, dimethametryn, dimethenamid, dimethenamid-P, 3-(2,6-dimethylphenyl)-6-[(2-hydroxy-6-oxocyclohex-1-en-1-yl)carbonyl]-1-methylquinazoline-2,4(1H,3H)-dione, 1,3-dimethyl-4-[2-(methylsulfonyl)-4-(trifluoromethyl)benzoyl]-1H-pyrazol-5-yl 1,3-dimethyl-1H-pyrazole-4-carboxylate, dimetrasulfuron, dinitramine, dinoterb, diphenamid, diquat, diquat-dibromid, dithiopyr, diuron, DMPA, DNOC, endothal, EPTC, esprocarb, ethalfluralin, ethametsulfuron, ethametsulfuron-methyl, ethiozin, ethofumesate, ethoxyfen, ethoxyfen-ethyl, ethoxysulfuron, etobenzanid, ethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, F-9960, F-5231, i.e. N-[2-chloro-4-fluoro-5-[4-(3-fluoropropyl)-4,5-dihydro-5-oxo-1H-tetrazol-1-yl]-phenyl]ethanesulfonamide, F-7967, i.e. 3-[7-chloro-5-fluoro-2-(trifluoromethyl)-1H-benzimidazol-4-yl]-1-methyl-6-(trifluoromethyl)pyrimidine-2,4(1H,3H)-dione, fenoxaprop, fenoxaprop-P, fenoxaprop-ethyl, fenoxaprop-P-ethyl, fenoxasulfone, fenquinotrione, fentrazamide, flamprop, flamprop-M-isopropyl, flamprop-M-methyl, flazasulfuron, florasulam, florpyrauxifen, florpyrauxifen-benzyl, fluazifop, fluazifop-P, fluazifop-butyl, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, flurenol, flurenol-butyl, -dimethylammonium and -methyl, fluoroglycofen, fluoroglycofen-ethyl, flupropanate, flupyrsulfuron, flupyrsulfuron-methyl-sodium, fluridone, flurochloridone, fluroxypyr, fluroxypyr-meptyl, flurtamone, fluthiacet, fluthiacet-methyl, fomesafen, fomesafen-sodium, foramsulfuron, fosamine, glufosinate, glufosinate-ammonium, glufosinate-P-sodium, glufosinate-P-ammonium, glufosinate-P-sodium, glyphosate, glyphosate-ammonium, -isopropylammonium, -diammonium, -dimethylammonium, -potassium, -sodium and -trimesium, H-9201, i.e. O-(2,4-dimethyl-6-nitrophenyl)O-ethyl isopropylphosphoramidothioate, halauxifen, halauxifen-methyl, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, hexazinone, HW-02, i.e. 1-(dimethoxyphosphoryl)ethyl (2,4-dichlorophenoxy)acetate, 4-hydroxy-1-methoxy-5-methyl-3-[4-(trifluoromethyl)pyridin-2-yl]imidazolidin-2-one, 4-hydroxy-1-methyl-3-[4-(trifluoromethyl)pyridin-2-yl]imidazolidin-2-one, (5-hydroxy-1-methyl-1H-pyrazol-4-yl)(3,3,4-trimethyl-1,1-dioxido-2,3-dihydro-1-benzothiophen-5-yl)methanone, 6-[(2-hydroxy-6-oxocyclohex-1-en-1-yl)carbonyl]-1,5-dimethyl-3-(2-methylphenyl)quinazoline-2,4(1H,3H)-dione, imazamethabenz, imazamethabenz-methyl, imazamox, imazamox-ammonium, imazapic, imazapic-ammonium, imazapyr, imazapyr-isopropylammonium, imazaquin, imazaquin-ammonium, imazethapyr, imazethapyr-immonium, imazosulfuron, indanofan, indaziflam, iodosulfuron, iodosulfuron-methyl-sodium, ioxynil, ioxynil-octanoate, -potassium and sodium, ipfencarbazone, isoproturon, isouron, isoxaben, isoxaflutole, karbutilate, KUH-043, i.e. 3-({[5-(difluoromethyl)-1-methyl-3-(trifluoromethyl)-1H-pyrazol-4-yl]methyl}sulfonyl)-5,5-dimethyl-4,5-dihydro-1,2-oxazole, ketospiradox, lactofen, lenacil, linuron, MCPA, MCPA-butotyl, -dimethylammonium, -2-ethylhexyl, -isopropylammonium, -potassium and -sodium, MCPB, MCPB-methyl, -ethyl and -sodium, mecoprop, mecoprop-sodium, and -butotyl, mecoprop-P, mecoprop-P-butotyl, -dimethylammonium, -2-ethylhexyl and -potassium, mefenacet, mefluidide, mesosulfuron, mesosulfuron-methyl, mesotrione, methabenzthiazuron, metam, metamifop, metamitron, metazachlor, metazosulfuron, methabenzthiazuron, methiopyrsulfuron, methiozolin, 2-({2-[(2-methoxyethoxy)methyl]-6-(trifluoromethyl)pyridin-3-yl}carbonyl)cyclohexane-1,3-dione, methyl isothiocyanate, 1-methyl-4-[(3,3,4-trimethyl-1,1-dioxido-2,3-dihydro-1-benzothiophen-5-yl)carbonyl]-1H-pyrazol-5-yl propane-1-sulfonate, metobromuron, metolachlor, S-metolachlor, metosulam, metoxuron, metribuzin, metsulfuron, metsulfuron-methyl, molinat, monolinuron, monosulfuron, monosulfuron-ester, MT-5950, i.e. N-[3-chloro-4-(1-methylethyl)phenyl]-2-methylpentanamide, NGGC-011, napropamide, NC-310, i.e. 4-(2,4-dichlorobenzoyl)-1-methyl-5-benzyloxypyrazole, neburon, nicosulfuron, nonanoic acid (pelargonic acid), norflurazon, oleic acid (fatty acids), orbencarb, orthosulfamuron, oryzalin, oxadiargyl, oxadiazon, oxasulfuron, oxaziclomefon, oxotrione (lancotrione), oxyfluorfen, paraquat, paraquat dichloride, pebulate, pendimethalin, penoxsulam, pentachlorphenol, pentoxazone, pethoxamid, petroleum oils, phenmedipham, picloram, picolinafen, pinoxaden, piperophos, pretilachlor, primisulfuron, primisulfuron-methyl, prodiamine, profoxydim, prometon, prometryn, propachlor, propanil, propaquizafop, propazine, propham, propisochlor, propoxycarbazone, propoxycarbazone-sodium, propyrisulfuron, propyzamide, prosulfocarb, prosulfuron, pyraclonil, pyraflufen, pyraflufen-ethyl, pyrasulfotole, pyrazolynate (pyrazolate), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-P, quizalofop-P-ethyl, quizalofop-P-tefuryl, QYM-201, QYR-301, rimsulfuron, saflufenacil, sethoxydim, siduron, simazine, simetryn, sulcotrion, sulfentrazone, sulfometuron, sulfometuron-methyl, sulfosulfuron, SYN-523, SYP-249, i.e. 1-ethoxy-3-methyl-1-oxobut-3-en-2-yl 5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxoimidazolidine-4,5-dione, 2,3,6-TBA, TCA (trifluoroacetic acid), TCA-sodium, tebuthiuron, tefuryltrione, tembotrione, tepraloxydim, terbacil, terbucarb, terbumeton, terbuthylazin, terbutryn, tetflupyrolimet, thenylchlor, thiazopyr, thiencarbazone, thiencarbazone-methyl, thifensulfuron, thifensulfuron-methyl, thiobencarb, tiafenacil, tolpyralate, topramezone, tralkoxydim, triafamone, tri-allate, triasulfuron, triaziflam, tribenuron, tribenuron-methyl, triclopyr, trietazine, trifloxysulfuron, trifloxysulfuron-sodium, trifludimoxazin, trifluralin, triflusulfuron, triflusulfuron-methyl, tritosulfuron, urea sulfate, vernolate, ZJ-0862, i.e. 3,4-dichloro-N-{2-[(4,6-dimethoxypyrimidin-2-yl)oxy]benzyl}aniline.

Examples of plant growth regulators as possible mixing partners are:

acibenzolar, acibenzolar-S-methyl, 5-aminolevulinic acid, ancymidol, 6-benzylaminopurine, brassinolide, catechol, chlormequat chloride, cloprop, cyclanilide, 3-(cycloprop-1-enyl)propionic acid, daminozide, dazomet, n-decanol, dikegulac, dikegulac-sodium, endothal, endothal-dipotassium, -disodium, and mono(N,N-dimethylalkylammonium), ethephon, flumetralin, flurenol, flurenol-butyl, flurprimidol, forchlorfenuron, gibberellic acid, inabenfide, indole-3-acetic acid (IAA), 4-indol-3-ylbutyric acid, isoprothiolane, probenazole, jasmonic acid, jasmonic acid methyl ester, maleic hydrazide, mepiquat chloride, 1-methylcyclopropene, 2-(1-naphthyl)acetamide, 1-naphthylacetic acid, 2-naphthyloxyacetic acid, nitrophenolate mixture, 4-oxo-4[(2-phenylethyl)amino]butyric acid, paclobutrazole, N-phenylphthalamic acid, prohexadione, prohexadione-calcium, prohydrojasmone, salicylic acid, strigolactone, tecnazene, thidiazuron, triacontanol, trinexapac, trinexapac-ethyl, tsitodef, uniconazole, uniconazole-P.

Safeners which can be used in combination with the inventive compounds of the formula (I) and optionally in combinations with further active compounds such as insecticides, acaricides, herbicides, fungicides as listed above are preferably selected from the group consisting of:

S1) Compounds of the formula (S1)

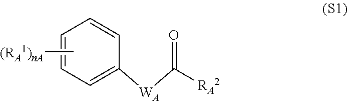

where the symbols and indices are defined as follows:

n_{A} is a natural number from 0 to 5, preferably from 0 to 3;

R_{A}¹ is halogen, (C₁-C₄)-alkyl, (C₁-C₄)-alkoxy, nitro or (C₁-C₄)-haloalkyl;

W_{A} is an unsubstituted or substituted divalent heterocyclic radical from the group of the partly unsaturated or aromatic five-membered heterocycles having 1 to 3 ring heteroatoms from the N and O group, where at least one nitrogen atom and at most one oxygen atom is present in the ring, preferably a radical from the group of (W_{A}¹) to (W_{A}⁴),

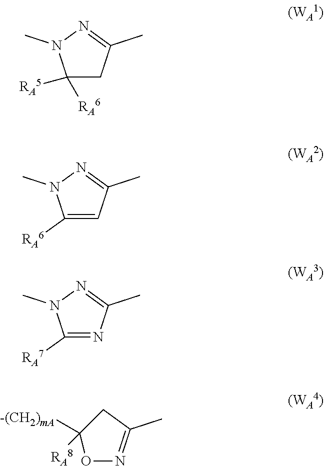

m_{A} is 0 or 1;

R_{A}² is OR_{A}³, SR_{A}³ or NR_{A}³R_{A}⁴ or a saturated or unsaturated 3- to 7-membered heterocycle having at least one nitrogen atom and up to 3 heteroatoms, preferably from the group consisting of O and S, which is joined to the carbonyl group in (S1) via the nitrogen atom and is unsubstituted or substituted by radicals from the group consisting of (C₁-C₄)-alkyl, (C₁-C₄)-alkoxy or optionally substituted phenyl, preferably a radical of the formula OR_{A}³, NHR_{A}⁴ or N(CH₃)₂, especially of the formula OR_{A}³;

R_{A}³ is hydrogen or an unsubstituted or substituted aliphatic hydrocarbon radical, preferably having a total of 1 to 18 carbon atoms;

R_{A}⁴ is hydrogen, (C₁-C₆)-alkyl, (C₁-C₆)-alkoxy or substituted or unsubstituted phenyl;

R_{A}⁵ is H, (C₁-C₈)-alkyl, (C₁-C₈)-haloalkyl, (C₁-C₄)-alkoxy-(C₁-C₈)-alkyl, cyano or COOR_{A}⁹, where R_{A}⁹ is hydrogen, (C₁-C₈)-alkyl, (C₁-C₈)-haloalkyl, (C₁-C₄)-alkoxy-(C₁-C₄)-alkyl, (C₁-C₆)-hydroxyalkyl, (C₃-C₁₂)-cycloalkyl or tri-(C₁-C₄)-alkylsilyl;

R_{A}⁶, R_{A}⁷, R_{A}⁸ are identical or different and are hydrogen, (C₁-C₈)-alkyl, (C₁-C₈)-haloalkyl, (C₃-C₁₂)-cycloalkyl or substituted or unsubstituted phenyl;

preferably:

a) compounds of the dichlorophenylpyrazoline-3-carboxylic acid type (S1^{a}), preferably compounds such as 1-(2,4-dichlorophenyl)-5-(ethoxycarbonyl)-5-methyl-2-pyrazoline-3-carboxylic acid, ethyl 1-(2,4-dichlorophenyl)-5-(ethoxycarbonyl)-5-methyl-2-pyrazoline-3-carboxylate (S1-1) (“mefenpyr-diethyl”), and related compounds as described in WO-A-91/07874;

b) derivatives of dichlorophenylpyrazolecarboxylic acid (S1^{b}), preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S1-4) and related compounds as described in EP-A-333 131 and EP-A-269 806;

c) derivatives of 1,5-diphenylpyrazole-3-carboxylic acid (S1^{c}), preferably compounds such as ethyl 1-(2,4-dichlorophenyl)-5-phenylpyrazole-3-carboxylate (S1-5), methyl 1-(2-chlorophenyl)-5-phenylpyrazole-3-carboxylate (S1-6) and related compounds as described in EP-A-268 554, for example;

d) compounds of the triazolecarboxylic acid type (S1^{d}), preferably compounds such as fenchlorazole(-ethyl ester), i.e. ethyl 1-(2,4-dichlorophenyl)-5-trichloromethyl-(1H)-1,2,4-triazole-3-carboxylate (S1-7), and related compounds as described in EP-A-174 562 and EP-A-346 620;

e) compounds of the 5-benzyl- or 5-phenyl-2-isoxazoline-3-carboxylic acid or of the 5,5-diphenyl-2-isoxazoline-3-carboxylic acid type (S1^{e}), preferably compounds such as ethyl 5-(2,4-dichlorobenzyl)-2-isoxazoline-3-carboxylate (S1-8) or ethyl 5-phenyl-2-isoxazoline-3-carboxylate (S1-9) and related compounds as described in WO-A-91/08202, or 5,5-diphenyl-2-isoxazoline-3-carboxylic acid (S1-10) or ethyl 5,5-diphenyl-2-isoxazoline-3-carboxylate (S1-11) (“isoxadifen-ethyl”) or n-propyl 5,5-diphenyl-2-isoxazoline-3-carboxylate (S1-12) or ethyl 5-(4-fluorophenyl)-5-phenyl-2-isoxazoline-3-carboxylate (S1-13), as described in patent application WO-A-95/07897.

S2) Quinoline derivatives of the formula (S2)

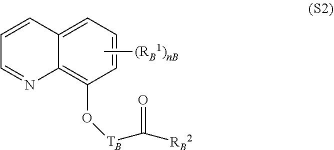

where the symbols and indices have the meanings below:

R_{A}¹ is halogen, (C₁-C₄)-alkyl, (C₁-C₄)-alkoxy, nitro or (C₁-C₄)-haloalkyl;

n_{B} is a natural number from 0 to 5, preferably from 0 to 3;

R_{B}² is OR_{B}³, SR_{B}³ or NR_{B}³R_{B}⁴ or a saturated

or unsaturated 3- to 7-membered heterocycle having at least one nitrogen atom and up to 3 heteroatoms, preferably from the group of O and S, which is joined via the nitrogen atom to the carbonyl group in (S2) and is unsubstituted or substituted by radicals from the group of (C₁-C₄)-alkyl, (C₁-C₄)-alkoxy or optionally substituted phenyl, preferably a radical of the formula OR_{B}³, NHR_{B}⁴ or N(CH₃)₂, especially of the formula OR_{B}³;

R_{B}³ is hydrogen or an unsubstituted or substituted aliphatic hydrocarbon radical, preferably having a total of 1 to 18 carbon atoms;

R_{B}⁴ is hydrogen, (C₁-C₆)-alkyl, (C₁-C₆)-alkoxy or substituted or unsubstituted phenyl;

T_{B} is a (C₁ or C₂)-alkanediyl chain which is unsubstituted or substituted by one or two (C₁-C₄)-alkyl radicals or by [(C₁-C₃)-alkoxy]carbonyl;

preferably:

a) compounds of the 8-quinolinoxyacetic acid type (S2^{a}), preferably
1-methylhexyl (5-chloro-8-quinolinoxy)acetate (“cloquintocet-mexyl”) (S2-1),
(1,3-dimethylbut-1-yl) (5-chloro-8-quinolinoxy)acetate (S2-2),
4-allyloxybutyl (5-chloro-8-quinolinoxy)acetate (S2-3),
1-allyloxyprop-2-yl (5-chloro-8-quinolinoxy)acetate (S2-4),
ethyl (5-chloro-8-quinolinoxy)acetate (S2-5),
methyl (5-chloro-8-quinolinoxy)acetate (S2-6),
allyl (5-chloro-8-quinolinoxy)acetate (S2-7),
2-(2-propylideneiminoxy)-1-ethyl (5-chloro-8-quinolinoxy)acetate (S2-8), 2-oxoprop-1-yl (5-chloro-8-quinolinoxy)acetate (S2-9) and related compounds, as described in EP-A-86 750, EP-A-94 349 and EP-A-191 736 or EP-A-0 492 366, and also (5-chloro-8-quinolinoxy)acetic acid (S2-10), hydrates and salts thereof, for example the lithium, sodium, potassium, calcium, magnesium, aluminium, iron, ammonium, quaternary ammonium, sulfonium or phosphonium salts thereof, as described in WO-A-2002/34048;

b) compounds of the (5-chloro-8-quinolinoxy)malonic acid type (S2^{b}), preferably compounds such as diethyl (5-chloro-8-quinolinoxy)malonate, diallyl (5-chloro-8-quinolinoxy)malonate, methyl ethyl (5-chloro-8-quinolinoxy)malonate and related compounds, as described in EP-A-0 582 198.

S3) Compounds of the formula (S3)

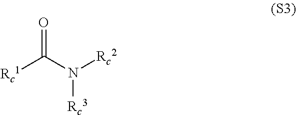

where the symbols and indices are defined as follows:

R_{C}¹ is (C₁-C₄)-alkyl, (C₁-C₄)-haloalkyl, (C₂-C₄)-alkenyl, (C₂-C₄)-haloalkenyl, (C₃-C₇)-cycloalkyl, preferably dichloromethyl;

R_{C}², R_{C}³ are identical or different and are hydrogen, (C₁-C₄)alkyl, (C₂-C₄)alkenyl, (C₂-C₄)alkynyl, (C₁-C₄)haloalkyl, (C₂-C₄)haloalkenyl, (C₁-C₄)alkylcarbamoyl-(C₁-C₄)alkyl, (C₂-C₄)alkenylcarbamoyl-(C₁-C₄)alkyl, (C₁-C₄)alkoxy-(C₁-C₄)alkyl, dioxolanyl-(C₁-C₄)alkyl, thiazolyl, furyl, furylalkyl, thienyl, piperidyl, substituted or unsubstituted phenyl, or R_{C}² and R_{C}³ together form a substituted or unsubstituted heterocyclic ring, preferably an oxazolidine, thiazolidine, piperidine, morpholine, hexahydropyrimidine or benzoxazine ring;

preferably:
active compounds of the dichloroacetamide type, which are frequently used as pre-emergence safeners (soil-acting safeners), for example
“dichlormid” (N,N-diallyl-2,2-dichloroacetamide) (S3-1),
“R-29148” (3-dichloroacetyl-2,2,5-trimethyl-1,3-oxazolidine) from Stauffer (S3-2),
“R-28725” (3-dichloroacetyl-2,2-dimethyl-1,3-oxazolidine) from Stauffer (S3-3),
“benoxacor” (4-dichloroacetyl-3,4-dihydro-3-methyl-2H-1,4-benzoxazine) (S3-4),
“PPG-1292” (N-allyl-N-[(1,3-dioxolan-2-yl)methyl]dichloroacetamide) from PPG Industries (S3-5),
“DKA-24” (N-allyl-N-[(allylaminocarbonyl)methyl]dichloroacetamide) from Sagro-Chem (53-6),
“AD-67” or “MON 4660” (3-dichloroacetyl-1-oxa-3-azaspiro[4.5]decane) from Nitrokemia or Monsanto (53-7),
“TI-35” (1-dichloroacetylazepane) from TRI-Chemical RT (53-8),
“diclonon” (dicyclonon) or “BAS145138” or “LAB145138” (53-9)
((RS)-1-dichloroacetyl-3,3,8a-trimethylperhydropyrrolo[1,2-a]pyrimidin-6-one) from BASF,
“furilazole” or “MON 13900” ((RS)-3-dichloroacetyl-5-(2-furyl)-2,2-dimethyloxazolidine) (53-10); and the (R) isomer thereof (S3-11).

S4) N-acylsulfonamides of the formula (S4) and salts thereof,

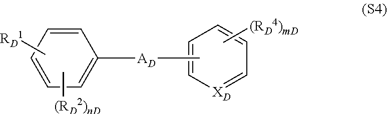

in which the symbols and indices are defined as follows:

A_{D} is SO₂—NR_{D}³—CO or CO—NR_{D}³—SO₂

X_{D} is CH or N;

R_{D}¹ is CO—NR_{D}⁵R_{D}⁶ or NHCO—R_{D}⁷;

R_{D}² is halogen, (C₁-C₄)-haloalkyl, (C₁-C₄)-haloalkoxy, nitro, (C₁-C₄)-alkyl, (C₁-C₄)-alkoxy, (C₁-C₄)-alkylsulfonyl, (C₁-C₄)-alkoxycarbonyl or (C₁-C₄)-alkylcarbonyl;

R_{D}³ is hydrogen, (C₁-C₄)-alkyl, (C₂-C₄)-alkenyl or (C₂-C₄)-alkynyl;

R_{D}⁴ is halogen, nitro, (C₁-C₄)-alkyl, (C₁-C₄)-haloalkyl, (C₁-C₄)-haloalkoxy, (C₃-C₆)-cycloalkyl, phenyl, (C₁-C₄)-alkoxy, cyano, (C₁-C₄)-alkylthio, (C₁-C₄)-alkylsulfinyl, (C₁-C₄)-alkylsulfonyl, (C₁-C₄)-alkoxycarbonyl or (C₁-C₄)-alkylcarbonyl;

R_{D}⁵ is hydrogen, (C₁-C₆)-alkyl, (C₃-C₆)-cycloalkyl, (C₂-C₆)-alkenyl, (C₂-C₆)-alkynyl, (C₅-C₆)-cycloalkenyl, phenyl or 3- to 6-membered heterocyclyl containing v_{D} heteroatoms from the group consisting of nitrogen, oxygen and sulfur, where the seven latter radicals are substituted by v_{D} substituents from the group consisting of halogen, (C₁-C₆)-alkoxy, (C₁-C₆)-haloalkoxy, (C₁-C₂)-alkylsulfinyl, (C₁-C₂)-alkylsulfonyl, (C₃-C₆)-cycloalkyl, (C₁-C₄)-alkoxycarbonyl, (C₁-C₄)-alkylcarbonyl and phenyl and, in the case of cyclic radicals, also (C₁-C₄)-alkyl and (C₁-C₄)-haloalkyl;

R_{D}⁶ is hydrogen, (C₁-C₆)-alkyl, (C₂-C₆)-alkenyl or (C₂-C₆)-alkynyl, where the three latter radicals are substituted by v_{D} radicals from the group consisting of halogen, hydroxyl, (C₁-C₄)-alkyl, (C₁-C₄)-alkoxy and (C₁-C₄)-alkylthio, or

R_{D}⁵ and R_{D}⁶ together with the nitrogen atom carrying them form a pyrrolidinyl or piperidinyl radical;

R_{D}⁷ is hydrogen, (C₁-C₄)-alkylamino, di-(C₁-C₄)-alkylamino, (C₁-C₆)-alkyl, (C₃-C₆)-cycloalkyl, where the 2 latter radicals are substituted by v_{D} substituents from the group consisting of halogen, (C₁-C₄)-alkoxy, (C₁-C₆)-haloalkoxy and (C₁-C₄)-alkylthio and, in the case of cyclic radicals, also (C₁-C₄)-alkyl and (C₁-C₄)-haloalkyl;

n_{D} is 0, 1 or 2;

m_{D} is 1 or 2;

v_{D} is 0, 1, 2 or 3;

among these, preference is given to compounds of the N-acylsulfonamide type, for example of the formula (S4a) below, which are known, for example, from WO-A-97/45016

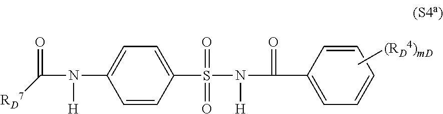

in which

R_{D}⁷ is (C₁-C₆)-alkyl, (C₃-C₆)-cycloalkyl, where the 2 latter radicals are substituted by v_{D} substituents from the group consisting of halogen, (C₁-C₄)-alkoxy, (C₁-C₆)-haloalkoxy and (C₁-C₄)-alkylthio and, in the case of cyclic radicals, also (C₁-C₄)-alkyl and (C₁-C₄)-haloalkyl;

R_{D}⁴ is halogen, (C₁-C₄)-alkyl, (C₁-C₄)-alkoxy, CF₃;

m_{D} is 1 or 2;

v_{D} is 0, 1, 2 or 3;

and also

acylsulfamoylbenzamides, for example of the formula (S4^{b}) below, which are known, for example, from WO-A-99/16744,

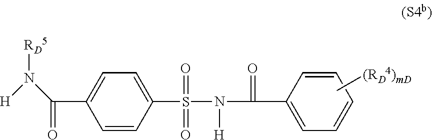

e.g. those in which

R_{D}⁵=cyclopropyl and (R_{D}⁴)=2-OMe (“cyprosulfamide”, S4-1),

R_{D}⁵=cyclopropyl and (R_{D}⁴)=5-Cl-2-OMe (S4-2),

R_{D}⁵=ethyl and (R_{D}⁴)=2-OMe (S4-3),

R_{D}⁵=isopropyl and (R_{D}⁴)=5-Cl-2-OMe (S4-4) and

R_{D}⁵=isopropyl and (R_{D}⁴)=2-OMe (S4-5)

and also

compounds of the N-acylsulfamoylphenylurea type of the formula (S4^{c}), which are known, for example, from EP-A-365484,

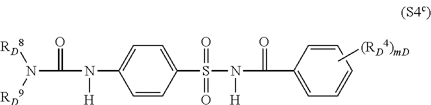

in which

R_{D}⁸ and R_{D}⁹ independently represent hydrogen, (C₁-C₈)-alkyl, (C₃-C₈)-cycloalkyl, (C₃-C₆)-alkenyl, (C₃-C₆)-alkynyl,

R_{D}⁴ is halogen, (C₁-C₄)-alkyl, (C₁-C₄)-alkoxy, CF₃,

m_{D} is 1 or 2;

for example
1-[4-(N-2-methoxybenzoylsulfamoyl)phenyl]-3-methylurea,
1-[4-(N-2-methoxybenzoylsulfamoyl)phenyl]-3,3-dimethylurea,
1-[4-(N-4,5-dimethylbenzoylsulfamoyl)phenyl]-3-methylurea,
   and also

N-phenylsulfonylterephthalamides of the formula (S4^{d}), which are known, for example, from CN 101838227,

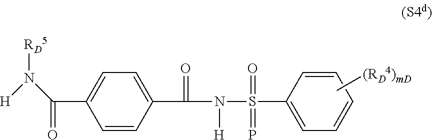

e.g. those in which

R_{D}⁴ is halogen, (C₁-C₄)-alkyl, (C₁-C₄)-alkoxy, CF₃;

m_{D} is 1 or 2;

R_{D}⁵ is hydrogen, (C₁-C₆)-alkyl, (C₃-C₆)-cycloalkyl, (C₂-C₆)-alkenyl, (C₂-C₆)-alkynyl, (C₅-C₆)-cycloalkenyl.

S5) Active ingredients from the class of the hydroxyaromatics and the aromatic-aliphatic carboxylic acid derivatives (S5), for example

ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicylic acid, 2-hydroxycinnamic acid, 2,4-dichlorocinnamic acid, as described in WO-A-2004/084631, WO-A-2005/015994, WO-A-2005/016001.

S6) Active ingredients from the class of the 1,2-dihydroquinoxalin-2-ones (S6), for example

1-methyl-3-(2-thienyl)-1,2-dihydroquinoxalin-2-one, 1-methyl-3-(2-thienyl)-1,2-dihydroquinoxaline-2-thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydroquinoxalin-2-one, as described in WO-A-2005/112630.

S7) Compounds of the formula (S7), as described in WO-A-1998/38856,

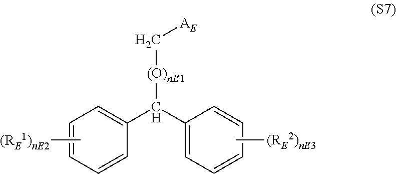

in which the symbols and indices are defined as follows:

R_{E}¹, R_{E}² are independently halogen, (C₁-C₄)-alkyl, (C₁-C₄)-alkoxy, (C₁-C₄)-haloalkyl, (C₁-C₄)-alkylamino, di-(C₁-C₄)-alkylamino, nitro;

A_{E} is COOR_{E}³ or COSR_{E}⁴

R_{E}³, R_{E}⁴ are independently hydrogen, (C₁-C₄)-alkyl, (C₂-C₆)-alkenyl, (C₂-C₄)-alkynyl, cyanoalkyl, (C₁-C₄)-haloalkyl, phenyl, nitrophenyl, benzyl, halobenzyl, pyridinylalkyl and alkylammonium,

n_{E}¹ is 0 or 1

n_{E}², n_{E}³ are independently 0, 1 or 2,

preferably:

diphenylmethoxyacetic acid,

ethyl diphenylmethoxyacetate,

methyl diphenylmethoxyacetate (CAS reg. no. 41858-19-9) (S7-1).

S8) Compounds of the formula (S8), as described in WO-A-98/27049,

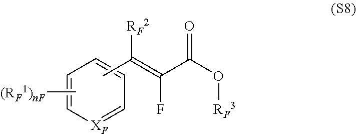

in which

X_{E} is CH or N,

n_{E} in the case that X_{E}═N is an integer from 0 to 4 and
in the case that X_{F}═CH is an integer from 0 to 5,

R_{F}¹ is halogen, (C₁-C₄)-alkyl, (C₁-C₄)-haloalkyl, (C₁-C₄)-alkoxy, (C₁-C₄)-haloalkoxy, nitro, (C₁-C₄)-alkylthio, (C₁-C₄)-alkylsulfonyl, (C₁-C₄)-alkoxycarbonyl, optionally substituted phenyl, optionally substituted phenoxy,

R_{F}² is hydrogen or (C₁-C₄)-alkyl,

R_{F}³ is hydrogen, (C₁-C₈)-alkyl, (C₂-C₄)-alkenyl, (C₂-C₄)-alkynyl or aryl, where each of the abovementioned carbon-containing radicals is unsubstituted or substituted by one or more, preferably up to three identical or different radicals from the group consisting of halogen and alkoxy; or salts thereof,

preferably compounds in which

X_{F} is CH,

n_{F} is an integer from 0 to 2,

R^{F} is halogen, (C₁-C₄)-alkyl, (C₁-C₄)-haloalkyl, (C₁-C₄)-alkoxy, (C₁-C₄)-haloalkoxy,

R_{F}² is hydrogen or (C₁-C₄)-alkyl,

R_{F}³ is hydrogen, (C₁-C₈)-alkyl, (C₂-C₄)-alkenyl, (C₂-C₄)-alkynyl or aryl, where each of the abovementioned carbon-containing radicals is unsubstituted or substituted by one or more, preferably up to three identical or different radicals from the group consisting of halogen and alkoxy,

or salts thereof.

S9) Active ingredients from the class of the 3-(5-tetrazolylcarbonyl)-2-quinolones (S9), for example

1,2-dihydro-4-hydroxy-1-ethyl-3-(5-tetrazolylcarbonyl)-2-quinolone (CAS reg. no. 219479-18-2), 1,2-dihydro-4-hydroxy-1-methyl-3-(5-tetrazolylcarbonyl)-2-quinolone (CAS Reg. No. 95855-00-8), as described in WO-A-1999/000020.

S10) Compounds of the formulae (S10a) or (S10^{b})
as described in WO-A-2007/023719 and WO-A-2007/023764

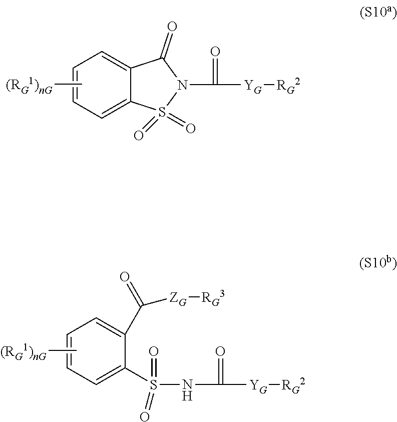

in which

R_{G}¹ is halogen, (C₁-C₄)-alkyl, methoxy, nitro, cyano, CF₃, OCF₃,

Y_{G}, Z_{G} independently of one another represent O or S,

n_{G} is an integer from 0 to 4,

R_{G}² is (C₁-C₁₆)-alkyl, (C₂-C₆)-alkenyl, (C₃-C₆)-cycloalkyl, aryl; benzyl, halobenzyl,

R_{G}³ is hydrogen or (C₁-C₆)-alkyl.

S11) Active ingredients of the oxyimino compounds type (S11), which are known as seed-dressing agents, for example

“oxabetrinil” ((Z)-1,3-dioxolan-2-ylmethoxyimino(phenyl)acetonitrile) (S11-1), which is known as a seed-dressing safener for millet/sorghum against metolachlor damage,

“fluxofenim” (1-(4-chlorophenyl)-2,2,2-trifluoro-1-ethanone O-(1,3-dioxolan-2-ylmethyl)oxime) (S11-2), which is known as a seed-dressing safener for millet/sorghum against metolachlor damage, and

“cyometrinil” or “CGA-43089” ((Z)-cyanomethoxyimino(phenyl)acetonitrile) (S11-3), which is known as a seed-dressing safener for millet/sorghum against metolachlor damage.

S12) Active ingredients from the class of the isothiochromanones (S12), for example methyl [(3-oxo-1H-2-benzothiopyran-4(3H)-ylidene)methoxy]acetate (CAS Reg. No. 205121-04-6) (S12-1) and related compounds from WO-A-1998/13361.

S13) One or more compounds from group (S13):

“naphthalic anhydride” (1,8-naphthalenedicarboxylic anhydride) (S13-1), which is known as a seed-dressing safener for maize against thiocarbamate herbicide damage,

“fenclorim” (4,6-dichloro-2-phenylpyrimidine) (S13-2), which is known as a safener for pretilachlor in sown rice,

“flurazole” (benzyl 2-chloro-4-trifluoromethyl-1,3-thiazole-5-carboxylate) (S13-3), which is known as a seed-dressing safener for millet/sorghum against alachlor and metolachlor damage,

“CL 304415” (CAS Reg. No. 31541-57-8)

(4-carboxy-3,4-dihydro-2H-1-benzopyran-4-acetic acid) (S13-4) from American Cyanamid, which is known as a safener for maize against damage by imidazolinones,

“MG 191” (CAS Reg. No. 96420-72-3) (2-dichloromethyl-2-methyl-1,3-dioxolane) (S13-5) from Nitrokemia, which is known as a safener for maize,

“MG 838” (CAS Reg. No. 133993-74-5)

(2-propenyl 1-oxa-4-azaspiro[4.5]decane-4-carbodithioate) (S13-6) from Nitrokemia,

“disulfoton” (O,O-diethyl S-2-ethylthioethyl phosphorodithioate) (S13-7),

“dietholate” (O,O-diethyl O-phenyl phosphorothioate) (S13-8),

“mephenate” (4-chlorophenyl methylcarbamate) (S13-9).

S14) Active ingredients which, in addition to herbicidal action against harmful plants, also have safener action on crop plants such as rice, for example

“dimepiperate” or “MY 93” (S-1-methyl 1-phenylethylpiperidine-1-carbothioate), which is known as a safener for rice against damage by the herbicide molinate,

“daimuron” or “SK 23” (1-(1-methyl-1-phenylethyl)-3-p-tolylurea), which is known as a safener for rice against damage by the herbicide imazosulfuron,

“cumyluron”=“JC 940” (3-(2-chlorophenylmethyl)-1-(1-methyl-1-phenylethyl)urea, see JP-A-60087254), which is known as safener for rice against damage by some herbicides,

“methoxyphenone” or “NK 049” (3,3′-dimethyl-4-methoxybenzophenone), which is known as a safener for rice against damage by some herbicides,

“CSB” (1-bromo-4-(chloromethylsulfonyl)benzene) from Kumiai, (CAS Reg. No. 54091-06-4), which is known as a safener against damage by some herbicides in rice.

S15) Compounds of the formula (S15) or tautomers thereof

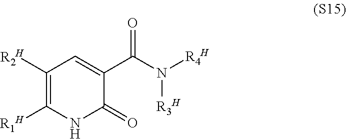

as described in WO-A-2008/131861 and WO-A-2008/131860 in which

R_{H}¹ is a (C₁-C₆)-haloalkyl radical and

R_{H}² is hydrogen or halogen and

R_{H}³, R_{H}⁴ are independently hydrogen, (C₁-C₁₆)-alkyl, (C₂-C₁₆)-alkenyl or (C₂-C₁₆)-alkynyl,

where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,

or (C₃-C₆)-cycloalkyl, (C₄-C₆)-cycloalkenyl, (C₃-C₆)-cycloalkyl fused on one side of the ring to a 4 to 6-membered saturated or unsaturated carbocyclic ring, or (C₄-C₆)-cycloalkenyl fused on one side of the ring to a 4 to 6-membered saturated or unsaturated carbocyclic ring,

where each of the 4 latter radicals is unsubstituted or substituted by one or more radicals from the group consisting of halogen, hydroxyl, cyano, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,

or

R_{H}³ is (C₁-C₄)-alkoxy, (C₂-C₄)-alkenyloxy, (C₂-C₆)-alkynyloxy or (C₂-C₄)-haloalkoxy and

R_{H}⁴ is hydrogen or (C₁-C₄)-alkyl or

R_{H}³ and R_{H}⁴ together with the directly attached nitrogen atom represent a four- to eight-membered heterocyclic ring which, as well as the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy and (C₁-C₄)-alkylthio.

S16) Active compounds which are used primarily as herbicides but also have safener action on crop plants, for example
(2,4-dichlorophenoxy)acetic acid (2,4-D),
(4-chlorophenoxy)acetic acid,
(R,S)-2-(4-chloro-o-tolyloxy)propionic acid (mecoprop),
4-(2,4-dichlorophenoxy)butyric acid (2,4-DB),
(4-chloro-o-tolyloxy)acetic acid (MCPA),
4-(4-chloro-o-tolyloxy)butyric acid,
4-(4-chlorophenoxy)butyric acid,
3,6-dichloro-2-methoxybenzoic acid (dicamba),
1-(ethoxycarbonyl)ethyl 3,6-dichloro-2-methoxybenzoate (lactidichlor-ethyl).

Particularly preferred safeners are mefenpyr-diethyl, cyprosulfamide, isoxadifen-ethyl, cloquintocet-mexyl, dichlormid and metcamifen.

Wettable powders are preparations uniformly dispersible in water which, in addition to the active ingredient and apart from a diluent or inert substance, also comprise surfactants of ionic and/or nonionic type (wetting agent, dispersant), e.g. polyethoxylated alkylphenols, polyethoxylated fatty alcohols, polyethoxylated fatty amines, fatty alcohol polyglycolethersulfates, alkanesulfonates, alkylbenzenesulfonates, sodium lignosulfonate, sodium 2,2′-dinaphthylmethane-6,6′-disulfonate, sodium dibutylnaphthalenesulfonate or else sodium oleoylmethyltaurate. To produce the wettable powders, the active herbicidal ingredients are finely ground, for example in customary apparatuses such as hammer mills, blower mills and air-jet mills, and simultaneously or subsequently mixed with the formulation auxiliaries.

Emulsifiable concentrates are produced by dissolving the active ingredient in an organic solvent, for example butanol, cyclohexanone, dimethylformamide, xylene, or else relatively high-boiling aromatics or hydrocarbons or mixtures of the organic solvents, with addition of one or more ionic and/or nonionic surfactants (emulsifiers). Examples of emulsifiers which may be used are: calcium alkylarylsulfonate salts such as calcium dodecylbenzenesulfonate, or nonionic emulsifiers such as fatty acid polyglycol esters, alkylaryl polyglycol ethers, fatty alcohol polyglycol ethers, propylene oxide/ethylene oxide condensation products, alkyl polyethers, sorbitan esters, for example sorbitan fatty acid esters, or polyoxyethylene sorbitan esters, for example polyoxyethylene sorbitan fatty acid esters.

Dusting products are obtained by grinding the active ingredient with finely distributed solids, for example talc, natural clays, such as kaolin, bentonite and pyrophyllite, or diatomaceous earth.

Suspension concentrates may be water- or oil-based. They may be produced, for example, by wet-grinding by means of commercial bead mills and optional addition of surfactants as already listed above, for example, for the other formulation types.

Emulsions, for example oil-in-water emulsions (EW), can be produced, for example, by means of stirrers, colloid mills and/or static mixers using aqueous organic solvents and optionally surfactants as already listed above, for example, for the other formulation types.

Granules can be produced either by spraying the active ingredient onto granular inert material capable of adsorption or by applying active ingredient concentrates to the surface of carrier substances, such as sand, kaolinites or granular inert material, by means of adhesives, for example polyvinyl alcohol, sodium polyacrylate or else mineral oils. Suitable active ingredients can also be granulated in the manner customary for the production of fertilizer granules—if desired as a mixture with fertilizers.

Water-dispersible granules are produced generally by the customary processes such as spray-drying, fluidized-bed granulation, pan granulation, mixing with high-speed mixers and extrusion without solid inert material.

For the production of pan granules, fluidized bed granules, extruder granules and spray granules, see, for example, processes in “Spray-Drying Handbook” 3rd ed. 1979, G. Goodwin Ltd., London, J. E. Browning, “Agglomeration”, Chemical and Engineering 1967, pages 147 ff.; “Perry's Chemical Engineer's Handbook”, 5th Ed., McGraw-Hill, New York 1973, pp. 8-57.

For further details regarding the formulation of crop protection compositions, see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

The agrochemical preparations contain generally 0.1% to 99% by weight, especially 0.1% to 95% by weight, of compounds of the invention. In wettable powders, the active ingredient concentration is, for example, about 10% to 90% by weight, the remainder to 100% by weight consisting of customary formulation constituents. In emulsifiable concentrates, the active ingredient concentration may be about 1% to 90% and preferably 5% to 80% by weight. Formulations in the form of dusts comprise 1% to 30% by weight of active ingredient, preferably usually 5% to 20% by weight of active ingredient; sprayable solutions contain about 0.05% to 80% by weight, preferably 2% to 50% by weight of active ingredient. In the case of water-dispersible granules, the active ingredient content depends partially on whether the active ingredient is in liquid or solid form and on which granulation auxiliaries, fillers, etc., are used. In the water-dispersible granules, the content of active ingredient is, for example, between 1% and 95% by weight, preferably between 10% and 80% by weight.

In addition, the active ingredient formulations mentioned optionally comprise the respective customary stickers, wetters, dispersants, emulsifiers, penetrants, preservatives, antifreeze agents and solvents, fillers, carriers and dyes, defoamers, evaporation inhibitors and agents which influence the pH and the viscosity.

On the basis of these formulations, it is also possible to produce combinations with other pesticidally active substances, for example insecticides, acaricides, herbicides, fungicides, and also with safeners, fertilizers and/or growth regulators, for example in the form of a finished formulation or as a tank mix.

For application, the formulations in the commercial form are diluted if appropriate in a customary manner, for example with water in the case of wettable powders, emulsifiable concentrates, dispersions and water-dispersible granules. Preparations in dust form, granules for soil application or granules for scattering and sprayable solutions are not normally diluted further with other inert substances prior to application.

The required application rate of the compounds of the formula (I) and their salts varies according to the external conditions such as, inter alia, temperature, humidity and the type of herbicide used. It can vary within wide limits, for example between 0.001 and 10.0 kg/ha or more of active substance, but it is preferably between 0.005 and 5 kg/ha, more preferably in the range of from 0.01 to 1.5 kg/ha, more preferably in the range of from 0.05 to 1 kg/ha. This applies both to pre-emergence and to post-emergence application.

A carrier is a natural or synthetic, organic or inorganic substance with which the active ingredients are mixed or combined for better applicability, in particular for application to plants or plant parts or seed. The carrier, which may be solid or liquid, is generally inert and should be suitable for use in agriculture.

Useful solid or liquid carriers include: for example ammonium salts and natural rock dusts, such as kaolins, clays, talc, chalk, quartz, attapulgite, montmorillonite or diatomaceous earth, and synthetic rock dusts, such as finely divided silica, alumina and natural or synthetic silicates, resins, waxes, solid fertilizers, water, alcohols, especially butanol, organic solvents, mineral and vegetable oils, and derivatives thereof. It is likewise possible to use mixtures of such carriers. Useful solid carriers for granules include: for example crushed and fractionated natural rocks such as calcite, marble, pumice, sepiolite, dolomite, and synthetic granules of inorganic and organic meals, and also granules of organic material such as sawdust, coconut shells, maize cobs and tobacco stalks.

Suitable liquefied gaseous extenders or carriers are liquids which are gaseous at standard temperature and under atmospheric pressure, for example aerosol propellants such as halogenated hydrocarbons, or else butane, propane, nitrogen and carbon dioxide.

In the formulations, it is possible to use tackifiers such as carboxymethylcellulose, natural and synthetic polymers in the form of powders, granules or latices, such as gum arabic, polyvinyl alcohol and polyvinyl acetate, or else natural phospholipids such as cephalins and lecithins, and synthetic phospholipids. Further additives may be mineral and vegetable oils.

When the extender used is water, it is also possible to use, for example, organic solvents as auxiliary solvents. Useful liquid solvents are essentially: aromatics such as xylene, toluene or alkylnaphthalenes, chlorinated aromatics or chlorinated aliphatic hydrocarbons such as chlorobenzenes, chloroethylenes or dichloromethane, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulfoxide, and also water.

The compositions of the invention may additionally comprise further components, for example surfactants. Useful surfactants are emulsifiers and/or foam formers, dispersants or wetting agents having ionic or nonionic properties, or mixtures of these surfactants. Examples thereof are salts of polyacrylic acid, salts of lignosulfonic acid, salts of phenolsulfonic acid or naphthalenesulfonic acid, polycondensates of ethylene oxide with fatty alcohols or with fatty acids or with fatty amines, substituted phenols (preferably alkylphenols or arylphenols), salts of sulfosuccinic esters, taurine derivatives (preferably alkyl taurates), phosphoric esters of polyethoxylated alcohols or phenols, fatty acid esters of polyols, and derivatives of the compounds containing sulfates, sulfonates and phosphates, for example alkylaryl polyglycol ethers, alkylsulfonates, alkyl sulfates, arylsulfonates, protein hydrolysates, lignosulfite waste liquors and methylcellulose. The presence of a surfactant is necessary if one of the active ingredients and/or one of the inert carriers is insoluble in water and when application is effected in water. The proportion of surfactants is between 5 and 40 percent by weight of the inventive composition. It is possible to use dyes such as inorganic pigments, for example iron oxide, titanium oxide and Prussian Blue, and organic dyes such as alizarin dyes, azo dyes and metal phthalocyanine dyes, and trace nutrients such as salts of iron, manganese, boron, copper, cobalt, molybdenum and zinc.

If appropriate, it is also possible for other additional components to be present, for example protective colloids, binders, adhesives, thickeners, thixotropic substances, penetrants, stabilizers, sequestrants, complexing agents. In general, the active ingredients can be combined with any solid or liquid additive commonly used for formulation purposes. In general, the compositions and formulations of the invention contain between 0.05% and 99% by weight, 0.01% and 98% by weight, preferably between 0.1% and 95% by weight, more preferably between 0.5% and 90% active ingredient, most preferably between 10 and 70 percent by weight. The active ingredients or compositions of the invention can be used as such or, depending on their respective physical and/or chemical properties, in the form of their formulations or the use forms prepared therefrom, such as aerosols, capsule suspensions, cold-fogging concentrates, warm-fogging concentrates, encapsulated granules, fine granules, flowable concentrates for the treatment of seed, ready-to-use solutions, dustable powders, emulsifiable concentrates, oil-in-water emulsions, water-in-oil emulsions, macrogranules, microgranules, oil-dispersible powders, oil-miscible flowable concentrates, oil-miscible liquids, foams, pastes, pesticide coated seed, suspension concentrates, suspoemulsion concentrates, soluble concentrates, suspensions, sprayable powders, soluble powders, dusts and granules, water-soluble granules or tablets, water-soluble powders for the treatment of seed, wettable powders, natural products and synthetic substances impregnated with active ingredient, and also microencapsulations in polymeric substances and in coating materials for seed, and also ULV cold-fogging and warm-fogging formulations.

The formulations mentioned can be produced in a manner known per se, for example by mixing the active ingredients with at least one customary extender, solvent or diluent, emulsifier, dispersant and/or binder or fixative, wetting agent, water repellent, optionally siccatives and UV stabilizers and optionally dyes and pigments, antifoams, preservatives, secondary thickeners, tackifiers, gibberellins and other processing auxiliaries.

The compositions of the invention include not only formulations which are already ready for use and can be deployed with a suitable apparatus onto the plant or the seed, but also commercial concentrates which have to be diluted with water prior to use.

The active ingredients of the invention may be present as such or in their (commercial standard) formulations, or else in the use forms prepared from these formulations as a mixture with other (known) active ingredients, such as insecticides, attractants, sterilants, bactericides, acaricides, nematicides, fungicides, growth regulators, herbicides, fertilizers, safeners or semiochemicals.

The inventive treatment of the plants and plant parts with the active ingredients or compositions is effected directly or by action on their surroundings, habitat or storage space by the customary treatment methods, for example by dipping, spraying, atomizing, irrigating, evaporating, dusting, fogging, broadcasting, foaming, painting, spreading-on, watering (drenching), drip irrigating and, in the case of propagation material, especially in the case of seeds, also by dry seed treatment, wet seed treatment, slurry treatment, incrustation, coating with one or more coats, etc. It is also possible to deploy the active ingredients by the ultra-low volume method or to inject the active ingredient preparation or the active ingredient itself into the soil.

As also described below, the treatment of transgenic seed with the active ingredients or compositions of the invention is of particular significance. This relates to the seed of plants containing at least one heterologous gene which enables the expression of a polypeptide or protein having insecticidal properties. The heterologous gene in transgenic seed can originate, for example, from microorganisms of the species Bacillus, Rhizobium, Pseudomonas, Serratia, Trichoderma, Clavibacter, Glomus or Gliocladium. This heterologous gene preferably originates from Bacillus sp., in which case the gene product is effective against the European corn borer and/or the Western corn rootworm. The heterologous gene more preferably originates from Bacillus thuringiensis.

In the context of the present invention, the inventive composition is applied to the seed alone or in a suitable formulation. Preferably, the seed is treated in a state in which it is sufficiently stable for no damage to occur in the course of treatment. In general, the seed can be treated at any time between harvest and sowing. It is customary to use seed which has been separated from the plant and freed from cobs, shells, stalks, coats, hairs or the flesh of the fruits. For example, it is possible to use seed which has been harvested, cleaned and dried down to a moisture content of less than 15% by weight. Alternatively, it is also possible to use seed which, after drying, for example, has been treated with water and then dried again.

In general, when treating the seed, it has to be ensured that the amount of the composition of the invention and/or further additives applied to the seed is chosen such that the germination of the seed is not impaired and the plant which arises therefrom is not damaged. This has to be ensured particularly in the case of active ingredients which can exhibit phytotoxic effects at certain application rates.

The compositions of the invention can be applied directly, i.e. without containing any other components and without having been diluted. In general, it is preferable to apply the compositions to the seed in the form of a suitable formulation. Suitable formulations and methods for seed treatment are known to those skilled in the art and are described, for example, in the following documents: U.S. Pat. Nos. 4,272,417 A, 4,245,432 A, 4,808,430, 5,876,739, US 2003/0176428 A1, WO 2002/080675 A1, WO 2002/028186 A2.

The active ingredients of the invention can be converted to the customary seed-dressing formulations, such as solutions, emulsions, suspensions, powders, foams, slurries or other coating compositions for seed, and also ULV formulations.

These formulations are produced in a known manner, by mixing the active ingredients with customary additives, for example customary extenders and solvents or diluents, dyes, wetting agents, dispersants, emulsifiers, antifoams, preservatives, secondary thickeners, adhesives, gibberellins, and also water.

Dyes which may be present in the seed-dressing formulations usable in accordance with the invention are all dyes which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1.

Useful wetting agents which may be present in the seed-dressing formulations usable in accordance with the invention are all substances which promote wetting and which are customary for the formulation of agrochemically active ingredients. Alkyl naphthalenesulfonates, such as diisopropyl or diisobutyl naphthalenesulfonates, can be used with preference.

Suitable dispersants and/or emulsifiers which may be present in the seed-dressing formulations usable in accordance with the invention are all nonionic, anionic and cationic dispersants customary for the formulation of agrochemically active ingredients. Preference can be given to using nonionic or anionic dispersants or mixtures of nonionic or anionic dispersants. Suitable nonionic dispersants include especially ethylene oxide/propylene oxide block polymers, alkylphenol polyglycol ethers and tristryrylphenol polyglycol ethers, and the phosphated or sulfated derivatives thereof. Suitable anionic dispersants are especially lignosulfonates, polyacrylic acid salts and arylsulfonate-formaldehyde condensates.

Antifoams which may be present in the seed-dressing formulations usable in accordance with the invention are all foam-inhibiting substances customary for the formulation of agrochemically active ingredients. Silicone antifoams and magnesium stearate can be used with preference.

Preservatives which may be present in the seed-dressing formulations usable in accordance with the invention are all substances usable for such purposes in agrochemical compositions. Examples include dichlorophene and benzyl alcohol hemiformal.

Secondary thickeners which may be present in the seed-dressing formulations usable in accordance with the invention are all substances usable for such purposes in agrochemical compositions. Preferred examples include cellulose derivatives, acrylic acid derivatives, xanthan, modified clays and finely divided silica.

Useful stickers which may be present in the seed-dressing formulations usable in accordance with the invention are all customary binders usable in seed-dressing products. Preferred examples include polyvinylpyrrolidone, polyvinyl acetate, polyvinyl alcohol and tylose.

The seed-dressing formulations usable in accordance with the invention can be used, either directly or after previously having been diluted with water, for the treatment of a wide range of different seed, including the seed of transgenic plants. In this case, additional synergistic effects may also occur in interaction with the substances formed by expression.

For the treatment of seed with the seed-dressing formulations usable in accordance with the invention or with the preparations prepared therefrom by addition of water, useful equipment is all mixing units usable customarily for seed dressing. Specifically, the seed dressing procedure is to place the seed into a mixer, to add the particular desired amount of seed-dressing formulations, either as such or after prior dilution with water, and to mix them until the formulation is distributed homogeneously on the seed. If appropriate, this is followed by a drying operation.

The active ingredients of the invention, given good plant compatibility, favourable homeotherm toxicity and good environmental compatibility, are suitable for protection of plants and plant organs, for increasing harvest yields, and for improving the quality of the harvested crop. They can preferably be used as crop protection agents. They are active against normally sensitive and resistant species and also against all or specific stages of development.

Plants which can be treated in accordance with the invention include the following main crop plants: maize, soya bean, cotton, Brassica oil seeds such as Brassica napus (e.g. Canola), Brassica rapa, B. juncea (e.g. (field) mustard) and Brassica carinata, rice, wheat, sugar beet, sugar cane, oats, rye, barley, millet and sorghum, triticale, flax, grapes and various fruit and vegetables from various botanic taxa, for example Rosaceae sp. (for example pome fruits such as apples and pears, but also stone fruits such as apricots, cherries, almonds and peaches, and berry fruits such as strawberries), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for example banana trees and plantations), Rubiaceae sp. (for example coffee), Theaceae sp., Sterculiceae sp., Rutaceae sp. (for example lemons, oranges and grapefruit); Solanaceae sp. (for example tomatoes, potatoes, peppers, aubergines), Liliaceae sp., Compositae sp. (for example lettuce, artichokes and chicory—including root chicory, endive or common chicory), Umbelliferae sp. (for example carrots, parsley, celery and celeriac), Cucurbitaceae sp. (for example cucumbers—including gherkins, pumpkins, watermelons, calabashes and melons), Alliaceae sp. (for example leeks and onions), Cruciferae sp. (for example white cabbage, red cabbage, broccoli, cauliflower, Brussels sprouts, pak choi, kohlrabi, radishes, horseradish, cress and chinese cabbage), Leguminosae sp. (for example peanuts, peas, and beans—for example common beans and broad beans), Chenopodiaceae sp. (for example Swiss chard, fodder beet, spinach, beetroot), Malvaceae (for example okra), Asparagaceae (for example asparagus); useful plants and ornamental plants in the garden and woods; and in each case genetically modified types of these plants.

As mentioned above, it is possible to treat all plants and their parts in accordance with the invention. In a preferred embodiment, wild plant species and plant cultivars, or those obtained by conventional biological breeding techniques, such as crossing or protoplast fusion, and parts thereof, are treated. In a further preferred embodiment, transgenic plants and plant cultivars obtained by genetic engineering methods, if appropriate in combination with conventional methods (genetically modified organisms), and parts thereof are treated. The term “parts” or “parts of plants” or “plant parts” has been explained above. Particular preference is given in accordance with the invention to treating plants of the respective commercially customary plant cultivars or those that are in use. Plant cultivars are understood to mean plants having new properties (“traits”) which have been grown by conventional breeding, by mutagenesis or by recombinant DNA techniques. They may be cultivars, varieties, biotypes and genotypes.

The treatment method of the invention can be used for the treatment of genetically modified organisms (GMOs), e.g. plants or seeds. Genetically modified plants (or transgenic plants) are plants in which a heterologous gene has been stably integrated into the genome. The term “heterologous gene” means essentially a gene which is provided or assembled outside the plant and which, upon introduction into the nuclear genome, the chloroplast genome or the mitochondrial genome, imparts to the transformed plant novel or improved agronomical or other traits because it expresses a protein or polypeptide of interest or another gene which is present in the plant, or other genes which are present in the plant are down-regulated or switched off (for example by means of antisense technology, co-suppression technology or RNAi technology [RNA interference]). A heterologous gene that is located in the genome is also called a transgene. A transgene that is defined by its specific presence in the plant genome is called a transformation or transgenic event.

Depending on the plant species or plant cultivars, their location and growth conditions (soils, climate, vegetation period, diet), the inventive treatment may also result in superadditive (“synergistic”) effects. For example, the following effects which exceed the effects actually to be expected are possible: reduced application rates and/or widened spectrum of activity and/or increased efficacy of the active ingredients and compositions which can be used in accordance with the invention, better plant growth, increased tolerance to high or low temperatures, increased tolerance to drought or to water or soil salinity, increased flowering performance, easier harvesting, accelerated maturation, higher harvest yields, bigger fruits, greater plant height, greener leaf colour, earlier flowering, higher quality and/or a higher nutritional value of the harvested products, higher sugar concentration within the fruits, better storage stability and/or processability of the harvested products.

Plants and plant cultivars which are preferably treated in accordance with the invention include all plants which have genetic material which imparts particularly advantageous, useful traits to these plants (whether obtained by breeding and/or biotechnological means).

Examples of nematode-resistant plants are described, for example, in the following U.S. patent application Ser. Nos. 11/765,491, 11/765,494, 10/926,819, 10/782,020, 12/032,479, 10/783,417, 10/782,096, 11/657,964, 12/192,904, 11/396,808, 12/166,253, 12/166,239, 12/166,124, 12/166,209, 11/762,886, 12/364,335, 11/763,947, 12/252,453, 12/209,354, 12/491,396 and 12/497,221.

Plants that may be treated according to the invention are hybrid plants that already express the characteristics of heterosis, or hybrid effect, which results generally in higher yield, vigour, better health and resistance towards biotic and abiotic stress factors. Such plants are typically produced by crossing an inbred male-sterile parent line (the female crossbreeding parent) with another inbred male-fertile parent line (the male crossbreeding parent). Hybrid seed is typically harvested from the male-sterile plants and sold to growers. Male-sterile plants can sometimes (e.g. in maize) be produced by detasselling (i.e. the mechanical removal of the male reproductive organs or male flowers) but, more typically, male sterility is the result of genetic determinants in the plant genome. In that case, and especially when seed is the desired product to be harvested from the hybrid plants, it is typically beneficial to ensure that male fertility in hybrid plants, which contain the genetic determinants responsible for male sterility, is fully restored. This can be accomplished by ensuring that the male crossbreeding parents have appropriate fertility restorer genes which are capable of restoring the male fertility in hybrid plants that contain the genetic determinants responsible for male sterility. Genetic determinants for male sterility may be located in the cytoplasm. Examples of cytoplasmic male sterility (CMS) were for instance described for Brassica species. However, genetic determinants for male sterility can also be located in the nuclear genome. Male-sterile plants can also be obtained by plant biotechnology methods such as genetic engineering. A particularly useful means of obtaining male-sterile plants is described in WO 89/10396 in which, for example, a ribonuclease such as a barnase is selectively expressed in the tapetum cells in the stamens. Fertility can then be restored by expression in the tapetum cells of a ribonuclease inhibitor such as barstar.

Plants or plant cultivars (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Herbicide-tolerant plants are for example glyphosate-tolerant plants, i.e. plants made tolerant to the herbicide glyphosate or salts thereof. Plants can be made tolerant to glyphosate by various methods. Thus, for example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium (Comai et al., 1983, Science, 221, 370-371), the CP4 gene of the bacterium Agrobacterium sp. (Barry et al., 1992, Curr. Topics Plant Physiol. 7, 139-145), the genes encoding a petunia EPSPS (Shah et al., 1986, Science 233, 478-481), a tomato EPSPS (Gasser et al., 1988, J. Biol. Chem. 263, 4280-4289) or an Eleusine EPSPS (WO 01/66704). It can also be a mutated EPSPS. Glyphosate-tolerant plants can also be obtained by expressing a gene that encodes a glyphosate oxidoreductase enzyme. Glyphosate-tolerant plants can also be obtained by expressing a gene that encodes a glyphosate acetyltransferase enzyme. Glyphosate-tolerant plants can also be obtained by selecting plants containing naturally-occurring mutations of the abovementioned genes. Plants which express EPSPS genes which impart glyphosate tolerance have been described. Plants which express other genes which impart glyphosate tolerance, for example decarboxylase genes, have been described.

Other herbicide-resistant plants are for example plants made tolerant to herbicides inhibiting the enzyme glutamine synthase, such as bialaphos, phosphinothricin or glufosinate. Such plants can be obtained by expressing an enzyme detoxifying the herbicide or a mutant of the glutamine synthase enzyme that is resistant to inhibition. One example of such an effective detoxifying enzyme is an enzyme encoding a phosphinothricin acetyltransferase (such as the bar or pat protein from Streptomyces species). Plants expressing an exogenous phosphinothricin acetyltransferase have been described.

Further herbicide-tolerant plants are also plants that have been made tolerant to the herbicides inhibiting the enzyme hydroxyphenylpyruvate dioxygenase (HPPD). Hydroxyphenylpyruvate dioxygenases are enzymes that catalyse the reaction in which para-hydroxyphenylpyruvate (HPP) is converted to homogentisate. Plants tolerant to HPPD inhibitors can be transformed with a gene encoding a naturally-occurring resistant HPPD enzyme, or a gene encoding a mutated or chimeric HPPD enzyme, as described in WO 96/38567, WO 99/24585, WO 99/24586, WO 2009/144079, WO 2002/046387 or U.S. Pat. No. 6,768,044. Tolerance to HPPD inhibitors can also be obtained by transforming plants with genes encoding certain enzymes enabling the formation of homogentisate despite inhibition of the native HPPD enzyme by the HPPD inhibitor. Such plants are described in WO 99/34008 and WO 02/36787. Tolerance of plants to HPPD inhibitors can also be improved by transforming plants with a gene encoding a prephenate dehydrogenase enzyme in addition to a gene encoding an HPPD-tolerant enzyme, as described in WO 2004/024928. In addition, plants can be made more tolerant to HPPD inhibitors by inserting into the genome thereof a gene which encodes an enzyme which metabolizes or degrades HPPD inhibitors, for example CYP450 enzymes (see WO 2007/103567 and WO 2008/150473).

Other herbicide-resistant plants are plants which have been rendered tolerant to acetolactate synthase (ALS) inhibitors. Known ALS inhibitors include, for example, sulfonylurea, imidazolinone, triazolopyrimidines, pyrimidinyloxy(thio)benzoates, and/or sulfonylaminocarbonyltriazolinone herbicides. It is known that different mutations in the ALS enzyme (also known as acetohydroxy acid synthase, AHAS) confer tolerance to different herbicides and groups of herbicides, as described, for example, in Tranel and Wright (Weed Science 2002, 50, 700-712). The production of sulfonylurea-tolerant plants and imidazolinone-tolerant plants has been described. Further sulfonylurea- and imidazolinone-tolerant plants have also been described.

Further plants tolerant to imidazolinones and/or sulfonylureas can be obtained by induced mutagenesis, by selection in cell cultures in the presence of the herbicide or by mutation breeding (cf., for example, for soya beans U.S. Pat. No. 5,084,082, for rice WO 97/41218, for sugar beet U.S. Pat. No. 5,773,702 and WO 99/057965, for lettuce U.S. Pat. No. 5,198,599 or for sunflower WO 01/065922).

Plants or plant cultivars (obtained by plant biotechnology methods such as genetic engineering) which may also be treated according to the invention are tolerant to abiotic stress factors. Such plants can be obtained by genetic transformation, or by selection of plants containing a mutation imparting such stress resistance. Particularly useful stress-tolerant plants include the following:

a. plants which contain a transgene capable of reducing the expression and/or the activity of the poly(ADP-ribose) polymerase (PARP) gene in the plant cells or plants;

b. plants which contain a stress tolerance-enhancing transgene capable of reducing the expression and/or the activity of the PARG-encoding genes of the plants or plant cells;

c. plants which contain a stress tolerance-enhancing transgene coding for a plant-functional enzyme of the nicotinamide adenine dinucleotide salvage biosynthesis pathway, including nicotinamidase, nicotinate phosphoribosyltransferase, nicotinic acid mononucleotide adenyltransferase, nicotinamide adenine dinucleotide synthetase or nicotinamide phosphoribosyltransferase.

Plants or plant cultivars (obtained by plant biotechnology methods such as genetic engineering) which may also be treated according to the invention show altered quantity, quality and/or storage stability of the harvested product and/or altered properties of specific components of the harvested product such as, for example:

1) Transgenic plants which synthesize a modified starch which, in its physicochemical characteristics, in particular the amylose content or the amylose/amylopectin ratio, the degree of branching, the average chain length, the side chain distribution, the viscosity behaviour, the gelling strength, the starch granule size and/or the starch granule morphology, is changed in comparison with the synthesized starch in wild-type plant cells or plants, so that this modified starch is better suited to specific applications.

2) Transgenic plants which synthesize non-starch carbohydrate polymers or which synthesize non-starch carbohydrate polymers with altered properties in comparison to wild-type plants without genetic modification. Examples are plants which produce polyfructose, especially of the inulin and levan type, plants which produce alpha-1,4-glucans, plants which produce alpha-1,6-branched alpha-1,4-glucans, and plants producing alternan.

3) Transgenic plants which produce hyaluronan.

4) Transgenic plants or hybrid plants such as onions with particular properties, such as “high soluble solids content”, “low pungency” (LP) and/or “long storage” (LS).

Plants or plant cultivars (obtained by plant biotechnology methods such as genetic engineering) which may also be treated according to the invention are plants, such as cotton plants, with altered fibre characteristics. Such plants can be obtained by genetic transformation, or by selection of plants containing a mutation imparting such altered fibre characteristics and include:

a) plants, such as cotton plants, containing an altered form of cellulose synthase genes;

b) plants, such as cotton plants, which contain an altered form of rsw2 or rsw3 homologous nucleic acids, such as cotton plants with an increased expression of sucrose phosphate synthase;

c) plants, such as cotton plants, with increased expression of sucrose synthase;

d) plants, such as cotton plants, wherein the timing of the plasmodesmatal gating at the basis of the fibre cell is altered, for example through downregulation of fibre-selective 0-1,3-glucanase;

e) plants, such as cotton plants, which have fibres with altered reactivity, for example through expression of the N-acetylglucosaminetransferase gene, including nodC, and chitin synthase genes.

Plants or plant cultivars (obtained by plant biotechnology methods such as genetic engineering) which may also be treated according to the invention are plants, such as oilseed rape or related Brassica plants, with altered oil profile characteristics. Such plants can be obtained by genetic transformation, or by selection of plants containing a mutation imparting such altered oil characteristics and include:

a) plants, such as oilseed rape plants, which produce oil having a high oleic acid content;

b) plants, such as oilseed rape plants, which produce oil having a low linolenic acid content;

c) plants, such as oilseed rape plants, which produce oil having a low level of saturated fatty acids.

Plants or plant cultivars (which can be obtained by plant biotechnology methods such as genetic engineering) which may also be treated according to the invention are plants such as potatoes which are virus-resistant, for example to the potato virus Y (SY230 and SY233 events from Tecnoplant, Argentina), or which are resistant to diseases such as potato late blight (e.g. RB gene), or which exhibit reduced cold-induced sweetness (which bear the genes Nt-Inh, II-INV) or which exhibit the dwarf phenotype (A-20 oxidase gene).

Plants or plant cultivars (obtained by plant biotechnology methods such as genetic engineering) which may also be treated according to the invention are plants, such as oilseed rape or related Brassica plants, with altered seed shattering characteristics. Such plants can be obtained by genetic transformation, or by selection of plants containing a mutation imparting such altered characteristics, and include plants such as oilseed rape with retarded or reduced seed shattering.

Particularly useful transgenic plants which can be treated according to the invention are plants with transformation events or combinations of transformation events which are the subject of granted or pending petitions for nonregulated status in the USA at the Animal and Plant Health Inspection Service (APHIS) of the United States Department of Agriculture (USDA). Information relating to this is available at any time from APHIS (4700 River Road Riverdale, Md. 20737, USA), for example via the website http://www.aphis.usda.gov/brs/not_reg.html. At the filing date of this application, the petitions with the following information were either granted or pending at APHIS:
Petition: Identification number of the petition. The technical description of the transformation event can be found in the specific petition document available from APHIS on the website via the petition number. These descriptions are hereby disclosed by reference.
Extension of a petition: Reference to an earlier petition for which an extension of scope or term is being requested.
Institution: Name of the person submitting the petition.
Regulated article: The plant species in question.
Transgenic phenotype: The trait imparted to the plant by the transformation event.
Transformation event or line: The name of the event(s) (sometimes also referred to as line(s)) for which nonregulated status is being requested.
APHIS documents: Various documents which have been published by APHIS with regard to the petition or can be obtained from APHIS on request.

Particularly useful transgenic plants which can be treated in accordance with the invention are plants which comprise one or more genes which code for one or more toxins, for example the transgenic plants which are sold under the following trade names: YIELD GARD® (for example maize, cotton, soya beans), KnockOut® (for example maize), BiteGard® (for example maize), BT-Xtra® (for example maize), StarLink® (for example maize), Bollgard® (cotton), Nucotn® (cotton), Nucotn 33B® (cotton), NatureGard® (for example maize), Protecta® and NewLeaf® (potato). Examples of herbicide-tolerant plants which may be mentioned include maize varieties, cotton varieties and soya bean varieties which are available under the following trade names: Roundup Ready® (tolerance to glyphosates, for example maize, cotton, soya beans), Liberty Link® (tolerance to phosphinothricin, for example oilseed rape), IMI® (tolerance to imidazolinone) and SCS® (tolerance to sulfonylurea), for example maize. Herbicide-resistant plants (plants bred in a conventional manner for herbicide tolerance) which may be mentioned include the varieties sold under the name Clearfield® (for example maize).

The examples which follow illustrate the present invention.

### EXAMPLES

The present invention is illustrated in detail by the examples which follow, but these examples do not restrict the invention in any way.

### A. Synthesis Examples

Methyl (2R)-2-{[4-bromo-1-(2,5-difluorophenyl)-5-(3,4-difluorophenyl)-1H-pyrazol-3-yl]oxy}propanoate (1-182): To a solution of 0.40 g (1.01 mmol) of methyl (2R)-2-{[1-(2,5-difluorophenyl)-5-(3,4-difluorophenyl)-1H-pyrazol-3-yl]oxy}propanoate in 14 ml of DMF was added 0.22 g (1.22 mmol) of N-bromosuccinimide, and the mixture was stirred at 65° C. for one hour. The solvent was removed under reduced pressure, the residue was taken up in water and extracted repeatedly with methylene chloride, the combined organic phases were dried over sodium sulfate, and the solvent was removed under reduced pressure. After purification by column chromatography on silica gel with a heptane/ethyl acetate gradient (start with heptane/ethyl acetate=95:5, to heptane/ethyl acetate=50:50 within 20 min), 0.36 g (71%) of a product with m/z (%)=473 (50) [M⁺], 475 (50) [M⁺] and a specific angle of rotation of [α]=+20° was obtained. ¹H-NMR (400 MHz, CDCl₃): δ=1.57 (d, 3H), 3.69 (s, 3H), 5.15 (q, 1H), 7.18 (m, 1H), 7.36 (m, 2H), 7.50 (m, 3H).

Synthesis of the Starting Compounds:

Methyl (2R)-2-{[1-(2,5-difluorophenyl)-5-(3,4-difluorophenyl)-1H-pyrazol-3-yl]oxy}propanoate: To a solution of 0.50 g (1.54 mmol) of 1-(2,5-difluorophenyl)-5-(3,4-difluorophenyl)-1H-pyrazol-3-ol in 31 g of dimethylformamide was added 0.75 g (2.31 mmol) of caesium carbonate, the mixture was stirred at room temperature for 10 minutes, 0.23 g (1.85 mmol) of methyl (2S)-2-chloropropanoate was added, and the mixture was stirred at 80° C. for a further hour. The solvent was removed under reduced pressure, the residue was taken up in water and extracted repeatedly with methylene chloride, the combined organic phases were dried over sodium sulfate, and the solvent was removed under reduced pressure. After purification by column chromatography on silica gel with heptane/ethyl acetate (start with heptane/ethyl acetate=95:5, to heptane/ethyl acetate=50:50 within 12 min), 0.46 g (72%) of a product with m/z=395 [M⁺] and a specific angle of rotation of [α]=+40° was obtained. ¹H-NMR (400 MHz, CDCl₃): δ=1.63 (d, 3H), 3.78 (s, 3H), 5.18 (q, 1H), 6.03 (s, 1H), 6.91 (m, 1H), 7.08 (m, 4H), 7.16 (m, 1H).
b) 1-(2,5-Difluorophenyl)-5-(3,4-difluorophenyl)-1H-pyrazol-3-ol: To a solution of 4.95 g (9.64 mmol) of N′-(2,5-difluorophenyl)-3-(3,4-difluorophenyl)prop-2-yne hydrazide in 10 ml of DMF was added 0.15 g (0.77 mmol) of copper(I) iodide, and the mixture was stirred at 80° C. for one hour. After purification by column chromatography on silica gel with heptane/ethyl acetate (start with heptane/ethyl acetate=95:5, to heptane/ethyl acetate=20:80 within 15 min), 1.06 g (28%) of a product with m/z=309 [M⁺] was obtained. ¹H-NMR (400 MHz, d₆-DMSO): δ=6.09 (s, 1H), 7.02 (m, 1H), 7.40 (m, 4H), 7.50 (m, 1H), 10.41 (s, 1H).
c) N′-(2,5-Difluorophenyl)-3-(3,4-difluorophenyl)prop-2-yne hydrazide: To a solution of 2.14 g (11.75 mmol) of 3-(3,4-difluorophenyl)prop-2-ynoic acid in 20 ml of tetrahydrofuran were successively added 1.86 g (12.92 mmol) of (2,5-difluorophenyl)hydrazine and 5.94 g (58.75 mmol) of triethylamine. 11.22 g (17.62 mmol) of propanephosphonic anhydride (T3P, 50% solution in tetrahydrofuran) was added dropwise, and the mixture was stirred at room temperature for one hour. The reaction mixture was poured onto water and extracted repeatedly with methyl acetate, the combined organic phases were dried over magnesium sulfate, the solvent was removed under reduced pressure, and 4.97 g (80%) of an oily product (HPLC/MS purity=80%, m/z=309 [M⁺]), which was converted further without purification.
d) 3-(3,4-Difluorophenyl)prop-2-ynoic acid: Under an argon atmosphere, the following were added successively to 5.00 g (20.83 mmol) of 1,2-difluoro-4-iodobenzene in 30 ml of dry tetrahydrofuran: 1.46 g (20.83 mmol) of propiolic acid, 0.29 g (0.42 mmol) of bis(triphenylphosphine)palladium(11) dichloride, 0.16 g (0.83 mmol) of copper(I)iodide and 7.38 g (72.92 mmol) of diisopropylamine. The mixture was stirred at room temperature for 2 hours, the reaction mixture was added to water, 15.00 ml of 2 N hydrochloric acid was added and extraction was effected repeatedly with ethyl acetate. The combined organic phases were dried over sodium sulfate and concentrated under reduced pressure. After purification by column chromatography on silica gel with heptane/ethyl acetate (start with heptane/ethyl acetate=95:5, to heptane/ethyl acetate=40:60 within 15 min), 2.89 g (76%) of a product with m/z=183 [M⁺] was obtained. ¹H-NMR (400 MHz, d₆-DMSO): δ=7.56 (m, 2H), 7.86 (m, 1H), 13.95 (bs, 1H).

Methyl (2R)-2-{[4-cyano-1-(2,5-difluorophenyl)-5-(3,4-difluorophenyl)-1H-pyrazol-3-yl]oxy}propanoate (I-185): A mixture consisting of 0.18 g (0.37 mmol) of methyl (2R)-2-{[4-bromo-1-(2,5-difluorophenyl)-5-(3,4-difluorophenyl)-1H-pyrazol-3-yl]oxy}propanoate (I-182), 0.04 g (0.35 mmol) of zinc cyanide and 0.04 g (0.04 mmol) of tetrakis(triphenylphosphine)palladium in 5 ml of dimethylacetamide was heated to 180° C. in a microwave under an argon atmosphere for 40 minutes. The solvent was removed under reduced pressure, the residue was taken up in water/methylene chloride, the aqueous phase was extracted repeatedly with methylene chloride, the combined organic phases were dried over sodium sulfate, and the solvent was removed under reduced pressure. After purification by column chromatography on silica gel with a heptane/ethyl acetate gradient (start with heptane/ethyl acetate=95:5, to heptane/ethyl acetate=60:40 within 15 min), 0.11 g (67%) of a product with m/z=420 [M⁺] and a specific angle of rotation of [α]=+170 was obtained. ¹H-NMR (400 MHz, CDCl₃): δ=1.68 (d, 3H), 3.78 (s, 3H), 5.18 (q, 1H), 7.12 (m, 6H).

Methyl 2-{[4-chloro-5-(3,5-difluorophenyl)-1-phenyl-1H-pyrazol-3-yl]oxy}propanoate (I-165): A reaction mixture consisting of 150.0 mg (0.35 mmol) of methyl 2-[(4-chloro-5-iodo-1-phenyl-1H-pyrazol-3-yl)oxy]propanoate, 83.0 mg (0.53 mmol) of (3,5-difluorophenyl)boronic acid, 12.3 mg (0.02 mmol) of bis(triphenylphosphine)palladium(II) dichloride, 228.4 mg (0.70 mmol) of caesium carbonate (2.5 molar aqueous solution) and 4.4 ml of 1,2-dimethoxyethane was heated to 80° C. for 3 hours. The solvent was removed under reduced pressure, the residue was taken up in water/ethyl acetate, the aqueous phase was extracted repeatedly with ethyl acetate, the combined organic phases were dried over sodium sulfate, and the solvent was removed under reduced pressure. After purification by column chromatography on silica gel with heptane/ethyl acetate, 0.11 g (67%) of a product with m/z (%)=393 (76) [M⁺], 395 (24) [M⁺] was obtained. ¹H-NMR (400 MHz, CDCl₃): δ=1.69 (d, 3H), 3.79 (s, 3H), 5.24 (q, 1H), 6.82 (m, 3H), 7.12 (m, 2H), 7.29 (m, 3H).

Synthesis of the Starting Compounds:
a) Methyl 2-[(4-chloro-5-iodo-1-phenyl-1H-pyrazol-3-yl)oxy]propanoate: To a solution of 2.80 g (9.47 mmol) of methyl 2-[(5-amino-4-chloro-1-phenyl-1H-pyrazol-3-yl)oxy]propanoate in 56 ml of acetonitrile were successively added 10.14 g (37.87 mmol) of diiodomethane and 2.22 g (18.94 mmol) of isopentyl nitrite, and the mixture was heated to 50° C. for 30 minutes. The solvent was removed under reduced pressure, the residue was taken up in water/ethyl acetate, the aqueous phase was extracted repeatedly with ethyl acetate, the combined organic phases were dried over sodium sulfate, and the solvent was removed under reduced pressure. After purification by column chromatography on silica gel with heptane/ethyl acetate, 2.59 g (64%) of a yellowish solid with m/z (%)=407 (76) [M⁺], 409 (24) [M⁺] was obtained. ¹H-NMR (400 MHz, CDCl₃): δ=1.65 (d, 3H), 3.75 (s, 3H), 5.17 (q, 1H), 7.41 (m, 2H), 7.45 (m, 3H).
b) Methyl 2-[(5-amino-4-chloro-1-phenyl-1H-pyrazol-3-yl)oxy]propanoate: To a solution of 0.80 g (2.76 mmol) of methyl 2-[(5-amino-1-phenyl-1H-pyrazol-3-yl)oxy]propanoate in 5.0 ml of dimethylformamide was added 0.44 g (3.31 mmol) of N-chlorosuccinimide, and the mixture was stirred at room temperature for 30 minutes. The solvent was removed under reduced pressure, the residue was taken up in water/methylene chloride, the aqueous phase was extracted repeatedly with methylene chloride, the combined organic phases were dried over sodium sulfate, and the solvent was removed under reduced pressure. After purification by column chromatography on silica gel with heptane/ethyl acetate, 0.50 g (59%) of a colourless solid with m/z (%)=296 (76) [M⁺], 298 (24) [M⁺] was obtained. ¹H-NMR (400 MHz, CDCl₃): δ=1.64 (d, 3H), 3.76 (s, 3H), 3.91 (bs, 2H), 5.20 (q, 1H), 7.30 (m, 1H), 7.45 (m, 4H).
c) Methyl 2-[(5-amino-1-phenyl-1H-pyrazol-3-yl)oxy]propanoate: To a solution of 0.20 g (1.09 mmol) of 5-amino-1-phenyl-1H-pyrazol-3-ol in 5.0 ml of dimethylformamide was added 0.53 g (1.63 mmol) of caesium carbonate, the mixture was stirred at room temperature for 10 minutes, 0.22 g (1.30 mmol) of methyl 2-bromopropanoate was added, and the mixture was stirred at room temperature for 2 hours. The mixture was filtered, the filtrate was concentrated under reduced pressure, and the residue was stirred with dimethyl ether. This gave 0.19 g (64%) of a crystalline product with m/z=262 [M⁺]. ¹H-NMR (400 MHz, CDCl₃): δ=1.59 (d, 3H), 3.77 (s, 3H), 3.80 (bs, 2H), 5.16 (q, 1H), 5.20 (s, 1H), 7.26 (m, 1H), 7.45 (m, 2H), 7.49 (m, 2H).

Methyl {[4-chloro-1-(2-fluorophenyl)-5-(5-fluoro-2-thienyl)-1H-pyrazol-3-yl]oxy}acetate (1-094): To a solution of 85.0 mg (0.24 mmol) of methyl {[1-(2-fluorophenyl)-5-(5-fluoro-2-thienyl)-1H-pyrazol-3-yl]oxy}acetate in 3.2 ml of dimethylformamide was added 42.0 mg (0.32 mmol) of N-chlorosuccinimide in portions, and the mixture was stirred at room temperature for 14 hours. The solvent was removed under reduced pressure, the residue was taken up in water/methylene chloride, the organic phase was washed with saturated aqueous sodium hydrogencarbonate solution and water, the combined aqueous phases were extracted repeatedly with methylene chloride, the combined organic phases were dried over sodium sulfate, and the solvent was removed under reduced pressure. This gave 78 g (67%) of a product with m/z (%)=385 (76) [M⁺], 387 (24) [M⁺]. ¹H-NMR (400 MHz, CDCl₃): δ=3.79 (s, 3H), 4.90 (s, 2H), 6.38 (d, 2H), 6.72 (d, 2H), 7.16 (t, 1H), 7.24 (m, 1H), 7.41 (m, 2H).

Synthesis of the Starting Compounds:
a) Methyl {[1-(2-fluorophenyl)-5-(5-fluoro-2-thienyl)-1H-pyrazol-3-yl]oxy}acetate: Under an argon atmosphere, the following were added successively to a solution of 0.40 g (1.06 mmol) of methyl {[1-(2-fluorophenyl)-5-iodo-1H-pyrazol-3-yl]oxy}acetate in 6.1 ml of dioxane: 37.0 mg (0.05 mmol) of bis(triphenylphosphine)palladium(II) dichloride, 0.24 g (1.06 mmol) of 2-(5-fluoro-2-thienyl)-4,4,5,5-tetramethyl-1,3,2-dioxaborolane, 0.44 g (3.19 mmol) of potassium carbonate, 0.4 ml of water, and the mixture was heated under reflux for 6 hours. The solvent was removed under reduced pressure, the residue was taken up in water/methylene chloride, the aqueous phase was extracted repeatedly with methylene chloride, the combined organic phases were dried over sodium sulfate, and the solvent was removed under reduced pressure. After purification by column chromatography on silica gel with heptane/ethyl acetate, 0.18 g (38%) of a product with m/z (%)=351 [M⁺] was obtained. ¹H-NMR (400 MHz, CDCl₃): δ=3.80 (s, 3H), 4.84 (s, 2H), 6.06 (s, 1H), 6.31 (d, 2H), 6.45 (d, 2H), 7.18 (t, 1H), 7.28 (m, 1H), 7.44 (m, 2H).
b) Methyl {[1-(2-fluorophenyl)-5-iodo-1H-pyrazol-3-yl]oxy}acetate To a solution of 2.69 g (8.85 mmol) of 1-(2-fluorophenyl)-5-iodo-1H-pyrazol-3-ol in 197 ml of acetonitrile was added 3.67 g (26.54 mmol) of potassium carbonate, the mixture was stirred at room temperature for 10 minutes, 1.35 g (8.85 mmol) of methyl bromoacetate was added, and the mixture was stirred under reflux for 8 hours. The mixture was filtered, the filtrate was concentrated under reduced pressure, the residue was taken up in water/methylene chloride, the aqueous phase was extracted repeatedly with methylene chloride, the combined organic phases were dried over sodium sulfate, and the solvent was removed under reduced pressure. After purification by column chromatography on silica gel with heptane/ethyl acetate, 1.81 g (54%) of a product with m/z (%)=377 [M⁺] was obtained. ¹H-NMR (400 MHz, CDCl₃): δ=3.78 (s, 3H), 4.80 (s, 2H), 6.18 (s, 1H), 7.22 (m, 2H), 7.39 (m, 1H), 7.45 (m, 1H).
c) 1-(2-Fluorophenyl)-5-iodo-1H-pyrazol-3-ol: To a solution of 4.70 g (11.24 mmol) of 3-{[tert-butyl(dimethyl)silyl]oxy}-1-(2-fluorophenyl)-5-iodo-1H-pyrazole in 105 ml of tetrahydrofuran was added 3.82 g (14.61 mmol) of tetra-n-butylammonium fluoride, and the mixture was stirred at room temperature for 4 hours. The solvent was removed under reduced pressure, the residue was taken up in water/ethyl acetate, the aqueous phase was extracted repeatedly with ethyl acetate, the combined organic phases were dried over sodium sulfate, and the solvent was removed under reduced pressure. After purification by column chromatography on silica gel with heptane/ethyl acetate, 2.37 g (69%) of a product with m/z (%)=305 [M⁺] was obtained. ¹H-NMR (400 MHz, CDCl₃): δ=6.01 (s, 1H), 7.30 (m, 2H), 7.42 (m, 1H), 7.48 (m, 1H), 10.56 (bs, 1H).
d) 3-{[tert-Butyl(dimethyl)silyl]oxy}-1-(2-fluorophenyl)-5-iodo-1H-pyrazole: Under argon, a solution of 6.00 g (20.52 mmol) of 3-{[tert-butyl(dimethyl)silyl]oxy}-1-(2-fluorophenyl)-1H-pyrazole in 98 ml of tetrahydrofuran was added to a mixture of 1.71 g (26.67 mmol) of n-butyllithium and 2.70 g (26.67 mmol) of diisopropylamine in 98 ml of tetrahydrofuran, and the mixture was stirred at −78° C. for one hour. A solution of 6.77 g (26.67 mmol) of iodine in 98 ml of tetrahydrofuran was added dropwise, and the mixture was stirred at −78° C. for a further hour. The mixture was allowed to warm up to room temperature, water was added, and extraction was effected repeatedly with ethyl acetate. The combined organic phases were dried over sodium sulfate, and the solvent was removed under reduced pressure. After purification by column chromatography on silica gel with heptane/ethyl acetate, 4.72 g (55%) of a product with m/z (%)=419 [M⁺] was obtained. ¹H-NMR (400 MHz, CDCl₃): δ=0.26 (s, 6H), 0.98 (s, 9H), 6.02 (s, 1H), 7.23 (m, 2H), 7.40 (m, 2H).
e) 3-{[tert-Butyl(dimethyl)silyl]oxy}-1-(2-fluorophenyl)-1H-pyrazole: To a solution of 20.00 g (112.26 mmol) of 1-(2-fluorophenyl)-1H-pyrazol-3-ol in 900 ml of dichloromethane were successively added 21.05 g (208.1 mmol) of triethylamine and 15.32 g (101.64 mmol) of tert-butyl(chloro)dimethylsilane, and the mixture was stirred at room temperature for 4 hours. The solvent was removed under reduced pressure, and 29.60 g (quant.) of a product with m/z=293 [M⁺] was obtained, which was converted further without purification. ¹H-NMR (400 MHz, CDCl₃): δ=0.31 (s, 6H), 1.00 (s, 9H), 5.85 (d, 1H), 7.17 (m, 3H), 7.84 (m, 2H).

Methyl 2-{[4-bromo-5-(4-fluorophenyl)-1-phenyl-1H-pyrazol-3-yl]oxy}propanoate (I-132): To a solution of 1.10 g (2.91 mmol) of methyl 2-{[5-(4-fluorophenyl)-1-phenyl-1H-pyrazol-3-yl]oxy}propanoate in 16 ml of DMF was added 0.57 g (3.20 mmol) of N-bromosuccinimide, and the mixture was stirred at 70° C. for three hours. The solvent was removed under reduced pressure, the residue was taken up in water and extracted repeatedly with methylene chloride, the combined organic phases were dried over sodium sulfate, and the solvent was removed under reduced pressure. After purification by column chromatography on silica gel with heptane/ethyl acetate, 1.04 g (81%) of a product with m/z (%)=419 (50) [M⁺], 421 (50) [M⁺] was obtained. ¹H-NMR (400 MHz, CDCl₃): δ=1.69 (d, 3H), 3.79 (s, 3H), 5.26 (q, 1H), 7.07 (m, 4H), 7.22 (m, 5H).

Synthesis of the starting compounds: The starting compounds were synthesized by the methods described in Example No. I-182.

### 2-{[4-Bromo-5-(4-fluorophenyl)-1-phenyl-1H-pyrazol-3-yl]oxy}propanoic acid (I-138)

A mixture of 1.00 g (2.27 mmol) of methyl 2-{[4-bromo-5-(4-fluorophenyl)-1-phenyl-1H-pyrazol-3-yl]oxy}propanoate (I-132), 0.73 g (18.25 mmol) of 2N sodium hydroxide solution and 5 ml of tetrahydrofuran was stirred at room temperature for three hours. The solvent was removed under reduced pressure, and the remaining organic phase was adjusted to pH 2 with 2 N HCl. The precipitated solids were dried at 40° C. under reduced pressure, and 0.90 g (93%) of a colourless solid with m/z (%)=405 (50) [M⁺], 407 (50) [M⁺] was obtained. ¹H-NMR (400 MHz, CDCl₃): δ=1.71 (d, 3H), 5.27 (q, 1H), 7.08 (m, 4H), 7.25 (m, 5H).

### Methyl N-(2-{[4-bromo-5-(4-fluorophenyl)-1-phenyl-1H-pyrazol-3-yl]oxy}propanoyl)glycinate (III-056)

Under argon, the following were added successively to a solution of 0.15 g (0.35 mmol) of 2-{[4-bromo-5-(4-fluorophenyl)-1-phenyl-1H-pyrazol-3-yl]oxy}propanoic acid (I-138) in 4 ml of tetrahydrofuran: 2.13 g (1.68 mmol) of 2-methoxy-2-oxoethanaminium chloride, 1.71 g (1.68 mmol) of propanephosphonic anhydride (T3P, 50% solution in tetrahydrofuran) and 0.34 g (3.36 mmol) of triethylamine, and the mixture was stirred at 50° C. for 6 hours. The solvent was removed under reduced pressure, the residue was taken up in water and extracted repeatedly with methylene chloride, the combined organic phases were dried over sodium sulfate, and the solvent was removed under reduced pressure. After purification by column chromatography on silica gel with heptane/ethyl acetate, 0.14 g (79%) of a colourless oil with m/z (%)=478 (50) [M⁺], 476 (50) [M⁺] was obtained. ¹H-NMR (400 MHz, CDCl₃): δ=1.71 (d, 3H), 3.77 (s, 3H), 4.12 (m, 2H), 5.35 (q, 1H), 7.08 (m, 2H), 7.15 (m, 3H), 7.26 (m, 5H).

NMR data of selected examples:

The ¹H NMR data of selected examples of compounds of the general formula (I) are stated in two different ways, namely (a) conventional NMR evaluation and interpretation or (b) in the form of ¹H NMR peak lists according to the method described further down.

a) Conventional NMR Interpretation

I-094: ¹H-NMR (400 MHz, CDCl₃): δ=3.79 (s, 3H), 4.90 (s, 2H), 6.38 (d, 2H), 6.72 (d, 2H), 7.16 (t, 1H), 7.24 (m, 1H), 7.41 (m, 2H).

I-125: ¹H-NMR (400 MHz, CDCl₃): δ=1.70 (d, 3H), 3.79 (s, 3H), 5.23 (q, 1H), 6.88 (m, 1H), 7.0 (m, 3H), 7.17 (m, 2H), 7.27 (m, 1H), 7.40 (m, 1H).

I-132: ¹H-NMR (400 MHz, CDCl₃): δ=1.69 (d, 3H), 3.79 (s, 3H), 5.26 (q, 1H), 7.07 (m, 4H), 7.22 (m, 5H).

I-138: ¹H-NMR (400 MHz, CDCl₃): δ=1.71 (d, 3H), 5.27 (q, 1H), 7.08 (m, 4H), 7.25 (m, 5H).

I-143: ¹H-NMR (400 MHz, CDCl₃): δ=1.28 (t, 3H), 4.27 (q, 2H), 4.98 (s, 2H), 7.11 (m, 5H), 7.33 (t, 1H), 7.40 (m, 1H).

I-145: ¹H-NMR (400 MHz, CDCl₃): δ=1.69 (d, 3H), 3.79 (s, 3H), 5.26 (q, 1H), 7.08 (m, 4H), 7.25 (m, 5H).

I-165: ¹H-NMR (400 MHz, CDCl₃): δ=1.69 (d, 3H), 3.79 (s, 3H), 5.24 (q, 1H), 6.82 (m, 3H), 7.12 (m, 2H), 7.29 (m, 3H).

I-181: ¹H-NMR (400 MHz, CDCl₃): δ=3.81 (s, 3H), 4.91 (s, 2H), 7.00 (m, 3H), 7.13 (m, 3H).

I-182: ¹H-NMR (400 MHz, CDCl₃): δ=1.57 (d, 3H), 3.69 (s, 3H), 5.15 (q, 1H), 7.18 (m, 1H), 7.36 (m, 2H), 7.50 (m, 3H).

I-183: ¹H-NMR (400 MHz, CDCl₃): δ=3.81 (s, 3H), 4.91 (s, 2H), 7.07 (m, 1H), 7.13 (m, 3H), 7.21 (m, 2H).

I-184: ¹H-NMR (400 MHz, CDCl₃): δ=1.67 (d, 3H), 3.77 (s, 3H), 5.22 (q, 1H), 6.98 (m, 1H), 7.02 (m, 1H), 7.12 (m, 2H), 7.17 (t, 1H), 7.32 (m, 2H).

I-185: ¹H-NMR (400 MHz, CDCl₃): δ=1.68 (d, 3H), 3.78 (s, 3H), 5.18 (q, 1H), 7.12 (m, 6H).

III-056: ¹H-NMR (400 MHz, CDCl₃): δ=1.71 (d, 3H), 3.77 (s, 3H), 4.12 (m, 2H), 5.35 (q, 1H), 7.08 (m, 2H), 7.15 (m, 3H), 7.26 (m, 5H).

b) NMR Peak List Method

The ¹H NMR data of selected examples may also be stated in the form of ¹H NMR peak lists. For each signal peak, first the δ value in ppm and then the signal intensity in round brackets are listed. The δ value—signal intensity number pairs for different signal peaks are listed with separation from one another by semicolons.

The peak list for one example therefore takes the form of:

δ₁ (intensity₁); δ₂ (intensity₂); . . . αᵢ (intensityᵢ); . . . ; δₙ (intensityₙ)

The intensity of sharp signals correlates with the height of the signals in a printed example of an NMR spectrum in cm and shows the true ratios of the signal intensities. In the case of broad signals, several peaks or the middle of the signal and the relative intensity thereof may be shown in comparison to the most intense signal in the spectrum.

To calibrate the chemical shift of ¹H-NMR spectra, we use tetramethylsilane and/or the chemical shift of the solvent, particularly in the case of spectra measured in DMSO. Therefore, the tetramethylsilane peak may but need not occur in NMR peak lists.

The lists of the ¹H NMR peaks are similar to the conventional ¹H NMR printouts and thus usually contain all peaks listed in a conventional NMR interpretation.

In addition, like conventional ¹H NMR printouts, they may show solvent signals, signals of stereoisomers of the target compounds which are likewise provided by the invention, and/or peaks of impurities.

In the reporting of compound signals within the delta range of solvents and/or water, our lists of ¹H NMR peaks show the standard solvent peaks, for example peaks of DMSO in DMSO-D₆ and the peak of water, which usually have a high intensity on average.

The peaks of stereoisomers of the target compounds and/or peaks of impurities usually have a lower intensity on average than the peaks of the target compounds (for example with a purity of >90%).

Such stereoisomers and/or impurities may be typical of the particular preparation process. Their peaks can thus help in identifying reproduction of our preparation process with reference to “by-product fingerprints”.

An expert calculating the peaks of the target compounds by known methods (MestreC, ACD simulation, but also with empirically evaluated expected values) can, if required, isolate the peaks of the target compounds, optionally using additional intensity filters. This isolation would be similar to the relevant peak picking in conventional 1H NMR interpretation.

Further details of ¹H NMR peak lists can be found in the Research Disclosure Database Number 564025.

I-022: ¹H-NMR(400.0 MHz, CDCl3):
δ = 7.4060 (1.0); 7.4002 (1.6); 7.3929 (0.8); 7.3856 (1.8); 7.3822 (2.4); 7.3778 (1.4); 7.3706
(0.8); 7.3639 (2.0); 7.3608 (0.9); 7.3585 (0.8); 7.3502 (0.9); 7.3458 (3.0); 7.3283 (1.7); 7.3250
(1.2); 7.3188 (0.5); 7.3139 (2.6); 7.3116 (2.2); 7.3100 (2.3); 7.2988 (0.5); 7.2936 (1.4); 7.2889
(1.1); 7.2593 (32.9); 7.2236 (0.7); 7.2063 (0.9); 7.2039 (1.2); 7.0878 (0.7); 7.0850 (0.6); 7.0662
(0.7); 7.0628 (1.2); 7.0602 (0.8); 7.0415 (0.6); 7.0378 (0.6); 4.9164 (9.0); 3.8062 (16.0); 1.5424
(0.8); −0.0002 (12.9)
I-054: ¹H-NMR(400.0 MHz, CDCl3):
δ = 7.3511 (0.6); 7.3467 (0.7); 7.3322 (1.4); 7.3244 (2.8); 7.3202 (2.8); 7.3139 (1.8); 7.3111
(1.5); 7.3064 (2.5); 7.3039 (2.4); 7.2988 (0.8); 7.2970 (1.0); 7.2940 (1.4); 7.2922 (1.0); 7.2845
(1.2); 7.2834 (1.2); 7.2793 (3.1); 7.2745 (2.3); 7.2702 (0.9); 7.2685 (1.0); 7.2637 (1.2); 7.2622
(1.3); 7.2582 (19.1); 7.2546 (1.2); 7.1584 (0.6); 7.1566 (0.6); 7.1550 (0.7); 7.1535 (0.6); 7.1356
(0.9); 7.0253 (0.7); 7.0220 (0.6); 7.0045 (0.6); 7.0006 (1.1); 6.9969 (0.7); 6.9795 (0.6); 6.9762
(0.5); 5.2961 (1.8); 4.9226 (8.9); 3.8029 (16.0); −0.0002 (7.2)
I-055: ¹H-NMR(400.0 MHz, CDCl3):
δ = 7.3769 (0.6); 7.3623 (1.1); 7.3578 (1.4); 7.3433 (1.0); 7.3391 (1.2); 7.3310 (0.7); 7.3229
(0.5); 7.3107 (0.5); 7.2597 (20.0); 7.2089 (0.6); 7.2073 (0.7); 7.2056 (0.7); 7.1896 (0.9); 7.1879
(1.0); 7.1862 (1.0); 7.1487 (0.6); 7.1434 (0.6); 7.1390 (0.6); 7.1300 (0.6); 7.1246 (0.7); 7.1216
(0.7); 7.1172 (1.2); 7.1142 (0.7); 7.1029 (0.6); 7.0976 (1.3); 7.0929 (0.9); 7.0725 (0.7); 7.0558
(0.7); 7.0525 (0.6); 7.0351 (0.6); 7.0311 (1.0); 7.0274 (0.6); 7.0099 (1.0); 7.0064 (1.0); 6.9993
(0.6); 6.9956 (0.7); 5.2980 (4.1); 4.9117 (9.0); 3.8010 (16.0); 2.9549 (0.6); 2.8837 (0.5); 2.8825
(0.5); 1.5420 (2.6); −0.0002 (7.6)
I-056: ¹H-NMR(400.0 MHz, CDCl3):
δ = 7.2897 (0.6); 7.2878 (0.8); 7.2826 (3.3); 7.2785 (1.1); 7.2770 (1.3); 7.2737 (0.9); 7.2689
(3.9); 7.2680 (4.0); 7.2662 (4.6); 7.2593 (62.1); 7.2518 (2.2); 7.2474 (6.0); 7.2386 (1.2); 7.2345
(2.3); 7.2307 (1.4); 7.2237 (0.6); 7.2174 (1.5); 7.1987 (0.5); 7.1342 (1.6); 7.1324 (2.9); 7.1285
(4.0); 7.1228 (1.0); 7.1156 (1.6); 7.1131 (1.2); 7.1112 (2.8); 7.1080 (2.2); 7.0752 (3.1); 7.0696
(0.9); 7.0609 (0.5); 7.0582 (1.0); 7.0536 (4.5); 7.0483 (0.9); 7.0369 (0.8); 7.0315 (2.4); 4.9188
(13.3); 4.3060 (1.7); 4.2881 (5.4); 4.2703 (5.5); 4.2525 (1.8); 4.2304 (0.5); 1.5403 (16.0); 1.3199
(7.1); 1.3021 (14.6); 1.2925 (0.8); 1.2842 (7.0); 0.0080 (0.6); −0.0002 (22.6); −0.0085 (0.6)
I-057: ¹H-NMR(400.0 MHz, CDCl3):
δ = 7.5182 (1.7); 7.3087 (1.0); 7.3026 (0.8); 7.2985 (1.0); 7.2970 (1.1); 7.2929 (0.8); 7.2882
(1.3); 7.2857 (3.0); 7.2808 (4.1); 7.2771 (2.0); 7.2724 (3.7); 7.2691 (3.1); 7.2660 (4.4); 7.2593
(288.4); 7.2536 (6.4); 7.2500 (4.5); 7.2456 (0.8); 7.2431 (1.4); 7.2370 (1.3); 7.2354 (1.3);
7.2265 (0.6); 7.1557 (0.6); 7.1515 (0.8); 7.1452 (2.8); 7.1410 (3.4); 7.1351 (1.3); 7.1311 (0.7);
7.1287 (1.4); 7.1241 (2.8); 7.1207 (2.3); 7.0826 (3.2); 7.0770 (1.0); 7.0683 (0.6); 7.0657 (1.0);
7.0612 (4.9); 7.0558 (1.0); 7.0443 (0.8); 7.0390 (2.5); 6.9953 (1.7); 4.9952 (16.0); 0.3308 (0.6);
0.2375 (0.6); 0.0079 (3.5); −0.0002 (130.0); −0.0085 (3.9)
I-058: ¹H-NMR(400.0 MHz, CDCl3):
δ = 7.3327 (0.7); 7.3289 (1.8); 7.3248 (1.5); 7.3208 (5.1); 7.3166 (5.2); 7.3101 (3.8); 7.3056
(3.4); 7.3028 (4.2); 7.3006 (3.6); 7.2942 (1.7); 7.2903 (2.2); 7.2868 (2.1); 7.2838 (1.2); 7.2818
(1.3); 7.2796 (1.8); 7.2750 (1.5); 7.2734 (1.4); 7.2683 (5.0); 7.2638 (5.2); 7.2592 (30.7); 7.2518
(1.8); 7.2491 (1.8); 7.2471 (1.7); 7.2435 (2.1); 7.1409 (1.0); 7.1392 (1.1); 7.1375 (1.2); 7.1181
(1.7); 7.1023 (0.7); 7.1006 (0.8); 7.0990 (0.7); 7.0117 (1.2); 7.0084 (1.1); 6.9909 (1.1); 6.9871
(1.8); 6.9834 (1.1); 6.9659 (1.0); 6.9627 (0.9); 4.8954 (16.0); 4.2908 (2.0); 4.2729 (6.3); 4.2551
(6.4); 4.2373 (2.1); 2.1688 (1.5); 1.5705 (3.9); 1.3044 (7.6); 1.2866 (15.5); 1.2687 (7.5); 1.2565
(0.6); −0.0002 (10.8)
I-059: ¹H-NMR(601.6 MHz, CD3CN):
δ = 7.4173 (0.4); 7.4144 (0.6); 7.4090 (0.5); 7.4059 (0.8); 7.4049 (0.8); 7.4007 (1.9); 7.3968
(1.2); 7.3954 (0.9); 7.3935 (1.1); 7.3924 (1.1); 7.3874 (2.5); 7.3847 (1.6); 7.3800 (1.0); 7.3743
(1.3); 7.3717 (0.9); 7.3492 (0.8); 7.3468 (1.1); 7.3407 (2.5); 7.3385 (5.6); 7.3354 (2.9); 7.3292
(1.9); 7.3266 (3.6); 7.3256 (3.4); 7.3213 (0.7); 7.3201 (0.8); 7.3172 (1.1); 7.3143 (0.5); 7.3116
(0.7); 7.2409 (3.8); 7.2398 (4.1); 7.2387 (4.1); 7.2265 (4.1); 7.2142 (0.9); 7.2132 (0.9); 7.1210
(1.0); 7.1194 (1.0); 7.1071 (1.0); 7.1041 (1.4); 7.1019 (1.1); 7.0900 (1.0); 7.0882 (1.0); 4.8184
(16.0); 4.1925 (2.0); 4.1806 (6.0); 4.1688 (6.1); 4.1569 (2.0); 2.0980 (6.3); 1.9225 (0.4); 1.9182
(0.5); 1.9145 (3.5); 1.9104 (6.2); 1.9062 (9.3); 1.9022 (6.5); 1.8981 (3.3); 1.2109 (7.1); 1.1990
(14.5); 1.1872 (7.1)
I-060: ¹H-NMR(601.6 MHz, CD3CN):
δ = 7.4611 (0.5); 7.4582 (0.7); 7.4511 (1.3); 7.4483 (1.9); 7.4467 (1.5); 7.4372 (3.5); 7.4242
(3.7); 7.4191 (1.0); 7.4150 (1.6); 7.4124 (2.9); 7.4096 (2.0); 7.4065 (3.0); 7.4040 (1.8); 7.3968
(1.9); 7.3943 (4.6); 7.3862 (0.8); 7.3833 (1.6); 7.3797 (1.3); 7.3410 (3.9); 7.3388 (4.2); 7.3360
(2.1); 7.3312 (1.2); 7.3278 (3.0); 7.3250 (2.4); 7.2759 (1.2); 7.2635 (1.9); 7.2621 (1.8); 7.2511
(0.8); 7.1598 (1.2); 7.1579 (1.0); 7.1454 (1.2); 7.1428 (1.6); 7.1278 (0.9); 4.8860 (16.0); 4.4421
(0.6); 4.2520 (2.0); 4.2401 (5.9); 4.2283 (5.9); 4.2165 (2.0); 4.1374 (0.4); 4.1257 (0.4); 2.1602
(655.3); 2.1329 (0.4); 2.0871 (0.5); 2.0831 (0.8); 2.0790 (1.2); 2.0749 (0.8); 2.0707 (0.4);
1.9927 (4.6); 1.9846 (10.1); 1.9804 (13.4); 1.9766 (81.7); 1.9725 (144.5); 1.9684 (199.9);
1.9642 (135.7); 1.9601 (71.1); 1.8618 (0.4); 1.8577 (0.8); 1.8536 (1.2); 1.8496 (0.8); 1.8454
(0.4); 1.3017 (1.4); 1.2717 (7.1); 1.2599 (14.8); 1.2481 (7.0); 1.1792 (0.5); 1.1673 (1.0); 1.1555
(0.5); 0.0606 (0.4)
I-061: ¹H-NMR(300.1 MHz, CDCl3):
δ = 7.4164 (0.3); 7.4114 (0.3); 7.4078 (0.5); 7.4023 (0.5); 7.3941 (1.2); 7.3829 (1.1); 7.3748
(1.9); 7.3719 (2.1); 7.3652 (2.3); 7.3613 (2.1); 7.3576 (1.6); 7.3508 (2.0); 7.3467 (2.0); 7.3447
(2.0); 7.3380 (3.2); 7.3351 (2.7); 7.3242 (1.4); 7.3164 (2.0); 7.3078 (3.2); 7.2993 (1.9); 7.2967
(1.4); 7.2883 (0.6); 7.2808 (1.1); 7.2741 (0.8); 7.2602 (3.6); 7.2124 (0.8); 7.2103 (0.8); 7.1861
(1.1); 7.1609 (0.4); 7.1591 (0.4); 7.0860 (0.7); 7.0824 (0.6); 7.0580 (0.7); 7.0534 (1.1); 7.0491
(0.7); 7.0254 (0.6); 7.0219 (0.5); 5.2451 (0.5); 5.2218 (1.6); 5.1986 (1.7); 5.1754 (0.5); 3.7711
(16.0); 2.0412 (0.9); 1.6902 (6.7); 1.6670 (6.6); 1.5789 (1.0); 1.2570 (0.6); −0.0001 (2.8)
I-062: ¹H-NMR(300.1 MHz, CDCl3):
δ = 7.2640 (0.6); 7.2493 (1.7); 7.2200 (4.6); 7.1952 (7.9); 7.1898 (8.1); 7.1777 (7.5); 7.0497
(1.4); 7.0249 (1.9); 6.9986 (0.8); 6.9475 (1.1); 6.9158 (1.6); 6.8870 (0.9); 4.8826 (9.9); 3.7244
(16.0); 3.6578 (15.8); 1.5397 (0.4); 1.1862 (0.6); 0.0002 (5.8); −0.0709 (2.0)
I-063: ¹H-NMR(300.1 MHz, CDCl3):
δ = 7.3913 (0.6); 7.3857 (0.7); 7.3649 (1.5); 7.3594 (1.9); 7.3263 (6.4); 7.3138 (6.3); 7.3073
(3.8); 7.2861 (1.8); 7.2600 (18.4); 7.2391 (0.4); 7.2246 (0.4); 7.1861 (1.0); 7.1607 (1.4); 7.1348
(0.6); 7.0591 (0.8); 7.0265 (1.3); 6.9982 (0.8); 4.9254 (9.2); 3.7975 (16.0); 3.1683 (5.1); 2.0437
(1.0); 1.5555 (6.5); 1.2822 (0.7); 1.2550 (4.4); 1.2349 (0.4); 0.8807 (0.3); 0.0696 (1.2); −0.0001
(14.4); −0.0108 (0.5)
I-064: ¹H-NMR(300.1 MHz, CDCl3):
δ = 7.6138 (0.3); 7.3225 (9.6); 7.2594 (49.0); 7.1697 (3.7); 7.0995 (2.9); 7.0707 (4.5); 7.0482
(1.9); 6.9081 (0.4); 5.2712 (0.7); 5.2470 (2.0); 5.2242 (1.9); 5.2028 (0.7); 3.7824 (16.0); 1.7600
(0.3); 1.6946 (8.0); 1.6712 (7.7); 1.6536 (0.7); 1.5499 (68.6); 1.3134 (0.4); 1.2583 (1.7); 0.8833
(0.3); 0.8586 (0.4); 0.0686 (4.2); −0.0006 (41.4)
I-065: ¹H-NMR(300.1 MHz, CDCl3):
δ = 7.2888 (0.4); 7.2713 (2.4); 7.2591 (19.5); 7.2537 (4.9); 7.2415 (6.3); 7.2347 (5.3); 7.2243
(2.9); 7.1085 (2.7); 7.0987 (1.8); 7.0942 (1.7); 7.0826 (2.0); 7.0758 (1.6); 7.0495 (2.2); 7.0207
(3.7); 6.9915 (1.7); 4.9778 (9.0); 3.8115 (16.0); 3.7362 (16.0); 2.0422 (0.6); 1.5567 (4.7);
1.2580 (0.7); 0.0106 (0.9); −0.0001 (14.4); −0.0110 (0.5)
I-066: ¹H-NMR(300.1 MHz, CDCl3):
δ = 7.3529 (1.6); 7.3352 (2.0); 7.3231 (2.6); 7.3129 (1.4); 7.3054 (2.8); 7.3002 (2.6); 7.2807
(2.6); 7.2740 (5.3); 7.2596 (44.7); 7.2331 (0.8); 7.2189 (0.4); 7.2069 (0.4); 7.1766 (2.5); 7.1697
(2.3); 7.1491 (1.9); 7.1440 (1.6); 7.0685 (0.4); 7.0591 (2.0); 7.0302 (3.4); 7.0009 (1.6); 5.5336
(0.3); 4.9415 (8.8); 3.8057 (16.0); 3.1592 (5.4); 1.5404 (12.3); 1.5124 (2.2); 1.2547 (2.6);
0.0106 (1.8); −0.0001 (35.9); −0.0110 (1.6)
I-067: ¹H-NMR(300.1 MHz, CDCl3):
δ = 7.4439 (0.4); 7.4248 (0.5); 7.4170 (0.8); 7.3992 (0.7); 7.3969 (0.7); 7.3904 (0.7); 7.3714
(0.6); 7.3061 (0.5); 7.2854 (0.6); 7.2785 (1.1); 7.2600 (12.0); 7.2516 (1.0); 7.2310 (0.7); 7.1975
(0.4); 7.1942 (0.6); 7.1890 (0.5); 7.1858 (0.6); 7.1695 (0.8); 7.1664 (1.1); 7.1583 (2.0); 7.1554
(1.4); 7.1532 (1.5); 7.1499 (0.9); 7.1413 (0.5); 7.1380 (0.6); 7.1331 (1.2); 7.1296 (1.4); 7.1279
(1.3); 7.1245 (0.8); 7.0701 (0.5); 7.0670 (0.5); 7.0618 (0.6); 7.0587 (0.6); 7.0425 (0.8); 7.0395
(0.8); 7.0342 (1.0); 7.0311 (1.0); 7.0250 (0.8); 7.0191 (0.9); 7.0158 (1.0); 7.0117 (1.0); 7.0066
(0.6); 7.0034 (0.6); 6.9949 (1.4); 6.9881 (1.8); 6.9809 (1.1); 6.9640 (0.7); 6.9569 (1.0); 6.9494
(0.6); 6.9025 (1.0); 6.8987 (0.8); 6.8779 (0.8); 6.8757 (0.8); 6.8722 (0.8); 5.2616 (0.5); 5.2383
(1.6); 5.2151 (1.6); 5.1918 (0.5); 3.7919 (16.0); 1.7023 (6.5); 1.6790 (6.4); 1.5453 (6.2); 0.0107
(0.4); −0.0001 (9.8); −0.0037 (1.6); −0.0111 (0.3)
I-068: ¹H-NMR(300.1 MHz, CDCl3):
δ = 7.3751 (0.4); 7.3550 (0.6); 7.3481 (0.9); 7.3287 (1.0); 7.3222 (0.8); 7.3024 (0.8); 7.2600
(16.6); 7.2204 (0.4); 7.1935 (1.0); 7.1726 (1.2); 7.1668 (0.8); 7.1439 (1.2); 7.1204 (1.0); 7.1149
(1.0); 7.0921 (0.5); 7.0868 (0.5); 7.0642 (1.5); 7.0354 (1.9); 7.0050 (0.9); 7.0004 (1.0); 6.9734
(0.6); 6.9678 (0.8); 6.9651 (0.7); 6.9399 (2.2); 6.9273 (0.6); 6.9100 (1.4); 6.9020 (1.2); 6.8951
(0.6); 6.8348 (1.2); 6.8313 (1.1); 6.8061 (1.1); 4.9754 (8.1); 3.8212 (16.0); 3.7329 (15.7);
1.5501 (4.4); 1.2572 (0.4); 0.0107 (0.6); −0.0001 (13.9); −0.0112 (0.5)
I-069: ¹H-NMR(300.1 MHz, CDCl3):
δ = 7.3714 (0.3); 7.3491 (0.8); 7.3309 (0.8); 7.3210 (0.8); 7.3088 (0.4); 7.3010 (0.7); 7.2602
(29.7); 7.2400 (1.2); 7.2167 (1.0); 7.2109 (0.8); 7.1892 (0.7); 7.1210 (4.2); 7.0922 (2.7); 7.0652
(0.7); 6.9931 (2.2); 6.9679 (2.1); 6.9634 (2.3); 6.9441 (0.9); 6.9021 (1.5); 6.8773 (1.0); 4.9367
(8.9); 3.8143 (16.0); 3.1835 (5.5); 2.0440 (0.9); 1.5530 (16.2); 1.2826 (0.6); 1.2549 (3.6);
1.2353 (0.3); 0.0700 (0.4); −0.0001 (21.8)
I-070: ¹H-NMR(400.4 MHz, d₆-DMSO):
δ = 7.4457 (6.7); 7.4389 (7.8); 7.4292 (7.0); 7.3610 (1.9); 7.3435 (5.3); 7.3245 (4.8); 7.3061
(5.1); 7.3034 (5.7); 7.3003 (7.0); 7.2914 (5.8); 7.2857 (5.4); 7.2677 (1.1); 7.1494 (4.0); 7.1457
(6.6); 7.1255 (4.8); 4.9533 (14.0); 4.7640 (0.4); 4.2791 (0.4); 4.2199 (2.2); 4.2022 (6.9); 4.1845
(7.0); 4.1667 (2.4); 3.3849 (0.5); 3.3349 (117.7); 2.6779 (1.5); 2.6036 (0.5); 2.5588 (1.5);
2.5135 (168.7); 2.5090 (244.4); 2.5046 (207.4); 2.3361 (1.4); 2.3319 (1.2); 1.9965 (0.9); 1.2472
(0.8); 1.2323 (7.8); 1.2145 (16.0); 1.1968 (7.7); 1.1817 (0.7); 0.9624 (0.4); 0.9459 (0.4); 0.8677
(1.0); 0.8594 (0.7); 0.8502 (0.6); 0.8406 (0.6); 0.8237 (0.3)
I-071: ¹H-NMR(400.4 MHz, d₆-DMSO):
δ = 7.5293 (6.5); 7.5080 (7.8); 7.3894 (2.0); 7.3849 (1.0); 7.3719 (5.6); 7.3526 (4.8); 7.3301
(9.7); 7.3088 (9.1); 7.2899 (0.8); 7.1626 (6.0); 7.1447 (4.9); 4.9501 (14.5); 4.7623 (0.7); 4.2779
(0.8); 4.2132 (2.2); 4.1955 (6.9); 4.1777 (7.0); 4.1601 (2.3); 3.3367 (64.9); 2.6755 (1.3); 2.5106
(154.6); 2.5068 (190.6); 2.5027 (133.8); 2.4574 (0.5); 2.3336 (1.3); 1.2368 (1.2); 1.2261 (7.9);
1.2083 (16.0); 1.1906 (7.6)
I-072: ¹H-NMR(400.2 MHz, d₆-DMSO):
δ = 7.4958 (0.6); 7.4424 (7.8); 7.4346 (9.7); 7.4264 (8.5); 7.3025 (4.8); 7.2961 (5.3); 7.2869
(4.7); 7.2005 (8.0); 7.1929 (8.8); 7.1790 (15.0); 4.9380 (16.0); 4.7548 (0.6); 4.2687 (0.8);
4.2080 (2.6); 4.1906 (7.1); 4.1729 (7.2); 4.1553 (2.5); 3.6598 (0.6); 3.5534 (0.7); 3.5120 (0.8);
3.4784 (0.9); 3.4603 (1.2); 3.4199 (1.4); 3.3880 (2.1); 3.3250 (971.4); 3.3223 (684.4); 3.2696
(1.7); 2.6694 (5.8); 2.6004 (1.0); 2.5710 (1.3); 2.5003 (885.0); 2.4967 (826.3); 2.4405 (3.2);
2.4169 (1.1); 2.3273 (5.9); 1.2313 (1.8); 1.2213 (7.7); 1.2036 (15.4); 1.1858 (7.5)
I-073: ¹H-NMR(400.3 MHz, d₆-DMSO):
δ = 7.4684 (9.8); 7.4613 (9.5); 7.4572 (7.5); 7.4456 (2.8); 7.4417 (3.0); 7.4310 (7.7); 7.4256
(3.0); 7.4190 (3.3); 7.4134 (6.4); 7.4090 (7.4); 7.4011 (1.1); 7.3218 (5.8); 7.3157 (5.4); 7.3106
(5.0); 7.3070 (5.0); 7.3032 (3.5); 7.2982 (2.8); 7.1556 (8.6); 7.1502 (3.0); 7.1383 (5.5); 7.1335
(6.3); 4.9566 (13.8); 4.2163 (2.2); 4.1986 (6.8); 4.1808 (6.8); 4.1631 (2.3); 3.3452 (15.7);
3.3352 (56.3); 2.6800 (1.5); 2.6755 (1.5); 2.5109 (202.9); 2.5066 (202.5); 2.5023 (126.4);
2.3380 (1.5); 2.3337 (1.5); 1.2393 (1.9); 1.2290 (8.0); 1.2218 (3.4); 1.2113 (16.0); 1.2043 (2.2);
1.1936 (7.4)
I-074: ¹H-NMR(400.2 MHz, d₆-DMSO):
δ = 7.5517 (6.3); 7.5307 (7.9); 7.4582 (6.5); 7.4365 (7.6); 7.3490 (7.7); 7.3279 (6.5); 7.1765
(7.6); 7.1548 (6.8); 4.9517 (13.8); 4.7590 (2.1); 4.2731 (2.3); 4.2107 (2.2); 4.1930 (6.6); 4.1752
(6.7); 4.1576 (2.5); 4.1436 (0.9); 4.1259 (0.3); 3.4327 (0.4); 3.3843 (1.9); 3.3348 (292.4);
2.6735 (1.1); 2.5083 (131.3); 2.5046 (165.9); 2.5009 (127.3); 2.3314 (1.1); 1.2641 (0.6); 1.2345
(3.5); 1.2244 (8.6); 1.2066 (16.0); 1.1889 (7.9); 1.1718 (0.9); 0.8531 (0.6); 0.8357 (0.4)
I-075: ¹H-NMR(400.0 MHz, CDCl3):
δ = 7.4024 (0.7); 7.3917 (0.6); 7.3849 (2.0); 7.3778 (0.8); 7.3712 (1.4); 7.3668 (2.3); 7.3626
(1.4); 7.3539 (1.0); 7.3518 (0.9); 7.3495 (1.0); 7.3474 (1.0); 7.3417 (2.7); 7.3375 (2.1); 7.3266
(2.0); 7.3223 (5.4); 7.3092 (1.7); 7.3045 (4.1); 7.3019 (2.5); 7.2905 (5.6); 7.2875 (6.2); 7.2707
(3.7); 7.2662 (2.9); 7.2599 (27.3); 7.1604 (1.2); 7.1415 (1.9); 7.1227 (0.8); 7.0452 (1.2); 7.0421
(1.2); 7.0243 (1.1); 7.0209 (2.1); 7.0176 (1.3); 6.9997 (1.0); 6.9965 (1.1); 5.2970 (9.0); 4.9859
(16.0); 4.2998 (2.1); 4.2820 (6.4); 4.2641 (6.5); 4.2463 (2.1); 1.5482 (4.3); 1.3028 (7.5); 1.2955
(0.8); 1.2849 (15.2); 1.2781 (0.7); 1.2671 (7.3); −0.0002 (10.0)
I-076: ¹H-NMR(400.3 MHz, d₆-DMSO):
δ = 10.8519 (0.6); 7.3806 (2.5); 7.3583 (5.7); 7.3446 (5.6); 7.3195 (5.3); 7.2973 (7.6); 7.2754
(3.7); 7.2469 (1.6); 7.2246 (6.6); 7.2113 (8.0); 7.2041 (10.0); 7.1984 (10.7); 4.9427 (14.5);
4.7619 (0.7); 4.2752 (0.8); 4.2120 (2.4); 4.1945 (7.1); 4.1766 (7.6); 4.1590 (3.7); 4.1083 (1.0);
4.0954 (1.2); 4.0842 (0.7); 3.4315 (0.6); 3.3318 (372.2); 3.2799 (1.5); 3.2247 (0.7); 3.1792
(3.4); 3.1661 (3.1); 3.1088 (0.6); 3.0815 (0.7); 3.0416 (0.8); 3.0255 (1.1); 2.9520 (1.1); 2.8550
(0.6); 2.6761 (6.7); 2.6068 (1.3); 2.5072 (1051.7); 2.3992 (1.1); 2.3746 (0.8); 2.3341 (6.7);
2.2686 (0.6); 2.2217 (0.6); 2.1319 (0.7); 1.5669 (0.7); 1.5031 (0.6); 1.4028 (0.7); 1.2668 (1.3);
1.2262 (7.8); 1.2085 (16.0); 1.1908 (9.0); 1.0907 (1.0); 0.8812 (0.6); 0.8563 (0.7); 0.8490
(0.7); −1.5794 (0.6); −3.1735 (0.6); −3.5033 (0.6); −3.6659 (0.6); −3.8138 (0.6)
I-077: ¹H-NMR(400.2 MHz, d₆-DMSO):
δ = 7.5334 (6.0); 7.5121 (7.9); 7.3342 (7.8); 7.3128 (6.7); 7.2552 (1.3); 7.2493 (0.8); 7.2327
(6.1); 7.2205 (7.0); 7.2125 (8.8); 7.2073 (8.8); 7.1988 (2.0); 7.1849 (0.8); 7.1570 (0.3); 4.9418
(14.4); 4.7596 (0.4); 4.2746 (0.5); 4.2085 (2.2); 4.1908 (6.9); 4.1730 (7.0); 4.1553 (2.3); 3.3294
(39.3); 2.6732 (0.8); 2.5539 (1.4); 2.5088 (86.7); 2.5044 (111.2); 2.5001 (82.8); 2.4537 (0.8);
2.3357 (0.5); 2.3312 (0.6); 1.2690 (0.3); 1.2343 (3.4); 1.2221 (8.0); 1.2044 (16.0); 1.1866 (7.6);
0.8531 (0.6)
I-078: ¹H-NMR(400.3 MHz, d₆-DMSO):
δ = 7.4489 (7.3); 7.4271 (8.8); 7.3949 (2.6); 7.3791 (4.3); 7.3733 (6.1); 7.3596 (5.4); 7.3378
(5.4); 7.3157 (7.5); 7.2940 (2.9); 7.1658 (8.3); 7.1443 (7.6); 4.9529 (16.0); 4.2139 (2.4); 4.1963
(7.2); 4.1785 (7.4); 4.1609 (2.7); 3.3662 (0.4); 3.3316 (94.9); 3.2939 (0.7); 3.1784 (0.9); 3.1658
(0.9); 2.6756 (1.6); 2.5859 (0.3); 2.5069 (264.9); 2.3340 (1.6); 1.2279 (7.7); 1.2101 (15.2);
1.1924 (8.0); 1.1509 (0.4)
I-080: ¹H-NMR(400.4 MHz, d₆-DMSO):
δ = 7.6418 (0.9); 7.6268 (1.1); 7.6198 (1.9); 7.6048 (1.9); 7.5979 (1.2); 7.5828 (1.0); 7.5179
(6.2); 7.5133 (2.8); 7.5014 (2.5); 7.4965 (8.2); 7.4267 (1.0); 7.4200 (1.0); 7.3977 (1.5); 7.3939
(1.4); 7.3780 (1.0); 7.3712 (1.0); 7.3249 (7.7); 7.3203 (3.0); 7.3083 (2.2); 7.3035 (6.5); 7.2411
(0.8); 7.2214 (1.5); 7.1989 (0.8); 4.9214 (13.7); 4.1954 (2.2); 4.1777 (6.9); 4.1599 (7.0); 4.1421
(2.2); 3.3332 (182.0); 2.6809 (0.8); 2.6765 (1.2); 2.6718 (0.9); 2.5565 (0.5); 2.5298 (3.9);
2.5160 (65.0); 2.5118 (140.8); 2.5074 (199.2); 2.5030 (154.0); 2.4987 (77.5); 2.3389 (0.8);
2.3344 (1.2); 2.3300 (1.0); 1.9948 (0.8); 1.4029 (1.9); 1.2669 (0.9); 1.2380 (1.1); 1.2067 (7.8);
1.1979 (0.9); 1.1890 (16.0); 1.1803 (1.1); 1.1712 (7.5); 1.0921 (0.5); 0.9606 (0.8); 0.9440 (0.8);
0.8776 (0.4); 0.8658 (1.0); 0.8573 (0.7); 0.8444 (0.6); 0.8387 (0.9); 0.8212 (0.5)
I-081: ¹H-NMR(400.2 MHz, d₆-DMSO):
δ = 7.6335 (0.9); 7.6185 (1.0); 7.6115 (1.8); 7.5966 (1.8); 7.5895 (1.0); 7.5745 (0.9); 7.4070
(1.0); 7.4000 (1.1); 7.3842 (1.3); 7.3805 (1.4); 7.3775 (1.4); 7.3738 (1.3); 7.3649 (2.4); 7.3587
(1.9); 7.3512 (3.6); 7.3426 (4.1); 7.3348 (1.5); 7.3290 (3.6); 7.3221 (0.4); 7.3001 (0.7); 7.2935
(4.2); 7.2877 (1.1); 7.2764 (1.4); 7.2712 (6.1); 7.2656 (1.1); 7.2544 (0.9); 7.2490 (2.2); 7.2323
(0.6); 7.2289 (0.7); 7.2252 (0.6); 7.2086 (1.2); 7.1897 (0.6); 7.1861 (0.6); 7.1826 (0.5); 4.9151
(12.8); 4.7822 (0.8); 4.7586 (0.3); 4.2701 (0.4); 4.1947 (2.1); 4.1769 (6.8); 4.1591 (6.8); 4.1414
(2.2); 3.4235 (0.6); 3.3745 (0.8); 3.3625 (0.4); 3.3236 (429.6); 3.2988 (0.6); 3.2936 (0.4);
3.2740 (0.8); 2.6787 (0.8); 2.6742 (1.0); 2.6697 (0.7); 2.5277 (3.2); 2.5229 (5.0); 2.5142 (62.8);
2.5097 (132.0); 2.5052 (180.0); 2.5006 (127.3); 2.4962 (58.8); 2.4611 (0.8); 2.3410 (0.4);
2.3367 (0.8); 2.3321 (1.2); 2.3275 (0.8); 1.4017 (0.7); 1.2656 (0.6); 1.2383 (0.7); 1.2325 (0.7);
1.2146 (1.0); 1.2061 (7.7); 1.1968 (0.9); 1.1884 (16.0); 1.1706 (7.6); 1.0910 (0.4)
I-082: ¹H-NMR(400.0 MHz, d₆-DMSO):
δ = 13.0004 (0.8); 7.5268 (1.1); 7.5227 (1.4); 7.5076 (2.3); 7.5032 (2.8); 7.4881 (1.4); 7.4838
(1.6); 7.4730 (0.7); 7.4686 (0.7); 7.4603 (0.8); 7.4540 (1.3); 7.4488 (1.2); 7.4412 (1.3); 7.4368
(1.2); 7.4337 (1.3); 7.4290 (1.0); 7.4208 (1.0); 7.4164 (0.9); 7.4047 (1.0); 7.3952 (7.5); 7.3881
(7.2); 7.3787 (6.5); 7.3721 (2.0); 7.2953 (1.7); 7.2926 (2.3); 7.2863 (1.0); 7.2798 (4.9); 7.2758
(5.2); 7.2736 (7.4); 7.2681 (3.6); 7.2649 (5.9); 7.2557 (3.9); 7.2452 (1.8); 7.2403 (2.1); 7.2369
(1.6); 7.2192 (1.3); 7.2161 (1.2); 4.8316 (0.9); 4.8209 (16.0); 4.0382 (0.6); 4.0205 (0.6); 3.3141
(10.9); 2.5231 (1.4); 2.5184 (2.0); 2.5097 (20.0); 2.5052 (40.9); 2.5006 (55.8); 2.4961 (38.8);
2.4916 (17.6); 1.9876 (2.5); 1.2360 (0.8); 1.1920 (0.7); 1.1742 (1.4); 1.1564 (0.7); 0.0080
(0.6); −0.0002 (19.0); −0.0085 (0.6)
I-083: ¹H-NMR(400.0 MHz, CDCl3):
δ = 7.6410 (2.5); 7.6207 (2.9); 7.4145 (2.8); 7.3943 (2.5); 7.2592 (32.4); 7.1139 (1.5); 7.1084
(0.8); 7.1020 (1.8); 7.0967 (1.3); 7.0912 (2.5); 7.0851 (1.0); 7.0793 (2.4); 7.0025 (2.3); 6.9967
(0.8); 6.9908 (0.5); 6.9821 (2.8); 6.9653 (0.7); 6.9596 (1.6); 4.9345 (9.3); 3.8483 (0.6); 3.8148
(16.0); 3.8015 (0.8); 1.5409 (0.7); 1.2546 (0.7); 1.0706 (0.9); −0.0002 (12.2); −0.0084 (0.7)
I-084: ¹H-NMR(400.0 MHz, CDCl3):
δ = 7.4709 (1.1); 7.4545 (1.2); 7.4384 (0.8); 7.3819 (0.8); 7.3639 (1.8); 7.3463 (1.4); 7.2734
(1.9); 7.2595 (17.6); 7.2337 (3.1); 7.2098 (1.4); 5.2978 (0.6); 4.8440 (10.3); 3.7706 (16.0);
2.7713 (0.6); 1.5422 (1.7); 1.2429 (0.5); −0.0002 (6.2)
I-085: ¹H-NMR(400.3 MHz, d₆-DMSO):
δ = 7.5852 (1.9); 7.5689 (2.7); 7.5642 (2.6); 7.5481 (2.7); 7.5264 (1.0); 7.4298 (2.2); 7.4245
(2.2); 7.4054 (3.9); 7.4001 (3.4); 7.3865 (4.8); 7.3815 (4.5); 7.3688 (7.4); 7.3531 (4.1); 7.3492
(4.8); 7.3199 (3.5); 7.3073 (2.6); 7.3022 (3.6); 7.2814 (2.5); 7.2600 (2.9); 7.2373 (1.2); 7.1615
(7.2); 7.1579 (7.4); 7.1438 (4.7); 7.1401 (4.7); 5.0105 (0.9); 4.9852 (4.0); 4.9678 (11.3); 4.2777
(0.4); 4.2562 (0.4); 4.2192 (3.7); 4.2013 (7.9); 4.1836 (7.0); 4.1660 (2.1); 3.4369 (0.6); 3.3826
(10.3); 3.3562 (50.7); 3.3419 (98.1); 3.3364 (183.0); 3.2985 (0.8); 3.0350 (0.4); 2.9853 (0.4);
2.9509 (0.4); 2.9008 (0.3); 2.8834 (0.3); 2.7341 (0.5); 2.7272 (0.5); 2.7144 (0.6); 2.6801 (3.4);
2.6757 (3.4); 2.6055 (1.4); 2.5505 (52.5); 2.5113 (502.8); 2.5067 (493.7); 2.5027 (303.6);
2.4041 (0.6); 2.3779 (0.5); 2.3382 (3.3); 2.3337 (3.3); 1.2275 (10.4); 1.2156 (8.8); 1.2097
(16.0); 1.1980 (4.0); 1.1920 (7.2); 1.1541 (0.7); 0.8615 (0.3)
I-086: ¹H-NMR(400.2 MHz, d₆-DMSO):
δ = 7.5866 (0.9); 7.5702 (1.1); 7.5653 (1.7); 7.5493 (1.7); 7.5443 (1.1); 7.5281 (1.0); 7.4279
(1.0); 7.4217 (1.0); 7.4023 (1.3); 7.3983 (1.4); 7.3793 (0.9); 7.3727 (0.9); 7.2782 (1.0); 7.2736
(0.9); 7.2572 (1.7); 7.2512 (2.5); 7.2439 (0.8); 7.2348 (1.7); 7.2276 (5.5); 7.2222 (1.5); 7.2127
(5.4); 7.2071 (6.4); 7.2033 (2.9); 7.1995 (5.7); 7.1906 (1.2); 7.1836 (0.5); 7.1770 (0.6); 4.9798
(0.3); 4.9573 (10.3); 4.7572 (0.7); 4.2696 (0.9); 4.2138 (2.0); 4.1960 (6.4); 4.1783 (6.4); 4.1606
(2.3); 4.1430 (0.3); 3.3684 (0.6); 3.3623 (0.6); 3.3599 (0.6); 3.3425 (4.7); 3.3360 (1.9); 3.3181
(360.9); 3.3051 (1.6); 3.3038 (1.4); 3.3025 (1.3); 3.3012 (1.3); 3.3000 (1.3); 3.2937 (3.9);
2.6810 (1.0); 2.6765 (2.2); 2.6719 (3.0); 2.6673 (2.2); 2.6628 (1.1); 2.5662 (1.3); 2.5256 (18.4);
2.5209 (19.4); 2.5120 (161.2); 2.5075 (344.7); 2.5029 (469.2); 2.4984 (332.3); 2.4938 (151.3);
2.4799 (10.9); 2.3389 (1.0); 2.3344 (2.1); 2.3298 (2.9); 2.3253 (2.0); 1.2349 (0.7); 1.2227 (7.6);
1.2049 (16.0); 1.1872 (7.3)
I-087: ¹H-NMR(400.4 MHz, d₆-DMSO):
δ = 7.6065 (0.8); 7.5903 (1.0); 7.5852 (1.7); 7.5691 (1.7); 7.5642 (1.1); 7.5480 (0.9); 7.4688
(0.6); 7.4610 (6.4); 7.4556 (2.9); 7.4485 (1.3); 7.4443 (2.4); 7.4389 (7.9); 7.4312 (2.2); 7.4251
(1.5); 7.4060 (1.0); 7.3996 (1.0); 7.3022 (0.8); 7.2970 (0.8); 7.2811 (1.5); 7.2756 (1.4); 7.2597
(0.8); 7.2547 (0.7); 7.1886 (0.8); 7.1810 (7.4); 7.1756 (2.2); 7.1641 (2.0); 7.1588 (6.4); 7.1511
(0.7); 4.9731 (9.8); 4.2754 (0.4); 4.2196 (2.0); 4.2019 (6.4); 4.1841 (6.5); 4.1664 (2.1); 3.3273
(115.4); 2.6809 (0.6); 2.6762 (0.9); 2.6717 (0.6); 2.5298 (2.9); 2.5250 (4.4); 2.5163 (49.8);
2.5119 (106.9); 2.5073 (147.4); 2.5028 (106.8); 2.4983 (50.9); 2.4621 (0.5); 2.4577 (0.5);
2.3433 (0.3); 2.3389 (0.7); 2.3343 (0.9); 2.3297 (0.7); 1.2430 (0.9); 1.2281 (7.7); 1.2104 (16.0);
1.1926 (7.3)
I-088: ¹H-NMR(400.2 MHz, d₆-DMSO):
δ = 7.5608 (0.9); 7.5459 (1.0); 7.5388 (1.7); 7.5239 (1.8); 7.5168 (1.0); 7.5019 (0.9); 7.4849
(0.9); 7.4686 (1.1); 7.4637 (1.7); 7.4476 (1.8); 7.4426 (1.2); 7.4266 (2.3); 7.4224 (1.7); 7.4030
(2.7); 7.4009 (2.7); 7.3966 (2.5); 7.3804 (1.3); 7.3779 (1.4); 7.3734 (1.4); 7.2170 (1.1); 7.2108
(1.6); 7.2033 (1.0); 7.1957 (2.0); 7.1906 (2.8); 7.1745 (1.0); 7.1705 (1.3); 7.1680 (1.3); 4.9293
(12.7); 4.1977 (2.1); 4.1800 (6.7); 4.1622 (6.9); 4.1444 (2.2); 3.3772 (0.7); 3.3629 (0.3); 3.3264
(227.7); 3.2910 (0.4); 3.2883 (0.4); 3.2770 (0.6); 2.6831 (0.5); 2.6788 (0.9); 2.6742 (1.3);
2.6697 (0.9); 2.5606 (0.6); 2.5561 (0.8); 2.5515 (0.8); 2.5464 (0.7); 2.5276 (5.4); 2.5226 (8.4);
2.5142 (76.5); 2.5097 (155.6); 2.5052 (209.3); 2.5007 (149.9); 2.4962 (71.4); 2.4561 (0.7);
2.4514 (0.4); 2.3366 (0.9); 2.3321 (1.3); 2.3275 (0.9); 1.2400 (0.4); 1.2073 (7.7); 1.1896 (16.0);
1.1719 (7.5)
I-089: ¹H-NMR(400.2 MHz, d₆-DMSO):
δ = 7.3952 (1.5); 7.3912 (1.7); 7.3742 (6.8); 7.3591 (4.4); 7.3554 (6.0); 7.3149 (0.3); 7.2859
(2.6); 7.2717 (4.0); 7.2638 (5.4); 7.2550 (3.1); 7.2501 (4.7); 7.2266 (5.2); 7.2045 (7.0); 7.1871
(1.7); 7.1825 (2.4); 7.0328 (2.1); 7.0112 (4.8); 7.0069 (4.2); 6.9917 (2.6); 6.9854 (3.3); 4.8806
(15.7); 4.1879 (2.4); 4.1701 (7.4); 4.1524 (7.4); 4.1347 (2.4); 3.4614 (0.9); 3.4361 (27.6);
3.3768 (0.4); 3.3704 (0.4); 3.3271 (178.3); 2.6775 (0.8); 2.6732 (0.9); 2.5488 (0.6); 2.5085
(135.6); 2.5043 (155.1); 2.5003 (105.1); 2.3352 (0.8); 2.3313 (0.9); 1.2382 (1.0); 1.2062 (8.3);
1.1884 (16.0); 1.1708 (7.7)
I-090: ¹H-NMR(400.3 MHz, d₆-DMSO):
δ = 7.4603 (3.2); 7.4537 (6.8); 7.4487 (2.4); 7.4370 (4.7); 7.4323 (7.5); 7.4261 (0.9); 7.4077
(1.4); 7.4034 (1.7); 7.3910 (4.7); 7.3870 (5.6); 7.3721 (3.9); 7.3679 (4.8); 7.2576 (3.9); 7.2511
(8.1); 7.2460 (2.2); 7.2345 (4.0); 7.2298 (5.8); 7.2231 (0.6); 7.0456 (2.0); 7.0428 (1.8); 7.0266
(4.4); 7.0237 (4.8); 7.0195 (3.7); 7.0110 (1.5); 7.0045 (2.6); 6.9978 (2.7); 4.8881 (13.5); 4.1923
(2.3); 4.1814 (2.9); 4.1744 (6.7); 4.1637 (2.9); 4.1567 (6.8); 4.1390 (2.2); 4.0273 (0.3); 3.6148
(0.6); 3.5402 (0.3); 3.4429 (10.8); 3.4362 (25.5); 3.3948 (0.6); 3.3878 (0.8); 3.3800 (1.0);
3.3729 (1.5); 3.3371 (214.4); 3.3300 (520.8); 3.3160 (3.0); 3.3083 (1.5); 3.3002 (0.7); 3.2962
(1.0); 3.2797 (0.4); 3.2308 (0.4); 3.0561 (0.4); 3.0106 (0.5); 2.9816 (0.6); 2.9511 (0.6); 2.9453
(0.5); 2.6819 (4.5); 2.6778 (4.8); 2.6733 (3.2); 2.6168 (0.5); 2.5857 (0.4); 2.5132 (713.7);
2.5088 (763.9); 2.5044 (482.6); 2.5001 (194.1); 2.4792 (3.2); 2.4634 (1.4); 2.4590 (1.5); 2.4544
(1.4); 2.4192 (1.0); 2.3767 (0.4); 2.3402 (4.4); 2.3358 (4.8); 2.3312 (3.0); 1.9964 (0.8); 1.7669
(0.5); 1.2690 (0.8); 1.2456 (1.7); 1.2172 (2.9); 1.2101 (7.8); 1.1996 (6.1); 1.1924 (16.0); 1.1818
(3.4); 1.1747 (7.2); 0.8686 (0.4); 0.8613 (0.5)
I-091: ¹H-NMR(400.3 MHz, d₆-DMSO):
δ = 7.3739 (7.3); 7.3583 (8.7); 7.3408 (3.4); 7.3314 (2.0); 7.2935 (1.5); 7.2749 (2.5); 7.2569
(2.4); 7.2362 (1.0); 7.1114 (2.2); 7.0910 (3.2); 7.0698 (1.6); 7.0360 (3.1); 7.0173 (4.6); 6.9953
(3.8); 6.9894 (4.7); 6.9680 (3.4); 4.9007 (16.0); 4.1981 (3.9); 4.1805 (8.2); 4.1628 (7.4); 4.1451
(2.4); 3.4607 (25.7); 3.4256 (0.4); 3.3323 (60.6); 3.3017 (0.4); 2.6821 (1.1); 2.5128 (136.3);
2.5089 (136.9); 2.4748 (1.1); 2.4697 (1.0); 2.3359 (1.0); 1.2140 (10.2); 1.1962 (16.0); 1.1785 (7.2)
I-092: ¹H-NMR(400.2 MHz, d₆-DMSO):
δ = 7.3816 (1.2); 7.3774 (1.6); 7.3586 (8.0); 7.3520 (6.0); 7.3433 (8.0); 7.3397 (9.9); 7.3360
(7.4); 7.2377 (0.6); 7.2276 (3.5); 7.2239 (2.3); 7.2201 (3.1); 7.2176 (2.6); 7.2142 (2.4); 7.2108
(2.7); 7.2036 (2.4); 7.0217 (1.5); 7.0189 (1.7); 7.0027 (2.4); 7.0001 (3.1); 6.9953 (2.6); 6.9925
(2.5); 6.9836 (1.3); 6.9808 (1.7); 6.9731 (2.5); 5.7580 (1.8); 4.8805 (13.4); 4.1892 (2.1); 4.1715
(6.6); 4.1537 (6.7); 4.1360 (2.2); 3.4296 (0.4); 3.4030 (25.5); 3.3802 (2.7); 3.3701 (0.7); 3.3545
(1.7); 3.3298 (523.0); 3.2975 (0.7); 3.2934 (0.8); 3.2848 (1.2); 3.2792 (2.6); 3.2723 (0.5);
2.6769 (0.8); 2.6724 (1.1); 2.6679 (0.8); 2.5541 (1.4); 2.5258 (3.8); 2.5209 (5.9); 2.5124 (63.4);
2.5079 (131.9); 2.5034 (179.4); 2.4989 (128.1); 2.4944 (60.1); 2.4536 (1.6); 2.3349 (0.9);
2.3304 (1.2); 2.3258 (0.8); 1.2394 (1.1); 1.2075 (7.6); 1.1898 (16.0); 1.1721 (7.4)
I-093: ¹H-NMR(400.0 MHz, CDCl3):
δ = 7.3830 (0.5); 7.3789 (0.9); 7.3771 (0.9); 7.3712 (5.9); 7.3668 (5.6); 7.3599 (2.3); 7.3577
(2.0); 7.3532 (3.3); 7.3518 (2.8); 7.3448 (0.9); 7.3410 (1.1); 7.2799 (0.9); 7.2710 (3.8); 7.2666
(4.2); 7.2595 (24.7); 7.2554 (3.0); 7.2466 (2.8); 7.2434 (1.2); 7.2387 (1.0); 7.2354 (1.1); 7.2185
(0.7); 6.9026 (0.7); 6.8852 (0.8); 6.8786 (1.3); 6.8612 (1.3); 6.8550 (0.8); 6.8377 (0.7); 5.2973
(3.1); 4.8815 (16.0); 4.8074 (3.9); 4.3030 (2.0); 4.2852 (6.3); 4.2674 (6.5); 4.2498 (2.5); 4.2332
(1.5); 1.5462 (0.9); 1.3149 (7.8); 1.2970 (16.0); 1.2869 (2.1); 1.2792 (7.6); 1.2690 (3.9); 1.2512
(1.9); −0.0002 (10.0)
I-094: ¹H-NMR(400.0 MHz, CDCl3):
δ = 7.4347 (0.5); 7.4326 (0.5); 7.4283 (0.6); 7.4203 (0.5); 7.4136 (1.1); 7.4084 (0.9); 7.3949
(1.3); 7.3899 (1.0); 7.3751 (0.8); 7.3707 (0.7); 7.2598 (43.6); 7.2507 (0.9); 7.2476 (1.0); 7.2459
(1.0); 7.2291 (1.2); 7.2104 (0.7); 7.1648 (0.7); 7.1615 (0.6); 7.1440 (0.7); 7.1404 (1.2); 7.1369
(0.7); 7.1194 (0.6); 7.1162 (0.6); 6.7312 (1.4); 6.7218 (2.0); 6.7117 (1.5); 6.3895 (1.7); 6.3847
(1.7); 6.3791 (1.6); 6.3743 (1.6); 6.1771 (1.0); 4.9050 (0.5); 4.8896 (8.9); 4.8481 (1.2); 4.8039
(1.4); 3.7940 (1.5); 3.7896 (16.0); 3.7810 (2.7); 3.7746 (2.2); 2.9186 (14.2); −0.0002
(17.8); −0.0085 (0.6)
I-095: ¹H-NMR(400.0 MHz, CDCl3):
δ = 7.4131 (0.6); 7.4112 (0.5); 7.4004 (1.0); 7.3962 (0.7); 7.3926 (0.6); 7.3847 (0.6); 7.3804
(1.6); 7.3766 (0.8); 7.3617 (1.0); 7.3575 (0.6); 7.2594 (27.0); 7.2536 (1.1); 7.2386 (0.7); 7.2371
(0.7); 7.2338 (1.0); 7.2185 (0.9); 7.2171 (1.0); 7.2155 (1.0); 7.1518 (0.7); 7.1486 (0.6); 7.1310
(0.6); 7.1275 (1.1); 7.1240 (0.6); 7.1064 (0.6); 7.1033 (0.5); 6.7232 (1.4); 6.7140 (1.8); 6.7131
(1.8); 6.7038 (1.4); 6.3851 (1.7); 6.3803 (1.7); 6.3747 (1.6); 6.3699 (1.6); 5.2978 (0.6); 4.8889
(9.0); 4.8440 (2.8); 3.7872 (16.0); 3.7705 (5.0); −0.0002 (11.8)
I-096: ¹H-NMR(400.0 MHz, CDCl3):
δ = 7.5179 (0.5); 7.3913 (0.6); 7.3860 (1.0); 7.3783 (4.6); 7.3746 (5.1); 7.3684 (2.0); 7.3642
(1.7); 7.3600 (3.2); 7.3584 (2.8); 7.3512 (1.0); 7.3470 (1.1); 7.3368 (0.6); 7.2912 (0.9); 7.2811
(0.6); 7.2739 (4.4); 7.2697 (4.7); 7.2646 (2.8); 7.2590 (92.5); 7.2497 (3.9); 7.2293 (0.9); 6.9950
(0.5); 6.9132 (0.7); 6.8958 (0.8); 6.8893 (1.2); 6.8720 (1.2); 6.8658 (0.8); 6.8484 (0.7); 5.2981
(0.5); 4.9621 (16.0); 4.8889 (2.0); 2.0451 (0.8); 1.3325 (1.8); 1.2842 (3.0); 1.2767 (1.2); 1.2552
(5.7); 1.2409 (0.7); 0.8799 (0.8); 0.0079 (0.9); −0.0002 (40.4); −0.0085 (1.6)
I-097: ¹H-NMR(400.0 MHz, CDCl3):
δ = 7.3575 (0.9); 7.3418 (1.2); 7.3366 (1.9); 7.3225 (2.0); 7.3166 (1.4); 7.3016 (1.9); 7.2814
(1.5); 7.2755 (1.3); 7.2601 (27.9); 7.2592 (28.9); 7.2385 (1.3); 7.1043 (1.0); 7.0861 (1.7);
7.0799 (2.0); 7.0651 (0.9); 7.0589 (1.0); 7.0270 (4.7); 7.0061 (3.2); 6.9985 (2.1); 6.9276 (0.9);
6.9102 (1.0); 6.9041 (1.8); 6.8867 (1.8); 6.8803 (1.2); 6.8629 (0.9); 5.2977 (0.5); 4.8779 (16.0);
4.3029 (2.0); 4.2850 (6.0); 4.2672 (6.2); 4.2494 (2.2); 1.3148 (6.3); 1.2976 (12.6); 1.2969
(12.7); 1.2797 (6.5); 1.2791 (6.5); 0.0007 (10.2); −0.0002 (10.6)
I-098: ¹H-NMR(400.0 MHz, CDCl3):
δ = 7.3715 (1.0); 7.3571 (1.0); 7.3499 (1.6); 7.3354 (1.6); 7.3291 (1.7); 7.3141 (2.0); 7.3087
(1.7); 7.2941 (1.5); 7.2904 (0.8); 7.2881 (0.8); 7.2742 (1.2); 7.2591 (31.0); 7.0780 (0.6); 7.0755
(0.7); 7.0716 (0.8); 7.0692 (0.8); 7.0569 (1.1); 7.0539 (1.2); 7.0481 (1.6); 7.0357 (0.6); 7.0332
(0.7); 7.0297 (1.9); 7.0267 (2.7); 7.0237 (1.6); 7.0142 (1.2); 7.0101 (3.1); 7.0055 (3.0); 6.9901
(1.3); 6.9861 (1.2); 6.9800 (0.8); 6.9372 (0.6); 6.9335 (0.6); 6.9304 (0.7); 6.9267 (0.7); 6.9180
(0.7); 6.9146 (1.1); 6.9112 (1.2); 6.9078 (1.2); 6.9045 (0.7); 6.8958 (0.6); 6.8921 (0.6); 6.8889
(0.6); 6.8852 (0.6); 6.8178 (1.1); 6.8109 (0.9); 6.7967 (1.1); 6.7930 (1.2); 6.7900 (1.1); 6.7862
(1.0); 6.7721 (1.0); 6.7652 (0.9); 4.8831 (16.0); 4.2944 (2.0); 4.2765 (6.3); 4.2587 (6.4); 4.2409
(2.1); 1.3054 (7.5); 1.2876 (15.3); 1.2697 (7.4); −0.0002 (11.4)
I-099: ¹H-NMR(400.0 MHz, CDCl3):
δ = 8.2827 (0.5); 8.2799 (0.5); 8.2771 (0.5); 8.2623 (0.8); 8.2592 (0.8); 8.2565 (0.6); 8.2084
(3.4); 8.2034 (1.0); 8.1912 (1.1); 8.1861 (3.6); 8.1512 (0.8); 8.1466 (1.1); 8.1418 (0.6); 7.6937
(0.6); 7.6742 (0.8); 7.6707 (0.7); 7.6360 (0.6); 7.5872 (1.0); 7.5668 (1.4); 7.5472 (0.7); 7.5188
(0.8); 7.5133 (0.6); 7.5077 (3.0); 7.5031 (1.2); 7.4905 (1.1); 7.4856 (2.7); 7.4176 (0.9); 7.4129
(0.7); 7.4101 (0.8); 7.4078 (0.7); 7.3999 (1.4); 7.3973 (1.3); 7.3846 (1.1); 7.3810 (1.4); 7.3766
(0.9); 7.3690 (0.8); 7.3647 (1.3); 7.3469 (0.8); 7.3423 (0.8); 7.2890 (0.7); 7.2599 (100.1);
7.2425 (0.6); 7.2350 (0.7); 7.2213 (1.0); 7.2150 (1.0); 7.1979 (0.5); 7.0625 (0.7); 7.0594 (0.7);
7.0416 (0.7); 7.0381 (1.2); 7.0346 (0.8); 7.0316 (0.5); 7.0282 (0.8); 7.0169 (0.7); 7.0139 (0.7);
6.9958 (0.6); 5.2986 (12.4); 5.0260 (6.6); 5.0221 (9.1); 5.0070 (0.6); 4.9412 (3.2); 3.8380 (1.2);
3.8347 (0.5); 3.8264 (4.1); 3.8202 (12.1); 3.8183 (16.0); 3.8108 (0.8); 3.7898 (1.2); 3.7746
(5.5); 3.6222 (0.5); 0.0079 (1.1); −0.0002 (36.6); −0.0085 (1.1)
I-100: ¹H-NMR(400.0 MHz, CDCl3):
δ = 7.3504 (1.4); 7.3481 (1.1); 7.3439 (5.5); 7.3392 (5.4); 7.3314 (3.4); 7.3298 (2.8); 7.3279
(3.5); 7.3264 (3.8); 7.3248 (3.2); 7.3226 (1.3); 7.3188 (1.1); 7.3149 (2.5); 7.3071 (1.4); 7.3025
(0.7); 7.2926 (1.1); 7.2728 (0.6); 7.2650 (3.6); 7.2587 (28.7); 7.2522 (2.4); 7.2502 (1.8); 7.2487
(1.8); 7.2475 (1.8); 7.2460 (1.6); 7.2429 (1.5); 7.2404 (2.4); 7.2353 (0.6); 6.9073 (0.6); 6.9035
(0.6); 6.9004 (0.6); 6.8967 (0.7); 6.8880 (0.7); 6.8846 (1.0); 6.8812 (1.2); 6.8778 (1.1); 6.8745
(0.7); 6.8658 (0.6); 6.8620 (0.6); 6.8589 (0.6); 6.8552 (0.6); 6.7965 (0.9); 6.7896 (0.8); 6.7753
(1.0); 6.7718 (1.1); 6.7686 (1.0); 6.7650 (0.9); 6.7507 (0.9); 6.7439 (0.8); 5.2966 (1.3); 4.8878
(14.8); 4.2952 (1.9); 4.2774 (5.9); 4.2595 (6.0); 4.2417 (2.0); 1.3060 (7.7); 1.2882 (16.0);
1.2703 (7.7); −0.0002 (10.6)
I-101: ¹H-NMR(400.0 MHz, CDCl3):
δ = 7.2865 (0.8); 7.2822 (2.0); 7.2768 (1.0); 7.2693 (4.0); 7.2656 (2.0); 7.2602 (19.7); 7.2547
(1.5); 7.2509 (3.0); 7.2489 (2.4); 7.2470 (2.5); 7.2420 (0.9); 7.2379 (1.7); 7.2341 (1.0); 7.2206
(1.2); 7.1328 (2.0); 7.1288 (2.9); 7.1232 (0.7); 7.1161 (1.0); 7.1115 (2.0); 7.1085 (1.7); 7.0752
(2.1); 7.0697 (0.7); 7.0583 (0.6); 7.0534 (3.3); 7.0485 (0.7); 7.0370 (0.6); 7.0317 (1.7); 4.9414
(9.1); 4.7549 (1.2); 3.8129 (16.0); 3.7951 (1.3); 2.9544 (3.4); 2.8824 (2.9); 2.7669 (2.8); 1.5676
(1.4); −0.0002 (6.1)
I-102: ¹H-NMR(400.0 MHz, CDCl3):
δ = 7.5185 (0.6); 7.3767 (0.7); 7.3624 (0.9); 7.3575 (1.3); 7.3506 (0.5); 7.3436 (0.9); 7.3424
(0.8); 7.3391 (1.1); 7.3379 (1.0); 7.3314 (0.8); 7.3300 (0.5); 7.3232 (0.6); 7.3110 (0.6); 7.2695
(0.5); 7.2687 (0.6); 7.2679 (0.7); 7.2671 (0.7); 7.2663 (0.8); 7.2655 (0.9); 7.2647 (1.1); 7.2638
(1.5); 7.2596 (105.1); 7.2093 (0.8); 7.2075 (0.6); 7.2058 (0.7); 7.2043 (0.6); 7.1899 (1.0);
7.1882 (1.0); 7.1863 (1.0); 7.1853 (0.9); 7.1689 (0.5); 7.1485 (0.6); 7.1431 (0.6); 7.1391 (0.6);
7.1296 (0.6); 7.1243 (0.7); 7.1212 (0.7); 7.1176 (1.0); 7.1141 (0.6); 7.1026 (0.6); 7.0975 (1.2);
7.0928 (0.8); 7.0726 (0.7); 7.0559 (0.6); 7.0529 (0.6); 7.0352 (0.6); 7.0315 (1.0); 7.0279 (0.6);
7.0098 (0.9); 7.0064 (0.9); 7.0008 (0.5); 6.9991 (0.6); 6.9956 (1.1); 6.9938 (0.5); 4.9228 (1.0);
4.9118 (8.1); 3.8066 (2.2); 3.8014 (16.0); 2.9618 (0.8); 2.9561 (2.2); 2.9552 (2.3); 2.8845 (2.0);
2.8830 (2.0); 2.7731 (6.2); 1.5415 (8.4); 0.0079 (1.4); 0.0046 (0.6); 0.0038 (0.7); 0.0021
(1.9); −0.0002 (44.3); −0.0028 (2.2); −0.0043 (1.0); −0.0052 (0.7); −0.0060 (0.6); −0.0085 (1.3)
I-104: ¹H-NMR(400.6 MHz, CDCl3):
δ = 7.4359 (0.5); 7.4215 (0.7); 7.4159 (0.9); 7.4016 (0.9); 7.3956 (0.8); 7.3813 (0.7); 7.2972
(0.7); 7.2819 (1.1); 7.2766 (1.7); 7.2604 (28.3); 7.2410 (1.0); 7.1907 (0.7); 7.1885 (0.7); 7.1844
(0.7); 7.1822 (0.7); 7.1697 (1.1); 7.1675 (1.2); 7.1633 (1.2); 7.1612 (1.2); 7.1551 (1.5); 7.1525
(1.5); 7.1512 (1.6); 7.1488 (1.4); 7.1423 (0.8); 7.1400 (0.8); 7.1359 (1.2); 7.1334 (1.2); 7.1320
(1.2); 7.1297 (0.8); 7.0635 (0.6); 7.0613 (0.6); 7.0572 (0.7); 7.0552 (0.6); 7.0428 (1.0); 7.0407
(1.0); 7.0365 (1.1); 7.0346 (1.0); 7.0197 (1.3); 7.0156 (1.4); 7.0093 (0.8); 6.9967 (1.2); 6.9906
(1.6); 6.9857 (1.6); 6.9793 (0.8); 6.9670 (0.9); 6.9616 (1.2); 6.9559 (0.7); 6.8979 (1.1); 6.8948
(1.0); 6.8790 (1.0); 6.8748 (0.9); 5.3002 (2.3); 5.2511 (0.5); 5.2336 (1.7); 5.2162 (1.7); 5.1988
(0.5); 3.7933 (16.0); 1.7003 (6.8); 1.6829 (6.7); 1.5400 (13.2); 0.0077 (2.4); −0.0002
(30.5); −0.0085 (1.6)
I-107: ¹H-NMR(400.0 MHz, CDCl3):
δ = 8.0573 (0.5); 7.3503 (0.5); 7.3445 (0.8); 7.3300 (0.8); 7.3256 (0.6); 7.3242 (0.6); 7.3102
(0.6); 7.2615 (26.6); 7.1267 (1.6); 7.1210 (0.6); 7.1148 (1.7); 7.1096 (1.4); 7.1037 (2.7); 7.0977
(0.7); 7.0955 (0.5); 7.0918 (2.6); 7.0884 (0.8); 7.0861 (0.6); 7.0822 (1.0); 7.0444 (0.8); 7.0418
(1.0); 7.0406 (1.2); 7.0381 (1.0); 7.0254 (0.9); 7.0230 (2.3); 7.0192 (1.5); 7.0015 (0.8); 6.9975
(0.9); 6.9953 (0.7); 6.9914 (2.6); 6.9855 (0.6); 6.9741 (0.7); 6.9710 (2.4); 6.9685 (1.8); 6.9653
(0.7); 6.9539 (0.5); 6.9482 (1.6); 4.9282 (8.3); 3.8127 (16.0); 2.9762 (4.3); 2.8980 (3.9); 2.7690
(10.4); −0.0002 (9.7)
I-108: ¹H-NMR(400.0 MHz, CDCl3):
δ = 7.3837 (0.5); 7.3774 (0.7); 7.3758 (0.6); 7.3632 (0.8); 7.3583 (0.6); 7.3570 (0.6); 7.3435
(0.6); 7.2628 (28.0); 7.1837 (0.6); 7.1812 (0.5); 7.1747 (0.5); 7.1625 (0.9); 7.1598 (0.8); 7.1563
(0.7); 7.1536 (0.9); 7.1349 (0.6); 7.1266 (1.6); 7.1208 (0.6); 7.1148 (1.7); 7.1093 (1.1); 7.1035
(2.3); 7.0977 (0.8); 7.0954 (0.5); 7.0918 (2.2); 7.0739 (0.9); 7.0713 (1.0); 7.0700 (1.2); 7.0675
(1.0); 7.0517 (2.2); 7.0485 (1.4); 7.0460 (0.8); 7.0305 (0.7); 7.0264 (0.6); 7.0249 (0.7); 7.0202
(0.7); 7.0119 (2.4); 7.0059 (0.7); 6.9945 (0.7); 6.9920 (2.5); 6.9889 (1.7); 6.9861 (0.7); 6.9748
(0.6); 6.9690 (1.5); 5.2991 (1.3); 5.0251 (7.9); 4.9111 (0.8); 3.8219 (16.0); 3.8069 (1.7); 3.0134
(14.3); 2.9409 (8.9); 2.0822 (9.8); −0.0002 (10.3)
I-109: ¹H-NMR(400.0 MHz, CDCl3):
δ = 8.0119 (1.2); 7.3489 (0.6); 7.3344 (0.6); 7.2610 (55.1); 7.1087 (1.4); 7.1055 (0.6); 7.1030
(0.6); 7.0969 (1.4); 7.0932 (0.8); 7.0910 (1.3); 7.0859 (2.2); 7.0798 (0.6); 7.0739 (1.8); 7.0656
(0.5); 7.0380 (0.7); 7.0354 (0.8); 7.0341 (1.0); 7.0316 (0.8); 7.0189 (0.6); 7.0162 (0.7); 7.0150
(1.0); 7.0125 (0.8); 7.0073 (0.6); 7.0018 (0.5); 6.9971 (0.6); 6.9850 (0.7); 6.9801 (0.6); 6.9769
(2.0); 6.9710 (0.6); 6.9597 (0.6); 6.9566 (1.9); 6.9539 (1.5); 6.9507 (0.6); 6.9336 (1.3); 4.9215
(6.5); 3.8097 (12.6); 3.0090 (16.0); 2.9580 (11.9); 2.9573 (12.6); 2.8825 (10.8); 2.8810 (11.0);
2.7716 (5.0); 1.5585 (7.2); 0.0080 (0.7); −0.0002 (22.0); −0.0085 (0.6)
I-110: ¹H-NMR(400.0 MHz, CDCl3):
δ = 8.0184 (0.8); 7.3499 (0.5); 7.3446 (0.8); 7.3301 (0.8); 7.3255 (0.6); 7.3243 (0.6); 7.3104
(0.7); 7.2607 (33.5); 7.1372 (1.6); 7.1314 (0.6); 7.1252 (1.7); 7.1199 (1.0); 7.1143 (2.3); 7.1083
(1.0); 7.1065 (0.9); 7.1024 (2.6); 7.0879 (0.7); 7.0851 (0.8); 7.0814 (0.6); 7.0789 (0.9); 7.0422
(0.9); 7.0395 (1.0); 7.0384 (1.3); 7.0358 (1.0); 7.0224 (1.0); 7.0202 (2.2); 7.0167 (1.4); 7.0148
(0.9); 7.0120 (0.5); 7.0003 (2.4); 6.9944 (1.3); 6.9884 (0.8); 6.9831 (0.8); 6.9800 (2.5); 6.9775
(1.9); 6.9742 (0.7); 6.9629 (0.6); 6.9571 (1.6); 4.9289 (8.6); 4.4776 (1.3); 3.8145 (16.0); 2.9550
(8.4); 2.8839 (7.1); 2.8825 (7.2); 2.7698 (1.2); 2.7048 (3.0); 1.5641 (1.0); −0.0002 (12.6)
I-111: ¹H-NMR(400.0 MHz, CDCl3):
δ = 7.2776 (3.1); 7.2708 (1.9); 7.2677 (1.6); 7.2643 (3.9); 7.2577 (38.2); 7.2527 (5.0); 7.2487
(3.0); 7.2420 (4.0); 7.2384 (2.8); 7.2343 (5.6); 7.2328 (4.8); 7.2315 (5.0); 7.2271 (2.3); 7.2230
(0.9); 7.2168 (2.5); 7.2143 (1.4); 7.1259 (1.7); 7.1245 (1.8); 7.1222 (1.3); 7.1091 (0.9); 7.1068
(1.3); 7.1054 (1.4); 7.0835 (0.7); 7.0762 (6.4); 7.0710 (4.2); 7.0656 (1.5); 7.0597 (3.1); 7.0549
(9.5); 7.0514 (4.6); 7.0431 (0.5); 7.0381 (1.6); 7.0328 (3.9); 7.0277 (1.0); 7.0126 (0.6); 7.0103
(0.7); 7.0062 (0.5); 6.9910 (1.1); 6.9897 (1.1); 6.9846 (0.9); 6.9833 (0.9); 6.9705 (0.5); 6.9681
(0.5); 5.2962 (9.2); 5.2420 (9.9); 4.9865 (16.0); 1.5364 (4.1); −0.0002 (15.1)
I-112: ¹H-NMR(400.0 MHz, CDCl3):
δ = 7.3629 (1.4); 7.3566 (1.6); 7.3550 (1.0); 7.3478 (2.4); 7.3451 (1.5); 7.3384 (2.7); 7.3346
(0.9); 7.3139 (1.1); 7.3126 (1.0); 7.3095 (1.3); 7.3044 (8.2); 7.3006 (2.8); 7.2981 (5.7); 7.2928
(2.2); 7.2908 (1.7); 7.2896 (2.0); 7.2876 (3.2); 7.2824 (0.7); 7.2785 (0.8); 7.2712 (3.2); 7.2655
(2.4); 7.2567 (35.5); 7.2519 (4.1); 7.2503 (5.0); 7.2489 (5.5); 7.2468 (2.2); 7.2440 (1.1); 7.2417
(2.3); 7.2356 (5.8); 7.2320 (4.9); 7.2306 (5.6); 7.2258 (2.3); 7.2220 (1.0); 7.2159 (1.5); 7.2129
(1.2); 7.0817 (0.6); 7.0746 (4.4); 7.0724 (4.4); 7.0703 (2.2); 7.0686 (2.8); 7.0675 (3.4); 7.0620
(1.4); 7.0604 (1.1); 7.0572 (2.3); 7.0531 (7.1); 7.0521 (6.7); 7.0478 (4.4); 7.0363 (1.0); 7.0309
(2.9); 5.2951 (1.9); 5.2592 (11.4); 4.9721 (16.0); 1.5355 (5.4); −0.0002 (15.1)
I-113: ¹H-NMR(400.0 MHz, CDCl3):
δ = 7.2884 (0.7); 7.2868 (1.0); 7.2813 (3.8); 7.2773 (1.2); 7.2757 (1.5); 7.2726 (0.9); 7.2675
(4.3); 7.2670 (4.5); 7.2651 (5.0); 7.2612 (3.4); 7.2586 (34.1); 7.2506 (2.7); 7.2465 (6.6); 7.2391
(2.0); 7.2350 (2.8); 7.2311 (1.8); 7.2244 (0.7); 7.2183 (1.5); 7.2174 (1.6); 7.1994 (0.6); 7.1317
(1.6); 7.1298 (3.4); 7.1258 (4.5); 7.1201 (1.1); 7.1132 (1.8); 7.1107 (1.3); 7.1087 (3.4); 7.1054
(2.7); 7.0753 (3.7); 7.0697 (1.1); 7.0610 (0.6); 7.0583 (1.1); 7.0538 (5.2); 7.0484 (1.1); 7.0370
(0.9); 7.0316 (2.9); 5.9782 (0.6); 5.9640 (1.2); 5.9520 (0.7); 5.9499 (0.7); 5.9379 (1.4); 5.9352
(0.8); 5.9237 (0.7); 5.9210 (1.5); 5.9090 (0.8); 5.9069 (0.8); 5.8948 (1.5); 5.8808 (0.8); 5.3723
(0.8); 5.3684 (2.1); 5.3648 (2.2); 5.3609 (0.8); 5.3292 (0.7); 5.3254 (1.9); 5.3217 (1.9); 5.3179
(0.7); 5.2966 (1.2); 5.2497 (0.9); 5.2465 (2.3); 5.2433 (2.2); 5.2401 (0.8); 5.2235 (0.8); 5.2203
(2.2); 5.2171 (2.2); 5.2139 (0.8); 4.9592 (16.0); 4.7282 (2.5); 4.7247 (4.0); 4.7211 (2.6); 4.7141
(2.5); 4.7106 (3.9); 4.7070 (2.4); 1.5465 (1.1); −0.0002 (12.0)
I-114: ¹H-NMR(400.0 MHz, CDCl3):
δ = 8.0183 (0.8); 7.4156 (0.6); 7.4140 (0.7); 7.4076 (4.0); 7.4034 (3.5); 7.3967 (1.5); 7.3941
(1.3); 7.3897 (2.0); 7.3881 (1.8); 7.3811 (0.6); 7.3773 (0.7); 7.3051 (2.3); 7.3007 (2.6); 7.2965
(0.7); 7.2946 (0.8); 7.2933 (0.9); 7.2916 (0.8); 7.2892 (1.0); 7.2869 (1.0); 7.2807 (1.7); 7.2603
(32.2); 7.1938 (0.5); 7.1781 (0.5); 7.1730 (1.1); 7.1574 (1.1); 7.1526 (0.7); 7.1371 (0.6); 6.9576
(0.5); 6.9520 (1.0); 6.9467 (0.7); 6.9327 (0.5); 6.9272 (1.0); 6.9211 (1.0); 6.9186 (0.6); 6.9001
(0.9); 6.8978 (1.0); 6.8937 (0.7); 6.8914 (0.7); 6.8564 (0.8); 6.8541 (0.8); 6.8513 (0.8); 6.8491
(0.7); 6.8361 (0.7); 6.8339 (0.7); 6.8310 (0.7); 6.8288 (0.6); 4.9429 (8.7); 4.7599 (0.7); 3.8242 (16.0);
3.7970 (1.3); 2.9554 (8.2); 2.8841 (7.3); 2.8826 (7.1); 2.7699 (1.3); 1.5731 (1.0); −0.0002 (13.4)
I-115: ¹H-NMR(400.0 MHz, CDCl3):
δ = 7.4102 (1.3); 7.4060 (1.2); 7.3923 (0.7); 7.3908 (0.6); 7.2901 (0.8); 7.2858 (0.9); 7.2658
(0.9); 7.2594 (33.9); 4.9363 (2.9); 3.8218 (5.6); 1.5379 (16.0); 0.0691 (1.0); −0.0002 (14.1)
I-116: ¹H-NMR(400.0 MHz, CDCl3):
δ = 7.5185 (1.2); 7.3437 (0.8); 7.3332 (8.5); 7.3299 (3.4); 7.3279 (4.9); 7.3251 (9.3); 7.3207
(7.1); 7.3167 (9.3); 7.3078 (5.7); 7.3036 (2.8); 7.2983 (5.7); 7.2909 (2.5); 7.2854 (5.5); 7.2781
(1.3); 7.2757 (1.0); 7.2751 (1.0); 7.2742 (0.9); 7.2734 (1.0); 7.2726 (1.0); 7.2718 (1.0); 7.2710
(1.2); 7.2702 (1.3); 7.2694 (1.4); 7.2686 (1.5); 7.2678 (1.7); 7.2670 (2.0); 7.2662 (2.3); 7.2653
(2.8); 7.2637 (4.3); 7.2596 (203.6); 7.2091 (1.0); 7.1916 (0.9); 7.1841 (1.3); 7.1801 (5.1);
7.1770 (2.8); 7.1725 (3.2); 7.1699 (3.0); 7.1658 (3.3); 7.1638 (2.8); 7.1614 (2.1); 7.1589 (2.0);
7.1557 (3.8); 7.1517 (0.7); 7.1040 (0.7); 7.0968 (5.4); 7.0913 (1.6); 7.0830 (0.8); 7.0798 (1.7);
7.0757 (7.5); 7.0704 (1.8); 7.0672 (0.7); 7.0589 (1.5); 7.0534 (4.3); 6.9956 (1.4); 5.9912 (0.5);
5.3318 (1.2); 5.3142 (4.6); 5.2967 (4.6); 5.2791 (1.2); 2.0049 (3.0); 1.7453 (16.0); 1.7277
(15.8); 1.6955 (0.7); 1.6782 (0.7); 0.0080 (3.0); −0.0002 (104.2); −0.0085 (3.0); −0.0506 (0.5)
I-117: ¹H-NMR(400.0 MHz, d₆-DMSO):
δ = 8.3124 (4.8); 7.5517 (3.9); 7.5369 (4.1); 7.5167 (2.9); 7.4667 (2.2); 7.4465 (5.1); 7.4300
(4.8); 7.4097 (3.9); 7.3783 (3.4); 7.2860 (4.9); 7.2666 (6.2); 7.2277 (4.8); 7.2073 (4.6); 7.1880
(4.0); 7.1688 (2.9); 7.1639 (3.9); 7.0516 (4.4); 7.0348 (3.8); 5.0096 (2.9); 4.9925 (3.1); 3.3146
(10.8); 2.6694 (2.9); 2.5094 (189.0); 2.5049 (389.4); 2.5003 (530.0); 2.4957 (370.7); 2.4912
(170.7); 2.3273 (3.4); 1.9874 (2.3); 1.5249 (16.0); 1.5076 (16.0); −0.0002 (28.3)
I-118: ¹H-NMR(400.0 MHz, CDCl3):
δ = 7.5182 (1.0); 7.3750 (0.7); 7.3612 (0.8); 7.3553 (1.2); 7.3407 (1.3); 7.3358 (1.0); 7.3211
(1.0); 7.2763 (0.6); 7.2755 (0.6); 7.2747 (0.6); 7.2739 (0.6); 7.2731 (0.7); 7.2723 (0.7); 7.2715
(0.8); 7.2707 (0.8); 7.2699 (0.9); 7.2691 (1.0); 7.2683 (1.2); 7.2675 (1.3); 7.2667 (1.5); 7.2659
(1.7); 7.2651 (2.0); 7.2643 (2.4); 7.2635 (3.1); 7.2593 (181.8); 7.1213 (2.7); 7.1159 (1.5);
7.1095 (2.6); 7.1040 (1.7); 7.1016 (1.6); 7.0986 (4.2); 7.0952 (1.6); 7.0926 (2.2); 7.0865 (3.3);
7.0805 (0.7); 7.0781 (1.0); 7.0740 (0.7); 7.0715 (0.6); 7.0396 (1.3); 7.0370 (1.6); 7.0358 (1.8);
7.0332 (1.6); 7.0205 (1.1); 7.0167 (1.8); 7.0141 (1.6); 7.0089 (1.2); 7.0026 (1.1); 6.9993 (0.8);
6.9953 (1.2); 6.9927 (0.6); 6.9844 (3.7); 6.9789 (1.6); 6.9762 (1.0); 6.9725 (0.6); 6.9672 (1.0);
6.9641 (3.6); 6.9616 (2.8); 6.9584 (1.1); 6.9470 (0.8); 6.9413 (2.4); 4.9737 (16.0); 2.9626 (1.2);
2.8902 (1.1); 1.8551 (0.6); 0.0079 (2.3); 0.0064 (0.6); 0.0055 (0.7); 0.0047 (0.8); −0.0002
(76.6); −0.0059 (1.2); −0.0067 (1.0); −0.0085 (2.4)
I-119: ¹H-NMR(400.0 MHz, CDCl3):
δ = 7.5182 (0.9); 7.3707 (0.7); 7.3558 (1.1); 7.3498 (1.7); 7.3354 (1.7); 7.3296 (1.3); 7.3154
(1.4); 7.2659 (1.8); 7.2592 (171.5); 7.1462 (0.7); 7.1374 (2.9); 7.1319 (1.1); 7.1256 (3.4);
7.1199 (2.7); 7.1145 (4.8); 7.1108 (1.8); 7.1028 (4.3); 7.0986 (1.8); 7.0957 (1.8); 7.0897 (2.0);
7.0775 (0.8); 7.0751 (0.8); 7.0710 (0.8); 7.0685 (0.9); 7.0460 (1.7); 7.0422 (2.6); 7.0397 (2.2);
7.0244 (4.7); 7.0207 (3.3); 7.0029 (1.8); 6.9971 (5.6); 6.9918 (1.9); 6.9854 (0.8); 6.9769 (4.8);
6.9711 (1.5); 6.9599 (1.0); 6.9541 (3.0); 4.9821 (16.0); 4.9283 (1.0); 3.8129 (1.9); 2.0446 (0.8);
1.2585 (0.6); 0.0079 (1.9); −0.0002 (69.8); −0.0085 (2.2)
I-120: ¹H-NMR(400.0 MHz, CDCl3):
δ = 7.5184 (0.5); 7.3704 (0.6); 7.3555 (0.8); 7.3524 (0.6); 7.3496 (1.2); 7.3351 (1.3); 7.3305
(0.9); 7.3154 (1.0); 7.2709 (0.5); 7.2693 (0.6); 7.2685 (0.7); 7.2677 (0.8); 7.2669 (0.8); 7.2661
(1.0); 7.2652 (1.2); 7.2644 (1.4); 7.2636 (1.8); 7.2595 (92.6); 7.2530 (0.6); 7.1479 (2.5); 7.1421
(1.0); 7.1360 (2.8); 7.1306 (1.6); 7.1250 (3.6); 7.1190 (1.2); 7.1132 (4.1); 7.1095 (0.9); 7.1070
(0.9); 7.1048 (0.5); 7.0948 (1.2); 7.0920 (1.2); 7.0882 (0.9); 7.0858 (1.4); 7.0737 (0.6); 7.0712
(0.6); 7.0671 (0.6); 7.0647 (0.7); 7.0440 (1.4); 7.0414 (1.6); 7.0401 (2.0); 7.0376 (1.7); 7.0219
(3.5); 7.0185 (2.2); 7.0135 (1.1); 7.0050 (3.6); 6.9995 (2.0); 6.9956 (1.6); 6.9933 (1.5); 6.9880
(1.3); 6.9848 (3.9); 6.9822 (2.9); 6.9790 (1.2); 6.9677 (0.9); 6.9619 (2.5); 4.9819 (16.0); 3.7571
(0.5); 2.9661 (1.9); 2.8941 (1.7); 2.8926 (1.8); 1.8576 (0.6); 1.4319 (0.8); 0.0080 (1.2); 0.0048
(0.5); 0.0040 (0.6); −0.0002 (39.1); −0.0084 (1.1)
I-121: ¹H-NMR(400.0 MHz, CDCl3):
δ = 7.5185 (1.1); 7.4037 (0.6); 7.3890 (1.0); 7.3857 (0.7); 7.3829 (1.3); 7.3690 (1.4); 7.3639
(1.0); 7.3492 (1.2); 7.2597 (198.1); 7.1909 (0.8); 7.1884 (0.8); 7.1842 (0.8); 7.1819 (0.9);
7.1697 (1.4); 7.1670 (1.3); 7.1634 (1.2); 7.1608 (1.5); 7.1485 (0.9); 7.1393 (3.3); 7.1335 (1.1);
7.1275 (3.0); 7.1218 (2.0); 7.1162 (4.0); 7.1104 (1.2); 7.1045 (3.9); 7.0962 (0.6); 7.0783 (1.5);
7.0744 (2.2); 7.0719 (2.0); 7.0561 (3.7); 7.0529 (2.4); 7.0350 (1.3); 7.0292 (1.2); 7.0253 (1.3);
7.0171 (3.9); 7.0112 (1.2); 7.0055 (0.6); 6.9972 (4.2); 6.9942 (3.3); 6.9914 (1.4); 6.9800 (1.0);
6.9743 (2.7); 5.0798 (16.0); 4.9644 (1.4); 3.8227 (0.7); 3.0213 (0.8); 2.9521 (0.7); 2.1027 (3.4);
2.0449 (1.1); 1.2587 (0.8); 0.0080 (2.4); −0.0002 (84.0); −0.0085 (2.3)
I-122: ¹H-NMR(400.0 MHz, CDCl3):
δ = 7.4991 (0.7); 7.4812 (0.6); 7.4744 (1.4); 7.4595 (0.8); 7.4560 (1.4); 7.4524 (0.7); 7.4304
(1.6); 7.4269 (0.8); 7.4154 (1.4); 7.4115 (2.9); 7.3980 (0.6); 7.3936 (1.4); 7.3903 (0.9); 7.3661
(0.6); 7.3634 (0.6); 7.3458 (0.6); 7.3229 (2.4); 7.3199 (2.8); 7.3161 (1.4); 7.3076 (0.7); 7.3025
(2.3); 7.2987 (1.6); 7.2602 (14.6); 7.2272 (0.6); 7.2119 (0.6); 7.2066 (1.3); 7.1913 (1.2); 7.1860
(0.8); 7.1708 (0.7); 7.0015 (0.5); 6.9993 (0.5); 6.9870 (0.8); 6.9850 (0.8); 6.9807 (0.9); 6.9787
(0.8); 6.9580 (0.6); 6.9356 (0.9); 6.9301 (1.3); 6.9246 (0.7); 6.9120 (0.7); 6.9066 (1.1); 6.9008
(0.6); 6.8438 (0.9); 6.8401 (0.8); 6.8235 (0.8); 6.8198 (0.7); 5.2972 (0.8); 5.0502 (2.0); 5.0368
(8.8); 3.8448 (3.7); 3.8317 (16.0); 3.8173 (0.8); −0.0002 (6.2)
I-123: ¹H-NMR(400.0 MHz, CDCl3):
δ = 7.3660 (0.7); 7.3638 (0.5); 7.3507 (1.0); 7.3480 (0.8); 7.3451 (1.4); 7.3307 (1.6); 7.3259
(1.1); 7.3108 (1.2); 7.2917 (0.8); 7.2771 (0.9); 7.2719 (1.7); 7.2586 (40.0); 7.2524 (1.8); 7.2375
(1.2); 7.1246 (1.9); 7.1234 (1.9); 7.1137 (0.9); 7.1112 (1.0); 7.1047 (2.3); 7.0926 (1.3); 7.0898
(1.4); 7.0862 (1.2); 7.0837 (1.6); 7.0714 (0.7); 7.0690 (0.8); 7.0650 (0.9); 7.0625 (1.0); 7.0585
(3.1); 7.0528 (2.2); 7.0467 (3.3); 7.0412 (2.1); 7.0356 (6.7); 7.0322 (3.1); 7.0297 (2.8); 7.0238
(5.7); 7.0166 (4.8); 7.0131 (3.0); 7.0020 (1.5); 6.9950 (2.4); 6.9910 (1.6); 6.9846 (1.2); 6.9774
(1.4); 6.9694 (4.3); 6.9635 (1.2); 6.9574 (0.7); 6.9491 (4.5); 6.9434 (1.3); 6.9321 (0.9); 6.9264
(2.4); 5.2973 (1.9); 5.2422 (11.0); 4.9719 (16.0); 4.9277 (0.5); 3.8122 (0.9); 2.2706 (0.9);
1.5396 (2.0); 1.4318 (8.6); −0.0002 (16.0); −0.0085 (0.5)
I-124: ¹H-NMR(400.0 MHz, CDCl3):
δ = 7.3669 (2.2); 7.3602 (2.0); 7.3517 (3.5); 7.3486 (3.3); 7.3419 (4.1); 7.3327 (2.3); 7.3272
(2.8); 7.3232 (2.0); 7.3178 (9.4); 7.3115 (7.0); 7.3060 (2.6); 7.3009 (3.1); 7.2960 (0.7); 7.2924
(0.7); 7.2576 (39.0); 7.1135 (0.8); 7.1111 (0.9); 7.1070 (0.9); 7.1047 (0.9); 7.0923 (1.3); 7.0899
(1.5); 7.0858 (1.4); 7.0835 (1.5); 7.0712 (0.6); 7.0688 (0.7); 7.0646 (0.7); 7.0624 (0.7); 7.0250
(3.1); 7.0206 (3.1); 7.0183 (2.2); 7.0138 (2.8); 7.0085 (2.0); 7.0029 (6.1); 6.9991 (2.4); 6.9971
(1.8); 6.9909 (5.5); 6.9840 (1.7); 6.9805 (1.2); 6.9673 (1.4); 6.9634 (1.4); 6.9612 (1.4); 6.9568
(1.4); 6.9484 (4.4); 6.9424 (1.2); 6.9362 (0.7); 6.9281 (4.7); 6.9224 (1.4); 6.9111 (0.9); 6.9053
(2.4); 5.2962 (1.1); 5.2548 (13.0); 4.9502 (16.0); 4.5006 (0.5); 1.5392 (2.0); 1.4317 (3.7);
0.0701 (2.0); −0.0002 (14.8)
I-127: ¹H-NMR(400.0 MHz, CDCl3):
δ = 7.5184 (2.0); 7.4222 (1.1); 7.4142 (5.0); 7.4107 (5.3); 7.4046 (2.1); 7.4004 (2.2); 7.3962
(3.4); 7.3873 (1.0); 7.3831 (1.2); 7.3774 (0.6); 7.3724 (0.6); 7.3072 (3.7); 7.3029 (4.1); 7.2960
(1.6); 7.2932 (1.8); 7.2906 (2.2); 7.2884 (2.3); 7.2829 (3.2); 7.2595 (362.7); 7.2239 (1.3);
7.2102 (0.6); 7.1940 (0.8); 7.1888 (1.6); 7.1734 (1.4); 7.1681 (1.0); 7.1531 (0.9); 6.9956 (2.1);
6.9611 (0.8); 6.9554 (1.4); 6.9497 (1.1); 6.9364 (1.5); 6.9305 (1.8); 6.9257 (1.2); 6.9157 (1.5);
6.9134 (1.6); 6.9094 (1.0); 6.8927 (0.9); 6.8886 (0.7); 6.8783 (1.3); 6.8582 (1.1); 6.8553 (1.0);
5.0006 (16.0); 2.0444 (0.5); 1.5663 (1.1); 1.2587 (0.7); 0.1579 (0.5); 0.0080 (4.5); −0.0002
(152.0); −0.0085 (5.7); −0.1495 (0.5)
I-128: ¹H-NMR(400.0 MHz, CDCl3):
δ = 7.5183 (0.7); 7.4275 (1.1); 7.4224 (1.7); 7.4145 (6.5); 7.4109 (7.2); 7.4048 (3.1); 7.4006
(2.8); 7.3964 (4.5); 7.3948 (4.0); 7.3876 (1.5); 7.3834 (1.7); 7.3775 (0.8); 7.3731 (1.0); 7.3094
(0.8); 7.2911 (5.0); 7.2869 (5.6); 7.2818 (2.8); 7.2798 (2.8); 7.2773 (2.7); 7.2744 (3.5); 7.2724
(3.8); 7.2669 (5.6); 7.2594 (119.9); 7.1937 (1.2); 7.1782 (1.3); 7.1733 (2.3); 7.1579 (2.1);
7.1532 (1.4); 7.1378 (1.2); 6.9954 (0.7); 6.9352 (1.0); 6.9299 (2.1); 6.9248 (1.7); 6.9163 (1.4);
6.9140 (1.6); 6.9104 (1.6); 6.9053 (2.2); 6.9002 (1.9); 6.8957 (2.3); 6.8933 (2.5); 6.8894 (1.5);
6.8870 (1.4); 6.8750 (1.1); 6.8728 (1.2); 6.8687 (1.0); 6.8641 (2.1); 6.8617 (2.1); 6.8594 (1.9);
6.8569 (1.6); 6.8436 (1.7); 6.8416 (1.7); 6.8389 (1.6); 4.9914 (16.0); 2.0450 (0.8); 1.2586 (0.6);
0.0080 (1.6); −0.0002 (49.6); −0.0085 (2.2)
I-129: ¹H-NMR(400.0 MHz, CDCl3):
δ = 10.5842 (0.5); 7.9716 (0.6); 7.9485 (0.7); 7.5190 (1.1); 7.5010 (1.3); 7.4975 (0.7); 7.4890
(0.7); 7.4822 (3.1); 7.4764 (0.9); 7.4675 (1.3); 7.4640 (2.5); 7.4605 (1.4); 7.4365 (2.6); 7.4332
(1.4); 7.4215 (2.3); 7.4177 (4.7); 7.4042 (0.8); 7.3998 (2.4); 7.3965 (1.5); 7.3672 (1.1); 7.3496
(1.2); 7.3325 (0.9); 7.3274 (3.8); 7.3245 (4.7); 7.3122 (1.1); 7.3070 (3.7); 7.3032 (2.6); 7.2600
(160.0); 7.2385 (0.9); 7.2235 (1.2); 7.2180 (1.9); 7.2103 (1.0); 7.2029 (1.9); 7.1974 (1.2);
7.1821 (1.2); 7.0335 (0.6); 7.0275 (0.7); 7.0177 (0.8); 7.0155 (0.8); 7.0092 (0.8); 6.9960 (2.0);
6.9906 (1.4); 6.9886 (1.4); 6.9761 (0.7); 6.9741 (0.7); 6.9701 (0.8); 6.9419 (1.2); 6.9366 (1.9);
6.9309 (1.1); 6.9184 (1.2); 6.9131 (1.8); 6.9073 (1.0); 6.8647 (1.5); 6.8612 (1.4); 6.8445 (1.2);
6.8410 (1.2); 6.0463 (0.5); 5.1063 (4.1); 5.0941 (16.0); 5.0017 (0.7); 4.9442 (1.0); 4.8496 (0.5);
3.7744 (0.7); 3.7639 (0.7); 3.7577 (1.8); 3.7411 (0.7); 3.6087 (0.6); 1.8751 (0.9); 1.8673 (0.8);
1.8586 (2.3); 1.8499 (0.8); 1.8420 (0.8); 0.0080 (2.2); −0.0002 (66.7); −0.0085 (2.1)
I-130: ¹H-NMR(400.0 MHz, CDCl3):
δ = 7.3316 (3.7); 7.3226 (5.0); 7.3158 (6.2); 7.3046 (2.9); 7.2955 (2.6); 7.2876 (1.4); 7.2825
(2.3); 7.2606 (8.2); 7.1666 (2.4); 7.1578 (2.5); 7.1536 (2.0); 7.1488 (2.0); 7.1426 (1.9); 7.0919
(2.2); 7.0705 (3.8); 7.0489 (1.8); 5.2969 (2.6); 5.2622 (0.6); 5.2448 (1.8); 5.2273 (1.8); 5.2099
(0.6); 3.7819 (16.0); 1.6914 (7.4); 1.6739 (7.3); 1.5622 (0.5); −0.0002 (3.0)
I-131: ¹H-NMR(400.0 MHz, CDCl3):
δ = 7.5184 (1.1); 7.3920 (0.9); 7.3878 (1.1); 7.3730 (1.7); 7.3684 (2.4); 7.3550 (1.4); 7.3494
(2.1); 7.3440 (1.1); 7.3363 (1.2); 7.3283 (1.0); 7.3234 (0.8); 7.3159 (0.9); 7.3091 (1.0); 7.2595
(207.3); 7.2122 (1.6); 7.1945 (2.0); 7.1731 (0.9); 7.1416 (1.9); 7.1369 (1.2); 7.1202 (2.2);
7.1095 (1.3); 7.0999 (1.6); 7.0957 (2.6); 7.0908 (1.3); 7.0750 (1.2); 7.0605 (1.3); 7.0571 (1.1);
7.0357 (2.0); 7.0145 (1.2); 7.0062 (1.0); 7.0023 (1.1); 6.9956 (2.3); 6.9916 (1.2); 6.9796 (0.8);
6.9708 (0.8); 4.8919 (16.0); 4.2949 (2.0); 4.2771 (6.3); 4.2592 (6.5); 4.2414 (2.3); 4.2306 (0.9);
4.0988 (0.5); 1.5367 (14.5); 1.3058 (7.5); 1.2879 (14.9); 1.2701 (7.8); 1.2543 (1.8); 0.0080
(2.5); −0.0002 (74.4); −0.0083 (3.0)
I-132: ¹H-NMR(400.0 MHz, CDCl3):
δ = 7.2775 (1.0); 7.2722 (0.9); 7.2593 (47.0); 7.2421 (1.1); 7.2364 (1.2); 7.2223 (0.7); 7.1101
(0.9); 7.1062 (1.3); 7.0888 (0.9); 7.0858 (0.7); 7.0730 (0.9); 7.0513 (1.4); 7.0294 (0.7); 5.2640
(0.8); 5.2466 (0.8); 3.7878 (7.6); 1.6968 (3.2); 1.6793 (3.2); 1.5355 (16.0); 0.0079 (0.6); −0.0002
(17.6); −0.0084 (0.7)
I-133: ¹H-NMR(400.0 MHz, CDCl3):
δ = 7.3813 (0.8); 7.3769 (1.0); 7.3621 (1.4); 7.3577 (2.0); 7.3480 (0.7); 7.3433 (1.4); 7.3392
(1.9); 7.3287 (1.2); 7.3242 (0.7); 7.3204 (1.0); 7.3160 (0.7); 7.3083 (0.8); 7.3039 (0.8); 7.2597
(38.3); 7.2558 (2.1); 7.2066 (0.9); 7.2031 (1.2); 7.1873 (1.5); 7.1856 (1.4); 7.1837 (1.4); 7.1681
(0.7); 7.1645 (0.6); 7.1485 (0.9); 7.1432 (1.1); 7.1377 (0.9); 7.1298 (1.0); 7.1244 (1.1); 7.1214
(1.1); 7.1164 (2.3); 7.1127 (1.0); 7.1027 (0.9); 7.0968 (1.7); 7.0917 (1.4); 7.0712 (1.1); 7.0532
(1.1); 7.0498 (1.0); 7.0325 (1.0); 7.0285 (1.7); 7.0247 (1.1); 7.0096 (1.0); 7.0066 (1.5); 7.0043
(1.6); 6.9991 (1.0); 6.9955 (1.2); 6.9902 (0.8); 6.9847 (0.7); 6.9793 (0.6); 6.9740 (0.6); 6.9688
(0.5); 4.8903 (14.9); 4.8862 (1.6); 4.2919 (1.9); 4.2741 (6.0); 4.2703 (0.7); 4.2563 (6.0); 4.2525
(0.6); 4.2385 (2.0); 1.5438 (13.1); 1.5400 (0.7); 1.3038 (8.0); 1.3005 (1.0); 1.2860 (16.0);
1.2826 (1.8); 1.2682 (9.1); 1.2647 (2.6); 0.8987 (1.1); 0.8817 (4.0); 0.8640 (1.5); −0.0002
(13.9); −0.0040 (0.8)
I-134: ¹H-NMR(400.0 MHz, CDCl3):
δ = 7.3790 (0.5); 7.3637 (1.0); 7.3572 (1.0); 7.3484 (0.6); 7.3423 (2.0); 7.3361 (0.7); 7.3268
(1.0); 7.3209 (1.2); 7.3057 (0.6); 7.2600 (30.0); 7.1873 (0.9); 7.1820 (1.0); 7.1686 (1.0); 7.1608
(1.2); 7.1553 (1.1); 7.1505 (1.0); 7.1418 (1.0); 7.1367 (1.1); 7.1293 (1.7); 7.1257 (1.0); 7.1091
(1.3); 7.1044 (1.6); 7.0843 (1.2); 7.0504 (0.8); 7.0459 (1.1); 7.0400 (1.2); 7.0352 (1.2); 7.0311
(0.9); 7.0251 (0.8); 7.0191 (0.7); 7.0145 (0.7); 6.9554 (3.0); 6.9370 (4.0); 6.9340 (4.0); 6.9156
(2.6); 4.8743 (16.0); 4.2811 (2.0); 4.2632 (6.3); 4.2454 (6.4); 4.2275 (2.1); 1.5445 (11.0);
1.3037 (0.7); 1.2900 (7.9); 1.2722 (16.0); 1.2543 (7.9); 0.8982 (1.2); 0.8815 (3.8); 0.8638 (1.6);
0.0079 (0.5); −0.0002 (15.1); −0.0084 (0.7)
I-135: ¹H-NMR(400.0 MHz, CDCl3):
δ = 7.2791 (1.0); 7.2736 (0.6); 7.2654 (1.6); 7.2594 (33.1); 7.2495 (1.1); 7.2437 (1.4); 7.2367
(1.5); 7.2317 (0.7); 7.2215 (0.6); 7.2176 (1.4); 7.2007 (0.8); 7.1971 (0.5); 7.1832 (0.7); 7.0837
(1.3); 7.0797 (2.0); 7.0778 (1.7); 7.0665 (0.7); 7.0595 (1.4); 7.0556 (2.1); 7.0508 (0.5); 7.0338
(1.0); 3.7461 (10.0); 2.9553 (0.6); 2.8833 (0.5); 2.7723 (1.4); 1.7553 (16.0); 1.5440 (1.2);
0.0080 (0.6); −0.0002 (16.8); −0.0084 (0.7)
I-136: ¹H-NMR(400.0 MHz, CDCl3):
δ = 7.3977 (0.9); 7.3935 (1.1); 7.3785 (1.7); 7.3744 (2.1); 7.3663 (0.7); 7.3591 (1.5); 7.3552
(1.8); 7.3518 (1.3); 7.3472 (0.9); 7.3392 (1.1); 7.3336 (1.0); 7.3311 (1.0); 7.3267 (0.8); 7.3188
(0.9); 7.3144 (0.7); 7.2597 (43.0); 7.2129 (1.3); 7.1935 (1.9); 7.1759 (0.8); 7.1506 (1.0); 7.1450
(1.7); 7.1319 (1.1); 7.1237 (2.8); 7.1190 (1.8); 7.1033 (1.8); 7.0993 (2.4); 7.0782 (1.1); 7.0597
(1.3); 7.0565 (1.2); 7.0388 (1.3); 7.0351 (2.0); 7.0314 (1.3); 7.0139 (1.9); 7.0105 (2.0); 7.0037
(1.2); 6.9996 (1.2); 6.9950 (1.0); 6.9886 (0.8); 6.9824 (0.8); 6.9780 (0.7); 6.9733 (0.5); 4.9654
(16.0); 4.1318 (1.2); 4.1140 (1.2); 2.0453 (5.5); 1.4318 (0.7); 1.2764 (1.5); 1.2585 (2.9); 1.2407
(1.4); 0.0079 (0.8); −0.0002 (22.6); −0.0083 (1.0)
I-137: ¹H-NMR(400.0 MHz, CDCl3):
δ = 7.3871 (0.5); 7.3718 (1.0); 7.3656 (0.9); 7.3565 (0.6); 7.3504 (1.9); 7.3442 (0.6); 7.3351
(0.9); 7.3290 (1.1); 7.3137 (0.6); 7.2607 (30.4); 7.1884 (0.9); 7.1831 (0.9); 7.1696 (0.9); 7.1643
(1.1); 7.1618 (1.1); 7.1562 (1.6); 7.1430 (0.9); 7.1349 (1.6); 7.1311 (0.9); 7.1147 (1.3); 7.1099
(1.4); 7.0900 (1.1); 7.0533 (0.8); 7.0497 (0.9); 7.0428 (1.0); 7.0393 (1.0); 7.0341 (0.8); 7.0284
(0.6); 7.0232 (0.6); 7.0179 (0.6); 6.9709 (0.5); 6.9655 (1.2); 6.9626 (2.8); 6.9442 (3.7); 6.9411
(3.5); 6.9360 (0.6); 6.9229 (2.4); 6.9198 (1.0); 5.2985 (4.2); 4.9432 (16.0); 3.7819 (0.8); 3.7783
(3.0); 3.7763 (2.0); 3.7720 (1.9); 3.7699 (1.6); 3.7679 (2.5); 3.7617 (7.5); 3.7556 (2.6); 3.7513
(1.9); 3.7470 (2.0); 3.7450 (3.1); 3.7415 (0.7); 1.8763 (3.0); 1.8685 (2.8); 1.8643 (2.0); 1.8597
(9.0); 1.8549 (2.1); 1.8508 (2.8); 1.8430 (3.0); 1.8391 (0.7); 1.4319 (0.8); −0.0002
(17.0); −0.0085 (0.7)
I-138: ¹H-NMR(400.0 MHz, CDCl3):
δ = 7.5182 (1.6); 7.3446 (0.8); 7.2593 (301.4); 7.2485 (9.6); 7.2465 (9.6); 7.2399 (4.8); 7.2360
(6.9); 7.2315 (7.1); 7.2273 (14.9); 7.2187 (8.3); 7.2140 (8.9); 7.2085 (2.4); 7.2024 (3.5); 7.1831
(1.1); 7.1687 (0.6); 7.1120 (6.8); 7.1079 (8.6); 7.1022 (2.5); 7.0955 (3.6); 7.0910 (6.9); 7.0877
(5.7); 7.0612 (6.7); 7.0558 (2.4); 7.0394 (10.5); 7.0231 (2.0); 7.0177 (5.3); 6.9952 (1.6); 5.2993
(1.2); 5.2821 (4.4); 5.2646 (4.5); 5.2472 (1.2); 3.8179 (1.1); 1.7194 (16.0); 1.7020 (16.0); 1.4318
(0.7); 1.2557 (1.0); 0.1462 (0.5); 0.0080 (5.0); −0.0002 (159.4); −0.0084 (5.4); −0.1498 (0.6)
I-139: ¹H-NMR(400.0 MHz, CDCl3):
δ = 19.9731 (0.8); 7.5183 (2.5); 7.3101 (0.9); 7.2930 (1.7); 7.2879 (2.5); 7.2711 (13.4); 7.2594
(475.1); 7.2526 (17.5); 7.2436 (7.6); 7.2360 (6.9); 7.2274 (3.8); 7.2094 (2.5); 7.1355 (6.0);
7.1314 (7.6); 7.1145 (6.1); 7.1113 (5.1); 7.0781 (6.1); 7.0564 (9.6); 7.0345 (4.8); 6.9954 (2.5);
5.3315 (1.2); 5.3139 (4.4); 5.2965 (4.5); 5.2788 (1.3); 1.7459 (15.8); 1.7284 (16.0); 1.2562
(0.8); 0.1458 (0.9); 0.0079 (8.1); −0.0002 (246.6); −0.0084 (8.8); −0.0499 (0.9); −0.1499 (0.9)
I-140: ¹H-NMR(400.0 MHz, CDCl3):
δ = 7.5189 (0.8); 7.2600 (132.6); 7.2336 (4.5); 7.2164 (2.5); 7.1101 (2.3); 7.0932 (2.1); 7.0732
(1.7); 7.0520 (2.8); 7.0304 (1.4); 6.9960 (0.8); 2.0043 (0.9); 1.7788 (16.0); 1.2553 (0.7); 0.0079
(3.3); −0.0002 (78.0); −0.0084 (4.2)
I-141: ¹H-NMR(400.0 MHz, CDCl3):
δ = 7.3390 (0.7); 7.3319 (2.8); 7.3229 (3.6); 7.3156 (4.9); 7.3046 (2.2); 7.2997 (1.2); 7.2952
(2.2); 7.2877 (1.0); 7.2823 (2.0); 7.2602 (10.9); 7.1667 (1.9); 7.1576 (1.9); 7.1533 (1.4); 7.1487
(1.5); 7.1459 (1.1); 7.1423 (1.6); 7.0922 (2.0); 7.0869 (0.7); 7.0707 (3.2); 7.0661 (0.9); 7.0542
(0.6); 7.0490 (1.6); 5.2973 (1.3); 5.2446 (1.7); 5.2271 (1.7); 5.2097 (0.5); 3.7821 (16.0); 1.6915
(6.8); 1.6740 (6.8); 1.5553 (0.5); −0.0002 (4.2)
I-146: ¹H-NMR(400.0 MHz, CDCl3):
δ = 7.3732 (1.0); 7.3668 (0.8); 7.3580 (0.6); 7.3518 (2.0); 7.3455 (0.7); 7.3363 (0.9); 7.3304
(1.2); 7.3151 (0.6); 7.2650 (0.5); 7.2592 (51.0); 7.1808 (0.8); 7.1756 (0.9); 7.1622 (0.9); 7.1545
(1.7); 7.1488 (1.1); 7.1347 (1.8); 7.1300 (1.8); 7.1135 (1.2); 7.1087 (1.4); 7.0887 (1.1); 7.0460
(0.7); 7.0410 (0.8); 7.0354 (1.0); 7.0318 (1.0); 7.0266 (0.8); 7.0209 (0.7); 7.0157 (0.6); 7.0104
(0.6); 6.9772 (0.9); 6.9730 (0.6); 6.9673 (1.1); 6.9644 (2.9); 6.9513 (0.6); 6.9460 (3.7); 6.9429
(3.7); 6.9247 (2.6); 6.9216 (1.1); 6.9164 (0.5); 5.9521 (0.6); 5.9380 (1.2); 5.9260 (0.7); 5.9239
(0.7); 5.9118 (1.3); 5.9092 (0.8); 5.8976 (0.7); 5.8950 (1.4); 5.8829 (0.8); 5.8809 (0.8); 5.8688
(1.4); 5.8547 (0.7); 5.3471 (0.7); 5.3433 (2.0); 5.3396 (2.1); 5.3358 (0.9); 5.3042 (0.6); 5.3003
(1.7); 5.2966 (1.9); 5.2928 (0.8); 5.2365 (0.8); 5.2334 (2.1); 5.2301 (2.2); 5.2269 (0.9); 5.2104
(0.8); 5.2072 (2.0); 5.2039 (2.1); 5.2007 (0.9); 5.0015 (0.7); 4.9177 (15.3); 4.7050 (2.4); 4.7015
(4.0); 4.6981 (2.7); 4.6909 (2.6); 4.6874 (4.0); 4.6839 (2.7); 2.2705 (1.7); 1.5390 (4.2); 1.4319
(16.0); 1.2559 (0.9); 0.0080 (0.5); −0.0002 (19.1); −0.0085 (0.9)
I-149: ¹H-NMR(400.0 MHz, CDCl3):
δ = 7.4454 (0.5); 7.4300 (1.1); 7.4238 (1.0); 7.4146 (0.6); 7.4085 (2.0); 7.4024 (0.7); 7.3931
(1.0); 7.3869 (1.2); 7.3716 (0.6); 7.2596 (76.5); 7.2239 (1.0); 7.2190 (0.9); 7.2057 (0.9); 7.1994
(1.3); 7.1931 (0.9); 7.1770 (1.3); 7.1559 (1.5); 7.1525 (1.0); 7.1362 (1.4); 7.1312 (1.4); 7.1119
(1.2); 7.0784 (1.0); 7.0726 (1.0); 7.0678 (1.0); 7.0569 (0.7); 7.0513 (0.6); 7.0467 (0.6); 6.9957
(0.5); 6.9870 (1.5); 6.9835 (2.6); 6.9654 (3.8); 6.9619 (3.8); 6.9438 (2.3); 6.9401 (1.3); 5.9436
(0.5); 5.9293 (1.1); 5.9174 (0.7); 5.9151 (0.6); 5.9032 (1.3); 5.9009 (0.8); 5.8887 (0.8); 5.8863
(1.4); 5.8744 (0.8); 5.8721 (0.8); 5.8602 (1.4); 5.8459 (0.7); 5.3474 (0.8); 5.3437 (2.2); 5.3400
(2.2); 5.3363 (0.9); 5.3044 (0.7); 5.3007 (1.9); 5.2971 (2.0); 5.2933 (0.8); 5.2412 (0.9); 5.2382
(2.3); 5.2351 (2.2); 5.2320 (0.9); 5.2151 (0.9); 5.2121 (2.1); 5.2090 (2.1); 5.2059 (0.8); 5.0047
(16.0); 4.7072 (2.9); 4.7038 (4.7); 4.7004 (2.9); 4.6929 (3.0); 4.6895 (4.6); 4.6861 (2.8); 1.5355
(14.9); 1.4319 (1.3); 0.0078 (0.8); −0.0002 (27.7); −0.0086 (1.2)
I-150: ¹H-NMR(300.1 MHz, d₆-DMSO):
δ = 7.6062 (2.4); 7.5988 (2.4); 7.5721 (2.3); 7.5649 (2.4); 7.5109 (1.6); 7.4967 (1.1); 7.4828
(3.8); 7.4756 (1.7); 7.4686 (2.2); 7.4555 (3.2); 7.4465 (3.0); 7.4401 (2.1); 7.4183 (1.8); 7.4054
(4.2); 7.4027 (4.1); 7.3983 (2.8); 7.3769 (1.9); 7.3732 (2.3); 7.2294 (1.0); 7.2230 (1.0); 7.2016
(1.8); 7.1950 (1.7); 7.1753 (0.8); 7.1669 (0.8); 4.9336 (12.6); 4.2054 (2.2); 4.1817 (6.8); 4.1580
(6.9); 4.1344 (2.2); 3.3214 (19.8); 2.5141 (10.6); 2.5085 (19.2); 2.5026 (24.1); 2.4968 (16.6);
1.9894 (0.8); 1.2348 (0.5); 1.2119 (7.8); 1.1883 (16.0); 1.1750 (1.2); 1.1646 (7.3); 1.1514 (0.4);
0.0107 (0.8); 0.0000 (15.2); −0.0110 (0.6)
I-151: ¹H-NMR(300.3 MHz, d₆-DMSO):
δ = 7.6216 (4.0); 7.5923 (3.8); 7.5816 (3.3); 7.5246 (2.0); 7.4965 (4.6); 7.4691 (3.5); 7.4334
(4.1); 7.4117 (13.2); 7.3817 (3.0); 4.9452 (15.7); 4.2088 (2.5); 4.1852 (7.7); 4.1615 (7.8);
4.1379 (2.6); 3.3717 (41.8); 3.3661 (39.4); 2.5111 (5.1); 2.0832 (0.4); 1.2304 (1.2); 1.2138
(8.0); 1.1902 (16.0); 1.1666 (7.7); −0.0001 (0.4)
I-152: ¹H-NMR(300.1 MHz, d₆-DMSO):
δ = 7.7620 (4.8); 7.7572 (4.5); 7.6027 (2.6); 7.5973 (2.6); 7.5672 (4.0); 7.5385 (8.3); 7.5283
(5.2); 7.5061 (0.7); 7.4997 (0.8); 7.4262 (0.9); 7.3983 (3.7); 7.3806 (8.3); 7.3586 (0.7); 7.3524
(0.6); 4.9164 (13.0); 4.1929 (2.3); 4.1692 (7.0); 4.1455 (7.1); 4.1219 (2.4); 3.3282 (74.2);
2.5100 (10.8); 2.5043 (13.2); 2.4986 (9.3); 1.2333 (1.1); 1.2086 (7.8); 1.1849 (16.0); 1.1612
(7.4); 0.0001 (5.4)
I-153: ¹H-NMR(300.1 MHz, d₆-DMSO):
δ = 7.7512 (6.5); 7.7478 (6.6); 7.5704 (1.7); 7.5419 (8.8); 7.5332 (7.4); 7.5040 (1.5); 7.4986
(1.5); 7.4798 (1.4); 7.4518 (2.9); 7.4287 (3.3); 7.4230 (3.4); 7.4018 (2.1); 7.3823 (3.1); 7.3547
(1.7); 7.3481 (1.7); 7.2075 (1.7); 7.2021 (1.7); 7.1796 (3.0); 7.1520 (1.6); 4.9238 (15.8); 4.1992
(2.6); 4.1757 (7.6); 4.1521 (7.8); 4.1286 (2.8); 3.3512 (38.7); 2.5127 (4.4); 2.0834 (0.4); 1.2335
(1.7); 1.2137 (8.4); 1.1901 (16.0); 1.1665 (8.0); 0.0003 (0.6)
I-154: ¹H-NMR(300.1 MHz, d₆-DMSO):
δ = 7.7772 (5.7); 7.7711 (5.5); 7.6052 (2.7); 7.5981 (2.7); 7.5709 (2.7); 7.5643 (2.8); 7.5520
(1.9); 7.5455 (1.6); 7.5242 (4.8); 7.5177 (4.6); 7.4951 (6.6); 7.4671 (2.8); 7.4343 (3.7); 7.4081
(3.4); 7.3831 (3.7); 7.3772 (3.2); 7.3539 (1.5); 7.3481 (1.4); 4.9380 (13.6); 4.2073 (2.3); 4.1836
(6.9); 4.1599 (7.0); 4.1363 (2.3); 3.3233 (22.0); 2.5089 (14.2); 2.5034 (17.3); 2.4980 (12.4);
1.2328 (0.8); 1.2144 (7.9); 1.1907 (16.0); 1.1671 (7.5); 0.0001 (7.2)
I-155: ¹H-NMR(3OO.1 MHz, d₆-DMSO):
δ = 7.6142 (2.7); 7.6058 (2.8); 7.5957 (2.9); 7.5878 (3.5); 7.5855 (3.4); 7.5763 (2.8); 7.5614
(2.9); 7.5549 (4.2); 7.5346 (2.2); 7.5262 (2.5); 7.5060 (2.3); 7.4557 (1.6); 7.4274 (3.5); 7.4013
(3.4); 7.3766 (3.4); 7.3695 (3.1); 7.3604 (1.9); 7.3514 (2.0); 7.3395 (1.7); 7.3322 (2.7); 7.3236
(2.3); 7.3038 (1.2); 7.2952 (1.0); 4.9360 (13.0); 4.2077 (2.2); 4.1840 (6.8); 4.1604 (6.9); 4.1367
(2.3); 3.3199 (10.7); 2.5089 (10.6); 2.5031 (13.2); 2.4973 (9.0); 2.0757 (2.8); 1.2151 (7.8);
1.1915 (16.0); 1.1678 (7.4); −0.0001 (7.8); −0.0111 (0.3)
I-156: ¹H-NMR(300.1 MHz, d₆-DMSO):
δ = 7.5759 (3.7); 7.5497 (6.4); 7.5414 (3.2); 7.5227 (2.9); 7.4206 (2.8); 7.4169 (2.6); 7.3919
(2.2); 7.3874 (2.2); 7.3776 (2.5); 7.3699 (1.5); 7.3586 (3.3); 7.3478 (5.6); 7.3370 (2.5); 7.3295
(5.0); 7.3073 (5.2); 7.2778 (6.9); 7.2482 (2.4); 4.9203 (14.1); 4.2024 (2.3); 4.1788 (7.1); 4.1551
(7.2); 4.1315 (2.4); 3.3288 (66.3); 2.5094 (9.8); 2.5039 (12.1); 2.4984 (8.8); 1.2341 (1.6);
1.2108 (8.0); 1.1872 (16.0); 1.1635 (7.6); 0.0001 (2.4)
I-157: ¹H-NMR(300.1 MHz, d₆-DMSO):
δ = 7.5937 (2.5); 7.5859 (3.2); 7.5593 (3.6); 7.5544 (5.7); 7.5258 (8.1); 7.5191 (2.6); 7.5030
(3.0); 7.4968 (8.3); 7.4889 (1.3); 7.4313 (2.3); 7.4278 (2.4); 7.4238 (2.2); 7.4026 (1.6); 7.3991
(1.7); 7.3952 (1.5); 7.3320 (8.2); 7.3256 (2.7); 7.3096 (2.6); 7.3034 (6.1); 4.9243 (12.8); 4.2026
(2.2); 4.1790 (6.9); 4.1553 (7.0); 4.1316 (2.3); 3.3336 (64.1); 2.5107 (6.4); 2.5049 (8.0); 2.4992
(5.6); 2.0768 (1.4); 1.2340 (0.7); 1.2105 (7.7); 1.1868 (16.0); 1.1631 (7.4); 0.0000 (1.3)
I-158: ¹H-NMR(300.1 MHz, d₆-DMSO):
δ = 7.7502 (5.8); 7.7429 (5.9); 7.6476 (3.9); 7.6192 (6.5); 7.5476 (4.1); 7.5401 (3.7); 7.5191
(2.5); 7.5117 (2.3); 7.3687 (2.8); 7.3618 (1.9); 7.3499 (4.0); 7.3394 (6.0); 7.3212 (5.2); 7.2903
(5.3); 7.2609 (7.3); 7.2315 (2.7); 5.0201 (0.4); 4.9040 (14.6); 4.1908 (2.5); 4.1672 (7.3); 4.1435
(7.5); 4.1199 (2.7); 4.0964 (0.3); 3.3330 (31.8); 2.5116 (6.2); 2.5066 (7.2); 1.2335 (1.6); 1.2083
(8.2); 1.1847 (16.0); 1.1611 (7.8); 1.1294 (0.4); 0.0003 (1.3)
I-159: ¹H-NMR(300.1 MHz, d₆-DMSO):
δ = 7.7576 (5.5); 7.7502 (5.6); 7.6480 (3.6); 7.6196 (6.3); 7.5537 (4.0); 7.5461 (3.6); 7.5253
(2.5); 7.5175 (2.9); 7.5070 (6.5); 7.5008 (2.6); 7.4845 (3.4); 7.4785 (8.5); 7.3172 (8.5); 7.3108
(2.8); 7.2947 (2.8); 7.2887 (6.2); 4.9041 (12.8); 4.1888 (2.2); 4.1652 (6.9); 4.1415 (7.0); 4.1178
(2.3); 3.6856 (0.3); 3.3222 (13.8); 2.5099 (7.6); 2.5041 (9.3); 2.4984 (6.5); 2.0767 (0.4); 1.2357
(0.6); 1.2062 (7.8); 1.1826 (16.0); 1.1589 (7.4); 0.0001 (4.6)
I-160: ¹H-NMR(300.1 MHz, d₆-DMSO):
δ = 7.6095 (2.5); 7.6034 (2.4); 7.5767 (2.5); 7.5709 (2.5); 7.5538 (1.6); 7.5265 (3.3); 7.5010
(3.0); 7.4685 (3.8); 7.4623 (3.2); 7.4406 (1.8); 7.4344 (1.6); 7.2690 (0.8); 7.2384 (8.9); 7.2330
(8.4); 7.2134 (16.0); 7.1843 (0.5); 7.1651 (0.4); 7.1563 (0.3); 7.1257 (0.3); 4.9655 (11.9);
4.2257 (2.2); 4.2020 (6.4); 4.1784 (6.5); 4.1547 (2.2); 3.3449 (33.4); 2.5140 (3.1); 2.5086 (3.7);
1.2300 (7.6); 1.2064 (14.3); 1.1827 (6.8); 0.0000 (0.5)
I-161: ¹H-NMR(300.1 MHz, d₆-DMSO):
δ = 7.6310 (2.4); 7.6246 (2.5); 7.5980 (2.4); 7.5920 (2.5); 7.5638 (1.5); 7.5363 (3.4); 7.5112
(3.2); 7.4849 (4.2); 7.4785 (4.2); 7.4679 (6.9); 7.4607 (2.9); 7.4576 (2.6); 7.4504 (2.5); 7.4455
(3.0); 7.4384 (8.2); 7.4284 (1.1); 7.2008 (1.2); 7.1908 (8.0); 7.1837 (2.5); 7.1680 (2.6); 7.1613
(6.5); 7.1509 (0.7); 4.9686 (11.8); 4.2225 (2.1); 4.1988 (6.9); 4.1751 (7.0); 4.1514 (2.3); 3.5005
(0.3); 3.3950 (0.6); 3.3352 (356.2); 2.5090 (22.0); 2.5032 (28.0); 2.4974 (19.7); 1.2275 (8.2);
1.2038 (16.0); 1.1802 (7.4); 0.8535 (0.4); 0.0000 (9.7); −0.0111 (0.5)
I-162: ¹H-NMR(300.1 MHz, d₆-DMSO):
δ = 7.6365 (1.6); 7.6176 (1.8); 7.6062 (2.6); 7.6009 (3.5); 7.5906 (5.4); 7.5724 (2.8); 7.5630
(4.3); 7.4204 (0.9); 7.3928 (3.3); 7.3732 (7.0); 7.3553 (1.8); 7.3457 (1.8); 7.3280 (2.0); 7.3185
(1.8); 7.2989 (1.2); 7.2894 (1.0); 4.9134 (12.5); 4.8877 (0.4); 4.1929 (2.2); 4.1693 (6.8); 4.1456
(6.8); 4.1220 (2.2); 3.3288 (67.9); 2.5101 (8.3); 2.5042 (10.5); 2.4984 (7.3); 1.2342 (1.5);
1.2086 (7.8); 1.1850 (16.0); 1.1613 (7.4); −0.0001 (2.0)
I-163: ¹H-NMR(300.1 MHz, d₆-DMSO):
δ = 7.6120 (2.4); 7.6063 (2.4); 7.5765 (2.8); 7.5730 (2.8); 7.5522 (1.2); 7.5428 (2.0); 7.5230
(2.0); 7.5134 (1.2); 7.4936 (1.1); 7.4446 (1.5); 7.4349 (1.5); 7.4209 (3.7); 7.4143 (2.3); 7.4005
(8.4); 7.3788 (1.5); 7.3695 (1.8); 7.2229 (0.9); 7.2181 (1.0); 7.2137 (0.9); 7.1912 (1.6); 7.1658
(0.8); 7.1613 (0.8); 7.1570 (0.7); 7.1525 (0.6); 4.9291 (12.8); 4.2037 (2.3); 4.1800 (6.9); 4.1564
(7.0); 4.1327 (2.2); 3.5477 (0.5); 3.3191 (18.5); 2.5137 (8.6); 2.5082 (15.6); 2.5024 (19.4);
2.4965 (13.3); 1.2098 (7.8); 1.1861 (16.0); 1.1625 (7.3); −0.0001 (11.6); −0.0112 (0.5)
I-164: ¹H-NMR(400.0 MHz, CDCl3):
δ = 7.7645 (0.6); 7.3332 (0.5); 7.3277 (0.8); 7.3131 (0.8); 7.3079 (0.7); 7.2928 (0.9); 7.2872
(0.8); 7.2701 (2.3); 7.2663 (1.2); 7.2638 (1.5); 7.2586 (26.8); 7.2515 (3.0); 7.2441 (1.1); 7.2400
(1.5); 7.2361 (1.0); 7.2231 (1.0); 7.1329 (1.8); 7.1289 (2.6); 7.1233 (0.7); 7.1162 (1.0); 7.1117
(1.9); 7.1088 (1.6); 7.0716 (0.6); 7.0692 (0.8); 7.0653 (0.8); 7.0628 (0.9); 7.0557 (0.9); 7.0518
(1.3); 7.0495 (1.0); 7.0417 (0.5); 7.0365 (0.7); 7.0330 (1.2); 7.0302 (0.9); 7.0269 (0.8); 7.0209
(0.7); 7.0034 (0.7); 6.9994 (0.7); 6.9936 (0.5); 5.2688 (1.6); 5.2513 (1.7); 3.7889 (16.0); 3.7719
(1.7); 1.6994 (6.7); 1.6868 (1.2); 1.6819 (6.7); 1.6695 (0.8); 1.5352 (2.4); −0.0002 (9.7)
I-165: ¹H-NMR(400.0 MHz, CDCl3):
δ = 7.7643 (0.9); 7.3183 (0.6); 7.3018 (2.1); 7.2981 (0.9); 7.2955 (0.9); 7.2871 (1.1); 7.2833
(2.5); 7.2784 (1.0); 7.2742 (1.7); 7.2703 (1.1); 7.2587 (17.6); 7.1355 (1.8); 7.1314 (2.4); 7.1258
(0.7); 7.1189 (1.0); 7.1144 (2.0); 7.1113 (1.6); 6.8371 (0.6); 6.8219 (2.1); 6.8165 (2.5); 6.8065
(1.3); 6.7991 (3.6); 5.2968 (1.1); 5.2595 (1.6); 5.2421 (1.7); 5.2247 (0.5); 3.7853 (16.0); 3.7721
(2.7); 1.6969 (6.7); 1.6869 (1.5); 1.6794 (6.6); 1.6695 (1.3); 1.5430 (2.1); −0.0002 (6.3)
I-166: ¹H-NMR(400.0 MHz, CDCl3):
δ = 7.4230 (0.7); 7.4202 (0.5); 7.4180 (0.6); 7.4094 (0.5); 7.4071 (0.5); 7.4049 (0.6); 7.4023
(0.7); 7.3169 (0.9); 7.3131 (0.9); 7.2991 (0.7); 7.2948 (0.6); 7.2586 (17.3); 7.2401 (2.3); 7.2361
(1.1); 7.2345 (1.1); 7.2250 (1.2); 7.2212 (2.8); 7.2116 (1.6); 7.2091 (1.6); 7.2071 (1.8); 7.2032
(1.1); 7.1929 (1.8); 7.1903 (2.6); 7.1743 (0.9); 7.1714 (0.9); 7.1467 (2.2); 7.1427 (3.1); 7.1370
(0.7); 7.1298 (1.1); 7.1254 (1.9); 7.1224 (1.5); 7.0927 (0.7); 7.0904 (0.7); 7.0690 (1.2); 7.0475
(0.6); 7.0454 (0.6); 5.2967 (2.0); 5.2928 (0.6); 5.2753 (1.8); 5.2579 (1.8); 5.2405 (0.5); 3.7949
(16.0); 1.7042 (7.4); 1.6868 (7.3); 1.5386 (1.0); −0.0002 (9.6)
I-169: ¹H-NMR(400.0 MHz, CDCl3):
δ = 7.3538 (0.6); 7.3476 (0.6); 7.3323 (1.0); 7.3171 (0.6); 7.3109 (0.6); 7.2602 (28.6); 7.2514
(0.8); 7.2481 (0.6); 7.1750 (0.6); 7.1536 (0.6); 7.1481 (0.6); 7.1445 (0.5); 7.1348 (0.5); 7.1296
(0.6); 7.1233 (0.9); 7.1195 (0.5); 7.1033 (0.7); 7.0983 (0.8); 7.0783 (0.6); 7.0355 (0.5); 7.0289
(0.6); 7.0248 (0.5); 6.9246 (0.7); 5.1950 (1.6); 5.1777 (1.6); 3.7529 (16.0); 1.6837 (6.5); 1.6663
(6.4); 1.5409 (1.9); 0.0079 (0.7); −0.0002 (18.0); −0.0086 (0.9)
I-176: ¹H-NMR(400.0 MHz, CDCl3):
δ = 7.4057 (3.0); 7.4014 (2.7); 7.3945 (1.1); 7.3923 (1.0); 7.3878 (1.6); 7.3864 (1.4); 7.3757
(0.6); 7.3010 (1.8); 7.2966 (2.0); 7.2903 (0.6); 7.2891 (0.7); 7.2877 (0.6); 7.2851 (0.8); 7.2829
(0.8); 7.2766 (1.4); 7.2601 (32.2); 7.1584 (0.9); 7.1428 (0.8); 7.1381 (0.5); 6.9279 (0.8); 6.9226
(0.6); 6.9029 (1.3); 6.8970 (0.8); 6.8845 (0.8); 6.8822 (0.9); 6.8781 (0.6); 6.8758 (0.5); 6.8323
(0.7); 6.8301 (0.7); 6.8274 (0.6); 6.8252 (0.6); 6.8120 (0.6); 6.8099 (0.6); 6.8070 (0.6); 5.2991
(5.6); 5.2536 (1.5); 5.2362 (1.5); 3.8008 (16.0); 2.7744 (3.0); 1.7044 (6.2); 1.6870 (6.1); 1.5997
(0.9); 0.0080 (0.6); −0.0002 (18.5)
I-177: ¹H-NMR(400.0 MHz, CDCl3):
δ = 7.4213 (1.2); 7.4167 (1.6); 7.4078 (7.3); 7.4045 (7.9); 7.3986 (2.9); 7.3937 (2.6); 7.3897
(4.8); 7.3806 (1.2); 7.3763 (1.6); 7.3707 (0.5); 7.3666 (0.8); 7.3456 (0.7); 7.3004 (1.1); 7.2951
(5.7); 7.2909 (5.8); 7.2862 (2.2); 7.2842 (1.9); 7.2812 (1.8); 7.2782 (2.6); 7.2761 (3.1); 7.2709
(3.8); 7.2600 (22.9); 7.1810 (1.3); 7.1655 (1.2); 7.1605 (2.6); 7.1449 (2.4); 7.1401 (1.4); 7.1247
(1.3); 6.9274 (1.3); 6.9219 (2.7); 6.9167 (2.0); 6.9061 (1.5); 6.9037 (2.3); 6.8973 (3.0); 6.8922
(2.2); 6.8855 (2.2); 6.8832 (2.6); 6.8792 (1.3); 6.8769 (1.3); 6.8622 (3.2); 6.8592 (2.6); 6.8570
(2.3); 6.8416 (1.9); 6.8395 (1.8); 6.8367 (1.6); 6.8346 (1.3); 6.0115 (1.1); 5.3340 (1.2); 5.3165
(4.5); 5.3024 (0.9); 5.2990 (4.5); 5.2815 (1.2); 1.7566 (16.0); 1.7391 (15.8); 1.7038 (1.2);
1.6864 (1.2); 0.0080 (0.8); −0.0002 (29.9); −0.0085 (0.9)
I-178: ¹H-NMR(400.0 MHz, CDCl3):
δ = 7.4466 (0.6); 7.4404 (0.6); 7.4251 (1.1); 7.4099 (0.6); 7.4036 (0.6); 7.2615 (16.5); 7.1948
(0.7); 7.1755 (0.6); 7.1708 (1.1); 7.1660 (0.6); 7.1527 (0.8); 7.1508 (0.6); 7.1477 (0.7); 7.1405
(0.6); 7.1327 (0.6); 7.1298 (0.6); 7.1269 (0.6); 7.1233 (0.9); 7.1214 (1.1); 7.0013 (0.6); 6.9976
(0.6); 6.9826 (0.6); 5.1792 (1.6); 5.1618 (1.6); 3.7555 (16.0); 1.6871 (6.6); 1.6697 (6.5); 1.5496
(1.0); −0.0002 (10.7)
I-179: ¹H-NMR(400.0 MHz, CDCl3):
δ = 7.4467 (0.6); 7.4406 (0.7); 7.4253 (1.2); 7.4100 (0.7); 7.4038 (0.7); 7.2617 (13.1); 7.2161
(0.5); 7.1948 (1.0); 7.1752 (0.9); 7.1708 (1.5); 7.1663 (0.9); 7.1526 (1.1); 7.1478 (1.0); 7.1406
(0.9); 7.1325 (0.8); 7.1270 (0.9); 7.1215 (1.5); 7.1139 (0.7); 7.1083 (0.5); 6.9976 (1.0); 6.9820
(1.0); 5.1967 (0.5); 5.1793 (1.7); 5.1619 (1.7); 5.1445 (0.5); 3.7555 (16.0); 1.6871 (7.0); 1.6697
(7.0); −0.0002 (8.0)
II-01: ¹H-NMR(400.0 MHz, d₆-DMSO):
δ = 7.4677 (1.3); 7.4591 (5.0); 7.4564 (4.7); 7.4541 (3.9); 7.4485 (8.2); 7.4424 (10.4); 7.4331
(1.3); 7.3315 (1.2); 7.3148 (1.5); 7.3108 (2.8); 7.2965 (4.9); 7.2944 (5.1); 7.2905 (4.1); 7.2876
(5.2); 7.2785 (4.2); 7.2728 (4.3); 7.0697 (1.1); 7.0654 (1.1); 7.0482 (1.8); 7.0442 (1.9); 7.0272
(0.9); 7.0230 (1.0); 6.9311 (1.3); 6.9257 (2.1); 6.9200 (1.5); 6.9052 (1.3); 6.9000 (2.2); 6.8940
(1.6); 6.8856 (2.4); 6.8835 (2.7); 6.8808 (2.0); 6.8785 (1.7); 6.8653 (2.1); 6.8633 (2.2); 6.8605
(2.0); 4.2659 (16.0); 3.2925 (0.7); 2.6742 (1.3); 2.6695 (1.8); 2.6650 (1.3); 2.5996 (0.6); 2.5955
(0.5); 2.5587 (0.9); 2.5541 (1.8); 2.5495 (2.6); 2.5448 (2.4); 2.5401 (2.1); 2.5230 (8.0); 2.5183
(11.4); 2.5095 (107.9); 2.5051 (223.3); 2.5005 (302.9); 2.4959 (216.2); 2.4914 (102.7); 2.4636
(1.0); 2.4585 (0.5); 2.3318 (1.4); 2.3273 (1.9); 2.3227 (1.4); 0.0080 (1.2); −0.0002
(38.8); −0.0085 (1.5)
II-02: ¹H-NMR(400.0 MHz, d₆-DMSO):
δ = 7.4592 (6.1); 7.4490 (9.5); 7.4426 (11.3); 7.3322 (1.4); 7.3117 (3.2); 7.2961 (6.6); 7.2910
(5.1); 7.2874 (6.1); 7.2785 (4.9); 7.2727 (4.4); 7.0728 (1.2); 7.0663 (1.4); 7.0511 (2.1); 7.0467
(2.3); 7.0320 (1.1); 7.0255 (1.2); 6.9360 (1.6); 6.9306 (2.5); 6.9249 (1.8); 6.9102 (1.6); 6.9049
(2.6); 6.8991 (1.9); 6.8843 (3.1); 6.8641 (2.5); 6.8613 (2.4); 4.2979 (16.0); 3.5676 (1.2); 3.2987
(5.8); 2.6747 (3.1); 2.6701 (4.3); 2.6655 (3.1); 2.6136 (0.8); 2.5879 (0.8); 2.5492 (2.1); 2.5447
(3.9); 2.5234 (27.4); 2.5188 (38.3); 2.5100 (276.3); 2.5055 (561.0); 2.5010 (754.3); 2.4964
(546.2); 2.4919 (268.5); 2.4351 (1.1); 2.4003 (0.8); 2.3956 (0.7); 2.3905 (0.5); 2.3323 (3.6);
2.3277 (4.8); 2.3232 (3.7); 0.0080 (2.2); −0.0002 (62.8); −0.0085 (3.1)
II-04: ¹H-NMR(400.0 MHz, d₆-DMSO):
δ = 7.4612 (9.0); 7.4559 (6.4); 7.4531 (10.2); 7.4452 (10.4); 7.4411 (3.6); 7.4352 (1.3); 7.4201
(0.6); 7.4097 (0.8); 7.4034 (0.9); 7.3498 (1.5); 7.3333 (1.9); 7.3292 (3.3); 7.3128 (3.9); 7.3082
(3.5); 7.3047 (5.6); 7.3010 (3.6); 7.2973 (5.1); 7.2943 (4.6); 7.2914 (5.1); 7.2882 (4.6); 7.2845
(3.1); 7.2807 (4.2); 7.1042 (1.4); 7.0997 (1.4); 7.0828 (2.4); 7.0784 (2.4); 7.0636 (1.1); 7.0616
(1.2); 7.0571 (1.2); 6.9641 (1.8); 6.9587 (2.8); 6.9529 (1.9); 6.9384 (1.8); 6.9332 (2.9); 6.9272
(2.0); 6.9054 (2.9); 6.9033 (3.2); 6.9006 (2.6); 6.8984 (2.4); 6.8852 (2.6); 6.8831 (2.7); 6.8802
(2.6); 6.8782 (2.2); 6.5483 (0.7); 6.5083 (0.6); 4.6410 (16.0); 3.7305 (1.2); 3.5917 (10.8);
3.5799 (12.4); 3.5679 (11.6); 3.3549 (2.2); 2.8119 (10.1); 2.7998 (11.2); 2.7880 (9.8); 2.6740
(1.0); 2.6694 (1.5); 2.6647 (1.1); 2.5228 (4.6); 2.5182 (6.6); 2.5094 (79.9); 2.5049 (168.4);
2.5003 (230.8); 2.4958 (164.0); 2.4912 (76.9); 2.4589 (0.5); 2.4546 (0.8); 2.4497 (1.2); 2.4453
(1.1); 2.3806 (0.5); 2.3316 (1.1); 2.3271 (1.4); 2.3226 (1.1); 0.0080 (1.5); −0.0002
(47.9); −0.0085 (1.8)
II-05: ¹H-NMR(400.0 MHz, d₆-DMSO):
δ = 7.4597 (5.9); 7.4516 (6.9); 7.4437 (7.2); 7.4341 (1.0); 7.3407 (1.0); 7.3241 (1.3); 7.3201
(2.2); 7.3117 (0.8); 7.3013 (4.7); 7.2939 (3.6); 7.2911 (3.1); 7.2877 (2.9); 7.2843 (3.8); 7.2774
(3.0); 7.0944 (1.0); 7.0897 (1.0); 7.0730 (1.7); 7.0683 (1.7); 7.0536 (0.8); 7.0517 (0.8); 7.0471
(0.8); 6.9550 (1.2); 6.9496 (2.0); 6.9439 (1.3); 6.9293 (1.2); 6.9241 (2.0); 6.9183 (1.4); 6.9003
(2.2); 6.8975 (1.8); 6.8819 (1.8); 6.8801 (1.9); 6.8772 (1.8); 5.9152 (0.6); 5.8889 (0.8); 5.8720
(0.9); 5.8458 (0.8); 5.7527 (16.0); 5.3459 (1.4); 5.3422 (1.5); 5.3026 (1.2); 5.2990 (1.2); 5.2357
(1.5); 5.2324 (1.5); 5.2095 (1.4); 5.2063 (1.4); 4.5838 (9.4); 3.4125 (2.8); 3.4092 (2.0); 3.3976
(2.9); 2.5101 (12.4); 2.5057 (28.0); 2.5011 (39.9); 2.4966 (30.1); 2.4922 (15.6); −0.0002 (7.0)
II-06: ¹H-NMR(400.0 MHz, d₆-DMSO):
δ = 7.4659 (1.8); 7.4600 (8.1); 7.4515 (9.7); 7.4477 (6.6); 7.4439 (11.4); 7.4409 (5.1); 7.4343
(2.1); 7.4096 (0.8); 7.4050 (0.8); 7.3970 (0.9); 7.3921 (0.9); 7.3381 (1.5); 7.3216 (1.8); 7.3175
(3.3); 7.3091 (1.1); 7.2992 (6.8); 7.2912 (5.2); 7.2893 (4.9); 7.2859 (4.2); 7.2817 (5.7); 7.2751
(5.0); 7.2689 (1.4); 7.0922 (1.2); 7.0903 (1.4); 7.0839 (1.4); 7.0689 (2.4); 7.0627 (2.5); 7.0496
(1.2); 7.0477 (1.3); 7.0432 (1.3); 7.0413 (1.3); 6.9524 (1.8); 6.9469 (2.8); 6.9412 (2.0); 6.9266
(1.8); 6.9213 (3.0); 6.9155 (2.1); 6.8948 (2.8); 6.8928 (3.2); 6.8900 (2.7); 6.8880 (2.6); 6.8746
(2.6); 6.8726 (2.8); 6.8697 (2.7); 6.8677 (2.4); 5.7531 (16.0); 4.5178 (12.9); 2.7229 (3.9);
2.7043 (4.8); 2.6853 (4.3); 2.6749 (0.6); 2.6702 (0.6); 2.5236 (1.0); 2.5189 (1.5); 2.5102 (22.4);
2.5057 (48.9); 2.5011 (69.0); 2.4965 (50.9); 2.4920 (26.2); 1.5622 (2.0); 1.5434 (3.6); 1.5246
(3.8); 1.5060 (2.3); 1.4874 (0.7); 0.8847 (7.7); 0.8661 (16.0); 0.8474 (7.2); −0.0002
(12.8); −0.0085 (0.6)
II-07: ¹H-NMR(400.0 MHz, d₆-DMSO):
δ = 7.6913 (1.0); 7.6823 (1.3); 7.6792 (1.0); 7.6728 (1.3); 7.6672 (1.2); 7.6036 (1.4); 7.5635
(1.6); 7.4630 (3.1); 7.4581 (2.1); 7.4551 (3.3); 7.4524 (2.0); 7.4469 (3.2); 7.4419 (1.1); 7.4296
(0.7); 7.4233 (2.1); 7.4137 (2.7); 7.4068 (2.9); 7.3572 (0.6); 7.3407 (0.7); 7.3366 (1.2); 7.3202
(1.3); 7.3159 (1.0); 7.3088 (1.9); 7.3018 (1.9); 7.2983 (1.6); 7.2947 (1.4); 7.2928 (1.6); 7.2885
(1.0); 7.2848 (1.4); 7.0949 (0.8); 7.0904 (0.8); 6.9767 (0.6); 6.9713 (0.9); 6.9654 (0.6); 6.9511
(0.6); 6.9458 (1.0); 6.9399 (0.6); 6.9103 (0.9); 6.9083 (1.0); 6.9055 (0.9); 6.9034 (0.8); 6.8902
(0.8); 6.8881 (0.9); 6.8852 (0.8); 6.8831 (0.7); 6.5494 (2.3); 6.5093 (2.1); 4.7574 (4.4); 3.5677
(3.8); 3.3165 (0.8); 3.2439 (0.6); 2.5232 (1.4); 2.5185 (2.0); 2.5098 (25.7); 2.5053 (54.0);
2.5007 (74.1); 2.4961 (53.5); 2.4916 (25.8); 2.4766 (2.5); 2.4565 (1.8); 1.4641 (1.6); 1.4454
(2.7); 1.4266 (2.8); 1.4080 (1.7); 1.2353 (0.9); 0.8610 (7.7); 0.8427 (16.0); 0.8242
(6.9); −0.0002 (8.7)
II-08: ¹H-NMR(400.0 MHz, d₆-DMSO):
δ = 7.6397 (1.8); 7.6343 (1.6); 7.6203 (2.3); 7.6159 (2.0); 7.4867 (2.0); 7.4678 (1.0); 7.4610
(4.1); 7.4519 (5.4); 7.4448 (7.4); 7.4351 (1.0); 7.4203 (0.6); 7.4112 (0.7); 7.4066 (0.6); 7.3979
(2.4); 7.3879 (1.8); 7.3823 (3.8); 7.3797 (3.7); 7.3697 (0.9); 7.3657 (0.7); 7.3378 (0.9); 7.3214
(1.2); 7.3171 (1.9); 7.3082 (0.6); 7.3008 (2.9); 7.2985 (3.6); 7.2927 (2.0); 7.2895 (3.0); 7.2856
(2.1); 7.2807 (3.4); 7.2744 (2.6); 7.0896 (0.7); 7.0876 (0.8); 7.0832 (0.8); 7.0812 (0.8); 7.0682
(1.2); 7.0662 (1.3); 7.0618 (1.3); 7.0599 (1.3); 7.0471 (0.6); 7.0450 (0.7); 7.0406 (0.6); 7.0386
(0.6); 6.9515 (1.0); 6.9460 (1.5); 6.9403 (1.0); 6.9257 (1.0); 6.9204 (1.6); 6.9145 (1.1); 6.8868
(1.5); 6.8847 (1.7); 6.8818 (1.4); 6.8798 (1.3); 6.8665 (1.4); 6.8644 (1.4); 6.8615 (1.4); 6.8594
(1.2); 6.5233 (3.3); 6.4833 (3.1); 4.4480 (8.9); 2.8181 (1.9); 2.8000 (5.9); 2.7819 (6.0); 2.7638
(2.0); 2.6701 (0.5); 2.5235 (1.3); 2.5189 (2.0); 2.5101 (30.5); 2.5056 (65.0); 2.5010 (89.4);
2.4964 (63.7); 2.4919 (30.1); 2.4388 (32.4); 2.3279 (0.6); 1.2344 (1.0); 1.1270 (7.5); 1.1089
(16.0); 1.0908 (7.2); −0.0002 (11.7)
II-09: ¹H-NMR(400.0 MHz, d₆-DMSO):
δ = 7.4605 (1.5); 7.4514 (1.8); 7.4443 (2.2); 7.3167 (0.7); 7.3005 (1.0); 7.2975 (1.3); 7.2886
(1.1); 7.2848 (0.7); 7.2800 (1.2); 7.2736 (0.8); 6.9426 (0.5); 6.9170 (0.5); 6.8864 (0.6); 6.8843
(0.6); 6.8815 (0.5); 6.8662 (0.5); 4.4522 (1.4); 3.3124 (3.9); 2.5228 (1.5); 2.5181 (2.1); 2.5093
(26.2); 2.5048 (54.8); 2.5002 (74.9); 2.4957 (53.0); 2.4911 (24.7); 1.2204 (16.0); −0.0002 (11.8)
II-10: ¹H-NMR(400.0 MHz, d₆-DMSO):
δ = 7.4582 (1.7); 7.4495 (2.0); 7.4421 (2.4); 7.4003 (0.6); 7.3293 (0.6); 7.3110 (1.7); 7.2918
(2.4); 7.2820 (0.9); 7.2751 (1.1); 7.2711 (1.0); 7.2387 (1.7); 7.2264 (1.0); 7.2196 (1.0); 7.0634
(0.5); 7.0589 (0.5); 6.9355 (0.6); 6.9103 (0.6); 6.8877 (0.7); 6.8672 (0.6); 4.4795 (1.5); 3.3118
(16.0); 2.9714 (0.6); 2.9534 (1.2); 2.9470 (0.7); 2.9322 (1.1); 2.8076 (1.0); 2.7866 (1.1); 2.7687
(0.6); 2.6740 (0.8); 2.6694 (1.1); 2.6648 (0.9); 2.5229 (3.4); 2.5181 (4.9); 2.5094 (65.6); 2.5049
(138.2); 2.5003 (189.2); 2.4958 (135.0); 2.4913 (63.8); 2.4507 (1.1); 2.4457 (0.7); 2.3317 (0.8);
2.3271 (1.1); 2.3225 (0.8); 0.0079 (0.6); −0.0002 (20.9); −0.0085 (0.8)
II-11: ¹H-NMR(400.0 MHz, d₆-DMSO):
δ = 7.4678 (1.1); 7.4612 (4.0); 7.4522 (5.1); 7.4450 (6.2); 7.4353 (1.1); 7.3381 (0.8); 7.3217
(0.9); 7.3175 (1.7); 7.3076 (0.6); 7.3010 (2.6); 7.2977 (3.6); 7.2920 (1.9); 7.2892 (2.9); 7.2850
(2.2); 7.2805 (3.5); 7.2739 (2.6); 7.0904 (0.6); 7.0884 (0.7); 7.0838 (0.7); 7.0820 (0.8); 7.0688
(1.1); 7.0670 (1.2); 7.0624 (1.3); 7.0608 (1.3); 7.0478 (0.6); 7.0458 (0.7); 7.0413 (0.7); 7.0394
(0.6); 6.9539 (0.9); 6.9484 (1.5); 6.9427 (1.0); 6.9281 (1.0); 6.9227 (1.5); 6.9169 (1.1); 6.8870
(1.4); 6.8849 (1.6); 6.8821 (1.4); 6.8800 (1.4); 6.8667 (1.3); 6.8647 (1.4); 6.8618 (1.4); 6.8597
(1.3); 5.7530 (16.0); 4.4682 (8.4); 2.9324 (2.6); 2.9191 (3.8); 2.9050 (3.1); 2.5188 (0.7); 2.5101
(11.8); 2.5055 (26.3); 2.5009 (37.8); 2.4964 (28.5); 2.4918 (15.1); 1.6250 (0.6); 1.5973 (3.0);
1.5847 (2.5); 1.5700 (1.3); 1.5030 (1.4); 1.4914 (1.5); −0.0002 (8.6)
II-12: ¹H-NMR(400.0 MHz, d₆-DMSO):
δ = 7.4608 (6.4); 7.4524 (7.3); 7.4485 (4.8); 7.4447 (8.3); 7.3453 (1.1); 7.3289 (1.3); 7.3246
(2.4); 7.3082 (2.8); 7.3024 (4.6); 7.2986 (2.7); 7.2945 (3.7); 7.2923 (3.4); 7.2885 (3.7); 7.2855
(3.7); 7.2783 (3.3); 7.0970 (1.0); 7.0907 (1.0); 7.0758 (1.8); 7.0698 (1.8); 7.0544 (0.9); 7.0501
(0.8); 6.9567 (1.3); 6.9512 (2.1); 6.9455 (1.4); 6.9309 (1.3); 6.9258 (2.1); 6.9198 (1.4); 6.8999
(2.1); 6.8979 (2.3); 6.8950 (1.9); 6.8796 (1.9); 6.8776 (2.0); 6.8748 (1.9); 6.8726 (1.7); 4.5654
(4.1); 3.5675 (4.6); 3.3120 (16.0); 3.1682 (0.6); 2.7559 (1.9); 2.7373 (2.2); 2.7180 (2.0); 2.6740
(1.4); 2.6694 (2.0); 2.6647 (1.4); 2.5988 (0.8); 2.5228 (5.9); 2.5181 (8.6); 2.5094 (116.8);
2.5049 (246.0); 2.5003 (336.5); 2.4957 (238.9); 2.4912 (112.9); 2.4397 (0.6); 2.4123 (0.5);
2.3317 (1.4); 2.3270 (2.0); 2.3225 (1.5); 1.5031 (1.1); 1.4842 (0.8); 1.2483 (9.4); 0.8748 (1.9);
0.8580 (6.5); 0.8404 (2.3); 0.0080 (1.9); −0.0002 (58.5); −0.0085 (2.3)
II-13: ¹H-NMR(400.0 MHz, d₆-DMSO):
δ = 7.4584 (8.7); 7.4500 (10.1); 7.4461 (6.9); 7.4423 (11.6); 7.4389 (4.9); 7.4341 (4.5); 7.4302
(5.0); 7.4133 (7.0); 7.3981 (1.0); 7.3930 (0.9); 7.3769 (2.5); 7.3729 (3.4); 7.3682 (1.5); 7.3558
(8.0); 7.3517 (3.4); 7.3407 (2.5); 7.3373 (4.7); 7.3260 (2.4); 7.3221 (4.0); 7.3184 (4.0); 7.3141
(4.1); 7.3047 (4.2); 7.2969 (8.7); 7.2933 (6.1); 7.2891 (6.0); 7.2868 (5.7); 7.2834 (4.8); 7.2798
(5.4); 7.2769 (5.3); 7.2728 (4.9); 7.0902 (1.3); 7.0882 (1.5); 7.0838 (1.5); 7.0819 (1.5); 7.0687
(2.3); 7.0668 (2.5); 7.0624 (2.5); 7.0476 (1.2); 7.0455 (1.3); 7.0411 (1.3); 7.0392 (1.2); 6.9493
(1.8); 6.9438 (2.9); 6.9381 (2.0); 6.9235 (1.9); 6.9183 (3.1); 6.9124 (2.1); 6.8967 (3.0); 6.8947
(3.4); 6.8918 (2.7); 6.8897 (2.5); 6.8764 (2.7); 6.8744 (2.9); 6.8716 (2.7); 6.8695 (2.4); 5.7530
(16.0); 4.5278 (14.7); 3.9323 (14.6); 2.5231 (0.9); 2.5184 (1.4); 2.5096 (30.8); 2.5051 (67.2);
2.5005 (93.6); 2.4960 (68.4); 2.4914 (33.5); 2.4359 (0.5); 2.3319 (0.6); 2.3274 (0.7); 2.3227
(0.6); −0.0002 (12.0); −0.0085 (0.5)
II-14: ¹H-NMR(400.0 MHz, CDCl3):
δ = 7.4021 (0.6); 7.3954 (3.7); 7.3906 (3.8); 7.3829 (2.3); 7.3779 (2.3); 7.3706 (0.6); 7.3669
(0.8); 7.2821 (2.2); 7.2775 (2.5); 7.2692 (1.7); 7.2618 (22.2); 7.2581 (2.4); 7.1197 (0.5); 7.1039
(0.6); 7.0991 (1.2); 7.0833 (1.2); 7.0786 (0.8); 7.0628 (0.7); 6.9710 (0.6); 6.9654 (1.0); 6.9598
(0.7); 6.9454 (0.6); 6.9399 (1.0); 6.9342 (0.7); 6.8362 (0.6); 6.8298 (0.6); 6.8209 (1.3); 6.8170
(1.9); 6.8110 (1.1); 6.8091 (0.8); 6.7998 (1.0); 6.7966 (1.3); 6.7904 (0.5); 5.2979 (11.6); 4.8230
(7.5); 3.0865 (2.1); 3.0682 (7.0); 3.0499 (7.2); 3.0316 (2.2); 1.2574 (7.5); 1.2392 (16.0); 1.2208
(7.2); −0.0002 (8.9)
III-001: ¹H-NMR(400.0 MHz, CDCl3):
δ = 7.3870 (1.0); 7.3827 (1.2); 7.3678 (2.0); 7.3637 (2.3); 7.3531 (1.1); 7.3493 (2.3); 7.3393
(5.0); 7.3364 (5.7); 7.3310 (2.8); 7.3211 (5.4); 7.3074 (2.5); 7.3004 (1.8); 7.2887 (1.7); 7.2830
(5.2); 7.2787 (4.7); 7.2741 (2.2); 7.2686 (1.5); 7.2592 (20.6); 7.1839 (1.4); 7.1649 (2.1); 7.1462
(0.9); 7.0343 (1.3); 7.0312 (1.2); 7.0135 (1.3); 7.0098 (2.1); 7.0061 (1.3); 6.9885 (1.1); 6.9854
(1.0); 6.7256 (0.7); 5.2952 (11.6); 4.8957 (3.1); 4.8718 (16.0); 4.8652 (3.6); 4.8618 (2.6);
3.9428 (3.6); 3.9276 (3.5); 1.7660 (11.9); 1.5854 (0.8); 1.2557 (0.8); −0.0002 (6.8)
III-002: ¹H-NMR(400.0 MHz, CDCl3):
δ = 7.3748 (0.7); 7.3705 (0.9); 7.3555 (1.5); 7.3517 (2.1); 7.3375 (4.2); 7.3356 (4.7); 7.3327
(3.0); 7.3301 (2.3); 7.3236 (1.9); 7.3197 (3.8); 7.3176 (3.4); 7.3089 (1.2); 7.3057 (1.7); 7.3005
(1.2); 7.2967 (1.1); 7.2881 (0.8); 7.2838 (1.1); 7.2774 (3.6); 7.2732 (3.6); 7.2688 (1.7); 7.2604
(15.8); 7.2530 (1.7); 7.1828 (0.9); 7.1812 (1.0); 7.1620 (1.4); 7.1442 (0.6); 7.1427 (0.6); 7.0377
(0.9); 7.0345 (0.9); 7.0169 (0.9); 7.0130 (1.5); 7.0094 (1.0); 6.9918 (0.8); 6.9886 (0.8); 6.6878
(0.6); 5.2956 (16.0); 4.8041 (11.3); 2.8234 (0.7); 2.8141 (1.0); 2.8055 (1.0); 2.7962 (0.8);
2.0419 (0.6); 1.6016 (0.5); 1.2572 (0.9); 0.8631 (0.8); 0.8491 (1.8); 0.8454 (2.8); 0.8426 (1.3);
0.8317 (2.6); 0.8276 (2.1); 0.8247 (1.2); 0.8143 (0.9); 0.6284 (0.9); 0.6184 (1.8); 0.6153 (2.0);
0.6113 (2.0); 0.6099 (2.0); 0.6057 (2.0); 0.6020 (1.8); 0.5883 (0.7); −0.0002 (5.3)
III-003: ¹H-NMR(400.0 MHz, CDCl3):
δ = 7.3866 (1.2); 7.3823 (1.4); 7.3742 (0.6); 7.3672 (2.2); 7.3630 (2.5); 7.3541 (1.4); 7.3504
(1.8); 7.3486 (1.9); 7.3423 (4.0); 7.3400 (5.4); 7.3372 (6.3); 7.3317 (3.3); 7.3296 (1.8); 7.3259
(2.7); 7.3219 (5.7); 7.3196 (5.1); 7.3141 (1.4); 7.3121 (1.5); 7.3103 (1.8); 7.3082 (2.8); 7.3064
(2.0); 7.3021 (1.8); 7.2989 (1.5); 7.2898 (2.0); 7.2836 (5.7); 7.2793 (5.3); 7.2747 (2.4); 7.2694
(1.6); 7.2663 (2.4); 7.2592 (37.8); 7.1877 (1.2); 7.1860 (1.4); 7.1844 (1.4); 7.1650 (2.0); 7.1491
(0.8); 7.1474 (0.9); 7.1458 (0.9); 7.1444 (0.8); 7.0377 (1.4); 7.0345 (1.3); 7.0169 (1.4); 7.0131
(2.2); 7.0094 (1.4); 6.9919 (1.2); 6.9887 (1.1); 6.7103 (0.6); 5.9472 (0.6); 5.9336 (1.3); 5.9213
(0.8); 5.9078 (1.4); 5.9043 (0.8); 5.8941 (0.8); 5.8906 (1.5); 5.8783 (0.9); 5.8772 (0.9); 5.8649
(1.5); 5.8512 (0.7); 5.2963 (9.6); 5.2791 (0.9); 5.2749 (2.1); 5.2717 (2.2); 5.2674 (1.1); 5.2362
(0.8); 5.2320 (1.8); 5.2287 (1.9); 5.2245 (1.0); 5.1885 (1.1); 5.1849 (2.5); 5.1816 (2.4); 5.1780
(1.0); 5.1628 (1.0); 5.1592 (2.3); 5.1558 (2.2); 5.1522 (0.9); 4.8601 (16.0); 4.0348 (1.4); 4.0309
(2.4); 4.0269 (1.6); 4.0202 (2.5); 4.0167 (4.0); 4.0131 (2.4); 4.0065 (1.4); 4.0025 (2.2); 3.9986
(1.2); 1.5654 (5.0); 0.0079 (0.6); −0.0002 (18.2); −0.0085 (0.7)
III-004: ¹H-NMR(400.0 MHz, CDCl3):
δ = 7.3122 (0.9); 7.3081 (1.2); 7.3068 (1.4); 7.3024 (0.9); 7.2981 (0.7); 7.2956 (0.8); 7.2920
(3.2); 7.2905 (4.9); 7.2866 (5.4); 7.2843 (3.0); 7.2810 (1.9); 7.2758 (2.8); 7.2719 (7.8); 7.2700
(5.6); 7.2667 (3.3); 7.2641 (6.6); 7.2625 (5.7); 7.2596 (38.6); 7.2510 (4.7); 7.2464 (2.1); 7.2449
(2.1); 7.2270 (0.6); 7.1570 (4.2); 7.1529 (5.2); 7.1472 (1.4); 7.1406 (2.2); 7.1379 (1.7); 7.1360
(4.2); 7.1326 (3.4); 7.0974 (0.5); 7.0902 (4.5); 7.0847 (1.3); 7.0761 (0.7); 7.0733 (1.4); 7.0688
(6.3); 7.0635 (1.4); 7.0608 (0.6); 7.0521 (1.2); 7.0466 (3.5); 6.6980 (0.6); 5.9491 (0.6); 5.9354
(1.3); 5.9232 (0.8); 5.9219 (0.8); 5.9096 (1.5); 5.9062 (0.8); 5.8960 (0.8); 5.8925 (1.6); 5.8803
(0.9); 5.8789 (0.9); 5.8667 (1.6); 5.8531 (0.8); 5.2970 (7.6); 5.2791 (0.9); 5.2748 (2.1); 5.2716
(2.2); 5.2673 (1.0); 5.2361 (0.8); 5.2318 (1.9); 5.2286 (1.9); 5.2243 (0.9); 5.1882 (1.0); 5.1846
(2.5); 5.1812 (2.4); 5.1776 (0.9); 5.1625 (0.9); 5.1589 (2.3); 5.1555 (2.2); 5.1519 (0.9); 4.8824
(16.0); 4.0374 (1.2); 4.0334 (2.1); 4.0295 (1.4); 4.0228 (2.2); 4.0192 (3.7); 4.0155 (2.2); 4.0089
(1.3); 4.0049 (2.1); 4.0009 (1.2); −0.0002 (16.4)
III-005: ¹H-NMR(400.0 MHz, CDCl3):
δ = 7.3116 (0.6); 7.3075 (0.8); 7.3062 (1.0); 7.3018 (0.6); 7.2977 (0.6); 7.2950 (0.6); 7.2909
(4.3); 7.2899 (4.4); 7.2859 (2.1); 7.2837 (1.8); 7.2778 (3.0); 7.2750 (2.3); 7.2713 (4.7); 7.2690
(4.8); 7.2654 (1.8); 7.2635 (1.6); 7.2603 (20.1); 7.2570 (3.0); 7.2555 (3.4); 7.2508 (0.8); 7.2483
(0.5); 7.2449 (1.3); 7.2436 (1.3); 7.1614 (2.9); 7.1574 (3.6); 7.1516 (1.0); 7.1449 (1.5); 7.1422
(1.1); 7.1403 (2.9); 7.1370 (2.2); 7.0926 (3.1); 7.0871 (0.9); 7.0783 (0.5); 7.0757 (1.0); 7.0711
(4.3); 7.0658 (0.9); 7.0544 (0.9); 7.0490 (2.4); 5.2965 (16.0); 4.8572 (10.2); 3.2647 (2.2);
3.2505 (2.5); 3.2471 (2.5); 3.2328 (2.2); 1.0479 (0.5); 1.0280 (0.9); 1.0080 (0.6); 0.5572 (0.8);
0.5454 (1.9); 0.5423 (2.4); 0.5400 (1.3); 0.5372 (1.0); 0.5309 (1.1); 0.5252 (2.3); 0.5222 (1.9);
0.5109 (0.9); 0.2743 (0.9); 0.2628 (2.6); 0.2604 (2.2); 0.2508 (1.9); 0.2480 (2.6); 0.2363
(0.6); −0.0002 (8.0)
III-006: ¹H-NMR(400.0 MHz, CDCl3):
δ = 7.3125 (0.6); 7.3084 (0.8); 7.3069 (0.9); 7.3025 (0.6); 7.2958 (0.7); 7.2907 (3.2); 7.2885
(3.0); 7.2842 (1.8); 7.2754 (3.4); 7.2722 (4.7); 7.2704 (3.6); 7.2661 (3.8); 7.2636 (3.3); 7.2596
(29.3); 7.2530 (3.3); 7.2476 (1.3); 7.2459 (1.4); 7.1591 (2.8); 7.1549 (3.2); 7.1492 (0.9); 7.1427
(1.4); 7.1399 (1.1); 7.1381 (2.7); 7.1347 (2.2); 7.0918 (2.9); 7.0863 (0.9); 7.0749 (0.9); 7.0704
(4.0); 7.0651 (1.0); 7.0537 (0.8); 7.0483 (2.3); 5.2972 (16.0); 4.8798 (10.2); 4.1930 (2.8);
4.1866 (2.9); 4.1793 (2.8); 4.1729 (2.8); 2.2746 (2.3); 2.2682 (4.9); 2.2618 (2.2); −0.0002 (11.6)
III-007: ¹H-NMR(400.0 MHz, CDCl3):
δ = 7.3056 (0.5); 7.3041 (0.6); 7.2895 (1.4); 7.2879 (2.2); 7.2848 (2.2); 7.2816 (1.2); 7.2794
(0.9); 7.2717 (2.2); 7.2694 (3.2); 7.2676 (2.5); 7.2625 (3.1); 7.2599 (19.0); 7.2559 (1.7); 7.2495
(2.3); 7.2440 (0.9); 7.2424 (1.0); 7.1546 (1.9); 7.1505 (2.3); 7.1449 (0.6); 7.1382 (1.0); 7.1355
(0.7); 7.1336 (1.9); 7.1302 (1.5); 7.0905 (2.0); 7.0849 (0.6); 7.0735 (0.6); 7.0690 (2.8); 7.0637
(0.6); 7.0523 (0.6); 7.0469 (1.6); 5.2970 (3.7); 4.8574 (7.0); 3.2283 (1.7); 3.2118 (2.5); 3.1960
(1.7); 1.8661 (0.7); 1.8493 (0.9); 1.8325 (0.8); 1.5713 (2.6); 0.9560 (16.0); 0.9513 (1.2); 0.9392
(15.4); 0.9343 (0.8); −0.0002 (7.5)
III-008: ¹H-NMR(400.0 MHz, CDCl3):
δ = 7.5185 (0.7); 7.3843 (0.9); 7.3692 (1.1); 7.3636 (1.6); 7.3490 (1.9); 7.3444 (1.3); 7.3293
(1.4); 7.2596 (116.5); 7.1371 (3.4); 7.1314 (1.9); 7.1252 (4.3); 7.1198 (2.5); 7.1142 (5.1);
7.1114 (2.3); 7.1084 (3.2); 7.1023 (6.2); 7.0939 (0.7); 7.0901 (1.0); 7.0877 (1.0); 7.0837 (1.0);
7.0812 (0.9); 7.0364 (1.9); 7.0326 (2.7); 7.0301 (2.2); 7.0139 (4.1); 7.0111 (3.2); 7.0077 (2.2);
6.9994 (4.9); 6.9955 (1.5); 6.9935 (1.8); 6.9905 (2.0); 6.9872 (2.1); 6.9792 (6.0); 6.9766 (4.1);
6.9734 (1.7); 6.9620 (1.3); 6.9563 (3.4); 6.6799 (0.7); 5.9590 (0.6); 5.9454 (1.4); 5.9330 (1.0);
5.9196 (1.5); 5.9161 (0.8); 5.9060 (0.8); 5.9024 (1.6); 5.8891 (1.0); 5.8767 (1.6); 5.8630 (0.8);
5.2906 (1.0); 5.2863 (2.2); 5.2831 (2.2); 5.2788 (1.0); 5.2476 (0.9); 5.2433 (2.0); 5.2402 (2.0);
5.2358 (0.9); 5.1969 (1.1); 5.1933 (2.5); 5.1899 (2.3); 5.1864 (1.0); 5.1711 (1.0); 5.1675 (2.3);
5.1642 (2.2); 5.1605 (0.9); 4.8613 (16.0); 4.0433 (1.3); 4.0393 (2.2); 4.0352 (1.4); 4.0251 (4.0);
4.0150 (1.5); 4.0109 (2.2); 1.5454 (4.1); 0.0080 (1.5); −0.0002 (43.8); −0.0085 (1.6)
III-009: ¹H-NMR(400.0 MHz, CDCl3):
δ = 7.5187 (0.5); 7.3846 (0.8); 7.3698 (1.2); 7.3640 (1.6); 7.3496 (1.8); 7.3447 (1.3); 7.3297
(1.3); 7.2599 (86.0); 7.1351 (3.4); 7.1299 (1.9); 7.1234 (4.3); 7.1179 (2.4); 7.1122 (5.8); 7.1091
(2.3); 7.1060 (2.8); 7.1005 (4.9); 7.0907 (0.9); 7.0881 (0.8); 7.0841 (0.8); 7.0817 (0.8); 7.0356
(1.7); 7.0318 (2.6); 7.0293 (2.1); 7.0131 (4.3); 7.0103 (3.1); 6.9975 (4.8); 6.9914 (2.7); 6.9856
(2.0); 6.9804 (2.5); 6.9773 (5.3); 6.9716 (1.6); 6.9602 (1.1); 6.9546 (3.2); 6.5958 (0.7); 5.2982
(0.6); 4.8225 (15.7); 3.4107 (1.8); 3.3931 (3.7); 3.3781 (3.6); 3.3605 (1.8); 1.6093 (0.8); 1.5909
(2.0); 1.5849 (0.9); 1.5727 (3.1); 1.5669 (1.8); 1.5505 (5.6); 1.5363 (1.3); 1.4439 (0.7); 1.4257
(2.0); 1.4066 (2.6); 1.3872 (2.6); 1.3692 (1.6); 1.3509 (0.6); 0.9602 (7.7); 0.9419 (16.0); 0.9236
(6.5); 0.0080 (1.0); −0.0002 (31.8); −0.0085 (1.0)
III-010: ¹H-NMR(400.0 MHz, CDCl3):
δ = 7.3737 (1.0); 7.3586 (1.5); 7.3533 (1.9); 7.3383 (2.7); 7.3337 (2.2); 7.3271 (4.7); 7.3239
(7.9); 7.3224 (6.9); 7.3176 (7.5); 7.3134 (5.0); 7.3114 (7.1); 7.3094 (6.9); 7.3030 (2.0); 7.2969
(1.9); 7.2930 (3.3); 7.2902 (2.5); 7.2865 (2.4); 7.2828 (1.6); 7.2781 (1.1); 7.2747 (1.4); 7.2713
(1.7); 7.2583 (44.3); 7.1235 (1.1); 7.1213 (1.3); 7.1169 (1.4); 7.1137 (3.9); 7.1078 (1.6); 7.1018
(4.8); 7.0962 (4.0); 7.0934 (3.0); 7.0907 (5.2); 7.0847 (1.9); 7.0788 (5.5); 7.0749 (1.4); 7.0724
(1.2); 7.0168 (2.0); 7.0130 (2.7); 7.0105 (2.3); 7.0035 (1.0); 6.9951 (7.2); 6.9913 (4.3); 6.9846
(2.2); 6.9816 (1.7); 6.9778 (2.1); 6.9749 (5.7); 6.9724 (4.3); 6.9690 (3.0); 6.9640 (2.1); 6.9624
(1.9); 6.9580 (2.4); 6.9521 (3.5); 6.9438 (0.6); 6.9008 (0.9); 5.2962 (6.9); 4.8965 (16.0); 4.5951
(6.8); 4.5802 (6.7); 1.5591 (3.8); 0.0079 (0.7); −0.0002 (17.1); −0.0085 (0.6)
III-011: ¹H-NMR(400.0MHz, CDCl3):
δ = 7.3826 (0.7); 7.3676 (1.1); 7.3620 (1.6); 7.3474 (1.7); 7.3420 (1.2); 7.3275 (1.3); 7.2605
(46.3); 7.1299 (3.6); 7.1245 (2.0); 7.1181 (3.3); 7.1127 (2.4); 7.1102 (2.2); 7.1073 (5.9); 7.1043
(2.1); 7.1013 (3.0); 7.0953 (4.4); 7.0891 (0.9); 7.0868 (1.2); 7.0827 (0.9); 7.0803 (0.8); 7.0312
(1.7); 7.0275 (2.5); 7.0249 (2.0); 7.0085 (4.0); 7.0059 (3.4); 7.0022 (1.9); 6.9979 (5.1); 6.9920
(1.5); 6.9859 (2.1); 6.9775 (5.5); 6.9720 (1.6); 6.9606 (1.1); 6.9548 (3.2); 6.6633 (0.8); 5.2982
(4.4); 4.8063 (16.0); 2.8459 (0.6); 2.8373 (1.0); 2.8280 (1.4); 2.8194 (1.4); 2.8101 (1.1); 2.8014
(0.6); 1.5620 (3.0); 1.4319 (0.5); 0.8758 (1.0); 0.8616 (2.6); 0.8583 (4.0); 0.8554 (1.9); 0.8445
(3.6); 0.8405 (3.0); 0.8375 (1.7); 0.8270 (1.3); 0.6308 (1.3); 0.6208 (2.6); 0.6176 (2.9); 0.6139
(2.8); 0.6119 (2.8); 0.6080 (2.9); 0.6044 (2.6); 0.5906 (0.9); 0.0079 (0.5); −0.0002
(18.7); −0.0085 (0.6)
III-012: ¹H-NMR(400.0 MHz, CDCl3):
δ = 7.3866 (0.9); 7.3850 (0.8); 7.3719 (1.3); 7.3681 (1.2); 7.3660 (1.7); 7.3515 (1.9); 7.3467
(1.4); 7.3317 (1.4); 7.2603 (53.4); 7.1415 (3.5); 7.1356 (1.8); 7.1344 (1.7); 7.1296 (4.2); 7.1245
(3.1); 7.1186 (5.0); 7.1128 (3.3); 7.1103 (3.1); 7.1068 (6.2); 7.0984 (0.9); 7.0919 (1.0); 7.0895
(1.0); 7.0854 (1.0); 7.0830 (1.0); 7.0406 (2.0); 7.0380 (2.5); 7.0369 (2.8); 7.0343 (2.3); 7.0180
(4.3); 7.0154 (3.4); 7.0129 (2.0); 7.0080 (1.5); 6.9994 (5.2); 6.9960 (2.5); 6.9935 (2.4); 6.9895
(2.0); 6.9856 (1.7); 6.9821 (2.1); 6.9792 (5.6); 6.9767 (4.3); 6.9734 (1.8); 6.9681 (0.7); 6.9621
(1.4); 6.9563 (3.5); 6.7201 (0.8); 5.2981 (2.3); 4.8349 (16.0); 4.8229 (0.6); 3.2716 (3.6); 3.2573
(4.2); 3.2540 (4.2); 3.2398 (3.7); 1.5602 (3.2); 1.0563 (0.8); 1.0542 (0.8); 1.0484 (0.8); 1.0443
(0.6); 1.0364 (1.5); 1.0284 (0.6); 1.0242 (0.8); 1.0184 (0.8); 1.0164 (1.0); 1.0043 (0.5); 0.5677
(1.3); 0.5559 (3.2); 0.5529 (3.8); 0.5506 (2.2); 0.5478 (1.7); 0.5414 (1.8); 0.5357 (3.7); 0.5328
(3.1); 0.5214 (1.6); 0.2840 (1.5); 0.2724 (4.2); 0.2701 (3.6); 0.2604 (3.4); 0.2577 (4.3); 0.2460
(1.1); 0.0080 (0.8); −0.0002 (21.5); −0.0085 (0.8)
III-013: ¹H-NMR(400.0 MHz, CDCl3):
δ = 7.3687 (0.6); 7.3631 (0.8); 7.3487 (0.9); 7.3433 (0.7); 7.3286 (0.7); 7.2601 (18.6); 7.1347
(1.6); 7.1293 (1.1); 7.1229 (2.1); 7.1176 (1.2); 7.1120 (2.6); 7.1080 (1.1); 7.1042 (1.3); 7.1004
(2.6); 7.0344 (0.8); 7.0309 (1.3); 7.0282 (1.0); 7.0117 (2.0); 7.0094 (1.6); 7.0051 (1.1); 7.0018
(0.6); 6.9966 (2.4); 6.9885 (1.0); 6.9845 (1.0); 6.9820 (0.9); 6.9764 (2.8); 6.9711 (0.8); 6.9595
(0.5); 6.9538 (1.6); 5.2975 (1.5); 4.8347 (8.3); 3.2365 (2.0); 3.2203 (3.3); 3.2044 (2.0); 1.8734
(0.8); 1.8566 (1.1); 1.8398 (0.9); 1.5647 (1.4); 0.9700 (16.0); 0.9532 (15.4); −0.0002 (7.2)
III-014: ¹H-NMR(400.0 MHz, CDCl3):
δ = 7.5184 (1.4); 7.3726 (0.6); 7.3626 (0.6); 7.3480 (0.7); 7.3418 (0.9); 7.3281 (1.1); 7.3226
(1.0); 7.3076 (1.0); 7.2868 (1.1); 7.2595 (250.5); 7.2092 (0.9); 7.1048 (1.2); 7.0983 (1.3);
7.0942 (1.2); 7.0832 (1.6); 7.0795 (1.4); 7.0770 (1.6); 7.0320 (1.0); 7.0127 (1.0); 6.9955 (2.0);
6.9731 (2.0); 6.9525 (2.0); 6.9303 (1.1); 5.2653 (0.6); 5.1635 (0.6); 5.0152 (2.3); 4.9941 (2.0);
4.0569 (0.8); 4.0424 (0.8); 3.9668 (0.7); 3.9543 (0.7); 3.0239 (3.7); 2.9833 (3.3); 1.5331 (16.0);
0.0080 (4.1); −0.0002 (102.1); −0.0085 (3.9)
III-015: ¹H-NMR(400.0 MHz, CDCl3):
δ = 7.3800 (1.3); 7.3652 (1.2); 7.3594 (2.4); 7.3447 (2.0); 7.3388 (1.7); 7.3250 (1.4); 7.2599
(47.4); 7.1604 (0.5); 7.1521 (3.4); 7.1463 (1.5); 7.1402 (3.7); 7.1347 (2.5); 7.1292 (5.3); 7.1233
(2.5); 7.1212 (2.3); 7.1173 (4.7); 7.1091 (2.2); 7.1066 (2.1); 7.1028 (1.9); 7.1002 (2.0); 7.0880
(1.0); 7.0855 (1.0); 7.0816 (1.1); 7.0791 (1.0); 7.0431 (1.8); 7.0393 (3.0); 7.0367 (2.6); 7.0325
(2.1); 7.0284 (2.3); 7.0262 (2.1); 7.0221 (3.5); 7.0203 (3.8); 7.0175 (1.9); 7.0118 (5.2); 7.0058
(2.8); 7.0029 (2.1); 6.9992 (1.8); 6.9946 (2.1); 6.9916 (5.5); 6.9890 (4.2); 6.9859 (1.8); 6.9805
(0.8); 6.9745 (1.4); 6.9687 (3.4); 6.6717 (0.7); 5.9486 (0.6); 5.9349 (1.3); 5.9227 (0.8); 5.9213
(0.8); 5.9091 (1.5); 5.9057 (0.8); 5.8954 (0.8); 5.8920 (1.6); 5.8798 (1.0); 5.8784 (0.9); 5.8662
(1.6); 5.8525 (0.8); 5.2975 (1.8); 5.2763 (1.0); 5.2720 (2.2); 5.2688 (2.3); 5.2645 (1.0); 5.2333
(0.9); 5.2291 (1.9); 5.2259 (2.0); 5.2216 (1.0); 5.1912 (1.1); 5.1876 (2.5); 5.1842 (2.4); 5.1807
(1.0); 5.1655 (1.0); 5.1618 (2.3); 5.1585 (2.3); 5.1549 (0.9); 4.8668 (16.0); 4.0373 (1.4); 4.0334
(2.4); 4.0294 (1.6); 4.0226 (2.6); 4.0191 (4.1); 4.0155 (2.5); 4.0088 (1.6); 4.0048 (2.3); 4.0009
(1.4); 1.5587 (2.9); 0.0079 (0.7); −0.0002 (19.0); −0.0085 (0.8)
III-016: ¹H-NMR(400.0 MHz, CDCl3):
δ = 7.3818 (1.1); 7.3668 (1.0); 7.3613 (2.2); 7.3464 (1.9); 7.3406 (1.5); 7.3267 (1.3); 7.2600
(35.0); 7.1544 (3.1); 7.1487 (1.2); 7.1425 (3.3); 7.1372 (2.1); 7.1317 (5.0); 7.1256 (2.3); 7.1233
(2.0); 7.1198 (4.4); 7.1111 (1.8); 7.1085 (1.7); 7.1046 (1.6); 7.1023 (1.8); 7.0900 (0.8); 7.0875
(0.8); 7.0835 (0.9); 7.0811 (0.8); 7.0453 (1.5); 7.0417 (2.7); 7.0391 (2.2); 7.0341 (1.6); 7.0300
(1.9); 7.0233 (3.6); 7.0201 (1.9); 7.0115 (5.3); 7.0058 (2.5); 7.0001 (1.6); 6.9944 (1.6); 6.9913
(5.0); 6.9858 (1.5); 6.9743 (1.1); 6.9687 (3.2); 6.7735 (0.7); 5.2975 (6.7); 4.9274 (0.8); 4.8645
(16.0); 4.1932 (4.3); 4.1868 (4.4); 4.1795 (4.3); 4.1731 (4.2); 3.8121 (1.4); 2.2781 (2.6); 2.2717
(5.4); 2.2652 (2.6); 1.5585 (2.7); −0.0002 (13.7)
III-017: ¹H-NMR(400.0 MHz, CDCl3):
δ = 7.3821 (0.8); 7.3672 (0.7); 7.3615 (1.5); 7.3470 (1.3); 7.3409 (1.1); 7.3272 (0.9); 7.2606
(19.7); 7.1540 (2.2); 7.1482 (0.9); 7.1421 (2.4); 7.1366 (1.6); 7.1311 (3.5); 7.1252 (1.7); 7.1230
(1.5); 7.1192 (3.1); 7.1108 (1.4); 7.1083 (1.3); 7.1044 (1.3); 7.1020 (1.3); 7.0898 (0.6); 7.0873
(0.6); 7.0833 (0.7); 7.0808 (0.6); 7.0460 (1.1); 7.0423 (2.0); 7.0397 (1.7); 7.0361 (1.4); 7.0321
(1.4); 7.0299 (1.2); 7.0260 (2.3); 7.0233 (2.0); 7.0204 (1.2); 7.0100 (3.6); 7.0067 (1.4); 7.0056
(1.5); 7.0044 (1.5); 6.9983 (0.8); 6.9927 (1.2); 6.9899 (3.6); 6.9874 (2.7); 6.9842 (1.2); 6.9728
(0.9); 6.9671 (2.3); 6.7110 (0.5); 5.2974 (16.0); 4.8486 (10.5); 4.1364 (0.8); 4.1303 (2.2);
4.1240 (2.6); 4.1171 (2.6); 4.1108 (2.2); 4.1047 (0.8); 3.8119 (0.8); 1.8344 (0.7); 1.8294 (6.1);
1.8233 (12.3); 1.8171 (6.0); 1.5722 (0.7); −0.0002 (7.9)
III-018: ¹H-NMR(400.0 MHz, CDCl3):
δ = 7.3783 (1.1); 7.3632 (1.0); 7.3578 (2.2); 7.3432 (1.8); 7.3372 (1.4); 7.3232 (1.2); 7.2604
(37.6); 7.1450 (3.0); 7.1394 (1.2); 7.1332 (3.2); 7.1277 (2.6); 7.1224 (5.1); 7.1162 (1.5); 7.1104
(4.4); 7.1057 (1.8); 7.1018 (1.9); 7.0994 (1.7); 7.0870 (0.7); 7.0846 (0.7); 7.0806 (0.8); 7.0782
(0.8); 7.0378 (1.4); 7.0342 (2.6); 7.0315 (2.2); 7.0277 (1.7); 7.0236 (1.8); 7.0215 (1.6); 7.0177
(3.3); 7.0152 (2.9); 7.0104 (4.8); 7.0043 (2.6); 6.9983 (2.0); 6.9933 (2.2); 6.9900 (4.9); 6.9879
(3.9); 6.9846 (1.5); 6.9731 (1.0); 6.9674 (3.0); 6.6448 (0.8); 5.2977 (5.6); 4.8113 (16.0); 2.8352
(0.6); 2.8265 (1.0); 2.8173 (1.4); 2.8086 (1.4); 2.7994 (1.1); 2.7907 (0.6); 1.5653 (4.7); 1.2577
(0.5); 0.8712 (1.0); 0.8570 (2.6); 0.8536 (3.8); 0.8508 (1.9); 0.8398 (3.6); 0.8358 (2.9); 0.8329
(1.7); 0.8224 (1.2); 0.6268 (1.3); 0.6167 (2.6); 0.6136 (2.9); 0.6098 (2.8); 0.6080 (2.8); 0.6040
(2.8); 0.6004 (2.6); 0.5865 (0.9); −0.0002 (14.7)
III-019: ¹H-NMR(400.0 MHz, CDCl3):
δ = 7.3766 (1.0); 7.3613 (1.0); 7.3559 (2.0); 7.3413 (1.8); 7.3354 (1.3); 7.3212 (1.2); 7.2607
(31.4); 7.1496 (2.8); 7.1442 (1.1); 7.1378 (3.0); 7.1325 (2.0); 7.1269 (4.7); 7.1203 (2.1); 7.1152
(4.5); 7.1054 (1.9); 7.1028 (2.0); 7.0987 (2.0); 7.0964 (2.2); 7.0842 (1.1); 7.0818 (1.2); 7.0777
(1.1); 7.0754 (1.1); 7.0426 (1.3); 7.0391 (2.5); 7.0366 (2.0); 7.0322 (1.5); 7.0280 (1.7); 7.0260
(1.5); 7.0204 (2.9); 7.0170 (1.6); 7.0080 (5.2); 7.0023 (2.4); 6.9978 (1.2); 6.9875 (4.4); 6.9856
(3.8); 6.9823 (1.4); 6.9707 (1.0); 6.9651 (2.9); 5.2977 (6.5); 4.8244 (15.2); 4.1832 (2.2); 4.1654
(6.8); 4.1475 (6.9); 4.1296 (2.3); 3.6791 (1.6); 3.6637 (4.0); 3.6484 (4.2); 3.6329 (1.6); 2.6151
(3.6); 2.6097 (0.7); 2.5997 (5.7); 2.5904 (0.6); 2.5846 (3.4); 1.5715 (2.5); 1.2793 (7.7); 1.2615
(16.0); 1.2436 (7.6); −0.0002 (12.3)
III-020: ¹H-NMR(400.0 MHz, CDCl3):
δ = 7.3711 (0.7); 7.3606 (7.3); 7.3572 (3.1); 7.3552 (4.0); 7.3524 (7.7); 7.3496 (4.2); 7.3469
(3.4); 7.3442 (7.9); 7.3401 (2.6); 7.3327 (4.3); 7.3273 (1.8); 7.3197 (4.2); 7.3153 (2.3); 7.3104
(4.8); 7.3030 (1.9); 7.2975 (4.5); 7.2900 (0.7); 7.2635 (33.4); 7.2140 (1.0); 7.2023 (4.3); 7.1993
(2.4); 7.1950 (3.0); 7.1921 (2.9); 7.1879 (3.0); 7.1859 (2.7); 7.1810 (1.9); 7.1779 (3.3); 7.1229
(0.5); 7.1156 (4.6); 7.1101 (1.4); 7.1018 (0.7); 7.0985 (1.5); 7.0943 (6.9); 7.0894 (1.6); 7.0778
(1.2); 7.0724 (3.7); 6.5837 (1.0); 5.9140 (0.6); 5.9003 (1.4); 5.8880 (0.9); 5.8746 (1.6); 5.8712
(0.9); 5.8608 (0.8); 5.8574 (1.7); 5.8450 (1.0); 5.8317 (1.7); 5.8180 (0.8); 5.3784 (1.1); 5.3615
(3.6); 5.3445 (3.6); 5.3275 (1.2); 5.2342 (1.0); 5.2301 (2.3); 5.2267 (2.6); 5.2226 (1.1); 5.1913
(0.9); 5.1872 (2.1); 5.1838 (2.2); 5.1797 (0.9); 5.1691 (1.0); 5.1656 (2.7); 5.1622 (2.6); 5.1588
(1.0); 5.1435 (1.0); 5.1399 (2.5); 5.1365 (2.5); 5.1331 (1.0); 3.9879 (1.5); 3.9840 (2.7); 3.9801
(1.7); 3.9734 (2.8); 3.9697 (4.8); 3.9660 (2.9); 3.9593 (1.7); 3.9554 (2.7); 3.9515 (1.6); 1.7097
(15.9); 1.6927 (16.0); 1.6645 (0.8); 1.6476 (0.8); 1.4323 (2.7); 0.9952 (0.5); 0.9913
(0.6); −0.0002 (14.3)
III-021: ¹H-NMR(400.0 MHz, CDCl3):
δ = 7.3629 (0.7); 7.3570 (3.0); 7.3543 (1.5); 7.3485 (3.3); 7.3439 (1.9); 7.3406 (3.8); 7.3363
(2.7); 7.3305 (1.2); 7.3231 (1.9); 7.3187 (1.2); 7.3137 (2.2); 7.3063 (1.0); 7.3009 (2.1); 7.2639
(20.6); 7.2003 (2.0); 7.1976 (1.1); 7.1917 (1.6); 7.1863 (1.3); 7.1832 (1.3); 7.1791 (0.9); 7.1759
(1.5); 7.1121 (2.0); 7.1067 (0.7); 7.0950 (0.8); 7.0908 (3.0); 7.0858 (0.9); 7.0742 (0.6); 7.0689
(1.7); 5.3017 (4.2); 5.2988 (1.8); 5.2816 (1.6); 3.6867 (16.0); 3.6294 (0.8); 3.6247 (1.2); 3.6099
(1.7); 3.5953 (1.3); 2.5996 (1.8); 2.5843 (3.2); 2.5689 (1.6); 1.6809 (6.5); 1.6730 (0.8); 1.6639
(6.4); 1.6561 (0.6); 1.4323 (1.4); −0.0002 (8.3)
III-022: ¹H-NMR(400.0 MHz, CDCl3):
δ = 7.3810 (1.2); 7.3662 (1.0); 7.3604 (2.3); 7.3457 (1.9); 7.3398 (1.6); 7.3261 (1.3); 7.2605
(37.7); 7.1610 (3.3); 7.1552 (1.2); 7.1491 (3.5); 7.1437 (2.2); 7.1381 (4.7); 7.1320 (1.5); 7.1262
(5.1); 7.1218 (1.1); 7.1191 (1.2); 7.1069 (1.5); 7.1044 (1.6); 7.1006 (1.6); 7.0981 (1.7); 7.0858
(0.7); 7.0834 (0.8); 7.0794 (0.8); 7.0769 (0.8); 7.0411 (1.6); 7.0374 (2.8); 7.0347 (2.4); 7.0306
(2.0); 7.0266 (2.2); 7.0243 (1.8); 7.0204 (7.5); 7.0148 (2.3); 7.0068 (1.8); 7.0031 (2.7); 7.0001
(6.3); 6.9974 (4.8); 6.9945 (1.7); 6.9830 (1.2); 6.9773 (3.3); 6.6686 (0.6); 5.9450 (0.6); 5.9312
(1.3); 5.9191 (0.8); 5.9176 (0.8); 5.9055 (1.5); 5.9021 (0.8); 5.8917 (0.8); 5.8883 (1.6); 5.8762
(0.9); 5.8746 (0.9); 5.8626 (1.6); 5.8488 (0.8); 5.2978 (10.2); 5.2728 (0.9); 5.2686 (2.1); 5.2653
(2.3); 5.2611 (1.0); 5.2299 (0.8); 5.2257 (1.9); 5.2224 (2.0); 5.2181 (0.9); 5.1903 (0.9); 5.1867
(2.5); 5.1833 (2.4); 5.1797 (0.9); 5.1645 (0.9); 5.1610 (2.3); 5.1576 (2.2); 5.1540 (0.8); 4.8669
(16.0); 4.0364 (1.2); 4.0324 (2.2); 4.0285 (1.4); 4.0218 (2.2); 4.0181 (3.8); 4.0144 (2.2); 4.0077
(1.3); 4.0038 (2.1); 3.9999 (1.2); 1.5879 (1.1); 1.4318 (0.9); −0.0002 (15.2)
III-023: ¹H-NMR(400.0 MHz, CDCl3):
δ = 7.3623 (0.7); 7.3475 (0.6); 7.2605 (10.0); 7.1634 (1.0); 7.1516 (1.0); 7.1462 (0.7); 7.1407
(1.4); 7.1289 (1.5); 7.1000 (0.5); 7.0396 (0.8); 7.0370 (0.7); 7.0320 (0.6); 7.0280 (0.7); 7.0204
(2.3); 7.0083 (0.7); 7.0000 (1.8); 6.9773 (1.0); 5.2980 (16.0); 4.8645 (5.0); 4.1933 (1.4); 4.1869
(1.4); 4.1796 (1.4); 4.1732 (1.3); 2.2780 (0.9); 2.2716 (1.9); 2.2652 (0.9); 1.5728 (0.5); −0.0002
(4.1)
III-024: ¹H-NMR(400.0 MHz, CDCl3):
δ = 7.3795 (0.5); 7.3589 (1.0); 7.3443 (0.8); 7.3383 (0.7); 7.3243 (0.6); 7.2610 (17.9); 7.1541
(1.4); 7.1485 (0.5); 7.1423 (1.5); 7.1369 (1.0); 7.1313 (2.1); 7.1250 (1.0); 7.1194 (2.2); 7.1060
(0.7); 7.1035 (0.7); 7.0996 (0.7); 7.0972 (0.7); 7.0358 (0.7); 7.0320 (1.2); 7.0294 (1.1); 7.0259
(0.9); 7.0215 (1.1); 7.0191 (2.6); 7.0157 (1.6); 7.0131 (1.8); 7.0018 (1.3); 6.9986 (2.7); 6.9963
(2.3); 6.9928 (1.0); 6.9760 (1.4); 5.2982 (16.0); 4.8113 (7.1); 2.8143 (0.6); 2.8058 (0.6); 1.5740
(1.3); 0.8564 (1.1); 0.8532 (1.7); 0.8502 (0.8); 0.8393 (1.6); 0.8353 (1.3); 0.8323 (0.7); 0.8218
(0.6); 0.6289 (0.6); 0.6189 (1.2); 0.6158 (1.3); 0.6120 (1.2); 0.6100 (1.2); 0.6061 (1.3); 0.6026
(1.1); −0.0002 (7.2)
III-025: ¹H-NMR(400.0 MHz, CDCl3):
δ = 7.3833 (1.2); 7.3685 (1.0); 7.3628 (2.3); 7.3482 (1.9); 7.3422 (1.6); 7.3283 (1.3); 7.2608
(36.5); 7.1653 (3.3); 7.1596 (1.2); 7.1534 (3.5); 7.1480 (2.2); 7.1425 (4.7); 7.1364 (1.5); 7.1306
(5.0); 7.1275 (1.3); 7.1234 (1.2); 7.1212 (1.2); 7.1087 (1.5); 7.1062 (1.7); 7.1023 (1.6); 7.0998
(1.8); 7.0876 (0.8); 7.0851 (0.8); 7.0811 (0.8); 7.0787 (0.8); 7.0451 (1.5); 7.0413 (2.8); 7.0387
(2.4); 7.0357 (2.0); 7.0317 (2.0); 7.0293 (2.0); 7.0254 (3.5); 7.0202 (5.9); 7.0141 (1.8); 7.0120
(2.0); 7.0083 (1.9); 7.0057 (1.8); 6.9999 (5.5); 6.9975 (4.2); 6.9943 (1.6); 6.9829 (1.2); 6.9772
(3.3); 6.6928 (0.7); 5.2979 (16.0); 4.8422 (15.8); 3.2639 (3.5); 3.2495 (4.0); 3.2463 (4.0);
3.2320 (3.5); 1.5787 (2.7); 1.0458 (0.8); 1.0438 (0.8); 1.0380 (0.7); 1.0339 (0.5); 1.0259 (1.4);
1.0180 (0.6); 1.0137 (0.7); 1.0080 (0.8); 1.0060 (0.9); 0.5591 (1.2); 0.5473 (3.0); 0.5442 (3.6);
0.5420 (2.0); 0.5392 (1.6); 0.5328 (1.6); 0.5272 (3.4); 0.5242 (2.9); 0.5128 (1.4); 0.2732 (1.4);
0.2616 (4.0); 0.2496 (3.1); 0.2468 (4.1); 0.2349 (1.0); 0.0701 (0.8); −0.0002 (14.8)
III-026: ¹H-NMR(400.0 MHz, CDCl3):
δ = 7.3791 (0.6); 7.3642 (0.5); 7.3586 (1.1); 7.3438 (1.0); 7.3379 (0.8); 7.3241 (0.6); 7.2614
(16.9); 7.1596 (1.6); 7.1539 (0.7); 7.1477 (1.8); 7.1423 (1.2); 7.1367 (2.3); 7.1306 (0.9); 7.1250
(2.6); 7.1191 (0.8); 7.1166 (1.0); 7.1043 (1.1); 7.1018 (1.2); 7.0979 (1.2); 7.0955 (1.3); 7.0832
(0.7); 7.0808 (0.7); 7.0768 (0.7); 7.0744 (0.6); 7.0429 (0.8); 7.0392 (1.4); 7.0366 (1.2); 7.0323
(1.0); 7.0282 (1.1); 7.0261 (1.2); 7.0179 (2.9); 7.0120 (0.9); 7.0083 (1.0); 7.0053 (1.0); 7.0010
(1.3); 6.9977 (3.1); 6.9920 (0.9); 6.9806 (0.7); 6.9749 (1.6); 5.2983 (4.1); 4.8241 (8.1); 3.6958
(16.0); 3.6787 (1.0); 3.6633 (2.3); 3.6480 (2.3); 3.6325 (1.0); 2.6318 (1.9); 2.6164 (2.9); 2.6012
(1.8); 1.5819 (2.0); −0.0002 (6.8)
III-027: ¹H-NMR(400.0 MHz, CDCl3):
δ = 7.3797 (0.6); 7.3649 (0.6); 7.3592 (1.1); 7.3444 (0.9); 7.3383 (0.6); 7.3246 (0.6); 7.2609
(19.2); 7.1705 (1.7); 7.1649 (0.6); 7.1587 (1.8); 7.1533 (1.1); 7.1477 (2.3); 7.1416 (0.8); 7.1359
(2.2); 7.1256 (0.6); 7.1230 (0.6); 7.1191 (0.6); 7.1166 (0.7); 7.1044 (1.1); 7.1017 (1.2); 7.0983
(1.0); 7.0955 (1.2); 7.0833 (0.6); 7.0808 (0.5); 7.0768 (0.5); 7.0469 (0.8); 7.0431 (1.3); 7.0405
(1.1); 7.0333 (0.8); 7.0291 (1.3); 7.0270 (1.6); 7.0235 (2.4); 7.0200 (2.6); 7.0141 (0.8); 7.0091
(1.0); 7.0028 (1.4); 6.9996 (3.0); 6.9940 (0.8); 6.9826 (0.6); 6.9769 (1.6); 5.2981 (4.6); 4.8950
(7.8); 4.1775 (3.6); 4.1639 (3.6); 3.7892 (16.0); 1.5695 (2.2); −0.0002 (7.7)
III-028: ¹H-NMR(400.0 MHz, CDCl3):
δ = 7.5194 (0.7); 7.5156 (0.9); 7.5127 (1.1); 7.5093 (0.7); 7.5007 (0.8); 7.4942 (2.9); 7.4881
(1.0); 7.4791 (1.5); 7.4756 (2.8); 7.4721 (1.5); 7.4514 (0.7); 7.4461 (3.2); 7.4427 (1.7); 7.4310
(2.9); 7.4272 (5.7); 7.4227 (1.6); 7.4137 (1.1); 7.4093 (3.0); 7.4061 (1.8); 7.3639 (0.7); 7.3593
(0.7); 7.3463 (0.7); 7.3412 (0.6); 7.3308 (0.9); 7.3257 (4.6); 7.3226 (5.6); 7.3189 (2.7); 7.3104
(1.4); 7.3053 (5.0); 7.3015 (3.4); 7.2606 (65.6); 7.2496 (1.7); 7.2484 (1.7); 7.2438 (3.0); 7.2287
(2.4); 7.2234 (1.9); 7.2092 (1.2); 7.2077 (1.2); 7.0346 (1.1); 7.0324 (1.3); 7.0283 (1.5); 7.0261
(1.5); 7.0138 (2.4); 7.0115 (2.7); 7.0074 (2.6); 7.0056 (2.7); 6.9966 (1.1); 6.9932 (1.2); 6.9910
(1.2); 6.9870 (1.2); 6.9847 (1.1); 6.9403 (1.4); 6.9350 (2.2); 6.9293 (1.5); 6.9168 (1.2); 6.9117
(2.0); 6.9072 (3.6); 6.9052 (3.7); 6.9023 (1.6); 6.8998 (1.4); 6.8869 (1.7); 6.8855 (1.8); 6.8821
(1.4); 5.9884 (0.9); 5.2981 (10.6); 4.9142 (1.7); 4.9026 (16.0); 4.7268 (1.1); 2.8681 (0.7); 2.8592
(1.2); 2.8501 (1.6); 2.8411 (1.6); 2.8320 (1.2); 2.8231 (0.8); 2.0433 (0.6); 1.5631 (4.3); 1.2584
(0.6); 0.8814 (1.2); 0.8757 (0.6); 0.8669 (2.6); 0.8640 (4.2); 0.8610 (2.3); 0.8501 (3.8); 0.8461
(3.1); 0.8430 (2.0); 0.8370 (0.7); 0.8326 (1.6); 0.6537 (1.4); 0.6438 (3.0); 0.6405 (3.3); 0.6349
(3.2); 0.6309 (3.3); 0.6272 (2.9); 0.6134 (1.3); 0.0079 (0.8); −0.0002 (26.2); −0.0085 (1.0)
III-029: ¹H-NMR(400.0 MHz, CDCl3):
δ = 7.5124 (0.5); 7.5090 (0.5); 7.4907 (1.4); 7.4845 (0.5); 7.4757 (0.8); 7.4722 (1.4); 7.4686
(0.8); 7.4446 (1.6); 7.4411 (0.9); 7.4296 (1.5); 7.4258 (2.9); 7.4212 (0.8); 7.4123 (0.8); 7.4078
(1.8); 7.4045 (1.2); 7.3558 (0.6); 7.3402 (0.6); 7.3349 (2.3); 7.3318 (2.8); 7.3281 (1.3); 7.3196
(0.8); 7.3145 (2.4); 7.3107 (1.7); 7.2610 (20.9); 7.2555 (0.7); 7.2513 (0.6); 7.2485 (0.8); 7.2428
(1.3); 7.2276 (1.2); 7.2222 (0.9); 7.2072 (0.7); 7.0340 (0.6); 7.0319 (0.6); 7.0278 (0.6); 7.0257
(0.6); 7.0135 (0.8); 7.0112 (0.9); 7.0072 (1.0); 7.0050 (0.9); 6.9865 (0.5); 6.9610 (0.7); 6.9556
(1.1); 6.9500 (0.6); 6.9375 (0.7); 6.9323 (1.0); 6.9267 (0.7); 6.9137 (0.9); 6.9120 (1.0); 6.9088
(0.8); 6.9068 (0.7); 6.8933 (0.8); 6.8920 (0.8); 6.8884 (0.7); 6.8869 (0.6); 5.2979 (9.4); 4.9951
(0.9); 4.9831 (7.6); 4.8328 (0.6); 4.1905 (3.8); 4.1766 (3.8); 4.1669 (0.5); 3.7927 (16.0); 3.7800
(1.5); −0.0002 (8.5)
III-030: ¹H-NMR(400.0 MHz, CDCl3):
δ = 7.5037 (1.4); 7.5004 (0.8); 7.4916 (0.8); 7.4852 (3.0); 7.4792 (1.1); 7.4701 (1.6); 7.4666
(2.9); 7.4631 (1.6); 7.4415 (0.8); 7.4363 (3.3); 7.4328 (1.8); 7.4212 (3.1); 7.4174 (5.8); 7.4129
(1.6); 7.4038 (1.2); 7.3994 (3.2); 7.3962 (1.9); 7.3448 (10.8); 7.3442 (10.8); 7.3378 (5.2);
7.3331 (13.0); 7.3248 (2.0); 7.3177 (2.1); 7.3139 (2.4); 7.3089 (6.3); 7.3058 (6.6); 7.3006 (5.1);
7.2933 (3.0); 7.2884 (6.5); 7.2847 (4.7); 7.2792 (2.8); 7.2737 (1.6); 7.2684 (1.9); 7.2587 (55.0);
7.2478 (2.6); 7.2420 (3.7); 7.2326 (1.7); 7.2269 (3.1); 7.2216 (2.2); 7.2134 (1.0); 7.2070 (1.7);
7.1927 (0.5); 7.0357 (0.9); 7.0334 (1.0); 7.0294 (1.0); 7.0272 (1.0); 7.0150 (1.7); 7.0127 (1.9);
7.0088 (1.7); 7.0066 (1.8); 6.9943 (1.3); 6.9921 (1.1); 6.9880 (1.1); 6.9858 (1.0); 6.9451 (0.6);
6.9345 (1.3); 6.9290 (2.2); 6.9235 (1.5); 6.9109 (1.2); 6.9059 (2.1); 6.8988 (2.8); 6.8961 (2.6);
6.8932 (1.6); 6.8908 (1.4); 6.8778 (1.8); 6.8764 (1.8); 6.8730 (1.5); 6.8712 (1.3); 5.9808 (1.3);
5.2961 (16.0); 4.9904 (2.1); 4.9819 (15.6); 4.8250 (1.6); 4.7655 (0.6); 4.6005 (7.1); 4.5854
(7.0); 4.5689 (0.9); 4.5540 (0.8); 1.5619 (3.4); 0.0080 (0.8); −0.0002 (22.3); −0.0085 (0.8)
III-031: ¹H-NMR(400.0 MHz, CDCl3):
δ = 7.5362 (0.6); 7.5167 (1.4); 7.5132 (1.2); 7.5098 (0.7); 7.5013 (0.8); 7.4988 (0.7); 7.4948
(3.0); 7.4887 (1.1); 7.4797 (1.6); 7.4762 (2.9); 7.4727 (1.6); 7.4529 (0.7); 7.4490 (2.8); 7.4476
(3.2); 7.4441 (1.8); 7.4325 (3.0); 7.4287 (5.9); 7.4240 (1.6); 7.4152 (1.2); 7.4108 (3.2); 7.4076
(1.9); 7.3713 (0.9); 7.3681 (1.0); 7.3647 (0.6); 7.3603 (0.8); 7.3553 (0.7); 7.3508 (1.1); 7.3469
(0.9); 7.3421 (0.8); 7.3353 (0.9); 7.3302 (4.8); 7.3271 (5.9); 7.3248 (2.6); 7.3233 (2.9); 7.3148
(1.5); 7.3097 (5.1); 7.3060 (3.6); 7.2665 (2.1); 7.2600 (90.9); 7.2520 (2.8); 7.2462 (3.2); 7.2350
(1.1); 7.2310 (2.6); 7.2256 (1.8); 7.2201 (0.6); 7.2153 (0.8); 7.2110 (1.6); 7.2012 (0.7); 7.0368
(1.4); 7.0346 (1.6); 7.0305 (1.6); 7.0283 (1.7); 7.0159 (2.4); 7.0138 (2.7); 7.0098 (2.6); 7.0077
(2.5); 6.9957 (1.8); 6.9932 (1.3); 6.9892 (1.4); 6.9869 (1.4); 6.9497 (1.4); 6.9444 (2.4); 6.9390
(1.6); 6.9263 (1.3); 6.9213 (2.2); 6.9141 (2.8); 6.9115 (2.6); 6.9085 (1.6); 6.9062 (1.4); 6.8932
(1.6); 6.8918 (1.7); 6.8884 (1.4); 6.0755 (0.5); 6.0122 (1.0); 5.9580 (0.7); 5.9441 (1.5); 5.9321
(0.9); 5.9304 (0.9); 5.9184 (1.7); 5.9151 (1.0); 5.9045 (1.0); 5.9012 (1.9); 5.8892 (1.1); 5.8875
(1.2); 5.8754 (1.9); 5.8616 (0.9); 5.3045 (1.0); 5.3003 (2.5); 5.2978 (3.5); 5.2931 (1.2); 5.2615
(1.0); 5.2573 (2.2); 5.2543 (2.3); 5.2501 (1.2); 5.2208 (0.5); 5.2154 (1.2); 5.2119 (2.6); 5.2087
(2.6); 5.2052 (1.2); 5.1950 (0.5); 5.1897 (1.1); 5.1862 (2.4); 5.1830 (2.3); 5.1795 (1.0); 5.1735
(0.5); 4.9628 (2.6); 4.9512 (16.0); 4.7881 (1.8); 4.7298 (0.6); 4.0499 (1.6); 4.0459 (2.4); 4.0418
(1.8); 4.0357 (3.0); 4.0317 (4.5); 4.0278 (2.7); 4.0214 (1.9); 4.0174 (2.6); 4.0136 (1.6); 4.0008
(0.7); 1.5559 (5.7); 0.0080 (1.2); −0.0002 (38.0); −0.0085 (1.4)
III-035: ¹H-NMR(400.0 MHz, CDCl3):
δ = 7.4378 (0.8); 7.4325 (1.4); 7.4250 (6.7); 7.4212 (7.1); 7.4150 (2.8); 7.4111 (2.5); 7.4069
(4.4); 7.4053 (3.9); 7.3981 (1.3); 7.3939 (1.5); 7.3879 (0.6); 7.3836 (0.7); 7.3186 (0.6); 7.3113
(4.7); 7.3071 (5.2); 7.3020 (2.0); 7.3002 (1.9); 7.2976 (1.8); 7.2948 (2.4); 7.2925 (2.6); 7.2871
(3.5); 7.2601 (36.3); 7.2271 (1.0); 7.2109 (1.0); 7.2067 (2.1); 7.1919 (1.7); 7.1905 (1.8); 7.1874
(1.3); 7.1724 (1.0); 7.1705 (1.0); 6.9607 (0.8); 6.9554 (2.0); 6.9506 (1.8); 6.9473 (1.5); 6.9447
(1.7); 6.9409 (0.8); 6.9380 (0.9); 6.9362 (0.9); 6.9309 (2.0); 6.9268 (3.7); 6.9241 (3.9); 6.9202
(1.7); 6.9175 (3.5); 6.9145 (2.3); 6.9101 (1.5); 6.9060 (1.1); 6.9037 (1.4); 6.8974 (2.5); 6.8951
(1.8); 6.8924 (1.7); 6.8901 (1.3); 6.7097 (0.7); 5.2973 (7.6); 4.8608 (16.0); 3.2684 (3.6); 3.2541
(4.2); 3.2509 (4.2); 3.2366 (3.6); 1.5736 (3.6); 1.0504 (0.8); 1.0484 (0.8); 1.0425 (0.7); 1.0385
(0.6); 1.0361 (0.5); 1.0305 (1.4); 1.0225 (0.6); 1.0183 (0.8); 1.0125 (0.8); 1.0105 (0.9); 0.5610
(1.2); 0.5491 (3.2); 0.5462 (3.7); 0.5440 (2.3); 0.5411 (1.7); 0.5347 (1.8); 0.5291 (3.6); 0.5261
(3.1); 0.5147 (1.4); 0.2767 (1.4); 0.2651 (4.2); 0.2531 (3.4); 0.2503 (4.3); 0.2384 (1.0); −0.0002
(14.4); −0.0084 (0.6)
III-038: ¹H-NMR(400.0 MHz, CDCl3):
δ = 8.9430 (1.5); 7.4486 (0.7); 7.4444 (0.8); 7.4322 (3.8); 7.4281 (4.7); 7.4193 (7.5); 7.4170
(12.4); 7.4148 (14.1); 7.4095 (9.7); 7.4031 (4.9); 7.3991 (6.9); 7.3974 (6.3); 7.3891 (2.5);
7.3852 (3.2); 7.3742 (4.3); 7.3655 (7.3); 7.3573 (3.9); 7.3514 (7.5); 7.3478 (6.9); 7.3404 (1.8);
7.2726 (1.2); 7.2655 (6.8); 7.2609 (10.1); 7.2581 (49.7); 7.2517 (3.4); 7.2484 (4.1); 7.2466
(4.4); 7.2415 (5.0); 7.2110 (1.1); 7.1904 (2.2); 7.1746 (2.2); 7.1702 (1.6); 7.1541 (1.2); 6.9328
(1.1); 6.9286 (1.4); 6.9267 (1.5); 6.9060 (3.6); 6.9000 (2.0); 6.8939 (1.7); 6.8875 (1.8); 6.8854
(1.8); 6.8754 (1.9); 6.8655 (3.0); 6.8639 (2.9); 6.8450 (2.1); 6.8412 (2.1); 5.2951 (16.0); 4.9969
(13.2); 4.9029 (5.9); 4.7024 (2.2); 1.5851 (0.9); 0.0079 (0.6); −0.0002 (20.3); −0.0085 (0.8)
III-039: ¹H-NMR(400.0 MHz, CDCl3):
δ = 7.4141 (1.2); 7.3996 (1.1); 7.3936 (2.4); 7.3790 (1.9); 7.3728 (1.7); 7.3593 (1.4); 7.2611
(47.7); 7.2040 (1.0); 7.2015 (1.1); 7.1977 (1.1); 7.1952 (1.1); 7.1829 (1.8); 7.1803 (2.0); 7.1767
(1.9); 7.1740 (2.0); 7.1624 (4.1); 7.1594 (1.8); 7.1567 (1.8); 7.1509 (3.9); 7.1453 (2.6); 7.1397
(5.2); 7.1338 (1.7); 7.1280 (5.0); 7.1197 (0.6); 7.0759 (1.8); 7.0721 (3.0); 7.0695 (2.6); 7.0641
(1.9); 7.0600 (2.3); 7.0560 (2.6); 7.0535 (4.3); 7.0412 (2.0); 7.0357 (6.6); 7.0302 (2.3); 7.0242
(0.9); 7.0158 (5.6); 7.0129 (4.2); 7.0102 (1.9); 7.0045 (0.6); 6.9987 (1.3); 6.9930 (3.6); 6.9289
(0.9); 5.2984 (1.2); 5.0248 (0.5); 4.8993 (16.0); 3.8220 (1.0); 3.4083 (1.8); 3.3907 (3.3); 3.3756
(3.3); 3.3577 (1.8); 1.6204 (1.7); 1.6028 (2.6); 1.5955 (1.5); 1.5844 (2.4); 1.5740 (2.6); 1.3826
(1.5); 1.3733 (3.8); 1.3644 (5.3); 1.3549 (6.6); 1.3460 (3.1); 1.3372 (1.7); 0.9288 (3.6); 0.9244
(2.1); 0.9113 (11.0); 0.9052 (2.4); 0.8971 (2.0); 0.8934 (3.0); 0.0080 (0.6); −0.0002
(19.1); −0.0085 (0.7)
III-040: ¹H-NMR(400.0 MHz, CDCl3):
δ = 7.4161 (1.3); 7.4017 (1.1); 7.3956 (2.4); 7.3908 (0.5); 7.3811 (1.9); 7.3747 (1.7); 7.3615
(1.4); 7.2611 (46.2); 7.2054 (1.0); 7.2029 (1.1); 7.1990 (1.1); 7.1965 (1.2); 7.1843 (1.8); 7.1816
(1.9); 7.1781 (1.8); 7.1754 (2.2); 7.1666 (3.7); 7.1632 (1.8); 7.1608 (2.3); 7.1549 (4.4); 7.1494
(2.8); 7.1437 (5.2); 7.1378 (1.8); 7.1320 (4.9); 7.1237 (0.8); 7.0801 (2.0); 7.0762 (3.2); 7.0737
(2.9); 7.0678 (2.4); 7.0638 (3.0); 7.0602 (3.3); 7.0574 (5.3); 7.0455 (2.9); 7.0375 (6.6); 7.0316
(2.3); 7.0259 (1.2); 7.0200 (2.2); 7.0176 (5.8); 7.0145 (4.3); 7.0118 (2.1); 7.0061 (0.8); 7.0004
(1.5); 6.9947 (3.6); 6.9863 (0.5); 5.2985 (3.1); 4.9118 (16.0); 3.2784 (3.6); 3.2642 (4.2); 3.2607
(4.4); 3.2466 (3.8); 1.5731 (2.4); 1.0671 (0.8); 1.0651 (0.8); 1.0592 (0.8); 1.0551 (0.6); 1.0528
(0.6); 1.0472 (1.6); 1.0392 (0.7); 1.0350 (0.8); 1.0292 (0.9); 1.0272 (1.0); 1.0151 (0.6); 0.5872
(1.2); 0.5753 (3.2); 0.5724 (4.0); 0.5700 (2.4); 0.5673 (1.8); 0.5608 (2.0); 0.5552 (3.8); 0.5523
(3.3); 0.5408 (1.7); 0.2972 (1.4); 0.2855 (4.3); 0.2832 (3.7); 0.2735 (3.5); 0.2708 (4.5); 0.2590
(1.3); 0.0080 (0.6); −0.0002 (19.7); −0.0084 (0.9)
III-041: ¹H-NMR(400.0 MHz, CDCl3):
δ = 7.4129 (1.2); 7.3984 (1.1); 7.3923 (2.3); 7.3778 (1.8); 7.3715 (1.6); 7.3582 (1.3); 7.2610
(53.3); 7.2035 (0.9); 7.2009 (1.1); 7.1972 (1.0); 7.1946 (1.1); 7.1824 (1.7); 7.1798 (1.8); 7.1762
(1.7); 7.1735 (1.8); 7.1611 (1.1); 7.1569 (3.5); 7.1513 (1.7); 7.1452 (3.5); 7.1397 (2.4); 7.1339
(4.8); 7.1281 (1.6); 7.1256 (1.4); 7.1223 (4.7); 7.1140 (0.6); 7.0721 (1.8); 7.0683 (2.9); 7.0657
(2.5); 7.0601 (1.9); 7.0561 (2.3); 7.0522 (2.6); 7.0496 (4.2); 7.0439 (1.1); 7.0355 (5.6); 7.0301
(2.4); 7.0278 (1.6); 7.0240 (1.0); 7.0181 (2.1); 7.0156 (5.5); 7.0126 (4.1); 7.0099 (2.1); 7.0041
(1.1); 6.9985 (2.1); 6.9927 (4.2); 6.9845 (1.0); 5.2986 (5.9); 4.8865 (16.0); 2.8649 (0.7); 2.8561
(1.1); 2.8469 (1.5); 2.8380 (1.6); 2.8288 (1.1); 2.8200 (0.7); 1.5641 (3.4); 1.2585 (0.5); 0.8827
(1.1); 0.8683 (2.8); 0.8654 (4.2); 0.8623 (2.0); 0.8514 (3.8); 0.8475 (3.1); 0.8444 (1.8); 0.8338
(1.3); 0.6509 (1.3); 0.6409 (2.9); 0.6377 (3.2); 0.6341 (2.9); 0.6319 (3.0); 0.6281 (3.2); 0.6246
(2.7); 0.6107 (1.0); 0.0080 (0.7); −0.0002 (22.6); −0.0085 (0.8)
III-042: ¹H-NMR(400.0 MHz, CDCl3):
δ = 7.4159 (1.1); 7.4010 (1.1); 7.3953 (2.2); 7.3911 (0.6); 7.3808 (1.8); 7.3744 (1.4); 7.3608
(1.4); 7.2612 (28.4); 7.2056 (1.0); 7.2032 (1.1); 7.1992 (1.1); 7.1969 (1.1); 7.1845 (1.7); 7.1819
(1.8); 7.1783 (1.8); 7.1757 (1.9); 7.1636 (3.8); 7.1577 (2.0); 7.1521 (3.6); 7.1467 (2.5); 7.1410
(4.8); 7.1351 (1.8); 7.1293 (4.8); 7.1210 (1.0); 7.0948 (1.1); 7.0788 (2.1); 7.0752 (3.2); 7.0726
(2.6); 7.0660 (2.0); 7.0619 (2.4); 7.0592 (3.1); 7.0559 (4.6); 7.0432 (2.1); 7.0377 (6.1); 7.0323
(2.3); 7.0263 (1.0); 7.0178 (5.1); 7.0151 (3.9); 7.0124 (1.9); 7.0066 (0.7); 7.0008 (1.3); 6.9952
(3.3); 5.2984 (5.3); 4.9386 (16.0); 4.2016 (4.4); 4.1953 (4.7); 4.1880 (4.6); 4.1816 (4.5); 3.8217
(0.7); 2.2867 (2.5); 2.2803 (5.2); 2.2739 (2.7); 2.0433 (0.6); 1.5721 (1.6); −0.0002
(12.3); −0.0085 (0.6)
III-043: ¹H-NMR(400.0 MHz, CDCl3):
δ = 7.4140 (1.2); 7.3995 (1.1); 7.3935 (2.3); 7.3890 (0.5); 7.3790 (1.8); 7.3726 (1.5); 7.3592
(1.3); 7.2613 (35.5); 7.2037 (1.0); 7.2011 (1.1); 7.1973 (1.0); 7.1948 (1.1); 7.1826 (1.7); 7.1799
(1.8); 7.1764 (1.8); 7.1736 (2.0); 7.1647 (3.5); 7.1615 (1.7); 7.1590 (2.1); 7.1529 (4.2); 7.1475
(2.5); 7.1418 (5.0); 7.1359 (1.6); 7.1301 (4.7); 7.1219 (0.6); 7.0767 (1.8); 7.0728 (2.8); 7.0703
(2.4); 7.0641 (1.8); 7.0600 (2.4); 7.0571 (2.8); 7.0538 (4.8); 7.0462 (1.0); 7.0409 (2.3); 7.0379
(6.2); 7.0319 (2.5); 7.0264 (1.1); 7.0181 (5.7); 7.0151 (4.3); 7.0124 (2.2); 7.0066 (1.1); 7.0009
(1.9); 6.9952 (4.0); 6.9869 (0.9); 5.9558 (0.6); 5.9419 (1.4); 5.9300 (0.8); 5.9282 (0.8); 5.9162
(1.6); 5.9128 (0.9); 5.9023 (0.9); 5.8990 (1.6); 5.8870 (0.9); 5.8853 (0.9); 5.8732 (1.7); 5.8594
(0.8); 5.3015 (1.3); 5.2981 (7.6); 5.2945 (2.6); 5.2903 (1.1); 5.2588 (0.9); 5.2546 (2.0); 5.2515
(2.0); 5.2473 (0.9); 5.2154 (1.1); 5.2119 (2.6); 5.2087 (2.4); 5.2052 (1.0); 5.1897 (1.0); 5.1861
(2.4); 5.1829 (2.3); 5.1794 (0.9); 4.9349 (16.0); 4.0468 (1.4); 4.0431 (2.3); 4.0391 (1.5); 4.0325
(2.7); 4.0288 (4.4); 4.0251 (2.6); 4.0185 (1.6); 4.0145 (2.3); 4.0107 (1.3); 1.5850 (1.2); −0.0002
(14.3); −0.0085 (0.6)
III-045: ¹H-NMR(400.0 MHz, CDCl3):
δ = 7.5181 (1.5); 7.3032 (1.2); 7.2976 (1.7); 7.2931 (1.2); 7.2831 (7.3); 7.2816 (6.9); 7.2777
(4.5); 7.2750 (3.8); 7.2699 (6.4); 7.2592 (256.6); 7.2500 (5.6); 7.2477 (5.8); 7.2440 (1.8);
7.2381 (2.3); 7.2365 (2.1); 7.2223 (0.5); 7.2187 (0.7); 7.2092 (0.6); 7.1748 (0.6); 7.1471 (4.5);
7.1430 (5.4); 7.1373 (1.6); 7.1307 (2.4); 7.1261 (4.4); 7.1227 (3.6); 7.0952 (0.6); 7.0881 (4.6);
7.0826 (1.4); 7.0711 (1.7); 7.0666 (6.7); 7.0614 (1.6); 7.0499 (1.3); 7.0445 (3.6); 6.9952 (1.4);
6.7413 (0.8); 5.9268 (0.6); 5.9133 (1.3); 5.9007 (0.9); 5.8875 (1.5); 5.8839 (0.8); 5.8740 (0.8);
5.8703 (1.6); 5.8577 (1.1); 5.8446 (1.6); 5.8311 (0.8); 5.3280 (1.0); 5.3109 (3.2); 5.2938 (3.3);
5.2768 (1.0); 5.2406 (1.0); 5.2363 (2.3); 5.2330 (2.4); 5.2287 (1.0); 5.1977 (0.9); 5.1933 (2.0);
5.1900 (2.1); 5.1858 (0.9); 5.1558 (1.1); 5.1522 (2.7); 5.1487 (2.6); 5.1451 (1.0); 5.1300 (1.0);
5.1264 (2.5); 5.1230 (2.4); 5.1193 (1.0); 3.9914 (0.8); 3.9872 (1.5); 3.9813 (1.7); 3.9772 (2.0);
3.9727 (2.6); 3.9674 (2.8); 3.9633 (2.0); 3.9592 (1.6); 3.9533 (1.5); 3.9490 (0.8); 1.7140 (16.0);
1.6970 (15.9); 1.5575 (6.2); 0.0080 (3.4); −0.0002 (99.9); −0.0085 (3.4)
III-046: ¹H-NMR(400.0 MHz, CDCl3):
δ = 7.5182 (0.7); 7.3013 (0.6); 7.2961 (0.8); 7.2877 (0.8); 7.2814 (3.6); 7.2798 (3.4); 7.2759
(2.3); 7.2733 (2.1); 7.2684 (3.4); 7.2593 (131.7); 7.2519 (4.6); 7.2475 (2.7); 7.2464 (2.7);
7.2415 (0.7); 7.2355 (1.1); 7.1457 (2.2); 7.1415 (2.7); 7.1359 (0.8); 7.1291 (1.2); 7.1246 (2.2);
7.1213 (1.8); 7.0879 (2.2); 7.0824 (0.7); 7.0709 (0.8); 7.0662 (3.3); 7.0612 (0.8); 7.0497 (0.6);
7.0443 (1.7); 6.9953 (0.7); 5.2764 (1.6); 5.2594 (1.6); 3.3302 (0.6); 3.3124 (1.2); 3.3002 (1.3);
3.2974 (1.2); 3.2857 (1.2); 3.2802 (0.6); 3.2680 (0.6); 1.6990 (7.7); 1.6820 (7.6); 1.6004 (1.2);
1.5823 (2.5); 1.5641 (2.8); 1.5389 (16.0); 0.9486 (4.0); 0.9302 (8.2); 0.9116 (3.5); 0.0080
(1.7); −0.0002 (49.5); −0.0084 (1.7)
III-047: ¹H-NMR(400.0 MHz, CDCl3):
δ = 7.3029 (0.7); 7.2988 (1.0); 7.2975 (1.0); 7.2932 (0.9); 7.2873 (3.0); 7.2813 (4.2); 7.2774
(2.2); 7.2744 (4.1); 7.2650 (6.0); 7.2594 (82.3); 7.2528 (5.6); 7.2491 (2.2); 7.2448 (0.7); 7.2427
(0.7); 7.2368 (1.4); 7.2354 (1.3); 7.1528 (3.0); 7.1487 (3.7); 7.1431 (1.0); 7.1363 (1.6); 7.1318
(2.9); 7.1285 (2.4); 7.0901 (3.0); 7.0846 (0.9); 7.0759 (0.6); 7.0731 (1.1); 7.0686 (4.4); 7.0633
(1.1); 7.0519 (0.8); 7.0465 (2.3); 6.7700 (0.6); 5.2980 (0.6); 5.2809 (2.1); 5.2639 (2.1); 5.2469
(0.6); 3.2195 (1.6); 3.2173 (1.6); 3.2027 (2.8); 3.1875 (1.6); 1.7060 (10.3); 1.6890 (10.1);
1.5458 (16.0); 1.0121 (0.6); 1.0098 (0.5); 0.9921 (1.0); 0.9799 (0.5); 0.9744 (0.6); 0.9721 (0.6);
0.5169 (0.8); 0.5116 (2.0); 0.5087 (2.6); 0.5060 (1.5); 0.4961 (1.0); 0.4915 (2.3); 0.4886 (2.1);
0.4837 (0.8); 0.2395 (0.9); 0.2331 (1.2); 0.2307 (1.3); 0.2275 (1.8); 0.2238 (2.0); 0.2184 (1.7);
0.2121 (1.2); 0.2060 (0.8); 0.0079 (1.1); −0.0002 (31.3); −0.0085 (1.0)
III-048: ¹H-NMR(400.0 MHz, CDCl3):
δ = 7.2782 (1.2); 7.2741 (0.8); 7.2715 (0.8); 7.2595 (48.7); 7.2465 (0.7); 7.2441 (0.8); 7.1435
(0.7); 7.1393 (0.8); 7.1224 (0.7); 7.1192 (0.6); 7.0878 (0.7); 7.0661 (1.0); 7.0443 (0.5); 1.7046
(2.3); 1.6876 (2.2); 1.5377 (16.0); 0.9226 (3.6); 0.9059 (3.5); 0.0079 (0.7); −0.0002
(19.6); −0.0085 (0.8)
III-049: ¹H-NMR(400.0 MHz, CDCl3):
δ = 7.5182 (0.7); 7.3099 (1.3); 7.3044 (1.9); 7.3000 (1.2); 7.2882 (5.9); 7.2819 (6.1); 7.2763
(3.0); 7.2733 (4.0); 7.2690 (10.6); 7.2593 (120.4); 7.2527 (4.8); 7.2465 (5.8); 7.2337 (0.6);
7.2288 (0.6); 7.2250 (0.7); 7.1562 (4.7); 7.1520 (5.9); 7.1464 (1.7); 7.1398 (2.4); 7.1352 (4.5);
7.1318 (3.8); 7.0970 (0.6); 7.0899 (4.5); 7.0844 (1.5); 7.0677 (7.5); 7.0632 (1.8); 7.0517 (1.3);
7.0463 (3.7); 6.9952 (0.7); 6.7242 (1.0); 5.9218 (0.6); 5.9082 (1.3); 5.8948 (1.1); 5.8824 (1.5);
5.8788 (0.9); 5.8687 (0.8); 5.8652 (1.6); 5.8519 (1.2); 5.8395 (1.6); 5.8259 (0.8); 5.3246 (1.1);
5.3076 (3.5); 5.2906 (3.5); 5.2736 (1.1); 5.2346 (1.0); 5.2303 (2.5); 5.2270 (2.4); 5.2227 (1.1);
5.1917 (0.9); 5.1874 (2.2); 5.1840 (2.1); 5.1832 (2.0); 5.1798 (1.0); 5.1490 (2.7); 5.1456 (2.6);
5.1420 (1.1); 5.1232 (2.5); 5.1198 (2.4); 5.1163 (1.0); 3.9846 (1.9); 3.9801 (2.1); 3.9753 (2.2);
3.9703 (3.5); 3.9659 (3.5); 3.9615 (2.2); 3.9562 (2.0); 3.9519 (1.9); 3.9477 (0.8); 1.7111 (16.0);
1.6941 (15.8); 1.5438 (11.2); 0.0080 (1.6); −0.0002 (45.9); −0.0085 (1.8)
III-050: ¹H-NMR(400.0 MHz, CDCl3):
δ = 7.5182 (0.7); 7.3083 (1.7); 7.3027 (1.6); 7.2982 (1.1); 7.2940 (1.0); 7.2863 (5.1); 7.2804
(5.4); 7.2751 (2.6); 7.2715 (3.5); 7.2676 (10.4); 7.2594 (124.2); 7.2545 (6.3); 7.2510 (3.3);
7.2452 (4.7); 7.2421 (2.4); 7.2227 (0.7); 7.1547 (4.2); 7.1506 (5.4); 7.1449 (1.5); 7.1382 (2.3);
7.1336 (4.2); 7.1303 (3.5); 7.0968 (0.6); 7.0897 (4.3); 7.0842 (1.4); 7.0755 (0.8); 7.0727 (1.5);
7.0682 (6.4); 7.0630 (1.6); 7.0515 (1.2); 7.0461 (3.4); 6.9954 (0.7); 6.6541 (0.8); 5.2893 (0.9);
5.2723 (3.1); 5.2553 (3.1); 5.2383 (0.9); 3.3270 (1.2); 3.3174 (1.2); 3.3093 (2.3); 3.3026 (1.5);
3.2997 (2.3); 3.2942 (2.2); 3.2849 (2.4); 3.2765 (1.1); 3.2673 (1.2); 1.6956 (15.0); 1.6786
(14.9); 1.6152 (0.6); 1.5968 (2.5); 1.5787 (5.1); 1.5604 (6.1); 1.5466 (5.9); 1.5426 (6.2); 1.5243
(0.9); 0.9427 (7.9); 0.9243 (16.0); 0.9057 (6.9); 0.0080 (1.6); −0.0002 (45.1); −0.0085 (1.5)
III-051: ¹H-NMR(400.0 MHz, CDCl3):
δ = 7.2870 (1.5); 7.2817 (1.0); 7.2733 (1.8); 7.2695 (2.4); 7.2678 (2.2); 7.2594 (66.9); 7.2437
(0.7); 7.1620 (1.0); 7.1580 (1.3); 7.1455 (0.6); 7.1410 (1.0); 7.1377 (0.8); 7.0922 (1.0); 7.0706
(1.6); 7.0486 (0.8); 5.2774 (0.7); 5.2604 (0.8); 3.2163 (0.8); 3.2020 (1.0); 3.1990 (1.0); 3.1847
(0.9); 1.7023 (3.6); 1.6853 (3.6); 1.5378 (16.0); 0.5081 (0.7); 0.5053 (0.9); 0.5027 (0.6); 0.4881
(0.8); 0.4852 (0.7); 0.2234 (0.7); 0.2196 (0.7); 0.2141 (0.6); 0.0080 (0.9); −0.0002
(25.2); −0.0084 (1.0)
III-052: ¹H-NMR(400.0 MHz, CDCl3):
δ = 7.3059 (0.9); 7.3017 (1.2); 7.3004 (1.3); 7.2960 (0.9); 7.2918 (0.8); 7.2842 (4.3); 7.2784
(4.4); 7.2730 (2.2); 7.2694 (3.1); 7.2655 (9.4); 7.2637 (7.7); 7.2595 (64.8); 7.2529 (3.9); 7.2488
(2.2); 7.2465 (1.7); 7.2431 (4.1); 7.2410 (2.3); 7.2213 (0.6); 7.1523 (3.7); 7.1481 (4.5); 7.1425
(1.3); 7.1358 (2.0); 7.1312 (3.7); 7.1279 (3.0); 7.0892 (3.8); 7.0837 (1.2); 7.0749 (0.8); 7.0722
(1.3); 7.0677 (5.5); 7.0625 (1.4); 7.0510 (1.1); 7.0456 (2.9); 6.6798 (0.7); 5.2943 (0.8); 5.2773
(2.6); 5.2603 (2.6); 5.2433 (0.8); 3.2352 (0.6); 3.2189 (1.0); 3.2022 (1.8); 3.1857 (2.2); 3.1694
(1.2); 3.1499 (1.4); 3.1351 (1.8); 3.1330 (1.7); 3.1180 (1.7); 3.1019 (0.8); 3.0998 (0.8); 3.0850
(0.7); 1.8452 (0.7); 1.8283 (1.4); 1.8115 (1.8); 1.7947 (1.5); 1.7780 (0.8); 1.7011 (12.6); 1.6840
(12.6); 1.5563 (2.4); 0.9167 (16.0); 0.9149 (15.6); 0.9000 (15.5); 0.8982 (15.0); 0.0079
(0.8); −0.0002 (23.7); −0.0085 (0.9)
III-058: ¹H-NMR(400.0 MHz, CDCl3):
δ = 7.3094 (0.6); 7.3045 (0.7); 7.2874 (3.7); 7.2817 (1.9); 7.2735 (2.9); 7.2690 (4.1); 7.2595
(62.6); 7.2542 (3.4); 7.2517 (3.4); 7.2478 (1.1); 7.2445 (0.7); 7.2413 (1.2); 7.1648 (2.2); 7.1608
(2.9); 7.1552 (1.1); 7.1482 (1.5); 7.1437 (2.5); 7.1405 (2.1); 7.0883 (2.1); 7.0828 (0.7); 7.0712
(0.8); 7.0667 (3.2); 7.0616 (0.8); 7.0501 (0.8); 7.0447 (1.7); 5.3521 (1.5); 5.3351 (1.5); 5.2981
(1.5); 4.1430 (1.7); 4.1293 (2.0); 4.1190 (1.9); 4.1057 (1.8); 3.7752 (2.5); 3.7680 (16.0); 1.7188
(7.2); 1.7017 (7.3); 1.6799 (0.8); 1.5424 (9.5); 0.0080 (0.7); −0.0002 (23.2); −0.0085 (0.9)
III-059: ¹H-NMR(400.0 MHz, CDCl3):
δ = 7.2989 (1.0); 7.2938 (1.0); 7.2909 (1.6); 7.2807 (3.2); 7.2776 (4.2); 7.2673 (3.8); 7.2600
(66.7); 7.2516 (3.5); 7.2476 (3.4); 7.2450 (3.0); 7.2348 (1.4); 7.2313 (1.6); 7.2158 (0.6); 7.1569
(1.7); 7.1529 (2.1); 7.1456 (1.9); 7.1414 (2.7); 7.1358 (2.1); 7.1326 (1.4); 7.1289 (1.1); 7.1244
(1.9); 7.1212 (1.5); 7.0870 (2.7); 7.0825 (1.1); 7.0656 (4.6); 7.0444 (2.3); 7.0361 (0.6); 7.0253
(0.7); 5.2978 (3.2); 5.2467 (1.2); 5.2298 (1.4); 5.2142 (1.6); 5.1973 (1.4); 4.3949 (0.7); 4.3779
(0.8); 3.6839 (1.7); 3.6745 (1.5); 3.6659 (16.0); 3.6614 (13.9); 2.8912 (0.7); 2.8847 (0.7);
2.8696 (0.8); 2.2657 (0.6); 2.2543 (0.5); 2.2486 (0.8); 2.2317 (0.7); 2.2260 (0.5); 2.0075 (0.5);
1.9977 (1.2); 1.9860 (0.9); 1.9815 (1.6); 1.9656 (1.3); 1.9558 (0.8); 1.9448 (1.1); 1.9309 (0.7);
1.9146 (0.6); 1.8564 (0.8); 1.8371 (0.7); 1.8301 (0.5); 1.8236 (0.6); 1.7699 (0.6); 1.7530 (0.5);
1.7360 (0.7); 1.7204 (0.7); 1.7051 (0.6); 1.6867 (6.4); 1.6791 (5.4); 1.6696 (6.3); 1.6621 (5.4);
1.6484 (0.8); 1.6414 (0.6); 1.5600 (14.0); 0.0080 (0.8); −0.0002 (23.2); −0.0085 (0.8)
III-063: ¹H-NMR(400.0 MHz, CDCl3):
δ = 7.3021 (0.5); 7.2857 (2.3); 7.2795 (1.3); 7.2719 (2.2); 7.2675 (2.9); 7.2660 (2.8); 7.2593
(23.0); 7.2501 (1.7); 7.2453 (0.9); 7.2434 (0.9); 7.1572 (1.4); 7.1528 (1.5); 7.1472 (0.6); 7.1409
(0.9); 7.1363 (1.4); 7.1329 (1.2); 7.0883 (1.2); 7.0712 (0.5); 7.0665 (1.9); 7.0448 (1.0); 5.2854
(0.6); 5.2819 (0.6); 5.2424 (0.6); 5.2388 (0.5); 5.1413 (0.7); 5.1378 (0.7); 5.1155 (0.7); 5.1120
(0.6); 3.9979 (0.5); 3.9938 (0.8); 3.9897 (0.6); 3.9835 (0.8); 3.9798 (1.3); 3.9761 (0.8); 3.9700
(0.6); 3.9658 (0.8); 1.7663 (16.0); 1.5476 (6.4); −0.0002 (8.0)
III-066: ¹H-NMR(400.0 MHz, CDCl3):
δ = 7.4567 (0.6); 7.4530 (0.7); 7.4443 (0.7); 7.4390 (1.2); 7.4345 (1.3); 7.4220 (1.2); 7.4205
(1.2); 7.4182 (1.2); 7.4132 (1.0); 7.4044 (0.9); 7.3998 (1.8); 7.3953 (1.2); 7.3798 (2.2); 7.3764
(1.9); 7.3616 (1.7); 7.3573 (1.3); 7.2609 (29.4); 7.2601 (28.7); 7.2400 (2.5); 7.2386 (2.5);
7.2199 (1.2); 7.2064 (1.1); 7.2011 (1.1); 7.1879 (1.1); 7.1815 (1.6); 7.1751 (1.3); 7.1710 (1.2);
7.1621 (1.1); 7.1568 (1.2); 7.1501 (1.9); 7.1296 (1.6); 7.1255 (1.8); 7.1051 (2.7); 7.0837 (1.7);
7.0805 (2.6); 7.0593 (1.4); 7.0563 (1.3); 7.0460 (1.0); 7.0419 (1.4); 7.0366 (1.5); 7.0315 (1.4);
7.0265 (1.1); 7.0206 (1.1); 7.0154 (1.2); 7.0104 (1.1); 6.9968 (0.9); 6.9810 (0.8); 5.9546 (0.5);
5.9409 (1.0); 5.9278 (0.9); 5.9151 (1.2); 5.9120 (0.8); 5.9010 (0.8); 5.8980 (1.3); 5.8848 (1.0);
5.8722 (1.3); 5.8583 (0.6); 5.2979 (4.2); 5.2554 (1.9); 5.2515 (1.9); 5.2483 (0.9); 5.2124 (2.3);
5.2089 (2.1); 5.1866 (2.1); 5.1832 (2.0); 4.9068 (16.0); 4.7635 (0.8); 4.0409 (2.5); 4.0373 (1.7);
4.0267 (4.6); 4.0161 (1.7); 4.0125 (2.4); 1.5684 (7.0); 1.2557 (0.5); −0.0002 (10.8); −0.0009
(10.1); −0.0083 (0.5)
III-068: ¹H-NMR(400.0 MHz, CDCl3):
δ = 7.3990 (0.8); 7.3930 (0.8); 7.3776 (1.4); 7.3616 (1.0); 7.3561 (1.0); 7.3405 (0.7); 7.3108
(0.8); 7.2595 (56.5); 7.2116 (0.6); 7.1876 (0.9); 7.1825 (0.9); 7.1695 (1.3); 7.1627 (1.2); 7.1558
(1.0); 7.1495 (1.4); 7.1373 (0.9); 7.1251 (1.2); 7.1043 (0.8); 7.0424 (0.9); 7.0365 (1.0); 7.0314
(0.9); 7.0152 (0.8); 6.9953 (0.9); 6.9866 (1.8); 6.9677 (2.6); 6.9466 (1.6); 6.6319 (0.5); 5.9223
(0.5); 5.8968 (0.6); 5.8798 (0.7); 5.8670 (0.6); 5.8539 (0.6); 5.2631 (1.2); 5.2202 (1.1); 5.1851
(1.2); 5.1821 (1.3); 5.1565 (1.2); 4.8347 (8.3); 4.0253 (1.5); 4.0110 (2.6); 3.9969 (1.5); 1.5380
(16.0); −0.0002 (21.3)
III-069: ¹H-NMR(400.0 MHz, CDCl3):
δ = 7.4723 (0.6); 7.4569 (1.2); 7.4507 (1.1); 7.4414 (0.7); 7.4354 (2.3); 7.4293 (0.8); 7.4199
(1.1); 7.4138 (1.3); 7.3984 (0.6); 7.2612 (32.0); 7.2350 (1.0); 7.2298 (1.0); 7.2168 (1.0); 7.2105
(1.6); 7.2043 (1.2); 7.1937 (1.2); 7.1914 (1.2); 7.1860 (1.1); 7.1727 (1.7); 7.1695 (1.1); 7.1531
(1.6); 7.1482 (1.7); 7.1287 (1.4); 7.0892 (0.8); 7.0852 (1.1); 7.0796 (1.2); 7.0751 (1.2); 7.0701
(0.9); 7.0637 (0.8); 7.0584 (0.8); 7.0536 (0.8); 7.0101 (3.0); 6.9918 (4.4); 6.9885 (4.4); 6.9703
(3.0); 5.9519 (0.6); 5.9381 (1.3); 5.9259 (0.8); 5.9123 (1.5); 5.9090 (0.8); 5.8985 (0.8); 5.8952
(1.6); 5.8817 (1.0); 5.8694 (1.6); 5.8556 (0.7); 5.3019 (1.0); 5.2979 (3.3); 5.2949 (2.3); 5.2907
(1.0); 5.2590 (0.8); 5.2549 (1.9); 5.2519 (1.9); 5.2477 (0.9); 5.2151 (1.1); 5.2117 (2.5); 5.2085
(2.3); 5.2051 (1.0); 5.1894 (1.0); 5.1859 (2.3); 5.1827 (2.2); 5.1793 (0.9); 4.8934 (16.0); 4.0443
(1.4); 4.0405 (2.3); 4.0367 (1.6); 4.0300 (2.7); 4.0263 (4.3); 4.0226 (2.6); 4.0160 (1.6); 4.0121
(2.3); 4.0083 (1.3); 1.5740 (7.8); −0.0002 (11.3)

### B. Formulation Examples

a) A dusting product is obtained by mixing 10 parts by weight of a compound of the formula (I) and/or salts thereof and 90 parts by weight of talc as inert substance and comminuting the mixture in an impact mill.

b) A readily water-dispersible, wettable powder is obtained by mixing 25 parts by weight of a compound of the formula (I) and/or salts thereof, 64 parts by weight of kaolin-containing quartz as inert substance, 10 parts by weight of potassium lignosulfonate and 1 part by weight of sodium oleoylmethyltaurate as wetting agent and dispersant and grinding in a pinned-disc mill.

c) A readily water-dispersible dispersion concentrate is obtained by mixing 20 parts by weight of a compound of the formula (I) and/or salts thereof with 6 parts by weight of alkylphenol polyglycol ether (®Triton X 207), 3 parts by weight of isotridecanol polyglycol ether (8 EO) and 71 parts by weight of paraffinic mineral oil (boiling range e.g. about 255 to more than 277° C.) and grinding to a fineness of below 5 microns in an attrition ball mill.

d) An emulsifiable concentrate is obtained from 15 parts by weight of a compound of the formula (I) and/or salts thereof, 75 parts by weight of cyclohexanone as solvent and 10 parts by weight of oxethylated nonylphenol as emulsifier.

e) Water-dispersible granules are obtained by mixing

75 parts by weight of a compound of the formula (I) and/or salts thereof,

10 parts by weight of calcium lignosulfonate,

5 parts by weight of sodium lauryl sulfate,

3 parts by weight of polyvinyl alcohol and

7 parts by weight of kaolin,

grinding the mixture in a pinned-disc mill, and granulating the powder in a fluidized bed by spray application of water as a granulating liquid.

f) Water-dispersible granules are also obtained by homogenizing and precomminuting, in a colloid mill,

25 parts by weight of a compound of the formula (I) and/or salts thereof,

5 parts by weight of sodium 2,2′-dinaphthylmethane-6,6′-disulfonate,

2 parts by weight of sodium oleoylmethyltaurate,

1 part by weight of polyvinyl alcohol,

17 parts by weight of calcium carbonate and

50 parts by weight of water,

then grinding the mixture in a bead mill and atomizing and drying the resulting suspension in a spray tower by means of a one-phase nozzle.

### C. Biological Examples

1. Pre-Emergence Herbicidal Action and Crop Plant Compatibility

Seeds of mono- and dicotyledonous weed plants are placed in plastic pots in sandy loam soil (doubly sown with one species each of mono- or dicotyledonous weed plants per pot) and covered with soil. The compounds of the invention, formulated in the form of wettable powders (WP) or as emulsion concentrates (EC), are then applied onto the surface of the covering soil as aqueous suspension or emulsion with addition of 0.5% additive at a water application rate equivalent to 600 litres per hectare. After the treatment, the pots are placed in a greenhouse and kept under good growth conditions for the trial plants. After about 3 weeks, the effect of the preparations is scored visually in comparison with untreated controls as percentages. For example, 100% activity=the plants have died, 0% activity=like control plants.

In Tables 4.1-4.15 and 5.1-5.16 below, the following abbreviations are used:

| | | | |
|---|---|---|---|
| ALOMY: | Alopecurus myosuroides | AMARE: | Amaranthus retroflexus |
| AVEFA: | Avena fatua | CYPES: | Cyperus esculentus |
| ECHCG: | Echinochloa crus-galli | HORMU: | Hordeum murinum |
| LOLRI: | Lolium rigidum | MATIN: | Matricaria inodora |
| PHBPU: | Pharbitis purpurea | POLCO: | Polygonum convolvulus |
| SETVI: | Setaria viridis | STEME: | Stellaria media |
| VERPE: | Veronica persica | VIOTR: | Viola tricolor |

Tables 4.1-4.15 below show the effects of selected compounds of the general formula (I) according to Tables 1 to 3 on various harmful plants at an application rate corresponding to 1280 or 320 g/ha, which have been obtained by the aforementioned experimental method.

TABLE 4.1
Pre-emergence action at
1280 g/ha against ALOMY in %

| Ex. | Dosage | |
|---|---|---|
| No. | [g/ha] | ALOMY |
| I-002 | 1280 | 80 |
| I-007 | 1280 | 80 |
| I-008 | 1280 | 100 |
| I-056 | 1280 | 90 |
| I-057 | 1280 | 90 |
| I-061 | 1280 | 90 |
| I-067 | 1280 | 90 |
| I-070 | 1280 | 90 |
| I-072 | 1280 | 80 |

TABLE 4.2
Pre-emergence action at
1280 g/ha against CYPES in %

| Ex. | Dosage | |
|---|---|---|
| No. | [g/ha] | CYPES |
| I-003 | 1280 | 100 |
| I-006 | 1280 | 100 |
| I-012 | 1280 | 100 |
| I-021 | 1280 | 100 |
| I-022 | 1280 | 100 |
| I-056 | 1280 | 90 |
| I-061 | 1280 | 90 |
| I-067 | 1280 | 90 |
| I-070 | 1280 | 80 |

TABLE 4.3
Pre-emergence action at
1280 g/ha against ECHCG in %

| Ex. | Dosage | |
|---|---|---|
| No. | [g/ha] | ECHCG |
| I-011 | 1280 | 80 |
| I-022 | 1280 | 80 |
| I-054 | 1280 | 90 |
| I-055 | 1280 | 100 |
| I-057 | 1280 | 80 |
| I-061 | 1280 | 90 |
| I-067 | 1280 | 80 |
| I-069 | 1280 | 80 |
| I-070 | 1280 | 90 |
| I-076 | 1280 | 90 |
| I-079 | 1280 | 90 |
| I-081 | 1280 | 90 |
| I-085 | 1280 | 100 |
| I-093 | 1280 | 100 |
| I-097 | 1280 | 100 |
| I-098 | 1280 | 100 |
| I-100 | 1280 | 100 |
| I-101 | 1280 | 100 |
| I-102 | 1280 | 100 |
| I-107 | 1280 | 100 |
| I-111 | 1280 | 100 |
| I-114 | 1280 | 90 |
| I-120 | 1280 | 90 |
| I-121 | 1280 | 100 |
| I-122 | 1280 | 100 |
| I-125 | 1280 | 100 |
| I-130 | 1280 | 100 |
| I-131 | 1280 | 100 |
| I-133 | 1280 | 90 |
| I-134 | 1280 | 100 |
| I-136 | 1280 | 100 |
| I-137 | 1280 | 100 |
| I-138 | 1280 | 100 |
| I-139 | 1280 | 100 |
| I-140 | 1280 | 90 |
| I-141 | 1280 | 100 |
| I-142 | 1280 | 100 |
| I-145 | 1280 | 100 |
| I-164 | 1280 | 100 |
| I-167 | 1280 | 100 |
| I-168 | 1280 | 90 |
| I-169 | 1280 | 100 |
| I-171 | 1280 | 90 |
| II-04 | 1280 | 100 |
| III-001 | 1280 | 90 |
| III-003 | 1280 | 100 |
| III-004 | 1280 | 100 |
| III-005 | 1280 | 100 |
| III-006 | 1280 | 100 |
| III-015 | 1280 | 100 |
| III-016 | 1280 | 100 |
| III-022 | 1280 | 100 |
| III-023 | 1280 | 90 |
| III-024 | 1280 | 90 |
| III-027 | 1280 | 100 |
| III-033 | 1280 | 90 |
| III-053 | 1280 | 100 |
| III-058 | 1280 | 90 |
| III-067 | 1280 | 100 |
| III-068 | 1280 | 100 |
| III-069 | 1280 | 100 |

TABLE 4.4
Pre-emergence action at
1280 g/ha against SETVI in %

| Ex. | Dosage | |
|---|---|---|
| No. | [g/ha] | SETVI |
| I-008 | 1280 | 90 |
| I-015 | 1280 | 90 |
| I-022 | 1280 | 100 |
| I-025 | 1280 | 80 |
| I-036 | 1280 | 80 |
| I-054 | 1280 | 90 |
| I-055 | 1280 | 100 |
| I-056 | 1280 | 100 |
| I-057 | 1280 | 90 |
| I-061 | 1280 | 100 |
| I-063 | 1280 | 100 |
| I-067 | 1280 | 100 |
| I-069 | 1280 | 90 |
| I-070 | 1280 | 90 |
| I-072 | 1280 | 80 |
| I-076 | 1280 | 100 |
| I-078 | 1280 | 100 |
| I-079 | 1280 | 100 |
| I-081 | 1280 | 100 |
| I-085 | 1280 | 100 |
| I-086 | 1280 | 90 |
| I-088 | 1280 | 90 |
| I-091 | 1280 | 90 |
| I-092 | 1280 | 100 |
| I-093 | 1280 | 100 |
| I-094 | 1280 | 100 |
| I-095 | 1280 | 100 |
| I-097 | 1280 | 100 |
| I-098 | 1280 | 90 |
| I-100 | 1280 | 100 |
| I-101 | 1280 | 100 |
| I-102 | 1280 | 100 |
| I-107 | 1280 | 90 |
| I-108 | 1280 | 100 |
| I-111 | 1280 | 90 |
| I-114 | 1280 | 100 |
| I-118 | 1280 | 90 |
| I-119 | 1280 | 100 |
| I-120 | 1280 | 100 |
| I-121 | 1280 | 100 |
| I-123 | 1280 | 90 |
| I-124 | 1280 | 100 |
| I-125 | 1280 | 100 |
| I-126 | 1280 | 100 |
| I-127 | 1280 | 100 |
| I-129 | 1280 | 100 |
| I-130 | 1280 | 100 |
| I-131 | 1280 | 100 |
| I-132 | 1280 | 100 |
| I-133 | 1280 | 100 |
| I-134 | 1280 | 100 |
| I-136 | 1280 | 100 |
| I-137 | 1280 | 100 |
| I-138 | 1280 | 100 |
| I-139 | 1280 | 100 |
| I-140 | 1280 | 100 |
| I-141 | 1280 | 100 |
| I-142 | 1280 | 100 |
| I-143 | 1280 | 100 |
| I-144 | 1280 | 100 |
| I-145 | 1280 | 100 |
| I-156 | 1280 | 90 |
| I-164 | 1280 | 100 |
| I-165 | 1280 | 100 |
| I-166 | 1280 | 90 |
| I-167 | 1280 | 100 |
| I-169 | 1280 | 100 |
| I-170 | 1280 | 100 |
| I-171 | 1280 | 100 |
| III-001 | 1280 | 100 |
| III-003 | 1280 | 100 |
| III-004 | 1280 | 100 |
| III-005 | 1280 | 100 |
| III-006 | 1280 | 100 |
| III-015 | 1280 | 100 |
| III-016 | 1280 | 100 |
| III-017 | 1280 | 100 |
| III-018 | 1280 | 90 |
| III-019 | 1280 | 100 |
| III-022 | 1280 | 100 |
| III-023 | 1280 | 100 |
| III-024 | 1280 | 100 |
| III-026 | 1280 | 90 |
| III-027 | 1280 | 90 |
| III-033 | 1280 | 100 |
| III-053 | 1280 | 100 |
| III-054 | 1280 | 90 |
| III-056 | 1280 | 90 |
| III-058 | 1280 | 90 |
| III-066 | 1280 | 90 |
| III-067 | 1280 | 100 |
| III-068 | 1280 | 100 |
| III-069 | 1280 | 100 |

TABLE 4.5
Pre-emergence action at
1280 g/ha against AMARE in %

| Ex. | Dosage | |
|---|---|---|
| No. | [g/ha] | AMARE |
| I-002 | 1280 | 100 |
| I-003 | 1280 | 100 |
| I-004 | 1280 | 90 |
| I-006 | 1280 | 100 |
| I-007 | 1280 | 100 |
| I-008 | 1280 | 100 |
| I-011 | 1280 | 90 |
| I-012 | 1280 | 100 |
| I-015 | 1280 | 90 |
| I-016 | 1280 | 100 |
| I-021 | 1280 | 100 |
| I-022 | 1280 | 100 |
| I-023 | 1280 | 90 |
| I-025 | 1280 | 90 |
| I-026 | 1280 | 80 |
| I-036 | 1280 | 80 |
| I-046 | 1280 | 90 |
| I-051 | 1280 | 90 |
| I-054 | 1280 | 100 |
| I-055 | 1280 | 100 |
| I-056 | 1280 | 100 |
| I-057 | 1280 | 100 |
| I-061 | 1280 | 100 |
| I-063 | 1280 | 80 |
| I-066 | 1280 | 100 |
| I-067 | 1280 | 100 |
| I-069 | 1280 | 90 |
| I-070 | 1280 | 100 |
| I-072 | 1280 | 90 |
| I-076 | 1280 | 100 |
| I-078 | 1280 | 90 |
| I-079 | 1280 | 100 |
| I-081 | 1280 | 100 |
| I-085 | 1280 | 100 |
| I-086 | 1280 | 100 |
| I-088 | 1280 | 90 |
| I-091 | 1280 | 100 |
| I-092 | 1280 | 100 |
| I-093 | 1280 | 100 |
| I-094 | 1280 | 100 |
| I-095 | 1280 | 100 |
| I-097 | 1280 | 100 |
| I-098 | 1280 | 100 |
| I-099 | 1280 | 90 |
| I-100 | 1280 | 100 |
| I-101 | 1280 | 90 |
| I-102 | 1280 | 100 |
| I-107 | 1280 | 90 |
| I-108 | 1280 | 90 |
| I-112 | 1280 | 100 |
| I-114 | 1280 | 100 |
| I-118 | 1280 | 100 |
| I-119 | 1280 | 100 |
| I-120 | 1280 | 100 |
| I-121 | 1280 | 90 |
| I-122 | 1280 | 100 |
| I-123 | 1280 | 100 |
| I-125 | 1280 | 100 |
| I-126 | 1280 | 100 |
| I-127 | 1280 | 100 |
| I-128 | 1280 | 100 |
| I-129 | 1280 | 100 |
| I-130 | 1280 | 100 |
| I-131 | 1280 | 100 |
| I-132 | 1280 | 100 |
| I-133 | 1280 | 100 |
| I-134 | 1280 | 100 |
| I-136 | 1280 | 100 |
| I-137 | 1280 | 100 |
| I-138 | 1280 | 100 |
| I-139 | 1280 | 100 |
| I-140 | 1280 | 90 |
| I-141 | 1280 | 100 |
| I-142 | 1280 | 100 |
| I-143 | 1280 | 100 |
| I-144 | 1280 | 100 |
| I-145 | 1280 | 90 |
| I-155 | 1280 | 90 |
| I-156 | 1280 | 100 |
| I-164 | 1280 | 100 |
| I-165 | 1280 | 100 |
| I-166 | 1280 | 90 |
| I-167 | 1280 | 100 |
| I-168 | 1280 | 100 |
| I-169 | 1280 | 100 |
| I-170 | 1280 | 100 |
| I-171 | 1280 | 100 |
| II-04 | 1280 | 100 |
| II-06 | 1280 | 90 |
| II-07 | 1280 | 90 |
| II-09 | 1280 | 90 |
| II-13 | 1280 | 100 |
| II-14 | 1280 | 100 |
| III-003 | 1280 | 100 |
| III-004 | 1280 | 100 |
| III-005 | 1280 | 90 |
| III-006 | 1280 | 90 |
| III-015 | 1280 | 100 |
| III-016 | 1280 | 100 |
| III-017 | 1280 | 100 |
| III-018 | 1280 | 90 |
| III-019 | 1280 | 90 |
| III-022 | 1280 | 90 |
| III-023 | 1280 | 90 |
| III-024 | 1280 | 100 |
| III-026 | 1280 | 90 |
| III-027 | 1280 | 100 |
| III-029 | 1280 | 90 |
| III-033 | 1280 | 90 |
| III-053 | 1280 | 90 |
| III-054 | 1280 | 90 |
| III-056 | 1280 | 100 |
| III-058 | 1280 | 100 |
| III-066 | 1280 | 90 |
| III-067 | 1280 | 100 |
| III-068 | 1280 | 90 |
| III-069 | 1280 | 90 |

TABLE 4.6
Pre-emergence action at 320 g/ha against AMARE in %

| Ex. No. | Dosage [g/ha] | AMARE |
|---|---|---|
| I-002 | 320 | 100 |
| I-003 | 320 | 100 |
| I-006 | 320 | 80 |
| I-007 | 320 | 80 |
| I-008 | 320 | 100 |
| I-012 | 320 | 100 |
| I-014 | 320 | 100 |
| I-015 | 320 | 90 |
| I-021 | 320 | 100 |
| I-022 | 320 | 100 |
| I-023 | 320 | 90 |
| I-025 | 320 | 90 |
| I-046 | 320 | 90 |
| I-047 | 320 | 90 |
| I-054 | 320 | 100 |
| I-054 | 320 | 100 |
| I-055 | 320 | 100 |
| I-056 | 320 | 80 |
| I-057 | 320 | 100 |
| I-057 | 320 | 90 |
| I-061 | 320 | 90 |
| I-064 | 320 | 100 |
| I-067 | 320 | 90 |
| I-069 | 320 | 90 |
| I-070 | 320 | 100 |
| I-072 | 320 | 80 |
| I-079 | 320 | 80 |
| I-081 | 320 | 80 |
| I-082 | 320 | 90 |
| I-100 | 320 | 100 |
| I-101 | 320 | 90 |
| I-102 | 320 | 90 |
| I-125 | 320 | 100 |
| I-130 | 320 | 100 |
| I-137 | 320 | 90 |
| I-139 | 320 | 100 |
| I-141 | 320 | 90 |
| I-165 | 320 | 90 |
| III-003 | 320 | 100 |
| III-004 | 320 | 80 |
| III-006 | 320 | 90 |
| HI-069 | 320 | 100 |

TABLE 4.7
Pre-emergence action at 1280 g/ha against MATIN in %

| Ex. No. | Dosage [g/ha] | MATIN |
|---|---|---|
| I-002 | 1280 | 100 |
| I-003 | 1280 | 100 |
| I-008 | 1280 | 90 |
| I-012 | 1280 | 100 |
| I-015 | 1280 | 90 |
| I-016 | 1280 | 90 |
| I-021 | 1280 | 90 |
| I-022 | 1280 | 90 |
| I-023 | 1280 | 100 |
| I-026 | 1280 | 90 |
| I-046 | 1280 | 90 |
| I-048 | 1280 | 80 |
| I-056 | 1280 | 90 |
| I-057 | 1280 | 100 |
| I-061 | 1280 | 90 |
| I-067 | 1280 | 90 |
| I-069 | 1280 | 90 |
| I-070 | 1280 | 90 |
| I-072 | 1280 | 80 |

TABLE 4.8
Pre-emergence action at 1280 g/ha against AVEFA in %

| Ex. No. | Dosage [g/ha] | AVEFA |
|---|---|---|
| I-056 | 1280 | 80 |
| I-057 | 1280 | 80 |
| I-067 | 1280 | 80 |

TABLE 4.9
Pre-emergence action at 1280 g/ha against PHBPU in %

| Ex. No. | Dosage [g/ha] | PHBPU |
|---|---|---|
| I-003 | 1280 | 80 |
| I-015 | 1280 | 100 |
| I-057 | 1280 | 90 |
| I-061 | 1280 | 90 |
| I-069 | 1280 | 80 |
| I-070 | 1280 | 90 |
| I-072 | 1280 | 90 |

TABLE 4.10
Pre-emergence action at 320 g/ha against POLCO in %

| Ex. No. | Dosage [g/ha] | POLCO |
|---|---|---|
| I-001 | 320 | 90 |
| I-008 | 320 | 80 |
| I-054 | 320 | 100 |
| I-055 | 320 | 90 |
| I-056 | 320 | 80 |
| I-057 | 320 | 90 |
| I-064 | 320 | 100 |
| I-070 | 320 | 80 |
| I-072 | 320 | 90 |
| I-075 | 320 | 80 |
| I-079 | 320 | 80 |
| I-081 | 320 | 100 |
| I-082 | 320 | 80 |
| I-101 | 320 | 100 |
| I-102 | 320 | 100 |
| I-125 | 320 | 100 |
| I-130 | 320 | 90 |
| I-137 | 320 | 100 |
| I-139 | 320 | 90 |
| III-003 | 320 | 100 |
| III-006 | 320 | 90 |
| III-015 | 320 | 90 |
| III-069 | 320 | 90 |
| | 320 | 90 |

TABLE 4.11
Pre-emergence action at 1280 g/ha against STEME in %

| Ex. No. | Dosage [g/ha] | STEME |
|---|---|---|
| I-002 | 1280 | 100 |
| I-003 | 1280 | 100 |
| I-006 | 1280 | 100 |
| I-007 | 1280 | 90 |
| I-008 | 1280 | 100 |
| I-015 | 1280 | 90 |
| I-016 | 1280 | 80 |
| I-020 | 1280 | 80 |
| I-021 | 1280 | 90 |
| I-022 | 1280 | 90 |
| I-054 | 1280 | 100 |
| I-055 | 1280 | 100 |
| I-056 | 1280 | 100 |
| I-057 | 1280 | 100 |
| I-061 | 1280 | 100 |
| I-063 | 1280 | 80 |
| I-067 | 1280 | 90 |
| I-069 | 1280 | 90 |
| I-070 | 1280 | 90 |
| I-072 | 1280 | 90 |
| I-076 | 1280 | 100 |
| I-079 | 1280 | 100 |
| I-081 | 1280 | 100 |
| I-086 | 1280 | 90 |
| I-088 | 1280 | 100 |
| I-092 | 1280 | 100 |
| I-093 | 1280 | 100 |
| I-094 | 1280 | 100 |
| I-095 | 1280 | 100 |
| I-097 | 1280 | 100 |
| I-098 | 1280 | 100 |
| I-100 | 1280 | 100 |
| I-101 | 1280 | 100 |
| I-102 | 1280 | 90 |
| I-107 | 1280 | 100 |
| I-109 | 1280 | 100 |
| I-111 | 1280 | 100 |
| I-112 | 1280 | 100 |
| I-114 | 1280 | 100 |
| I-119 | 1280 | 100 |
| I-120 | 1280 | 100 |
| I-121 | 1280 | 100 |
| I-123 | 1280 | 100 |
| I-124 | 1280 | 100 |
| I-125 | 1280 | 100 |
| I-126 | 1280 | 100 |
| I-127 | 1280 | 100 |
| I-128 | 1280 | 90 |
| I-129 | 1280 | 100 |
| I-130 | 1280 | 100 |
| I-132 | 1280 | 100 |
| I-133 | 1280 | 100 |
| I-134 | 1280 | 100 |
| I-135 | 1280 | 100 |
| I-136 | 1280 | 100 |
| I-137 | 1280 | 100 |
| I-138 | 1280 | 100 |
| I-139 | 1280 | 100 |
| I-140 | 1280 | 100 |
| I-141 | 1280 | 90 |
| I-142 | 1280 | 100 |
| I-143 | 1280 | 90 |
| I-145 | 1280 | 100 |
| I-150 | 1280 | 100 |
| I-155 | 1280 | 100 |
| I-156 | 1280 | 100 |
| I-164 | 1280 | 100 |
| I-165 | 1280 | 100 |
| I-166 | 1280 | 90 |
| I-167 | 1280 | 100 |
| I-168 | 1280 | 100 |
| I-169 | 1280 | 100 |
| I-170 | 1280 | 100 |
| I-171 | 1280 | 100 |
| II-04 | 1280 | 100 |
| II-06 | 1280 | 90 |
| II-07 | 1280 | 90 |
| II-09 | 1280 | 100 |
| II-13 | 1280 | 90 |
| III-001 | 1280 | 100 |
| III-003 | 1280 | 100 |
| III-004 | 1280 | 100 |
| III-005 | 1280 | 100 |
| III-006 | 1280 | 100 |
| III-008 | 1280 | 100 |
| III-015 | 1280 | 100 |
| III-016 | 1280 | 100 |
| III-017 | 1280 | 100 |
| III-018 | 1280 | 100 |
| III-019 | 1280 | 100 |
| III-022 | 1280 | 100 |
| III-023 | 1280 | 100 |
| III-024 | 1280 | 100 |
| III-025 | 1280 | 100 |
| III-027 | 1280 | 100 |
| III-033 | 1280 | 100 |
| III-053 | 1280 | 100 |
| III-056 | 1280 | 100 |
| III-058 | 1280 | 100 |
| III-066 | 1280 | 90 |
| III-067 | 1280 | 90 |
| III-068 | 1280 | 90 |
| III-069 | 1280 | 90 |

TABLE 4.12
Pre-emergence action at 320 g/ha against STEME in %

| Ex. No. | Dosage [g/ha] | STEME |
|---|---|---|
| I-002 | 320 | 80 |
| I-054 | 320 | 100 |
| I-055 | 320 | 100 |
| I-057 | 320 | 80 |
| I-061 | 320 | 90 |
| I-064 | 320 | 90 |
| I-070 | 320 | 80 |
| I-075 | 320 | 80 |
| I-081 | 320 | 80 |
| I-100 | 320 | 90 |
| I-102 | 320 | 90 |
| I-125 | 320 | 90 |
| I-130 | 320 | 90 |
| I-137 | 320 | 90 |
| I-139 | 320 | 100 |
| I-141 | 320 | 80 |
| III-003 | 320 | 100 |
| III-069 | 320 | 100 |

TABLE 4.13
Pre-emergence action at 1280 g/ha against VIOTR in %

| Ex. No. | Dosage [g/ha] | VIOTR |
|---|---|---|
| I-021 | 1280 | 90 |
| I-022 | 1280 | 90 |
| I-046 | 1280 | 90 |
| I-051 | 1280 | 90 |
| I-056 | 1280 | 80 |
| I-057 | 1280 | 100 |
| I-061 | 1280 | 90 |
| I-063 | 1280 | 100 |
| I-066 | 1280 | 90 |
| I-067 | 1280 | 100 |
| I-069 | 1280 | 100 |
| I-070 | 1280 | 90 |
| I-072 | 1280 | 80 |

TABLE 4.14
Pre-emergence action at 320 g/ha against VERPE in %

| Ex. No. | Dosage [g/ha] | VERPE |
|---|---|---|
| I-002 | 320 | 100 |
| I-003 | 320 | 100 |
| I-008 | 320 | 90 |
| I-014 | 320 | 100 |
| I-015 | 320 | 90 |
| I-021 | 320 | 90 |
| I-022 | 320 | 100 |
| I-023 | 320 | 90 |
| I-025 | 320 | 80 |
| I-047 | 320 | 90 |
| I-054 | 320 | 100 |
| I-055 | 320 | 90 |
| I-057 | 320 | 90 |
| I-081 | 320 | 80 |
| I-082 | 320 | 80 |
| I-100 | 320 | 100 |
| I-102 | 320 | 80 |
| I-125 | 320 | 90 |
| I-130 | 320 | 100 |
| I-137 | 320 | 90 |
| I-139 | 320 | 100 |
| I-165 | 320 | 100 |
| III-003 | 320 | 100 |
| III-004 | 320 | 100 |
| III-006 | 320 | 90 |
| III-015 | 320 | 90 |
| III-069 | 320 | 100 |

TABLE 4.15
Pre-emergence action at 320 g/ha against HORMU in %

| Ex. No. | Dosage [g/ha] | HORMU |
|---|---|---|
| I-056 | 320 | 80 |
| I-064 | 320 | 90 |
| I-125 | 320 | 90 |
| I-130 | 320 | 100 |
| I-139 | 320 | 90 |
| III-069 | 320 | 90 |

As the results show, inventive compounds, for example compound nos. I-002, I-003, I-008, I-021, I-022, I-056, I-057, I-061, I-067, I-069, I-070, I-072, I-121, I-125, I-138, I-139, II-013, III-003, III-022, III-027, III-053, III-069 and other compounds from tables 4.1-4.15, show good herbicidal efficacy against harmful plants in the case of pre-emergence treatment. For example, compounds no. I-008, I-056, I-057, I-061, I-067, I-070 and I-072 applied by the pre-emergence method have very good action (80% to 100% herbicidal action) against harmful plants such as Alopecurus myosuroides, Amaranthus retroflexus, Matricaria inodora, Polygonum convolvulus, Setaria viridis and Stellaria media at an application rate of 1.28 kg of active substance or less per hectare.

2. Post-Emergence Herbicidal Action and Crop Plant Compatibility

Seeds of mono- and dicotyledonous weed plants are placed in plastic pots in sandy loam soil (twin sowing with one species each of mono- or dicotyledonous weed plants per pot), covered with soil and cultivated in a greenhouse under controlled growth conditions. 2 to 3 weeks after sowing, the test plants are treated at the one-leaf stage. The compounds of the invention, formulated in the form of wettable powders (WP) or as emulsion concentrates (EC), are applied to the green parts of the plants as aqueous suspension or emulsion with addition of 0.5% additive at a water application rate equivalent to 600 litres per hectare. After the test plants have been kept in the greenhouse under optimum growth conditions for about 3 weeks, the activity of the preparations is rated visually in comparison to untreated controls. For example, 100% activity=the plants have died, 0% activity=like control plants.

Tables 5.1-5.16 below show the effects of selected compounds of the general formula (I) according to Tables 1 to 3 on various harmful plants at an application rate corresponding to 1280 or 320 g/ha, which have been obtained by the aforementioned experimental method.

TABLE 5.1
Post-emergence action at 1280 g/ha against ALOMY in %

| Ex. No. | Dosage [g/ha] | ALOMY |
|---|---|---|
| I-002 | 1280 | 80 |
| I-006 | 1280 | 80 |
| I-021 | 1280 | 80 |
| I-022 | 1280 | 80 |
| I-046 | 1280 | 80 |
| I-051 | 1280 | 80 |
| I-056 | 1280 | 90 |
| I-057 | 1280 | 90 |
| I-061 | 1280 | 90 |
| I-067 | 1280 | 100 |
| I-070 | 1280 | 80 |
| I-072 | 1280 | 80 |

TABLE 5.2
Post-emergence action at 1280 g/ha against AVEFA in %

| Ex. No. | Dosage [g/ha] | AVEFA |
|---|---|---|
| I-009 | 1280 | 80 |
| I-012 | 1280 | 80 |
| I-022 | 1280 | 80 |
| I-025 | 1280 | 80 |
| I-036 | 1280 | 80 |
| I-051 | 1280 | 80 |
| I-056 | 1280 | 90 |
| I-057 | 1280 | 90 |

TABLE 5.3
Post-emergence action at 1280 g/ha against ECHCG in %

| Ex. No. | Dosage [g/ha] | ECHCG |
|---|---|---|
| I-002 | 1280 | 80 |
| I-003 | 1280 | 80 |
| I-016 | 1280 | 80 |
| I-021 | 1280 | 80 |
| I-022 | 1280 | 80 |
| I-054 | 1280 | 90 |
| I-055 | 1280 | 100 |
| I-061 | 1280 | 80 |
| I-067 | 1280 | 90 |
| I-070 | 1280 | 80 |
| I-072 | 1280 | 80 |
| I-079 | 1280 | 100 |
| I-081 | 1280 | 90 |
| I-093 | 1280 | 100 |
| I-097 | 1280 | 100 |
| I-098 | 1280 | 100 |
| I-100 | 1280 | 90 |
| I-107 | 1280 | 90 |
| I-120 | 1280 | 90 |
| I-123 | 1280 | 90 |
| I-125 | 1280 | 90 |
| I-126 | 1280 | 90 |
| I-130 | 1280 | 100 |
| I-136 | 1280 | 100 |
| I-137 | 1280 | 100 |
| I-141 | 1280 | 100 |
| I-143 | 1280 | 100 |
| I-164 | 1280 | 90 |
| I-167 | 1280 | 90 |
| I-169 | 1280 | 90 |
| I-171 | 1280 | 100 |
| III-003 | 1280 | 100 |
| III-019 | 1280 | 90 |
| III-022 | 1280 | 90 |
| III-027 | 1280 | 90 |
| III-053 | 1280 | 100 |

TABLE 5.4
Post-emergence action at 1280 g/ha against LOLRI in %

| Ex. No. | Dosage [g/ha] | LOLRI |
|---|---|---|
| I-057 | 1280 | 80 |
| I-067 | 1280 | 90 |
| I-130 | 1280 | 90 |
| I-171 | 1280 | 100 |

TABLE 5.5
Post-emergence action at 1280 g/ha against SETVI in %

| Ex. No. | Dosage [g/ha] | SETVI |
|---|---|---|
| I-002 | 1280 | 80 |
| I-006 | 1280 | 90 |
| I-009 | 1280 | 80 |
| I-016 | 1280 | 80 |
| I-022 | 1280 | 80 |
| I-054 | 1280 | 100 |
| I-056 | 1280 | 80 |
| I-061 | 1280 | 80 |
| I-063 | 1280 | 80 |
| I-072 | 1280 | 80 |
| I-079 | 1280 | 100 |
| I-081 | 1280 | 90 |
| I-089 | 1280 | 100 |
| I-097 | 1280 | 90 |
| I-098 | 1280 | 90 |
| I-125 | 1280 | 90 |
| I-126 | 1280 | 90 |
| I-137 | 1280 | 90 |
| I-145 | 1280 | 90 |
| I-164 | 1280 | 100 |
| I-165 | 1280 | 90 |
| I-169 | 1280 | 90 |
| I-171 | 1280 | 100 |
| III-001 | 1280 | 100 |
| III-003 | 1280 | 100 |
| III-019 | 1280 | 90 |
| III-022 | 1280 | 100 |
| III-023 | 1280 | 100 |
| III-026 | 1280 | 100 |
| III-027 | 1280 | 100 |
| III-033 | 1280 | 100 |
| III-053 | 1280 | 100 |
| III-054 | 1280 | 100 |
| III-058 | 1280 | 100 |
| III-066 | 1280 | 100 |
| III-067 | 1280 | 100 |
| III-068 | 1280 | 90 |

TABLE 5.6
Post-emergence action at
1280 g/ha against AMARE in %

| Ex. | Dosage | |
|---|---|---|
| No. | [g/ha] | AMARE |
| I-002 | 1280 | 90 |
| I-003 | 1280 | 80 |
| I-004 | 1280 | 80 |
| I-005 | 1280 | 80 |
| I-006 | 1280 | 100 |
| I-007 | 1280 | 80 |
| I-008 | 1280 | 80 |
| I-009 | 1280 | 80 |
| I-012 | 1280 | 80 |
| I-015 | 1280 | 80 |
| I-016 | 1280 | 80 |
| I-021 | 1280 | 80 |
| I-022 | 1280 | 80 |
| I-023 | 1280 | 80 |
| I-025 | 1280 | 80 |
| I-026 | 1280 | 80 |
| I-027 | 1280 | 80 |
| I-038 | 1280 | 90 |
| I-046 | 1280 | 80 |
| I-054 | 1280 | 100 |
| I-055 | 1280 | 90 |
| I-056 | 1280 | 80 |
| I-057 | 1280 | 80 |
| I-061 | 1280 | 80 |
| I-069 | 1280 | 90 |
| I-070 | 1280 | 80 |
| I-072 | 1280 | 80 |
| I-076 | 1280 | 100 |
| I-078 | 1280 | 100 |
| I-079 | 1280 | 100 |
| I-081 | 1280 | 100 |
| I-085 | 1280 | 100 |
| I-086 | 1280 | 100 |
| I-088 | 1280 | 100 |
| I-089 | 1280 | 100 |
| I-091 | 1280 | 100 |
| I-092 | 1280 | 100 |
| I-093 | 1280 | 100 |
| I-094 | 1280 | 100 |
| I-095 | 1280 | 100 |
| I-098 | 1280 | 100 |
| I-099 | 1280 | 90 |
| I-100 | 1280 | 90 |
| I-101 | 1280 | 100 |
| I-102 | 1280 | 90 |
| I-107 | 1280 | 100 |
| I-109 | 1280 | 100 |
| I-111 | 1280 | 100 |
| I-114 | 1280 | 100 |
| I-119 | 1280 | 100 |
| I-123 | 1280 | 90 |
| I-125 | 1280 | 90 |
| I-126 | 1280 | 100 |
| I-127 | 1280 | 90 |
| I-128 | 1280 | 90 |
| I-129 | 1280 | 100 |
| I-130 | 1280 | 90 |
| I-133 | 1280 | 90 |
| I-134 | 1280 | 90 |
| I-136 | 1280 | 90 |
| I-137 | 1280 | 90 |
| I-141 | 1280 | 90 |
| I-143 | 1280 | 100 |
| I-156 | 1280 | 100 |
| I-164 | 1280 | 100 |
| I-165 | 1280 | 100 |
| I-166 | 1280 | 100 |
| I-169 | 1280 | 100 |
| I-170 | 1280 | 90 |
| I-171 | 1280 | 90 |
| III-001 | 1280 | 100 |
| III-003 | 1280 | 100 |
| III-004 | 1280 | 100 |
| III-006 | 1280 | 100 |
| III-016 | 1280 | 90 |
| III-017 | 1280 | 100 |
| III-018 | 1280 | 100 |
| III-019 | 1280 | 100 |
| III-022 | 1280 | 90 |
| III-025 | 1280 | 90 |
| III-026 | 1280 | 90 |
| III-027 | 1280 | 90 |
| III-033 | 1280 | 90 |
| III-058 | 1280 | 90 |

TABLE 5.7
Post-emergence action at
320 g/ha against AMARE in %

| Ex. | Dosage | |
|---|---|---|
| No. | [g/ha] | AMARE |
| I-002 | 320 | 80 |
| I-005 | 320 | 80 |
| I-008 | 320 | 80 |
| I-012 | 320 | 80 |
| I-014 | 320 | 80 |
| I-015 | 320 | 80 |
| I-021 | 320 | 80 |
| I-022 | 320 | 80 |
| I-025 | 320 | 80 |
| I-047 | 320 | 80 |
| I-054 | 320 | 90 |
| I-055 | 320 | 90 |
| I-056 | 320 | 80 |
| I-057 | 320 | 80 |
| I-061 | 320 | 80 |
| I-079 | 320 | 80 |
| I-081 | 320 | 90 |
| I-082 | 320 | 80 |
| I-100 | 320 | 80 |
| I-101 | 320 | 80 |
| I-102 | 320 | 80 |
| I-125 | 320 | 90 |
| I-130 | 320 | 90 |
| I-137 | 320 | 80 |
| I-139 | 320 | 90 |
| I-141 | 320 | 90 |
| III-003 | 320 | 80 |
| III-004 | 320 | 80 |
| III-006 | 320 | 80 |
| III-015 | 320 | 80 |
| III-069 | 320 | 90 |

TABLE 5.8
Post-emergence action at
1280 g/ha against MATIN in %

| Ex. | Dosage | |
|---|---|---|
| No. | [g/ha] | MATIN |
| I-002 | 1280 | 80 |
| I-003 | 1280 | 80 |
| I-006 | 1280 | 80 |
| I-008 | 1280 | 90 |
| I-011 | 1280 | 90 |
| I-015 | 1280 | 90 |
| I-021 | 1280 | 80 |
| I-022 | 1280 | 80 |
| I-023 | 1280 | 80 |
| I-025 | 1280 | 80 |
| I-036 | 1280 | 80 |
| I-038 | 1280 | 80 |
| I-046 | 1280 | 80 |
| I-051 | 1280 | 90 |
| I-054 | 1280 | 100 |
| I-055 | 1280 | 100 |
| I-056 | 1280 | 80 |
| I-057 | 1280 | 90 |
| I-063 | 1280 | 80 |
| I-066 | 1280 | 80 |
| I-067 | 1280 | 80 |
| I-070 | 1280 | 80 |
| I-072 | 1280 | 80 |
| I-076 | 1280 | 90 |
| I-079 | 1280 | 100 |
| I-081 | 1280 | 100 |
| I-085 | 1280 | 90 |
| I-088 | 1280 | 90 |
| I-093 | 1280 | 100 |
| I-095 | 1280 | 100 |
| I-097 | 1280 | 100 |
| I-098 | 1280 | 90 |
| I-100 | 1280 | 90 |
| I-101 | 1280 | 90 |
| I-102 | 1280 | 100 |
| I-107 | 1280 | 100 |
| I-111 | 1280 | 90 |
| I-114 | 1280 | 90 |
| I-119 | 1280 | 90 |
| I-120 | 1280 | 90 |
| I-121 | 1280 | 90 |
| I-123 | 1280 | 90 |
| I-124 | 1280 | 90 |
| I-125 | 1280 | 90 |
| I-127 | 1280 | 90 |
| I-129 | 1280 | 100 |
| I-130 | 1280 | 90 |
| I-131 | 1280 | 90 |
| I-132 | 1280 | 90 |
| I-133 | 1280 | 90 |
| I-136 | 1280 | 90 |
| I-137 | 1280 | 90 |
| I-139 | 1280 | 90 |
| I-141 | 1280 | 90 |
| I-142 | 1280 | 90 |
| I-143 | 1280 | 100 |
| I-145 | 1280 | 90 |
| I-156 | 1280 | 90 |
| I-164 | 1280 | 100 |
| I-165 | 1280 | 100 |
| I-166 | 1280 | 100 |
| I-168 | 1280 | 90 |
| I-169 | 1280 | 90 |
| I-171 | 1280 | 90 |
| II-13 | 1280 | 100 |
| III-019 | 1280 | 90 |
| III-026 | 1280 | 90 |
| III-027 | 1280 | 100 |
| III-053 | 1280 | 90 |
| III-056 | 1280 | 90 |
| III-058 | 1280 | 100 |
| III-067 | 1280 | 90 |
| III-068 | 1280 | 90 |

TABLE 5.9
Post-emergence action at
320 g/ha against MATIN in %

| Ex. | Dosage | |
|---|---|---|
| No. | [g/ha] | MATIN |
| I-003 | 320 | 80 |
| I-008 | 320 | 90 |
| I-011 | 320 | 90 |
| I-015 | 320 | 90 |
| I-022 | 320 | 80 |
| I-036 | 320 | 80 |
| I-047 | 320 | 80 |
| I-051 | 320 | 90 |
| I-054 | 320 | 90 |
| I-056 | 320 | 80 |
| I-057 | 320 | 80 |
| I-064 | 320 | 80 |
| I-066 | 320 | 80 |
| I-075 | 320 | 80 |
| I-079 | 320 | 80 |
| I-081 | 320 | 80 |
| I-082 | 320 | 80 |
| I-100 | 320 | 80 |
| I-101 | 320 | 90 |
| I-102 | 320 | 80 |
| I-125 | 320 | 80 |
| I-130 | 320 | 90 |
| I-137 | 320 | 80 |
| I-139 | 320 | 80 |
| I-141 | 320 | 80 |
| III-069 | 320 | 80 |

TABLE 5.10
Post-emergence action at
320 g/ha against PHBPU in %

| Ex. | Dosage | |
|---|---|---|
| No. | [g/ha] | PHBPU |
| I-002 | 320 | 80 |
| I-003 | 320 | 80 |
| I-004 | 320 | 80 |
| I-005 | 320 | 80 |
| I-006 | 320 | 80 |
| I-012 | 320 | 80 |
| I-014 | 320 | 80 |
| I-015 | 320 | 80 |
| I-016 | 320 | 80 |
| I-021 | 320 | 80 |
| I-023 | 320 | 80 |
| I-026 | 320 | 90 |
| I-029 | 320 | 80 |
| I-036 | 320 | 80 |
| I-038 | 320 | 80 |
| I-046 | 320 | 80 |
| I-047 | 320 | 80 |
| I-051 | 320 | 80 |
| I-054 | 320 | 100 |
| I-055 | 320 | 80 |
| I-056 | 320 | 80 |
| I-057 | 320 | 90 |
| I-061 | 320 | 80 |
| I-064 | 320 | 80 |
| I-066 | 320 | 90 |
| I-069 | 320 | 80 |
| I-070 | 320 | 90 |
| I-075 | 320 | 90 |
| I-079 | 320 | 90 |
| I-081 | 320 | 90 |
| I-082 | 320 | 90 |
| I-100 | 320 | 90 |
| I-101 | 320 | 90 |
| I-102 | 320 | 90 |
| I-125 | 320 | 80 |
| I-137 | 320 | 90 |
| I-165 | 320 | 80 |
| III-003 | 320 | 80 |
| III-004 | 320 | 80 |
| III-006 | 320 | 80 |
| III-015 | 320 | 80 |

TABLE 5.11
Post-emergence action at
320 g/ha against POLCO in %

| Ex. | Dosage | |
|---|---|---|
| No. | [g/ha] | POLCO |
| I-004 | 320 | 80 |
| I-008 | 320 | 80 |
| I-011 | 320 | 80 |
| I-014 | 320 | 80 |
| I-022 | 320 | 80 |
| I-051 | 320 | 80 |
| I-054 | 320 | 80 |
| I-055 | 320 | 80 |
| I-056 | 320 | 80 |
| I-057 | 320 | 80 |
| I-061 | 320 | 80 |
| I-064 | 320 | 80 |
| I-075 | 320 | 80 |
| I-079 | 320 | 80 |
| I-081 | 320 | 80 |
| I-100 | 320 | 90 |
| I-125 | 320 | 80 |
| I-130 | 320 | 90 |
| I-137 | 320 | 90 |
| I-139 | 320 | 90 |
| I-165 | 320 | 80 |
| III-004 | 320 | 80 |
| III-015 | 320 | 80 |
| III-069 | 320 | 80 |

TABLE 5.12
Post-emergence action at
1280 g/ha against STEME in %

| Ex. | Dosage | |
|---|---|---|
| No. | [g/ha] | STEME |
| I-002 | 1280 | 90 |
| I-003 | 1280 | 80 |
| I-006 | 1280 | 80 |
| I-008 | 1280 | 90 |
| I-011 | 1280 | 80 |
| I-012 | 1280 | 80 |
| I-016 | 1280 | 80 |
| I-021 | 1280 | 80 |
| I-022 | 1280 | 80 |
| I-023 | 1280 | 80 |
| I-036 | 1280 | 80 |
| I-046 | 1280 | 80 |
| I-051 | 1280 | 80 |
| I-054 | 1280 | 100 |
| I-055 | 1280 | 100 |
| I-056 | 1280 | 90 |
| I-057 | 1280 | 90 |
| I-061 | 1280 | 80 |
| I-067 | 1280 | 90 |
| I-069 | 1280 | 80 |
| I-070 | 1280 | 90 |
| I-072 | 1280 | 80 |
| I-079 | 1280 | 100 |
| I-081 | 1280 | 100 |
| I-085 | 1280 | 100 |
| I-088 | 1280 | 100 |
| I-093 | 1280 | 100 |
| I-097 | 1280 | 100 |
| I-098 | 1280 | 90 |
| I-100 | 1280 | 90 |
| I-102 | 1280 | 100 |
| I-107 | 1280 | 90 |
| I-109 | 1280 | 90 |
| I-111 | 1280 | 90 |
| I-119 | 1280 | 90 |
| I-121 | 1280 | 100 |
| I-125 | 1280 | 90 |
| I-126 | 1280 | 100 |
| I-130 | 1280 | 100 |
| I-131 | 1280 | 90 |
| I-132 | 1280 | 100 |
| I-133 | 1280 | 100 |
| I-134 | 1280 | 100 |
| I-136 | 1280 | 100 |
| I-137 | 1280 | 100 |
| I-138 | 1280 | 100 |
| I-139 | 1280 | 90 |
| I-140 | 1280 | 100 |
| I-141 | 1280 | 90 |
| I-142 | 1280 | 100 |
| I-143 | 1280 | 100 |
| I-145 | 1280 | 100 |
| I-150 | 1280 | 100 |
| I-164 | 1280 | 100 |
| I-165 | 1280 | 100 |
| I-167 | 1280 | 100 |
| I-168 | 1280 | 100 |
| I-169 | 1280 | 100 |
| I-170 | 1280 | 100 |
| I-171 | 1280 | 100 |
| II-04 | 1280 | 100 |
| II-06 | 1280 | 100 |
| II-08 | 1280 | 90 |
| II-09 | 1280 | 100 |
| III-008 | 1280 | 100 |
| III-022 | 1280 | 100 |
| III-025 | 1280 | 100 |
| III-027 | 1280 | 90 |
| III-053 | 1280 | 100 |
| III-058 | 1280 | 100 |
| III-067 | 1280 | 100 |

TABLE 5.13
Post-emergence action at
320 g/ha against STEME in %

| Ex. | Dosage | |
|---|---|---|
| No. | [g/ha] | STEME |
| I-002 | 320 | 80 |
| I-003 | 320 | 80 |
| I-008 | 320 | 90 |
| I-012 | 320 | 80 |
| I-014 | 320 | 80 |
| I-021 | 320 | 80 |
| I-022 | 320 | 80 |
| I-023 | 320 | 80 |
| I-036 | 320 | 80 |
| I-046 | 320 | 80 |
| I-047 | 320 | 80 |
| I-054 | 320 | 90 |
| I-055 | 320 | 80 |
| I-057 | 320 | 80 |
| I-064 | 320 | 90 |
| I-067 | 320 | 80 |
| I-081 | 320 | 80 |
| I-100 | 320 | 80 |
| I-130 | 320 | 90 |
| I-137 | 320 | 90 |
| I-139 | 320 | 80 |
| I-141 | 320 | 90 |
| III-069 | 320 | 90 |

TABLE 5.14
Post-emergence action at
320 g/ha against VIOTR in %

| Ex. | Dosage | |
|---|---|---|
| No. | [g/ha] | VIOTR |
| I-005 | 320 | 80 |
| I-012 | 320 | 80 |
| I-023 | 320 | 80 |
| I-025 | 320 | 80 |
| I-026 | 320 | 80 |
| I-036 | 320 | 80 |
| I-046 | 320 | 80 |
| I-047 | 320 | 90 |
| I-051 | 320 | 90 |
| I-054 | 320 | 90 |
| I-055 | 320 | 100 |
| I-061 | 320 | 80 |
| I-063 | 320 | 90 |
| I-064 | 320 | 100 |
| I-066 | 320 | 80 |
| I-067 | 320 | 80 |
| I-075 | 320 | 100 |
| I-079 | 320 | 100 |
| I-081 | 320 | 100 |
| I-082 | 320 | 100 |
| I-100 | 320 | 100 |
| I-101 | 320 | 100 |
| I-102 | 320 | 90 |
| I-125 | 320 | 90 |
| I-130 | 320 | 90 |
| I-137 | 320 | 80 |
| I-139 | 320 | 80 |
| I-141 | 320 | 90 |
| I-165 | 320 | 80 |
| III-006 | 320 | 80 |
| III-015 | 320 | 80 |

TABLE 5.15
Post-emergence action at
320 g/ha against VERPE in %

| Ex. | Dosage | |
|---|---|---|
| No. | [g/ha] | VERPE |
| I-002 | 320 | 80 |
| I-007 | 320 | 90 |
| I-008 | 320 | 90 |
| I-011 | 320 | 80 |
| I-015 | 320 | 90 |
| I-022 | 320 | 80 |
| I-025 | 320 | 80 |
| I-029 | 320 | 80 |
| I-036 | 320 | 80 |
| I-038 | 320 | 80 |
| I-046 | 320 | 80 |
| I-047 | 320 | 80 |
| I-048 | 320 | 80 |
| I-051 | 320 | 80 |
| I-054 | 320 | 80 |
| I-055 | 320 | 80 |
| I-056 | 320 | 90 |
| I-057 | 320 | 80 |
| I-075 | 320 | 80 |
| I-079 | 320 | 80 |
| I-081 | 320 | 80 |
| I-082 | 320 | 80 |
| I-100 | 320 | 90 |
| I-101 | 320 | 80 |
| I-102 | 320 | 80 |
| I-125 | 320 | 80 |
| I-130 | 320 | 90 |
| I-137 | 320 | 90 |
| I-165 | 320 | 80 |
| III-003 | 320 | 80 |
| III-006 | 320 | 80 |
| III-015 | 320 | 90 |
| III-069 | 320 | 90 |

TABLE 5.16
Post-emergence action at
320 g/ha against HORMU in %

| Ex. | Dosage | |
|---|---|---|
| No. | [g/ha] | HORMU |
| I-055 | 320 | 80 |
| I-057 | 320 | 80 |
| I-064 | 320 | 90 |
| I-067 | 320 | 80 |
| I-075 | 320 | 80 |
| I-125 | 320 | 90 |
| I-130 | 320 | 80 |
| I-137 | 320 | 80 |
| I-139 | 320 | 80 |
| I-141 | 320 | 80 |
| III-069 | 320 | 80 |

As the results from Tables 5.1-5.16 show, compounds of the invention have good herbicidal post-emergence efficacy against a broad spectrum of weed grasses and weeds.

As the results show, compounds of the invention, for example compound Nos. I-002, I-003, I-006, I-014, 1-021, 1-054, 1-072, 11-04, 111-058 and other compounds from Tables 5.1-5.16, when applied post-emergence, have very good herbicidal efficacy against harmful plants. For example, compounds Nos. I-002, I-006, I-022, I-056 and I-072 applied by the post-emergence method have very good herbicidal action (80% to 100% herbicidal action) against harmful plants such as Alopecurus myosuroides, Amaranthus retroflexus, Matricaria inodora, Setaria viridis, Pharbitis purpurea and Stellaria media at an application rate of 1.28 kg of active substance or less per hectare.

1. A compound comprising a 1,5-Diphenylpyrazolyl-3-oxyalkyl acid and/or a 1-phenyl-5-thienylpyrazolyl-3-oxyalkyl acid of formula (I), and/or an agrochemically acceptable salt thereof, 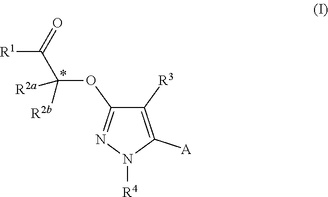
where
A is selected from the group consisting of A1-A3, 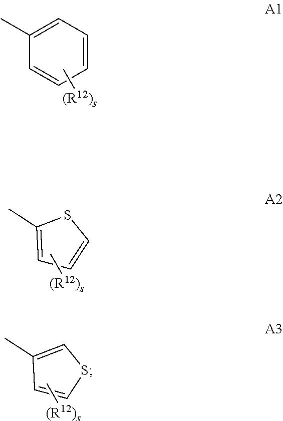
R¹ is selected from the group consisting of
OR^{1a} and
NR⁹R¹⁰; where
R^{1a} is selected from the group consisting of
hydrogen;
methyl, ethyl which is unsubstituted or substituted by one or more substituents selected from the group consisting of halogen, (C₃-C₆)-cycloalkyl, (C₁-C₄)-trialkylsilyl, (C₁-C₆)-alkoxy, cyano and nitro;
(C₂-C₆)-alkenyl, (C₂-C₆)-haloalkenyl;
(C₂-C₆)-alkynyl,
(C₃-C₆)-cycloalkyl which is unsubstituted or substituted by one or more substituents selected from the group consisting of halogen, (C₁-C₆)-alkyl, (C₃-C₆)-cycloalkyl;
(C₁-C₄)-alkyl-SO—(C₁-C₄)-alkyl, (C₁-C₄)-alkyl-SO₂—(C₁-C₄)-alkyl;
heterocyclyl, heteroaryl and aryl which is unsubstituted or substituted by one or more substituents selected from the group consisting of halogen, (C₁-C₆)-alkyl, (C₁-C₆)-haloalkyl;
heterocyclyl-(C₁-C₄)-alkyl, heteroaryl-(C₁-C₄)-alkyl and aryl-(C₁-C₄)-alkyl, where the heterocyclyl, heteroaryl and aryl is unsubstituted or substituted by one or more substituents selected from the group consisting of halogen, (C₁-C₆)-alkyl, (C₁-C₆)-haloalkyl;
R⁹ is selected from the group consisting of hydrogen, (C₁-C₁₂)-alkyl;
R¹⁰ is selected from the group consisting of
hydrogen;
aryl, heteroaryl, heterocyclyl, which is unsubstituted or substituted by one or more substituents selected from the group consisting of halogen, (C₁-C₆)-alkyl, (C₁-C₆)-haloalkyl;
(C₃-C₇)-cycloalkyl-(C₁-C₄)-alkyl, heterocyclyl-(C₁-C₄)-alkyl, heteroaryl-(C₁-C₄)-alkyl, aryl-(C₁-C₄)-alkyl, aryl-(C₁-C₄)-alkoxy;
where the cycloalkyl, heterocyclyl, heteroaryl and aryl is unsubstituted or substituted by one or more substituents selected from the group consisting of halogen, (C₁-C₆)-alkyl, (C₁-C₆)-haloalkyl;
(C₁-C₁₂)-alkyl; (C₃-C₈)-cycloalkyl, (C₂-C₁₂)-alkenyl, (C₅-C₈)-cycloalkenyl, (C₂-C₁₂)-alkynyl;
where the abovementioned alkyl, cycloalkenyl, alkenyl, cycloalkenyl and alkynyl radicals are unsubstituted or each independently substituted by m radicals selected from the group consisting of cyano, nitro, OR⁵, S(O)ₙR⁵, SO₂NR⁶R⁷, C(O)OR⁸, CONR⁶R⁸, COR⁶, NR⁶R⁸, NR⁶COR⁸, NR⁶CONR⁸R⁸, NR⁶CO₂R⁸, NR⁶SO₂R⁸, NR⁶SO₂NR⁶R⁸, C(R⁶)═NOR⁸;
(C₁-C₁₂)-haloalkyl;
S(O)ₙR⁵, cyano, nitro, OR⁵, SO₂NR⁶R⁷, C₀₂R⁸, COR⁸, NR⁶R⁸, NR⁶COR⁸, NR⁶CO₂R⁸, NR⁶SO₂R⁸;
or
R⁹ and R¹⁰ together with the nitrogen atom to which they are attached form a saturated, partially or fully unsaturated five-, six- or seven-membered ring which is optionally mono- to hexasubstituted by radicals from the group consisting of halogen, (C₁-C₆)-alkyl, halo-(C₁-C₆)-alkyl, OR⁵, S(O)ₙR⁵, CO₂R⁸, CONR⁶R⁸, COR⁶ and C(R⁶)═NOR⁸ and which, in addition to this nitrogen atom, contains r carbon atoms, o oxygen atoms, p sulfur atoms and q elements from the group consisting of NR⁷, CO and NCOR⁷ as ring atoms;
R⁵ is (C₁-C₆)-alkyl, (C₃-C₆)-cycloalkyl, (C₁-C₆)-haloalkyl or aryl;
R⁶ is hydrogen or R⁵;
R⁷ is hydrogen, (C₁-C₆)-alkyl, (C₃-C₆)-cycloalkyl, (C₃-C₄)-alkenyl or (C₃-C₄)-alkynyl;
R⁸ is hydrogen, (C₁-C₆)-alkyl, (C₃-C₆)-cycloalkyl, (C₃-C₄)-alkenyl or (C₃-C₄)-alkynyl;
R^{2a} is selected from the group consisting of
hydrogen;
methyl;
R^{2b} is hydrogen;
R³ is selected from the group consisting of
halogen, cyano, isocyano, NO₂;
(C₁-C₆)-alkyl, (C₃-C₆)-cycloalkyl, (C₁-C₆)-haloalkyl, (C₁-C₆)-alkylcarbonyl, (C₁-C₆)-haloalkylcarbonyl, (C₁-C₄)-alkyloxycarbonyl;
(C₂-C₃)-alkenyl, (C₂-C₃)-haloalkenyl;
(C₂-C₃)-alkynyl, (C₂-C₃)-haloalkynyl;
(C₁-C₂)-alkyl-S(O)ₙ and (C₁-C₂)-haloalkyl-S(O)ₙ;
CHO;
NH₂;
R⁴ is phenyl, where the phenyl radical is unsubstituted or mono- or polysubstituted by a radical selected from the group consisting of
halogen, cyano, isocyano, nitro;
(C₁-C₆)-alkyl, (C₁-C₆)-haloalkyl, (C₁-C₃)-haloalkoxy;
(C₂-C₃)-alkenyl, (C₂-C₃)-haloalkenyl, (C₁-C₆)-alkoxy;
(C₂-C₃)-alkynyl, (C₂-C₃)-haloalkynyl, (C₁-C₄)-alkyl-S(O)ₙ;
CHO, (C₁-C₄)-alkyloxycarbonyl and NH₂;
R¹² is selected from the group consisting of
halogen, cyano, isocyano, NO₂;
(C₁-C₆)-alkyl, (C₁-C₆)-haloalkyl, (C₁-C₆)-alkylcarbonyl, (C₁-C₆)-haloalkylcarbonyl, (C₁-C₄)-alkyloxycarbonyl, (C₁-C₆)-alkoxy, (C₁-C₃)-haloalkoxy, (C₁-C₄)-alkyl-S(O)ₙ;
(C₂-C₃)-alkenyl, (C₂-C₃)-haloalkenyl;
(C₂-C₃)-alkynyl, (C₂-C₃)-haloalkynyl;
NH₂;
and where the indices are as follows:
m is 0, 1 or 2;
n is 0, 1 or 2;
o is 0, 1 or 2;
p is 0 or 1;
q is 0 or 1;
r is 3, 4, 5 or 6; and
s is 0, 1, 2, 3, 4 or 5, excluding the following compounds:
[(4-chloro-1,5-diphenyl-1H-pyrazol-3-yl)oxy]acetic acid, {[4-chloro-5-(4-methylphenyl)-1-phenyl-1H-pyrazol-3-yl]oxy}acetic acid, {[4-chloro-5-(3-chlorophenyl)-1-phenyl-1H-pyrazol-3-yl]oxy}acetic acid, ethyl [(4-chloro-1,5-diphenyl-1H-pyrazol-3-yl)oxy]acetate, ethyl {[4-chloro-5-(4-methylphenyl)-1-phenyl-1H-pyrazol-3-yl]oxy}acetate, ethyl {[4-chloro-5-(3-chlorophenyl)-1-phenyl-1H-pyrazol-3-yl]oxy}acetate, 2-(4-chloro-1,5-diphenyl-pyrazol-3-yl)oxypropanoic acid, 2-(4-bromo-1,5-diphenylpyrazol-3-yl)oxypropanoic acid.

2. The compound of the formula (I) according to claim 1 and/or an agrochemically acceptable salt thereof, where
A is selected from the group consisting of A1-A3, 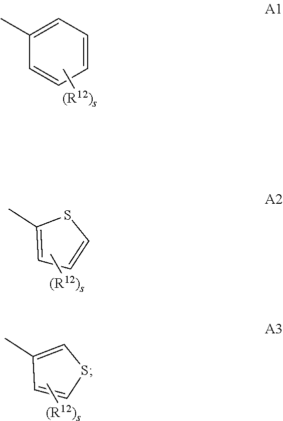
R¹ is selected from the group consisting of
OR^{1a} and
NR⁹R¹⁰; where
R^{1a} is selected from the group consisting of
hydrogen;
methyl, ethyl which is unsubstituted or substituted by one or more substituents selected from the group consisting of halogen, (C₃-C₆)-cycloalkyl, (C₁-C₄)-trialkylsilyl, (C₁-C₄)-alkoxy, cyano and nitro;
(C₂-C₆)-alkenyl, (C₂-C₆)-haloalkenyl;
aryl-(C₁-C₄)-alkyl, where the aryl is unsubstituted or substituted by one or more substituents selected from the group consisting of halogen, (C₁-C₄)-alkyl, (C₁-C₄)-haloalkyl;
R⁹ is selected from the group consisting of hydrogen, (C₁-C₆)-alkyl;
R¹⁰ is selected from the group consisting of
hydrogen;
(C₃-C₇)-cycloalkyl-(C₁-C₄)-alkyl, aryl-(C₁-C₄)-alkyl, aryl-(C₁-C₄)-alkoxy, where the cycloalkyl and aryl is unsubstituted or substituted by one or more substituents selected from the group consisting of halogen, (C₁-C₄)-alkyl, (C₁-C₄)-haloalkyl;
(C₁-C₆)-alkyl, (C₂-C₆)-alkenyl, (C₂-C₆)-alkynyl; (C₃-C₈)-cycloalkyl;
where the abovementioned alkyl, alkenyl, alkynyl and cycloalkenyl radicals are unsubstituted or are each independently substituted by m radicals selected from the group consisting of Cyano, C(O)OR⁸;
(C₁-C₆)-haloalkyl
R⁸ is hydrogen, (C₁-C₆)-alkyl, (C₃-C₆)-cycloalkyl;
R^{2a} is selected from the group consisting of
hydrogen;
methyl;
R^{2b} is hydrogen;
R³ is selected from the group consisting of
fluorine, chlorine, bromine, iodine, cyano, isocyano, NO₂;
(C₁-C₆)-alkyl, (C₃-C₆)-cycloalkyl, (C₁-C₆)-haloalkyl, (C₁-C₄)-alkyloxycarbonyl;
(C₂-C₃)-alkynyl, (C₂-C₃)-haloalkynyl;
R⁴ is phenyl, where the phenyl radical is unsubstituted or mono- or polysubstituted by a radical selected from the group consisting of
fluorine, chlorine, bromine;
methyl, ethyl;
methoxy, ethoxy;
R¹² is selected from the group consisting of
halogen, cyano, nitro;
(C₁-C₆)-alkyl, (C₁-C₆)-haloalkyl, (C₁-C₃)-haloalkoxy;
(C₁-C₆)-alkoxy;
and where the indices are as follows:
m is 0, 1 or 2;
s is 0, 1, 2, 3,
excluding the compounds:
[(4-chloro-1,5-diphenyl-1H-pyrazol-3-yl)oxy]acetic acid, {[4-chloro-5-(4-methylphenyl)-1-phenyl-1H-pyrazol-3-yl]oxy}acetic acid, {[4-chloro-5-(3-chlorophenyl)-1-phenyl-1H-pyrazol-3-yl]oxy}acetic acid, ethyl [(4-chloro-1,5-diphenyl-1H-pyrazol-3-yl)oxy]acetate, ethyl {[4-chloro-5-(4-methylphenyl)-1-phenyl-1H-pyrazol-3-yl]oxy}acetate, ethyl {[4-chloro-5-(3-chlorophenyl)-1-phenyl-1H-pyrazol-3-yl]oxy}acetate, 2-(4-chloro-1,5-diphenyl-pyrazol-3-yl)oxypropanoic acid, 2-(4-bromo-1,5-diphenylpyrazol-3-yl)oxypropanoic acid.

3. The compound of the formula (I) according to claim 1 and/or an agrochemically acceptable salt thereof, where
A is A1; 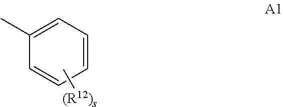
R¹ is selected from the group consisting of
OR^{1a} and
NR⁹R¹⁰; where
R^{1a} is selected from the group consisting of
hydrogen;
methyl, ethyl, trimethylsilylmethyl;
1-propenyl, 2-propenyl;
benzyl, 1-phenylethyl, 2-phenylethyl, where the phenyl radical in each of the three groups mentioned is unsubstituted or substituted by halogen;
R⁹ is hydrogen;
R¹⁰ is selected from the group consisting of
hydrogen;
cyclopropylmethyl;
benzyl, 1-phenylethyl, 2-phenylethyl, benzyloxy, where the phenyl radical in each of the four groups mentioned is unsubstituted or substituted by halogen;
methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, where the abovementioned radicals are unsubstituted or are each independently monosubstituted by a C(O)OR⁸ radical;
cyclopropyl, cyclobutyl, cyclopentyl, where the three radicals mentioned are unsubstituted or are each independently monosubstituted by a C(O)OR⁸ radical;
1-propenyl, 2-propenyl, 2-methyl-2-propenyl, prop-2-yn-1-yl, but-2-yn-1-yl;
R⁸ is hydrogen, methyl, ethyl;
R^{2a} is selected from the group consisting of
hydrogen;
methyl;
R^{2b} is hydrogen;
R³ is selected from the group consisting of
fluorine, chlorine, bromine, iodine, cyano, NO₂;
trifluoromethyl;
ethynyl;
C(O)Omethyl;
R⁴ is phenyl, where the phenyl radical is unsubstituted or mono- or polysubstituted by a radical selected from the group consisting of
fluorine, chlorine, bromine;
methyl, ethyl;
methoxy, ethoxy;
R¹² is selected from the group consisting of
fluorine, chlorine, NO₂;
trifluoromethyl, methoxy, ethoxy;
and where the indices are as follows:
s 1, 2, 3,
excluding the compounds:
[(4-chloro-1,5-diphenyl-1H-pyrazol-3-yl)oxy]acetic acid, {[4-chloro-5-(4-methylphenyl)-1-phenyl-1H-pyrazol-3-yl]oxy}acetic acid, {[4-chloro-5-(3-chlorophenyl)-1-phenyl-1H-pyrazol-3-yl]oxy}acetic acid, ethyl [(4-chloro-1,5-diphenyl-1H-pyrazol-3-yl)oxy]acetate, ethyl {[4-chloro-5-(4-methylphenyl)-1-phenyl-1H-pyrazol-3-yl]oxy}acetate, ethyl {[4-chloro-5-(3-chlorophenyl)-1-phenyl-1H-pyrazol-3-yl]oxy}acetate, 2-(4-chloro-1,5-diphenyl-pyrazol-3-yl)oxypropanoic acid, 2-(4-bromo-1,5-diphenylpyrazol-3-yl)oxypropanoic acid.

4. The compound of formula (I) according claim 1 and/or an agrochemically acceptable salt thereof, where
A is A1 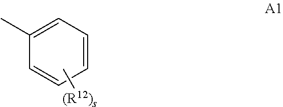
R¹ is selected from the group consisting of
OR^{1a} and
NR⁹R¹⁰; where
R^{1a} is selected from the group consisting of
hydrogen;
methyl, ethyl;
2-propenyl;
R⁹ is hydrogen;
R¹⁰ is selected from the group consisting of
cyclopentyl monosubstituted by C(O)OR⁸;
cyclopropylmethyl;
CH₂C(O)OR⁸, CH₂CH₂C(O)OR⁸;
2-propenyl, prop-2-yn-1-yl;
R⁸ is hydrogen, methyl, ethyl;
R^{2a} is selected from the group consisting of
hydrogen;
methyl;
R^{2b} is hydrogen;
R³ is selected from the group consisting of
chlorine, bromine, iodine, cyano, NO₂;
R⁴ is phenyl, where the phenyl radical is unsubstituted or mono- or polysubstituted by a radical selected from the group consisting of
fluorine, chlorine;
R¹² is selected from the group consisting of
fluorine, chlorine;
and where the indices are as follows:
s is 1, 2,
excluding the compounds:
[(4-chloro-1,5-diphenyl-1H-pyrazol-3-yl)oxy]acetic acid, {[4-chloro-5-(4-methylphenyl)-1-phenyl-1H-pyrazol-3-yl]oxy}acetic acid, {[4-chloro-5-(3-chlorophenyl)-1-phenyl-1H-pyrazol-3-yl]oxy}acetic acid, ethyl [(4-chloro-1,5-diphenyl-1H-pyrazol-3-yl)oxy]acetate, ethyl {[4-chloro-5-(4-methylphenyl)-1-phenyl-1H-pyrazol-3-yl]oxy}acetate, ethyl {[4-chloro-5-(3-chlorophenyl)-1-phenyl-1H-pyrazol-3-yl]oxy}acetate, 2-(4-chloro-1,5-diphenyl-pyrazol-3-yl)oxypropanoic acid, 2-(4-bromo-1,5-diphenylpyrazol-3-yl)oxypropanoic acid.

5. A process for preparing one or more compounds of formula (Ia) and/or an agrochemically acceptable salt thereof according to claim 1 comprising reacting one or more compounds of formulae (II) and (III) to give one or more compounds of formula (IV), which are reacted with one or more compounds of formula (V) to give one or more compounds of formula (Ia), 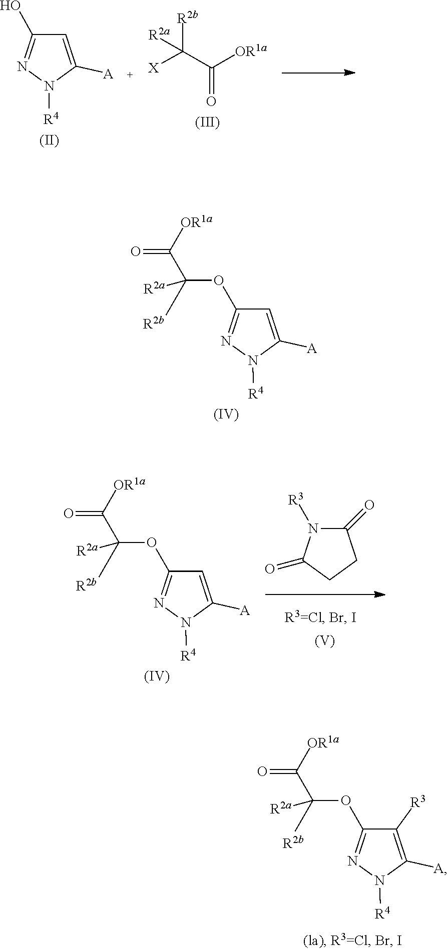
in which R⁴, R^{2a}, R^{2b}, R^{1a}, R³, and A have the definition given above, and X is chlorine, bromine or iodine.

6. A process for preparing one or more compounds of formula (Ib) and/or an agrochemically acceptable salt thereof according to claim 1 comprising reacting one or more compounds of formula (Ia) in which R⁴, R^{2a}, R^{2b}, R^{1a}, R³, and A have the definitions given above further with one or more compounds of formula (VI) to give one or more compounds of formula (Ib).

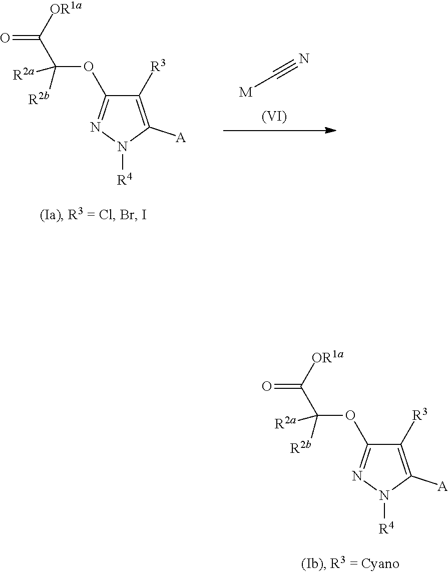

7. An agrochemical composition comprising a) at least one compound of formula (I) or an agrochemically acceptable salt thereof as defined in claim 1, and b) one or more auxiliaries and/or additives customary in crop protection.

8. The agrochemical composition comprising
a) at least one compound of the formula (I) or an agrochemically acceptable salt thereof as defined in claim 1,
b) one or more active agrochemical ingredients other than component a), and optionally
c) one or more auxiliaries and/or additives customary in crop protection.

9. A method of controlling unwanted one or more plants or for regulating the growth of one or more plants, comprising applying an effective amount of at least one compound of the formula (I) and/or an agrochemically acceptable salt thereof, as defined in claim 1, to the plants, seed and/or an area in which the plants grow.

10. A product comprising one or more compounds of the formula (I) and/or an agrochemically acceptable salt thereof, as defined in claim 1, wherein said product is an herbicide and/or a plant growth regulator.

11. The product according to claim 10, wherein the compound of formula (I) or an agrochemically acceptable salt thereof is used for controlling harmful one or more plants and/or for regulating growth in one or more plant crops.

12. The product according to claim 11, wherein the crop plants are transgenic or nontransgenic crop plants.
